SEQUENCE LISTING (877 sequences)

SEQ ID NO: 1 - protein, 397 aa, synthetic construct
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 2 - DNA, 3168 bp, synthetic construct
  source 1..3168: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcctgccggcggctctggaggaggcggctccggaggcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggatctgggtccggcagcgggtccgggtcccaagagcggagagccaaagagaagctgcaagagcagcagcgcgacctggaacagagaaaggccgacaccaagaagaacctggaacgcaagaaagaacacggcgacgtgctggccgaggacctgtatggcagactggaaatccctgccatcgagctgcccagcgagaacgagagaggctactacatccctcaccagagcagcctgcctcaggacaacagaggcaacagccgggacagcaaagagatcagcatcatcgagaacaccaaccgcgagagcatcaccaccaacgtggaaggcagacgggacatccacaagggccacctggaagaaaagaaggacggcagcatcaagcccgagcagaaagaggacaagagcgccgacatccagaatcacaccctggaaaccgtgaacatcagcgacgtgaacgacttccagatctctaagtacgaggacgagatcagcgccgagtacgacgacagcctgatcgacgaggaagaaggcggtagctccgggggtggctcctctggtgacgacgaggacctggacgagttcaagcccatcgtgcagtacgacaacttccaggacgaggaaaacatcggcatctacaaagagcttgaggacctgatcgagaagaacgagaacctggatgatctggacgagggcatcgagaagtccagcgaggaactgagcgaggaaaagatcaagaagggcaagaagtacgaaaagaccaaggacaacaacttcaagccgaacgacaagagcctgtacgacgagcacatcaagaagtataagaacgataagcaagtgaacaaagagaaagagaagttcatcaagtccctgttccacatcttcgacggcgacaacgagatcctccagatcgtggatgagctgtccgaggacatcaccaagtactttatgaagctgggtggatcatccggtggaggttcatcaggaatgaaggacgccgtggaaatcaacgacatcaccagcaagctgatcgaggaaacccaagagctgaacgaggtggaagccgacctgatcaaggacatggaaaagctgaaagagctggaaaaggccctgagcgaggacagcaaagagatcatcgacgccaaggacgacaccctggaaaaagtgattgaggaagaacacgatatcaccaccacactggacgaggtggtggaactgaaggacgtggaagaagataagatcgagaaggtgtccgacctgaaggaccttgaggaagatatcctgaaagaagtgaaagaaatcaaagagcttgagagcgagatccttgaggactacaaagaactcaagaccatcgagacagacatcctggaagaaaagaaagagattgagaaggaccacttcgagaagttcgaggaagaagctgaggaaatcaaggacctcgaagccgacatcggcggatcttctggcggagggtcatccggcaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 3 - protein, 1054 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGSGSGSGSGSQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSSGGGSSGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSSGGGSSGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSSGGGSSGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGIVGIVAGLAVLAVVVIGAVVATVMCRRKSSGGKGGSYSQAASSDSAQGSDVSLTA

SEQ ID NO: 4 - RNA, 3168 nt, synthetic construct
  source 1..3168: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcctgccggcggctctggaggaggcggctccggaggcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggatctgggtccggcagcgggtccgggtcccaagagcggagagccaaagagaagctgcaagagcagcagcgcgacctggaacagagaaaggccgacaccaagaagaacctggaacgcaagaaagaacacggcgacgtgctggccgaggacctgtatggcagactggaaatccctgccatcgagctgcccagcgagaacgagagaggctactacatccctcaccagagcagcctgcctcaggacaacagaggcaacagccgggacagcaaagagatcagcatcatcgagaacaccaaccgcgagagcatcaccaccaacgtggaaggcagacgggacatccacaagggccacctggaagaaaagaaggacggcagcatcaagcccgagcagaaagaggacaagagcgccgacatccagaatcacaccctggaaaccgtgaacatcagcgacgtgaacgacttccagatctctaagtacgaggacgagatcagcgccgagtacgacgacagcctgatcgacgaggaagaaggcggtagctccgggggtggctcctctggtgacgacgaggacctggacgagttcaagcccatcgtgcagtacgacaacttccaggacgaggaaaacatcggcatctacaaagagcttgaggacctgatcgagaagaacgagaacctggatgatctggacgagggcatcgagaagtccagcgaggaactgagcgaggaaaagatcaagaagggcaagaagtacgaaaagaccaaggacaacaacttcaagccgaacgacaagagcctgtacgacgagcacatcaagaagtataagaacgataagcaagtgaacaaagagaaagagaagttcatcaagtccctgttccacatcttcgacggcgacaacgagatcctccagatcgtggatgagctgtccgaggacatcaccaagtactttatgaagctgggtggatcatccggtggaggttcatcaggaatgaaggacgccgtggaaatcaacgacatcaccagcaagctgatcgaggaaacccaagagctgaacgaggtggaagccgacctgatcaaggacatggaaaagctgaaagagctggaaaaggccctgagcgaggacagcaaagagatcatcgacgccaaggacgacaccctggaaaaagtgattgaggaagaacacgatatcaccaccacactggacgaggtggtggaactgaaggacgtggaagaagataagatcgagaaggtgtccgacctgaaggaccttgaggaagatatcctgaaagaagtgaaagaaatcaaagagcttgagagcgagatccttgaggactacaaagaactcaagaccatcgagacagacatcctggaagaaaagaaagagattgagaaggaccacttcgagaagttcgaggaagaagctgaggaaatcaaggacctcgaagccgacatcggcggatcttctggcggagggtcatccggcaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 5 - DNA, 3168 bp, synthetic construct
  source 1..3168: mol_type = other DNA
atggggggggctgccgccaggttgggggccgtgattttgtttgtcgtcatagtgggcctccatggggtccgcagccaagagcggagagccaaagagaagctgcaagagcagcagcgcgacctggaacagagaaaggccgacaccaagaagaacctggaacgcaagaaagaacacggcgacgtgctggccgaggacctgtatggcagactggaaatccctgccatcgagctgcccagcgagaacgagagaggctactacatccctcaccagagcagcctgcctcaggacaacagaggcaacagccgggacagcaaagagatcagcatcatcgagaacaccaaccgcgagagcatcaccaccaacgtggaaggcagacgggacatccacaagggccacctggaagaaaagaaggacggcagcatcaagcccgagcagaaagaggacaagagcgccgacatccagaatcacaccctggaaaccgtgaacatcagcgacgtgaacgacttccagatctctaagtacgaggacgagatcagcgccgagtacgacgacagcctgatcgacgaggaagaaggcggctctggaggaggcggctccggaggcgacgacgaggacctggacgagttcaagcccatcgtgcagtacgacaacttccaggacgaggaaaacatcggcatctacaaagagcttgaggacctgatcgagaagaacgagaacctggatgatctggacgagggcatcgagaagtccagcgaggaactgagcgaggaaaagatcaagaagggcaagaagtacgaaaagaccaaggacaacaacttcaagccgaacgacaagagcctgtacgacgagcacatcaagaagtataagaacgataagcaagtgaacaaagagaaagagaagttcatcaagtccctgttccacatcttcgacggcgacaacgagatcctccagatcgtggatgagctgtccgaggacatcaccaagtactttatgaagctgggatctgggtccggcagcgggtccgggtccaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggcggtagctccgggggtggctcctctggtatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcctgccggtggatcatccggtggaggttcatcaggaatgaaggacgccgtggaaatcaacgacatcaccagcaagctgatcgaggaaacccaagagctgaacgaggtggaagccgacctgatcaaggacatggaaaagctgaaagagctggaaaaggccctgagcgaggacagcaaagagatcatcgacgccaaggacgacaccctggaaaaagtgattgaggaagaacacgatatcaccaccacactggacgaggtggtggaactgaaggacgtggaagaagataagatcgagaaggtgtccgacctgaaggaccttgaggaagatatcctgaaagaagtgaaagaaatcaaagagcttgagagcgagatccttgaggactacaaagaactcaagaccatcgagacagacatcctggaagaaaagaaagagattgagaaggaccacttcgagaagttcgaggaagaagctgaggaaatcaaggacctcgaagccgacatcggcggatcttctggcggagggtcatccggcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 6 - protein, 1054 aa, synthetic construct
MGGAAARLGAVILFVVIVGLHGVRSQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGSGSGSGSGSNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSSGGGSSGMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSSGGGSSGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSSGGGSSGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGIVGIVAGLAVLAVVVIGAVVATVMCRRKSSGGKGGSYSQAASSDSAQGSDVSLTA

SEQ ID NO: 7 - RNA, 3168 nt, synthetic construct
  source 1..3168: mol_type = other RNA
atggggggggctgccgccaggttgggggccgtgattttgtttgtcgtcatagtgggcctccatggggtccgcagccaagagcggagagccaaagagaagctgcaagagcagcagcgcgacctggaacagagaaaggccgacaccaagaagaacctggaacgcaagaaagaacacggcgacgtgctggccgaggacctgtatggcagactggaaatccctgccatcgagctgcccagcgagaacgagagaggctactacatccctcaccagagcagcctgcctcaggacaacagaggcaacagccgggacagcaaagagatcagcatcatcgagaacaccaaccgcgagagcatcaccaccaacgtggaaggcagacgggacatccacaagggccacctggaagaaaagaaggacggcagcatcaagcccgagcagaaagaggacaagagcgccgacatccagaatcacaccctggaaaccgtgaacatcagcgacgtgaacgacttccagatctctaagtacgaggacgagatcagcgccgagtacgacgacagcctgatcgacgaggaagaaggcggctctggaggaggcggctccggaggcgacgacgaggacctggacgagttcaagcccatcgtgcagtacgacaacttccaggacgaggaaaacatcggcatctacaaagagcttgaggacctgatcgagaagaacgagaacctggatgatctggacgagggcatcgagaagtccagcgaggaactgagcgaggaaaagatcaagaagggcaagaagtacgaaaagaccaaggacaacaacttcaagccgaacgacaagagcctgtacgacgagcacatcaagaagtataagaacgataagcaagtgaacaaagagaaagagaagttcatcaagtccctgttccacatcttcgacggcgacaacgagatcctccagatcgtggatgagctgtccgaggacatcaccaagtactttatgaagctgggatctgggtccggcagcgggtccgggtccaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggcggtagctccgggggtggctcctctggtatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcctgccggtggatcatccggtggaggttcatcaggaatgaaggacgccgtggaaatcaacgacatcaccagcaagctgatcgaggaaacccaagagctgaacgaggtggaagccgacctgatcaaggacatggaaaagctgaaagagctggaaaaggccctgagcgaggacagcaaagagatcatcgacgccaaggacgacaccctggaaaaagtgattgaggaagaacacgatatcaccaccacactggacgaggtggtggaactgaaggacgtggaagaagataagatcgagaaggtgtccgacctgaaggaccttgaggaagatatcctgaaagaagtgaaagaaatcaaagagcttgagagcgagatccttgaggactacaaagaactcaagaccatcgagacagacatcctggaagaaaagaaagagattgagaaggaccacttcgagaagttcgaggaagaagctgaggaaatcaaggacctcgaagccgacatcggcggatcttctggcggagggtcatccggcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 8 - DNA, 2811 bp, synthetic construct
  source 1..2811: mol_type = other DNA
atggggggggctgccgccaggttgggggccgtgattttgtttgtcgtcatagtgggcctccatggggtccgcagctccaagaagaagaacaagaaaggctccggcgagcccctgatcgacgtgcacgatctgatctccgacatgatcaagaaagaggaagaactggtcgaagtcaacaagcggaagtccaagtacaagctggccacctctgtgctggctggcggctctggaggaggcggctccggaggcctgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggatctgggtccggcagcgggtccgggtccatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggtagctccgggggtggctcctctggtatgaagaccatcatcatcgtgaccctgttcatcctgatcctcaacacgatcatcatcaacccctgcacctgtgtgcccaacacactgcccatcgagtacttcgactacgagacactgaaagagaacgtcaagatccgggacagcaacaccaagacacagctgatcatcctgagcgccgtgtgcgcctctatcctgctgagcctttctagcctgtggggcatcagaatccactacaaccacggtggatcatccggtggaggttcatcaggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggcggatcttctggcggagggtcatccggctgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 9 - protein, 935 aa, synthetic construct
MGGAAARLGAVILFVVIVGLHGVRSSKKKNKKGSGEPLIDVHDLISDMIKKEEELVEVNKRKSKYKLATSVLAGGSGGGGSGGLNASKVNKKGKIAEEKKRKNIKNIDKAIEEHNKRKKLIYYSLIASGAIASVAAILGGSGSGSGSGSMKVSRHTVLLNIILIVSLLGCVLTLNLFSANDDKRALKDIDRTLEKLLRKKQIIISTAAVALAITLGGLFGSLGYKSWKNKNKSKDKVNGGSSGGGSSGMKTIIIVTLFILILNTIIINPCTCVPNTLPIEYFDYETLKENVKIRDSNTKTQLIILSAVCASILLSLSSLWGIRIHYNHGGSSGGGSSGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSSGGGSSGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 10 - RNA, 2811 nt, synthetic construct
  source 1..2811: mol_type = other RNA
atggggggggctgccgccaggttgggggccgtgattttgtttgtcgtcatagtgggcctccatggggtccgcagctccaagaagaagaacaagaaaggctccggcgagcccctgatcgacgtgcacgatctgatctccgacatgatcaagaaagaggaagaactggtcgaagtcaacaagcggaagtccaagtacaagctggccacctctgtgctggctggcggctctggaggaggcggctccggaggcctgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggatctgggtccggcagcgggtccgggtccatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggtagctccgggggtggctcctctggtatgaagaccatcatcatcgtgaccctgttcatcctgatcctcaacacgatcatcatcaacccctgcacctgtgtgcccaacacactgcccatcgagtacttcgactacgagacactgaaagagaacgtcaagatccgggacagcaacaccaagacacagctgatcatcctgagcgccgtgtgcgcctctatcctgctgagcctttctagcctgtggggcatcagaatccactacaaccacggtggatcatccggtggaggttcatcaggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggcggatcttctggcggagggtcatccggctgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 11 - DNA, 2811 bp, synthetic construct
  source 1..2811: mol_type = other DNA
atggggggggctgccgccaggttgggggccgtgattttgtttgtcgtcatagtgggcctccatggggtccgcagcctgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggcggctctggaggaggcggctccggaggcatgaagaccatcatcatcgtgaccctgttcatcctgatcctcaacacgatcatcatcaacccctgcacctgtgtgcccaacacactgcccatcgagtacttcgactacgagacactgaaagagaacgtcaagatccgggacagcaacaccaagacacagctgatcatcctgagcgccgtgtgcgcctctatcctgctgagcctttctagcctgtggggcatcagaatccactacaaccacggatctgggtccggcagcgggtccgggtcctgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggcggtagctccgggggtggctcctctggttccaagaagaagaacaagaaaggctccggcgagcccctgatcgacgtgcacgatctgatctccgacatgatcaagaaagaggaagaactggtcgaagtcaacaagcggaagtccaagtacaagctggccacctctgtgctggctggtggatcatccggtggaggttcatcaggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggatcttctggcggagggtcatccggcttctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 12 - protein, 935 aa, synthetic construct
MGGAAARLGAVILFVVIVGLHGVRSLNASKVNKKGKIAEEKKRKNIKNIDKAIEEHNKRKKLIYYSLIASGAIASVAAILGGGSGGGGSGGMKTIIIVTLFILILNTIIINPCTCVPNTLPIEYFDYETLKENVKIRDSNTKTQLIILSAVCASILLSLSSLWGIRIHYNHGSGSGSGSGSCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSSGGGSSGSKKKNKKGSGEPLIDVHDLISDMIKKEEELVEVNKRKSKYKLATSVLAGGSSGGGSSGMKVSRHTVLLNIILIVSLLGCVLTLNLFSANDDKRALKDIDRTLEKLLRKKQIIISTAAVALAITLGGLFGSLGYKSWKNKNKSKDKVNGGSSGGGSSGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGIVGIVAGLAVLAVVVIGAVVATVMCRRKSSGGKGGSYSQAASSDSAQGSDVSLTA

SEQ ID NO: 13 - RNA, 2811 nt, synthetic construct
  source 1..2811: mol_type = other RNA
atggggggggctgccgccaggttgggggccgtgattttgtttgtcgtcatagtgggcctccatggggtccgcagcctgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggcggctctggaggaggcggctccggaggcatgaagaccatcatcatcgtgaccctgttcatcctgatcctcaacacgatcatcatcaacccctgcacctgtgtgcccaacacactgcccatcgagtacttcgactacgagacactgaaagagaacgtcaagatccgggacagcaacaccaagacacagctgatcatcctgagcgccgtgtgcgcctctatcctgctgagcctttctagcctgtggggcatcagaatccactacaaccacggatctgggtccggcagcgggtccgggtcctgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggcggtagctccgggggtggctcctctggttccaagaagaagaacaagaaaggctccggcgagcccctgatcgacgtgcacgatctgatctccgacatgatcaagaaagaggaagaactggtcgaagtcaacaagcggaagtccaagtacaagctggccacctctgtgctggctggtggatcatccggtggaggttcatcaggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggatcttctggcggagggtcatccggcttctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 14 - DNA, 2223 bp, synthetic construct
  source 1..2223: mol_type = other DNA
atgggcagactgacatctggcgtgggaacagctgctctgctggtggttgctgtgggcctgagagtcgtgtgtgccaaatacgccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 15 - protein, 739 aa, synthetic construct
MGRLTSGVGTAALLVVAVGLRVVCAKYAMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGIVGIVAGLAVLAVVVIGAVVATVMCRRKSSGGKGGSYSQAASSDSAQGSDVSLTA

SEQ ID NO: 16 - RNA, 2223 nt, synthetic construct
  source 1..2223: mol_type = other RNA
atgggcagactgacatctggcgtgggaacagctgctctgctggtggttgctgtgggcctgagagtcgtgtgtgccaaatacgccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 17 - DNA, 3672 bp, synthetic construct
  source 1..3672: mol_type = other DNA
atgggcagactgacatctggcgtgggaacagctgctctgctggtggttgctgtgggcctgagagtcgtgtgtgccaaatacgccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggttcaggtggcggcggttcaggcggacaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggatcaggcggcggaggatctggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 18 - protein, 1222 aa, synthetic construct
MGRLTSGVGTAALLVVAVGLRVVCAKYAMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGXGGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSGGGGSGGQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGIVGIVAGLAVLAVVVIGAVVATVMCRRKSSGGKGGSYSQAASSDSAQGSDVSLTA

SEQ ID NO: 19 - RNA, 3672 nt, synthetic construct
  source 1..3672: mol_type = other RNA
atgggcagactgacatctggcgtgggaacagctgctctgctggtggttgctgtgggcctgagagtcgtgtgtgccaaatacgccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggttcaggtggcggcggttcaggcggacaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggatcaggcggcggaggatctggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 20 - DNA, 3360 bp, synthetic construct
  source 1..3360: mol_type = other DNA
atgggcagactgacatctggcgtgggaacagctgctctgctggtggttgctgtgggcctgagagtcgtgtgtgccaaatacgccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggttcaggtggcggcggttcaggcggattctgcaatcggtacatgacagaggaaccctacaacaatatcttcgagatcatcaagcccgagaacctctacagcagcctcctgctgctgtccaacgagaaagagaacgacttccccagcagcaccaactgcaacggctatatgaagtgcatccccttctacaacaacgtgtccgagagatggaagcggtacaacttcatccagctgtacatcatcagaagcgccctgaatatccacgtgatgagcaagtacaacatgctgaacaagtataacaaagaaacgaaccgcctgctgaaacggaacaacaatgtggaaaaccgcatcaacaacatctccaaccactacctgtgcagcggcttcaagaaagaaaaccggctgtttttcctgctgttttacaagacgatcaagatgatgaagctctacatccgcaacctgtttatgaagtatatcaagatctactataagaccaagcacttcgaaaagaacattgagacaaacaagaaagtggtgtacgtcgagcgggacaacctgttcgacatcgagagaaacaatcttttcgacatcctgtatatgctgaagcggatcgacagctacgtcaagaacatctactccggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 21 - protein, 1118 aa, synthetic construct
MGRLTSGVGTAALLVVAVGLRVVCAKYAMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGIVGIVAGLAVLAVVVIGAVVATVMCRRKSSGGKGGSYSQAASSDSAQGSDVSLTA

SEQ ID NO: 22 - RNA, 3360 nt, synthetic construct
  source 1..3360: mol_type = other RNA
atgggcagactgacatctggcgtgggaacagctgctctgctggtggttgctgtgggcctgagagtcgtgtgtgccaaatacgccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggttcaggtggcggcggttcaggcggattctgcaatcggtacatgacagaggaaccctacaacaatatcttcgagatcatcaagcccgagaacctctacagcagcctcctgctgctgtccaacgagaaagagaacgacttccccagcagcaccaactgcaacggctatatgaagtgcatccccttctacaacaacgtgtccgagagatggaagcggtacaacttcatccagctgtacatcatcagaagcgccctgaatatccacgtgatgagcaagtacaacatgctgaacaagtataacaaagaaacgaaccgcctgctgaaacggaacaacaatgtggaaaaccgcatcaacaacatctccaaccactacctgtgcagcggcttcaagaaagaaaaccggctgtttttcctgctgttttacaagacgatcaagatgatgaagctctacatccgcaacctgtttatgaagtatatcaagatctactataagaccaagcacttcgaaaagaacattgagacaaacaagaaagtggtgtacgtcgagcgggacaacctgttcgacatcgagagaaacaatcttttcgacatcctgtatatgctgaagcggatcgacagctacgtcaagaacatctactccggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 23 - DNA, 4794 bp, synthetic construct
  source 1..4794: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggaggttcaggtggcggcggttcaggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggcggatcaggcggcggaggatctggcggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggtggaagcggcggaggcggaagcggaggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 24 - protein, 1596 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 25 - RNA, 4794 nt, synthetic construct
  source 1..4794: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggaggttcaggtggcggcggttcaggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggcggatcaggcggcggaggatctggcggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggtggaagcggcggaggcggaagcggaggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 26 - DNA, 4152 bp, synthetic construct
  source 1..4152: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggaggttcaggtggcggcggttcaggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggcggatcaggcggcggaggatctggcggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 27 - protein, 1382 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 28 - RNA, 4152 nt, synthetic construct
  source 1..4152: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggaggttcaggtggcggcggttcaggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggcggatcaggcggcggaggatctggcggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 29 - DNA, 3840 bp, synthetic construct
  source 1..3840: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcttctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggttcaggtggcggcggttcaggcggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 30 - protein, 1278 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 31 - RNA, 3840 nt, synthetic construct
  source 1..3840: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcttctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggttcaggtggcggcggttcaggcggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 32 - DNA, 3624 bp, synthetic construct
  source 1..3624: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcgatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggaggttcaggtggcggcggttcaggcggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 33 - protein, 1206 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 34 - RNA, 3624 nt, synthetic construct
  source 1..3624: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcgatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggaggttcaggtggcggcggttcaggcggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 35 - DNA, 5289 bp, synthetic construct
  source 1..5289: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggaggttcaggtggcggcggttcaggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggcggatcaggcggcggaggatctggcggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggtggaagcggcggaggcggaagcggaggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggtagcggtggcggaggctctggtggaatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 36 - protein, 1761 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSGGGGSGGIVGIVAGLAVLAVVVIGAVVATVMCRRKSSGGKGGSYSQAASSDSAQGSDVSLTA

SEQ ID NO: 37 - RNA, 5289 nt, synthetic construct
  source 1..5289: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggaggttcaggtggcggcggttcaggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggcggatcaggcggcggaggatctggcggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggtggaagcggcggaggcggaagcggaggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggtagcggtggcggaggctctggtggaatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 38 - DNA, 2214 bp, synthetic construct
  source 1..2214: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 39 - protein, 736 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGIVGIVAGLAVLAVVVIGAVVATVMCRRKSSGGKGGSYSQAASSDSAQGSDVSLTA

SEQ ID NO: 40 - RNA, 2214 nt, synthetic construct
  source 1..2214: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 41 - DNA, 3663 bp, synthetic construct
  source 1..3663: mol_type = other DNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggttcaggtggcggcggttcaggcggacaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggatcaggcggcggaggatctggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 42 - protein, 1219 aa, synthetic construct
MGGAAARLGAVILFVVIVGLHGVRSMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSGGGGSGGQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGIVGIVAGLAVLAVVVIGAVVATVMCRRKSSGGKGGSYSQAASSDSAQGSDVSLTA

SEQ ID NO: 43 - RNA, 3663 nt, synthetic construct
  source 1..3663: mol_type = other RNA
atggggggggctgccgccaggttgggggccgtgattttgtttgtcgtcatagtgggcctccatggggtccgcagcatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgccggcggctctggaggaggcggctccggaggcaatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgtggtggaagcggcggaggcggaagcggaggactgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggcggaggtagcggtggcggaggctctggtggaatgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaatggcggcagcggaggtggtggctctggcggaaatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaatggcggttctggtggtggcggtagtggcggcatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggttcaggtggcggcggttcaggcggacaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggatcaggcggcggaggatctggcggagatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 44 - DNA, 2826 bp, synthetic construct
  source 1..2826: mol_type = other DNA
atggggggggctgccgccaggttgggggccgtgattttgtttgtcgtcatagtgggcctccatggggtccgcagccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggctctggaggaggcggctccggaggcgatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggtggaagcggcggaggcggaagcggaggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggtagcggtggcggaggctctggtggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 45 - protein, 940 aa, synthetic construct
MGGAAARLGAVILFVVIVGLHGVRSQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 46 - RNA, 2826 nt, synthetic construct
  source 1..2826: mol_type = other RNA
atggggggggctgccgccaggttgggggccgtgattttgtttgtcgtcatagtgggcctccatggggtccgcagccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggctctggaggaggcggctccggaggcgatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggtggaagcggcggaggcggaagcggaggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggtagcggtggcggaggctctggtggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 47 - DNA, 3321 bp, synthetic construct
  source 1..3321: mol_type = other DNA
atggggggggctgccgccaggttgggggccgtgattttgtttgtcgtcatagtgggcctccatggggtccgcagccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggctctggaggaggcggctccggaggcgatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggtggaagcggcggaggcggaagcggaggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggtagcggtggcggaggctctggtggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggcggcagcggaggtggtggctctggcggaatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 48 - protein, 1105 aa, synthetic construct
MGGAAARLGAVILFVVIVGLHGVRSQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADIGGSGGGGSGGIVGIVAGLAVLAVVVIGAVVATVMCRRKSSGGKGGSYSQAASSDSAQGSDVSLTA

SEQ ID NO: 49 - RNA, 3321 nt, synthetic construct
  source 1..3321: mol_type = other RNA
atggggggggctgccgccaggttgggggccgtgattttgtttgtcgtcatagtgggcctccatggggtccgcagccaagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaaggcggctctggaggaggcggctccggaggcgatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctcggtggaagcggcggaggcggaagcggaggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggaggtagcggtggcggaggctctggtggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggcggcagcggaggtggtggctctggcggaatgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatcggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 50 - DNA, 4470 bp, synthetic construct
  source 1..4470: mol_type = other DNA
atggggggggctgccgccaggttgggggccgtgattttgtttgtcgtcatagtgggcctccatggggtccgcagcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggtggaagcggcggaggcggaagcggaggactgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggaggtagcggtggcggaggctctggtggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggcagcggaggtggtggctctggcggaaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggcggttctggtggtggcggtagtggcggccaagagcggagagccaaagagaagctgcaagagcagcagcgcgacctggaacagagaaaggccgacaccaagaagaacctggaacgcaagaaagaacacggcgacgtgctggccgaggacctgtatggcagactggaaatccctgccatcgagctgcccagcgagaacgagagaggctactacatccctcaccagagcagcctgcctcaggacaacagaggcaacagccgggacagcaaagagatcagcatcatcgagaacaccaaccgcgagagcatcaccaccaacgtggaaggcagacgggacatccacaagggccacctggaagaaaagaaggacggcagcatcaagcccgagcagaaagaggacaagagcgccgacatccagaatcacaccctggaaaccgtgaacatcagcgacgtgaacgacttccagatctctaagtacgaggacgagatcagcgccgagtacgacgacagcctgatcgacgaggaagaaggaggttcaggtggcggcggttcaggcggagacgacgaggacctggacgagttcaagcccatcgtgcagtacgacaacttccaggacgaggaaaacatcggcatctacaaagagcttgaggacctgatcgagaagaacgagaacctggatgatctggacgagggcatcgagaagtccagcgaggaactgagcgaggaaaagatcaagaagggcaagaagtacgaaaagaccaaggacaacaacttcaagccgaacgacaagagcctgtacgacgagcacatcaagaagtataagaacgataagcaagtgaacaaagagaaagagaagttcatcaagtccctgttccacatcttcgacggcgacaacgagatcctccagatcgtggatgagctgtccgaggacatcaccaagtactttatgaagctgggcggatcaggcggcggaggatctggcggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggtggaagcggcggaggcggaagcggaggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 51 - protein, 1488 aa, synthetic construct
MGGAAARLGAVILFVVIVGLHGVRSNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEGGSGGGGSGGDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKLGGSGGGGSGGFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSGGSGGGGSGGCNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNGGSGGGGSGGIVGIVAGLAVLAVVVIGAVVATVMCRRKSSGGKGGSYSQAASSDSAQGSDVSLTA

SEQ ID NO: 52 - RNA, 4470 nt, synthetic construct
  source 1..4470: mol_type = other RNA
atggggggggctgccgccaggttgggggccgtgattttgtttgtcgtcatagtgggcctccatggggtccgcagcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggtggaagcggcggaggcggaagcggaggactgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggaggtagcggtggcggaggctctggtggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggcagcggaggtggtggctctggcggaaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggcggttctggtggtggcggtagtggcggccaagagcggagagccaaagagaagctgcaagagcagcagcgcgacctggaacagagaaaggccgacaccaagaagaacctggaacgcaagaaagaacacggcgacgtgctggccgaggacctgtatggcagactggaaatccctgccatcgagctgcccagcgagaacgagagaggctactacatccctcaccagagcagcctgcctcaggacaacagaggcaacagccgggacagcaaagagatcagcatcatcgagaacaccaaccgcgagagcatcaccaccaacgtggaaggcagacgggacatccacaagggccacctggaagaaaagaaggacggcagcatcaagcccgagcagaaagaggacaagagcgccgacatccagaatcacaccctggaaaccgtgaacatcagcgacgtgaacgacttccagatctctaagtacgaggacgagatcagcgccgagtacgacgacagcctgatcgacgaggaagaaggaggttcaggtggcggcggttcaggcggagacgacgaggacctggacgagttcaagcccatcgtgcagtacgacaacttccaggacgaggaaaacatcggcatctacaaagagcttgaggacctgatcgagaagaacgagaacctggatgatctggacgagggcatcgagaagtccagcgaggaactgagcgaggaaaagatcaagaagggcaagaagtacgaaaagaccaaggacaacaacttcaagccgaacgacaagagcctgtacgacgagcacatcaagaagtataagaacgataagcaagtgaacaaagagaaagagaagttcatcaagtccctgttccacatcttcgacggcgacaacgagatcctccagatcgtggatgagctgtccgaggacatcaccaagtactttatgaagctgggcggatcaggcggcggaggatctggcggattctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagcggtggaagcggcggaggcggaagcggaggatgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 53 - DNA, 1890 bp, synthetic construct
  source 1..1890: mol_type = other DNA
atggggggggctgccgccaggttgggggccgtgattttgtttgtcgtcatagtgggcctccatggggtccgcagcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggtggaagcggcggaggcggaagcggaggactgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggaggtagcggtggcggaggctctggtggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggcagcggaggtggtggctctggcggaaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 54 - protein, 628 aa, synthetic construct
MGGAAARLGAVILFVVIVGLHGVRSNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGGIVGIVAGLAVLAVVVIGAVVATVMCRRKSSGGKGGSYSQAASSDSAQGSDVSLTA

SEQ ID NO: 55 - RNA, 1890 nt, synthetic construct
  source 1..1890: mol_type = other RNA
atggggggggctgccgccaggttgggggccgtgattttgtttgtcgtcatagtgggcctccatggggtccgcagcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggtggaagcggcggaggcggaagcggaggactgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggaggtagcggtggcggaggctctggtggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggcagcggaggtggtggctctggcggaaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaacggaggatccggtggtggcggcagcggcggcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgataa

SEQ ID NO: 56 - DNA, 2175 bp, synthetic construct
  source 1..2175: mol_type = other DNA
atgcagattttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggccggatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcctgccggcggctctggaggaggcggctccggaggcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggtggaagcggcggaggcggaagcggaggactgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggaggtagcggtggcggaggctctggtggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggcagcggaggtggtggctctggcggaaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaactgataa

SEQ ID NO: 57 - protein, 733 aa, synthetic construct
MQIFVKTLTGKTITLEVEPSDTIENVKAKIQDKEGIPPDQQRLIFAGKQLEDGRTLSDYNIQKESTLHLVLRLRGGRMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPAGGSGGGGSGGNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCGGSGGGGSGGLNASKVNKKGKIAEEKKRKNIKNIDKAIEEHNKRKKLIYYSLIASGAIASVAAILGGGSGGGGSGGMKVSRHTVLLNIILIVSLLGCVLTLNLFSANDDKRALKDIDRTLEKLLRKKQIIISTAAVALAITLGGLFGSLGYKSWKNKNKSKDKVNGGSGGGGSGGNSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTNGGSGGGGSGG

SEQ ID NO: 58 - RNA, 2175 nt, synthetic construct
  source 1..2175: mol_type = other RNA
atgcagattttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggccggatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcctgccggcggctctggaggaggcggctccggaggcaaccacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaacgccatccacctgtacgccagcgtgttcagcaacaacgccagagagatcatccggctgcacagcgacgccagcaagaacaaagagaaggccctgatcatcatcaagagcctgctgagcaccaacctgccttacggcaagaccaacctgacagacgccctgctgcaagtgcggaagcacctgaatgaccggatcaacagagagaacgccaatcagctggtggtcatcctgaccgacggcatccctgacagcatccaggacagcctgaaagagagccggaagctgagcgacagaggcgtgaagatcgccgtgtttggcatcggccagggcatcaacgtggccttcaatagattcctcgtgggctgcggtggaagcggcggaggcggaagcggaggactgaacgccagcaaagtgaacaagaagggcaagatcgccgaggaaaagaagcggaagaacatcaagaatatcgacaaggccatcgaggaacacaacaagcgcaagaagctgatctactacagcctgatcgccagcggcgccattgcttctgtggctgctattctgggaggaggtagcggtggcggaggctctggtggaatgaaggtgtccagacacaccgtgctgctgaacatcatcctgatcgtgtccctgctgggctgcgtgctgaccctgaacctgttcagcgccaacgacgacaagagagccctgaaggacatcgacagaaccctggaaaagctgctgcggaagaagcagatcatcatcagcacagccgccgtggctctggccattacactcggaggactgtttggcagcctgggctacaagagctggaagaacaagaacaagtccaaggacaaagtgaacggcggcagcggaggtggtggctctggcggaaacagcagcgacagcgattgcgagcctctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggaaagctggcccacgagcacaagctgcccttcaagattaagatgaagaaatggtggaagtgctgcggccacgtgaccgacgagctgctgatcaaagagcacgacgactacaacagcatctacaactacatcaacaacgagagcagcagccgcgagcagttcctgatctttctgaacatgatcaagcacagctggaccaccttcaccatggaaaccttcatcaagtgcaagatcagcctggaaaacaacatgcggaacgtgaccaactgataa

SEQ ID NO: 59 - DNA, 72 bp, unidentified
  source 1..72: mol_type = other DNA; note = Plasmodium falciparum
aacgccaatcctaacgtggaccccaacgctaaccccaatgtggacccaaatgccaatccaaatgtcgatccc

SEQ ID NO: 60 - protein, 24 aa, unidentified
  source 1..24: note = Plasmodium falciparum
NANPNVDPNANPNVDPNANPNVDP

SEQ ID NO: 61 - RNA, 72 nt, unidentified
  source 1..72: mol_type = other RNA; note = Plasmodium falciparum
aacgccaatcctaacgtggaccccaacgctaaccccaatgtggacccaaatgccaatccaaatgtcgatccc

SEQ ID NO: 62 - DNA, 375 bp, unidentified
  source 1..375: mol_type = other DNA; note = Plasmodium falciparum
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaac

SEQ ID NO: 63 - protein, 125 aa, unidentified
  source 1..125: note = Plasmodium falciparum
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 64 - RNA, 375 nt, unidentified
  source 1..375: mol_type = other RNA; note = Plasmodium falciparum
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaac

SEQ ID NO: 65-67 - skipped (3 entries)

SEQ ID NO: 68 - DNA, 309 bp, synthetic construct
  source 1..309: mol_type = other DNA
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagc

SEQ ID NO: 69 - protein, 103 aa, synthetic construct
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCS

SEQ ID NO: 70 - RNA, 309 nt, synthetic construct
  source 1..309: mol_type = other RNA
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagc

SEQ ID NO: 71 - DNA, 42 bp, synthetic construct
  source 1..42: mol_type = other DNA
aagcacatcaaagagtacctgaacaagatccagaacagcctg

SEQ ID NO: 72 - protein, 14 aa, synthetic construct
KHIKEYLNKIQNSL

SEQ ID NO: 73 - RNA, 42 nt, synthetic construct
  source 1..42: mol_type = other RNA
aagcacatcaaagagtacctgaacaagatccagaacagcctg

SEQ ID NO: 74 - DNA, 36 bp, synthetic construct
  source 1..36: mol_type = other DNA
aacaagcctaaggatgagctggactacgccaatgac

SEQ ID NO: 75 - protein, 12 aa, synthetic construct
NKPKDELDYAND

SEQ ID NO: 76 - RNA, 36 nt, synthetic construct
  source 1..36: mol_type = other RNA
aacaagcctaaggatgagctggactacgccaatgac

SEQ ID NO: 77 - DNA, 147 bp, synthetic construct
  source 1..147: mol_type = other DNA
agcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgc

SEQ ID NO: 78 - protein, 49 aa, synthetic construct
SLSTEWSPCSVTCGNGIQVRIKPGSANKPKDELDYANDIEKKICKMEKC

SEQ ID NO: 79 - RNA, 147 nt, synthetic construct
  source 1..147: mol_type = other RNA
agcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgc

SEQ ID NO: 80 - DNA, 42 bp, synthetic construct
  source 1..42: mol_type = other DNA
atcgagaagaaaatctgcaagatggaaaagtgcagcagcgtg

SEQ ID NO: 81 - protein, 14 aa, synthetic construct
IEKKICKMEKCSSV

SEQ ID NO: 82 - RNA, 42 nt, synthetic construct
  source 1..42: mol_type = other RNA
atcgagaagaaaatctgcaagatggaaaagtgcagcagcgtg

SEQ ID NO: 83 - DNA, 504 bp, synthetic construct
  source 1..504: mol_type = other DNA
aacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 84 - protein, 168 aa, synthetic construct
NANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 85 - RNA, 504 nt, synthetic construct
  source 1..504: mol_type = other RNA
aacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 86 - DNA, 36 bp, synthetic construct
  source 1..36: mol_type = other DNA
aagctgaagcagcccgccgacggcaatcccgatcct

SEQ ID NO: 87 - protein, 12 aa, synthetic construct
KLKQPADGNPDP

SEQ ID NO: 88 - RNA, 36 nt, synthetic construct
  source 1..36: mol_type = other RNA
aagctgaagcagcccgccgacggcaatcccgatcct

SEQ ID NO: 89 - DNA, 24 bp, synthetic construct
  source 1..24: mol_type = other DNA
cccgccgacggcaatcccgatcct

SEQ ID NO: 90 - protein, 8 aa, synthetic construct
PADGNPDP

SEQ ID NO: 91 - RNA, 24 nt, synthetic construct
  source 1..24: mol_type = other RNA
cccgccgacggcaatcccgatcct

SEQ ID NO: 92 - DNA, 21 bp, synthetic construct
  source 1..21: mol_type = other DNA
gccgacggcaatcccgatcct

SEQ ID NO: 93 - protein, 7 aa, synthetic construct
ADGNPDP

SEQ ID NO: 94 - RNA, 21 nt, synthetic construct
  source 1..21: mol_type = other RNA
gccgacggcaatcccgatcct

SEQ ID NO: 95 - DNA, 15 bp, synthetic construct
  source 1..15: mol_type = other DNA
aagctgaagcagccc

SEQ ID NO: 96 - protein, 5 aa, synthetic construct
KLKQP

SEQ ID NO: 97 - RNA, 15 nt, synthetic construct
  source 1..15: mol_type = other RNA
aagctgaagcagccc

SEQ ID NO: 98 - DNA, 36 bp, synthetic construct
  source 1..36: mol_type = other DNA
gaggacaacgagaagctgcggaagcccaagcacaag

SEQ ID NO: 99 - protein, 12 aa, synthetic construct
EDNEKLRKPKHK

SEQ ID NO: 100 - RNA, 36 nt, synthetic construct
  source 1..36: mol_type = other RNA
gaggacaacgagaagctgcggaagcccaagcacaag

SEQ ID NO: 101 - DNA, 186 bp, synthetic construct
  source 1..186: mol_type = other DNA
ctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaac

SEQ ID NO: 102 - protein, 62 aa, synthetic construct
LFQEYQCYGSSSNTRVLNELNYDNAGTNLYNELEMNYYGKQENWYSLKKNSRSLGENDDGNN

SEQ ID NO: 103 - RNA, 186 nt, synthetic construct
  source 1..186: mol_type = other RNA
ctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaac

SEQ ID NO: 104 - protein, 4 aa, synthetic construct
NPNA

SEQ ID NO: 105 - protein, 4 aa, synthetic construct
NVDP

SEQ ID NO: 106-107 - skipped (2 entries)

SEQ ID NO: 108 - protein, 4 aa, synthetic construct
NANP

SEQ ID NO: 109-110 - skipped (2 entries)

SEQ ID NO: 111 - protein, 6 aa, synthetic construct
NPNANP

SEQ ID NO: 112-113 - skipped (2 entries)

SEQ ID NO: 114 - protein, 6 aa, synthetic construct
NANPNA

SEQ ID NO: 115 - skipped

SEQ ID NO: 116 - DNA, 432 bp, synthetic construct
  source 1..432: mol_type = other DNA
aatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 117 - protein, 144 aa, synthetic construct
NANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 118 - RNA, 432 nt, synthetic construct
  source 1..432: mol_type = other RNA
aatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 119 - DNA, 60 bp, synthetic construct
  source 1..60: mol_type = other DNA
gaactcgatggctccaaaaatgagtggggatggtcaaaatccaagctcggggcaaatgcc

SEQ ID NO: 120 - protein, 20 aa, synthetic construct
ELDGSKNEWGWSKSKLGANA

SEQ ID NO: 121 - RNA, 60 nt, synthetic construct
  source 1..60: mol_type = other RNA
gaactcgatggctccaaaaatgagtggggatggtcaaaatccaagctcggggcaaatgcc

SEQ ID NO: 122 - DNA, 498 bp, unidentified
  source 1..498: mol_type = other DNA; note = Plasmodium falciparum
gagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgctgagaatgtcgaggaatccgtc

SEQ ID NO: 123 - protein, 190 aa, unidentified
  source 1..190: note = Plasmodium falciparum
ENVQVSDELFNELLNSVDVNGEVKENILEESQVNDDIFNSLVKSVQQEQQHNVEEKVEESVEENDEESVEENVEENVEENDDESVASSVEESIASSVDESIDSSIEENVAPTVEEIVAPTVEEIVAPSVVESVAPSVEESVEENVEESVAENVEESVAENVEESVAENVEESVAENVEESVAENVEESVA

SEQ ID NO: 124 - RNA, 498 nt, unidentified
  source 1..498: mol_type = other RNA; note = Plasmodium falciparum
gagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgctgagaatgtcgaggaatccgtc

SEQ ID NO: 125 - skipped

SEQ ID NO: 126 - protein, 391 aa, Anopheles gambiae
MCRGLSAVLILLVSLSAQLHVVVGEEAPKPEKEICGLKVGRLLDSVKGWLSVSQQEKCPLNKYCENKIQADQYNLVPLTCIRWRSLNPASPTGSLGGKDVVSKIDAAMSNFKTLFEPMKADLAKLEEEVKRQVLDAWKALEPLQKEVYRSTLASGRIERAVFYSFMEMGDNVKLDNYFQPANVEELLKYAWALPMHKKQRSMYDLIGQLVQSSKSPMLQTLHAVELATVVNPELENRENLLNDQVVQLRDNLYKNSFATLVSIARHFPDHFDTLRQRLFKLPDGSKPGADTLPNIVNFIAQLPSDELRLSSVDLLLQSLTAENGTLVQDPEYVYRLSQLAHAMPSLVDVKAHPDLQQSVDDLMAKFNTPIDGKTLQYFQNIGISPSSSVAT

SEQ ID NO: 127 - skipped

SEQ ID NO: 128 - DNA, 1197 bp, synthetic construct
  source 1..1197: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 129 - RNA, 1197 nt, synthetic construct
  source 1..1197: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 130-131 - skipped (2 entries)

SEQ ID NO: 132 - DNA, 294 bp, synthetic construct
  source 1..294: mol_type = other DNA
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcg

SEQ ID NO: 133 - protein, 98 aa, synthetic construct
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPA

SEQ ID NO: 134 - RNA, 294 nt, synthetic construct
  source 1..294: mol_type = other RNA
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcg

SEQ ID NO: 135 - DNA, 294 bp, synthetic construct
  source 1..294: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgcc

SEQ ID NO: 136 - skipped

SEQ ID NO: 137 - RNA, 294 nt, synthetic construct
  source 1..294: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcaacctgcc

SEQ ID NO: 138 - protein, 9 aa, synthetic construct
AILSVSSFL

SEQ ID NO: 139 - protein, 8 aa, synthetic construct
LSVSSFLF

SEQ ID NO: 140 - protein, 9 aa, synthetic construct
FVEALFQEY

SEQ ID NO: 141 - protein, 9 aa, synthetic construct
GSSSNTRVL

SEQ ID NO: 142 - protein, 12 aa, synthetic construct
ELNYDNAGTNLY

SEQ ID NO: 143 - DNA, 498 bp, synthetic construct
  source 1..498: mol_type = other DNA
caagagagacgtgctaaagaaaagttgcaagagcagcaaagagatttagaacaaaggaaggctgatacgaaaaaaaatttagaaagaaaaaaggaacatggagatgtattagcagaggatttatatggtcgtttagaaataccagctatagaacttccatcagaaaatgaacgtggatattatataccacatcaatcttctttacctcaggacaacagagggaatagtagagattcgaaggaaatatctataatagaaaatacaaatagagaatctattacaacaaatgttgaaggacgaagggatatacataaaggacatcttgaagaaaagaaagatggttcaataaaaccagaacaaaaagaagataaatctgctgacatacaaaatcatacattagagacagtaaatatttctgatgttaatgattttcaaataagtaagtatgaggatgaaataagtgctgaatatgacgattcattaatagatgaagaagaa

SEQ ID NO: 144 - protein, 166 aa, synthetic construct
QERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEE

SEQ ID NO: 145 - RNA, 498 nt, synthetic construct
  source 1..498: mol_type = other RNA
caagagagacgtgctaaagaaaagttgcaagagcagcaaagagatttagaacaaaggaaggctgatacgaaaaaaaatttagaaagaaaaaaggaacatggagatgtattagcagaggatttatatggtcgtttagaaataccagctatagaacttccatcagaaaatgaacgtggatattatataccacatcaatcttctttacctcaggacaacagagggaatagtagagattcgaaggaaatatctataatagaaaatacaaatagagaatctattacaacaaatgttgaaggacgaagggatatacataaaggacatcttgaagaaaagaaagatggttcaataaaaccagaacaaaaagaagataaatctgctgacatacaaaatcatacattagagacagtaaatatttctgatgttaatgattttcaaataagtaagtatgaggatgaaataagtgctgaatatgacgattcattaatagatgaagaagaa

SEQ ID NO: 146 - DNA, 498 bp, synthetic construct
  source 1..498: mol_type = other DNA
caagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaa

SEQ ID NO: 147 - skipped

SEQ ID NO: 148 - RNA, 498 nt, synthetic construct
  source 1..498: mol_type = other RNA
caagaaagacgggccaaagagaaactgcaagagcagcaacgcgacctggaacagagaaaggccgacaccaagaagaacctcgaacggaagaaagaacacggggacgtgctggccgaggatctgtatggcagactggaaatccccgccattgagctgccctccgagaacgagagaggctactacatccctcaccagagcagcctgcctcaggacaacagaggcaacagccgggactctaaagagatctccatcatcgagaacaccaaccgcgagagcatcaccacaaacgtggaaggcagacgggacatccacaagggccaccttgaggaaaagaaagacggcagcatcaagcccgagcagaaagaggacaagagcgccgacatccagaatcacacactggaaaccgtgaacatcagcgacgtgaacgactttcagatctctaagtacgaggatgagatcagcgccgagtacgacgacagcctgatcgatgaagaagaa

SEQ ID NO: 149 - protein, 10 aa, synthetic construct
HGDVLAEDLY

SEQ ID NO: 150 - protein, 10 aa, synthetic construct
VLAEDLYGRL

SEQ ID NO: 151 - protein, 10 aa, synthetic construct
KSADIQNHTL

SEQ ID NO: 152 - protein, 9 aa, synthetic construct
IQNHTLETV

SEQ ID NO: 153 - protein, 12 aa, synthetic construct
ISDVNDFQISKY

SEQ ID NO: 154 - DNA, 396 bp, synthetic construct
  source 1..396: mol_type = other DNA
gatgatgaagacttagacgaatttaagcctattgtgcaatatgacaatttccaagatgaagaaaacataggaatttataaagaactagaagatttgatagagaaaaatgaaaatttagatgatttagatgaaggaatagaaaaatcatcagaagaattatctgaagaaaaaataaaaaaaggaaagaaatatgaaaaaacaaaggataataattttaaaccaaatgataaaagtttgtatgatgagcatattaaaaaatataaaaatgataagcaggttaataaggaaaaggaaaaattcataaaatcattgtttcatatatttgacggagacaatgaaattttacagatcgtggatgagttatctgaagatataactaaatattttatgaaacta

SEQ ID NO: 155 - protein, 132 aa, synthetic construct
DDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 156 - RNA, 396 nt, synthetic construct
  source 1..396: mol_type = other RNA
gatgatgaagacttagacgaatttaagcctattgtgcaatatgacaatttccaagatgaagaaaacataggaatttataaagaactagaagatttgatagagaaaaatgaaaatttagatgatttagatgaaggaatagaaaaatcatcagaagaattatctgaagaaaaaataaaaaaaggaaagaaatatgaaaaaacaaaggataataattttaaaccaaatgataaaagtttgtatgatgagcatattaaaaaatataaaaatgataagcaggttaataaggaaaaggaaaaattcataaaatcattgtttcatatatttgacggagacaatgaaattttacagatcgtggatgagttatctgaagatataactaaatattttatgaaacta

SEQ ID NO: 157 - DNA, 396 bp, synthetic construct
  source 1..396: mol_type = other DNA
gatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctc

SEQ ID NO: 158 - skipped

SEQ ID NO: 159 - RNA, 396 nt, synthetic construct
  source 1..396: mol_type = other RNA
gatgatgaggatctggacgagttcaagcccatcgtgcagtacgacaacttccaggatgaggaaaacatcgggatctacaaagagttggaggacctgattgagaagaacgagaacctggacgacctggatgaaggcatcgagaagtcctccgaggaactgtcagaggaaaagatcaagaaggggaagaagtacgaaaagaccaaggataacaacttcaagccgaacgacaagtccctgtacgatgagcacattaagaagtacaagaacgataagcaagtcaacaaagaaaaagaaaagttcatcaaatccctgtttcacatcttcgacggcgacaatgagatcctccagatcgtggatgagctgtccgaggacatcacaaagtatttcatgaagctc

SEQ ID NO: 160 - protein, 9 aa, synthetic construct
DEFKPIVQY

SEQ ID NO: 161 - protein, 9 aa, synthetic construct
FQDEENIGI

SEQ ID NO: 162 - protein, 10 aa, synthetic construct
FQDEENIGIY

SEQ ID NO: 163 - protein, 11 aa, synthetic construct
KSLYDEHIKKY

SEQ ID NO: 164 - protein, 9 aa, synthetic construct
SLYDEHIKK

SEQ ID NO: 165 - protein, 11 aa, synthetic construct
SLYDEHIKKYK

SEQ ID NO: 166 - protein, 8 aa, synthetic construct
YDEHIKKY

SEQ ID NO: 167 - protein, 12 aa, synthetic construct
HIFDGDNEILQI

SEQ ID NO: 168 - protein, 12 aa, synthetic construct
IVDELSEDITKY

SEQ ID NO: 169 - protein, 9 aa, synthetic construct
SEDITKYFM

SEQ ID NO: 170 - DNA, 612 bp, synthetic construct
  source 1..612: mol_type = other DNA
aatcatcttgggaatgttaaatatttagtcattgtgtttttgattttctttgatttgtttctagttaatggtagagatgtgcaaaacaatatagtggatgaaataaaatatcgtgaagaagtatgtaatgatgaggtagatctttaccttctaatggattgttctggaagtatacgtcgtcataattgggtgaaccatgcagtacctctagctatgaaattgatacaacaattaaatcttaatgataatgcaattcacttatatgctagtgttttttcaaacaatgcaagagaaattattagattacatagtgatgcatctaaaaacaaagagaaggctttaattattataaagtcactcttaagtacaaatcttccatatggtaaaacaaacttaactgatgcactgttacaagtaagaaaacatttaaatgaccgaatcaatagagagaatgctaatcaattagttgttatattaacagatggaattccagatagtattcaagattcattaaaagaatcaagaaaattaagtgatcgtggtgttaaaatagctgtttttggtattggacaaggtattaatgtagctttcaacagatttcttgtaggttgt

SEQ ID NO: 171 - protein, 204 aa, synthetic construct
NHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGC

SEQ ID NO: 172 - RNA, 612 nt, synthetic construct
  source 1..612: mol_type = other RNA
aatcatcttgggaatgttaaatatttagtcattgtgtttttgattttctttgatttgtttctagttaatggtagagatgtgcaaaacaatatagtggatgaaataaaatatcgtgaagaagtatgtaatgatgaggtagatctttaccttctaatggattgttctggaagtatacgtcgtcataattgggtgaaccatgcagtacctctagctatgaaattgatacaacaattaaatcttaatgataatgcaattcacttatatgctagtgttttttcaaacaatgcaagagaaattattagattacatagtgatgcatctaaaaacaaagagaaggctttaattattataaagtcactcttaagtacaaatcttccatatggtaaaacaaacttaactgatgcactgttacaagtaagaaaacatttaaatgaccgaatcaatagagagaatgctaatcaattagttgttatattaacagatggaattccagatagtattcaagattcattaaaagaatcaagaaaattaagtgatcgtggtgttaaaatagctgtttttggtattggacaaggtattaatgtagctttcaacagatttcttgtaggttgt

SEQ ID NO: 173 - DNA, 612 bp, synthetic construct
  source 1..612: mol_type = other DNA
aatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgt

SEQ ID NO: 174 - skipped

SEQ ID NO: 175 - RNA, 612 nt, synthetic construct
  source 1..612: mol_type = other RNA
aatcacctgggcaacgtgaagtacctcgtgatcgtgttcctgatcttcttcgacctgttcctggtcaacggccgggacgtgcagaacaacatcgtggacgagatcaagtaccgcgaggaagtgtgcaacgacgaggtggacctgtacctgctgatggattgcagcggcagcatcagacggcacaactgggttaaccatgccgtgcctctggccatgaagctgatccagcagctgaacctgaacgacaatgccatccacctgtacgccagcgtgttcagcaacaacgccagagagatcatccggctgcacagcgacgccagcaagaacaaagagaaggccctgatcatcatcaagagcctgctgagcaccaatctgccttacggcaagaccaacctgacagacgccctgctgcaagtgcggaagcacctgaatgaccggatcaacagagagaacgccaatcagctggtggtcatcctgaccgacggcatccctgacagcatccaggactccctgaaagagagccggaagctgagcgacagaggcgtgaagatcgccgtgtttggcatcggccagggcatcaacgtggccttcaatagattcctcgtcggctgt

SEQ ID NO: 176 - protein, 9 aa, synthetic construct
LLMDCSGSI

SEQ ID NO: 177 - protein, 8 aa, synthetic construct
GSIRRHNW

SEQ ID NO: 178 - protein, 9 aa, synthetic construct
NLNDNAIHL

SEQ ID NO: 179 - protein, 10 aa, synthetic construct
NLNDNAIHLY

SEQ ID NO: 180 - protein, 9 aa, synthetic construct
LNDNAIHLY

SEQ ID NO: 181 - protein, 9 aa, synthetic construct
ALIIIKSLL

SEQ ID NO: 182 - protein, 9 aa, synthetic construct
SLLSTNLPY

SEQ ID NO: 183 - protein, 11 aa, synthetic construct
SLLSTNLPYGK

SEQ ID NO: 184 - protein, 9 aa, synthetic construct
NLTDALLQV

SEQ ID NO: 185 - protein, 13 aa, synthetic construct
HLNDRINRENANQ

SEQ ID NO: 186 - protein, 9 aa, synthetic construct
KLSDRGVKI

SEQ ID NO: 187 - protein, 11 aa, synthetic construct
KLSDRGVKIAV

SEQ ID NO: 188 - protein, 11 aa, synthetic construct
AVFGIGQGINV

SEQ ID NO: 189 - protein, 12 aa, synthetic construct
AVFGIGQGINVA

SEQ ID NO: 190 - protein, 9 aa, synthetic construct
FGIGQGINV

SEQ ID NO: 191 - DNA, 240 bp, synthetic construct
  source 1..240: mol_type = other DNA
atgaaaaccataataatagtaacccttttcattttaattttaaatacaattattataaatccatgtacttgtgtccctaatacactgcctattgaatatttcgattatgaaaccctcaaagaaaatgtcaaaattcgagatagtaacacaaaaactcagttaattattttatcagctgtatgtgctagtatcttactctctcttagttccttatggggtatcagaatacattataatcat

SEQ ID NO: 192 - protein, 80 aa, synthetic construct
MKTIIIVTLFILILNTIIINPCTCVPNTLPIEYFDYETLKENVKIRDSNTKTQLIILSAVCASILLSLSSLWGIRIHYNH

SEQ ID NO: 193 - RNA, 240 nt, synthetic construct
  source 1..240: mol_type = other RNA
atgaaaaccataataatagtaacccttttcattttaattttaaatacaattattataaatccatgtacttgtgtccctaatacactgcctattgaatatttcgattatgaaaccctcaaagaaaatgtcaaaattcgagatagtaacacaaaaactcagttaattattttatcagctgtatgtgctagtatcttactctctcttagttccttatggggtatcagaatacattataatcat

SEQ ID NO: 194 - DNA, 240 bp, synthetic construct
  source 1..240: mol_type = other DNA
atgaagaccatcatcatcgtgaccctgttcatcctgatcctcaacacgatcatcatcaacccctgcacctgtgtgcccaacacactgcccatcgagtacttcgactacgagacactgaaagagaacgtcaagatccgggacagcaacaccaagacacagctgatcatcctgagcgccgtgtgcgcctctatcctgctgagcctttctagcctgtggggcatcagaatccactacaaccac

SEQ ID NO: 195 - skipped

SEQ ID NO: 196 - RNA, 240 nt, synthetic construct
  source 1..240: mol_type = other RNA
atgaagaccatcatcatcgtgaccctgttcatcctgatcctcaacacgatcatcatcaacccctgcacctgtgtgcccaacacactgcccatcgagtacttcgactacgagacactgaaagagaacgtcaagatccgggacagcaacaccaagacacagctgatcatcctgagcgccgtgtgcgcctctatcctgctgagcctttctagcctgtggggcatcagaatccactacaaccac

SEQ ID NO: 197 - DNA, 477 bp, synthetic construct
  source 1..477: mol_type = other DNA
aattcaagtgattctgattgtgagcccttaccatttggattaaaaccttcagatttaaatagaaaagttacagaagaagatttagaaagaatgataatagaattaccaggaaaattagaaaggaaagatatgtatttaatatggcattatagtcattctcttttgagagataaatttaataaaatgaaaagttcgttatggagtatttgtgggaaattagctcatgaacataagttaccattcaaaattaaaatgaagaaatggtggaaatgttgtggtcatgttacagatgaattattaataaaagagcatgatgattataattctatatataattatattaataatgaatcatcaagtcgtgaacaatttcttatatttcttaatatgataaagcattcatggacaacatttactatggagacttttattaaatgtaagatttctttagaaaataacatgagaaatgttacaaat

SEQ ID NO: 198 - protein, 159 aa, synthetic construct
NSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTN

SEQ ID NO: 199 - RNA, 477 nt, synthetic construct
  source 1..477: mol_type = other RNA
aattcaagtgattctgattgtgagcccttaccatttggattaaaaccttcagatttaaatagaaaagttacagaagaagatttagaaagaatgataatagaattaccaggaaaattagaaaggaaagatatgtatttaatatggcattatagtcattctcttttgagagataaatttaataaaatgaaaagttcgttatggagtatttgtgggaaattagctcatgaacataagttaccattcaaaattaaaatgaagaaatggtggaaatgttgtggtcatgttacagatgaattattaataaaagagcatgatgattataattctatatataattatattaataatgaatcatcaagtcgtgaacaatttcttatatttcttaatatgataaagcattcatggacaacatttactatggagacttttattaaatgtaagatttctttagaaaataacatgagaaatgttacaaat

SEQ ID NO: 200 - DNA, 477 bp, synthetic construct
  source 1..477: mol_type = other DNA
aatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaat

SEQ ID NO: 201 - skipped

SEQ ID NO: 202 - RNA, 477 nt, synthetic construct
  source 1..477: mol_type = other RNA
aatagcagcgatagcgattgcgagcccctgccttttggcctgaagcctagcgacctgaaccggaaagtgaccgaggaagatctggaacgcatgatcatcgagctgcccggcaagctggaacggaaggacatgtacctgatctggcactacagccacagcctgctgcgggacaagttcaacaagatgaagtccagcctgtggtccatctgcggcaaactggcccacgagcacaagctgcccttcaagattaagatgaagaaatggtggaagtgctgcggccacgtgaccgacgagctgctgatcaaagagcacgacgactacaacagcatctacaactacatcaacaatgagagcagcagccgcgagcagtttctgatcttcctgaacatgatcaagcacagctggaccacgttcaccatggaaaccttcatcaagtgcaagatcagcctggaaaacaacatgcgcaacgtgaccaat

SEQ ID NO: 203 - protein, 10 aa, synthetic construct
GLKPSDLNRK

SEQ ID NO: 204 - protein, 9 aa, synthetic construct
VTEEDLERM

SEQ ID NO: 205 - protein, 9 aa, synthetic construct
LIWHYSHSL

SEQ ID NO: 206 - protein, 9 aa, synthetic construct
SLWSICGKL

SEQ ID NO: 207 - protein, 9 aa, synthetic construct
LAHEHKLPF

SEQ ID NO: 208 - protein, 9 aa, synthetic construct
HVTDELLIK

SEQ ID NO: 209 - protein, 8 aa, synthetic construct
HDDYNSIY

SEQ ID NO: 210 - protein, 10 aa, synthetic construct
HDDYNSIYNY

SEQ ID NO: 211 - DNA, 168 bp, synthetic construct
  source 1..168: mol_type = other DNA
ttaaatgcttctaaagtaaataaaaagggtaaaatagcagaagaaaagaaaagaaaaaatattaaaaacattgataaagcaatagaagaacacaacaaaaggaagaaactaatttattattcattgatagcatctggggcaatagcatcggttgcggcaatattggga

SEQ ID NO: 212 - protein, 56 aa, synthetic construct
LNASKVNKKGKIAEEKKRKNIKNIDKAIEEHNKRKKLIYYSLIASGAIASVAAILG

SEQ ID NO: 213 - RNA, 168 nt, synthetic construct
  source 1..168: mol_type = other RNA
ttaaatgcttctaaagtaaataaaaagggtaaaatagcagaagaaaagaaaagaaaaaatattaaaaacattgataaagcaatagaagaacacaacaaaaggaagaaactaatttattattcattgatagcatctggggcaatagcatcggttgcggcaatattggga

SEQ ID NO: 214 - DNA, 168 bp, synthetic construct
  source 1..168: mol_type = other DNA
ctgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggc

SEQ ID NO: 215 - skipped

SEQ ID NO: 216 - RNA, 168 nt, synthetic construct
  source 1..168: mol_type = other RNA
ctgaatgccagcaaagtgaacaagaagggcaagattgccgaggaaaagaagcggaagaacatcaagaatatcgacaaggccatcgaggaacacaacaagcggaaaaagctgatctactactccctgatcgccagcggcgccattgcttctgtggccgctattcttggc

SEQ ID NO: 217 - protein, 11 aa, synthetic construct
SLIASGAIASV

SEQ ID NO: 218 - DNA, 267 bp, synthetic construct
  source 1..267: mol_type = other DNA
atgaaggtttctaggcataccgttcttttaaacattatcctaatcgttagcttattaggatgtgttttaactcttaaccttttttctgcaaatgatgataagagagcgttgaaggatatcgacagaacattagaaaaacttttgaggaaaaagcaaatcatcatatctactgctgctgttgctttggctataaccttaggtggtttatttggaagtttaggatataagagctggaaaaacaaaaacaaatcaaaagataaggtgaat

SEQ ID NO: 219 - protein, 89 aa, synthetic construct
MKVSRHTVLLNIILIVSLLGCVLTLNLFSANDDKRALKDIDRTLEKLLRKKQIIISTAAVALAITLGGLFGSLGYKSWKNKNKSKDKVN

SEQ ID NO: 220 - RNA, 267 nt, synthetic construct
  source 1..267: mol_type = other RNA
atgaaggtttctaggcataccgttcttttaaacattatcctaatcgttagcttattaggatgtgttttaactcttaaccttttttctgcaaatgatgataagagagcgttgaaggatatcgacagaacattagaaaaacttttgaggaaaaagcaaatcatcatatctactgctgctgttgctttggctataaccttaggtggtttatttggaagtttaggatataagagctggaaaaacaaaaacaaatcaaaagataaggtgaat

SEQ ID NO: 221 - DNA, 267 bp, synthetic construct
  source 1..267: mol_type = other DNA
atgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaat

SEQ ID NO: 222 - skipped

SEQ ID NO: 223 - RNA, 267 nt, synthetic construct
  source 1..267: mol_type = other RNA
atgaaggtgtccagacacaccgtgctgctgaacatcatcctgattgtgtccctgctgggctgcgtgctgaccctgaacctgttcagcgccaacgacgacaagagagccctgaaggacatcgacagaaccctggaaaagctgctgaggaagaagcagatcatcattagcaccgccgctgtggccctggccattacacttggaggcctgtttggcagcctgggctacaagtcctggaagaacaagaacaagtccaaggacaaagtgaat

SEQ ID NO: 224 - protein, 9 aa, synthetic construct
SLLGCVLTL

SEQ ID NO: 225 - protein, 9 aa, synthetic construct
RTLEKLLRK

SEQ ID NO: 226 - protein, 10 aa, synthetic construct
RTLEKLLRKK

SEQ ID NO: 227 - protein, 9 aa, synthetic construct
GLFGSLGYK

SEQ ID NO: 228 - DNA, 954 bp, synthetic construct
  source 1..954: mol_type = other DNA
tgtaatattccaaaaaattatactgttggagatgttttaagatatgtaaaaaataaaataattgaagatactaatataagtatgttgaatgatttacaattaaaaataatacataatgataattatatcgatataccaaaagatatgaatatagaatatgcaattaattatttgtttatatataccccttttatagtatatccatcagatgatatattaaaatataataatataattgatttaattttatataataccattatagatataaaagaaaactcttttgttaaaaaagacattccgatatatgttaagaaaggtaatatttttaaaaatgttagtttaaaatatatacctataaatataaaagaagatgaattattaacatatttattacattatttaacagataatcatgaggttgtttcttttataagagatataacatgtatatgtgtttatcctttatgtgattcaatatatatatttcatgaaaagaaacaattatcattaataccttctatatcttatcatatacaattaaaaaatgtttattatatatctacagtagaaacatatgaaaatttttataatatattaggtcatatggaattaaatttgaataagttcttacatatacaaaatgatggaaataattataagaaaaaagaagcatataataatatttattatatgaataatgtggatgaattgatacatattgatgttaatataatatttccaagcattcaaaaaataatacgtgtaaagaatttgaatgtaaatataaaaatggaggaattgttgtataaaatatttggttgtataacgaacaaaatatataaatggggggaactttttaggaattttgtaggaaagaaagaaaaaattaatattgtcaataatgacataaagaacaatagtaataatattaatagtattaataatatgaac

SEQ ID NO: 229 - protein, 318 aa, synthetic construct
CNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMN

SEQ ID NO: 230 - RNA, 954 nt, synthetic construct
  source 1..954: mol_type = other RNA
tgtaatattccaaaaaattatactgttggagatgttttaagatatgtaaaaaataaaataattgaagatactaatataagtatgttgaatgatttacaattaaaaataatacataatgataattatatcgatataccaaaagatatgaatatagaatatgcaattaattatttgtttatatataccccttttatagtatatccatcagatgatatattaaaatataataatataattgatttaattttatataataccattatagatataaaagaaaactcttttgttaaaaaagacattccgatatatgttaagaaaggtaatatttttaaaaatgttagtttaaaatatatacctataaatataaaagaagatgaattattaacatatttattacattatttaacagataatcatgaggttgtttcttttataagagatataacatgtatatgtgtttatcctttatgtgattcaatatatatatttcatgaaaagaaacaattatcattaataccttctatatcttatcatatacaattaaaaaatgtttattatatatctacagtagaaacatatgaaaatttttataatatattaggtcatatggaattaaatttgaataagttcttacatatacaaaatgatggaaataattataagaaaaaagaagcatataataatatttattatatgaataatgtggatgaattgatacatattgatgttaatataatatttccaagcattcaaaaaataatacgtgtaaagaatttgaatgtaaatataaaaatggaggaattgttgtataaaatatttggttgtataacgaacaaaatatataaatggggggaactttttaggaattttgtaggaaagaaagaaaaaattaatattgtcaataatgacataaagaacaatagtaataatattaatagtattaataatatgaac

SEQ ID NO: 231 - DNA, 954 bp, synthetic construct
  source 1..954: mol_type = other DNA
tgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaac

SEQ ID NO: 232 - skipped

SEQ ID NO: 233 - RNA, 954 nt, synthetic construct
  source 1..954: mol_type = other RNA
tgcaacatccccaagaactacaccgtgggcgacgtgctgagatacgtgaagaacaagatcatcgaggacaccaacatctccatgctgaacgacctccagctgaagatcatccacaacgacaactacatcgacatcccgaaggacatgaacatcgagtacgccatcaactacctgttcatctacaccccgttcatcgtgtaccccagcgacgacatcctgaagtacaacaacatcattgacctgatcctgtacaataccatcatcgatatcaaagagaacagcttcgtcaagaaggacatccccatctacgtgaaaaagggcaacatcttcaagaacgtgtccctcaagtacatcccgatcaacatcaaagaggacgagctgctgacctacctgctgcactacctgaccgacaaccacgaggtggtgtccttcatccgggacatcacctgtatctgcgtgtaccctctgtgcgacagcatctacatcttccacgagaagaagcagctgtctctgatccccagcatcagctaccacatccagctcaagaatgtgtactacatcagcaccgtggaaacctacgagaacttctacaacatcctgggccacatggaactgaacctgaacaagttcctgcacatccagaacgacggcaacaactacaagaagaaagaggcctacaacaatatctactacatgaacaacgtcgacgagctgatccacatcgacgtgaacatcatcttcccgagcatccagaagatcattcgggtcaagaacctcaacgttaacatcaagatggaagaactgctgtacaagatcttcggctgcatcaccaacaagatctataagtggggcgagctgtttcggaacttcgtgggcaagaaagaaaagatcaatatcgtgaacaacgacatcaagaacaactccaacaacattaacagcatcaacaacatgaac

SEQ ID NO: 234 - protein, 9 aa, synthetic construct
YTVGDVLRY

SEQ ID NO: 235 - protein, 9 aa, synthetic construct
SIYIFHEKK

SEQ ID NO: 236 - protein, 9 aa, synthetic construct
KIFGCITNK

SEQ ID NO: 237 - DNA, 612 bp, synthetic construct
  source 1..612: mol_type = other DNA
ttttgtaatcgttacatgacagaagaaccatataataatatttttgagataataaaacctgagaatttatatagttctttattattattgtcaaatgaaaaggaaaatgattttcctagttctacaaattgtaacggatatatgaaatgtatccccttttataataatgtttctgaaagatggaagagatataattttatacaactttatataattagaagtgcattaaatatacatgtgatgagtaaatataatatgttaaataaatataataaggaaacgaacagattattaaaaagaaataataatgtagaaaatagaattaataatatatccaatcattatttatgttcaggttttaaaaaagaaaaccgtttattcttccttttgttttataaaacaattaagatgatgaagttatatataaggaatttgtttatgaaatatattaaaatttattataaaacaaaacattttgagaagaatatagaaactaataaaaaagtggtttatgttgaaagggataatctttttgatattgaaaggaataatctttttgatattttatatatgttaaaaagaatagatagttatgtaaagaatatatattca

SEQ ID NO: 238 - protein, 204 aa, synthetic construct
FCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYS

SEQ ID NO: 239 - RNA, 612 nt, synthetic construct
  source 1..612: mol_type = other RNA
ttttgtaatcgttacatgacagaagaaccatataataatatttttgagataataaaacctgagaatttatatagttctttattattattgtcaaatgaaaaggaaaatgattttcctagttctacaaattgtaacggatatatgaaatgtatccccttttataataatgtttctgaaagatggaagagatataattttatacaactttatataattagaagtgcattaaatatacatgtgatgagtaaatataatatgttaaataaatataataaggaaacgaacagattattaaaaagaaataataatgtagaaaatagaattaataatatatccaatcattatttatgttcaggttttaaaaaagaaaaccgtttattcttccttttgttttataaaacaattaagatgatgaagttatatataaggaatttgtttatgaaatatattaaaatttattataaaacaaaacattttgagaagaatatagaaactaataaaaaagtggtttatgttgaaagggataatctttttgatattgaaaggaataatctttttgatattttatatatgttaaaaagaatagatagttatgtaaagaatatatattca

SEQ ID NO: 240 - DNA, 612 bp, synthetic construct
  source 1..612: mol_type = other DNA
ttctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagc

SEQ ID NO: 241 - skipped

SEQ ID NO: 242 - RNA, 612 nt, synthetic construct
  source 1..612: mol_type = other RNA
ttctgcaaccggtacatgaccgaggaaccctacaacaacatcttcgagatcatcaagcccgagaacctgtacagcagcctgctgctgctgagcaacgagaaagagaacgacttccccagcagcaccaactgcaacggctatatgaagtgcatccccttttacaacaacgtgtccgagcggtggaagcggtacaacttcatccagctgtacatcatcagaagcgccctgaacatccacgtgatgagcaagtacaacatgctgaacaagtataacaaagagacaaaccggctgctgaagcggaacaacaatgtggaaaaccggatcaacaatatcagcaaccactacctgtgcagcggcttcaagaaagaaaaccgcctgttcttcctgctgttctacaagaccatcaagatgatgaagctctacattcggaacctgttcatgaagtacatcaagatctactacaagacgaagcacttcgagaagaacatcgagacaaacaagaaggtggtctacgtcgagcgggacaacctgttcgacatcgagaggaacaatcttttcgatatcctgtacatgctcaagcggatcgacagctacgtcaagaacatctacagc

SEQ ID NO: 243 - protein, 9 aa, synthetic construct
ALNIHVMSK

SEQ ID NO: 244 - protein, 10 aa, synthetic construct
ALNIHVMSKY

SEQ ID NO: 245 - protein, 13 aa, synthetic construct
NVENRINNISNHY

SEQ ID NO: 246 - protein, 9 aa, synthetic construct
RLFFLLFYK

SEQ ID NO: 247 - protein, 11 aa, synthetic construct
KIYYKTKHFEK

SEQ ID NO: 248 - DNA, 465 bp, synthetic construct
  source 1..465: mol_type = other DNA
atgaaggatgctgttgaaataaatgacattactagcaaacttattgaagaaactcaagagttaaatgaagtagaagcagatttaataaaagatatggaaaaattaaaagaattagagaaagcattatcagaagattctaaagaaataatagatgcaaaagatgatacattagaaaaagttattgaagaggaacatgatataacgacgacgttggatgaagttgtagaattaaaagatgtcgaagaagacaagatcgaaaaagtatctgatttaaaagatcttgaagaagatatattaaaagaagtaaaagaaatcaaagaacttgaaagtgaaattttagaagattataaagaattaaaaactattgaaacagatattttagaagagaaaaaagaaatagaaaaagatcattttgaaaaattcgaagaagaagctgaagaaataaaagatcttgaagcagatata

SEQ ID NO: 249 - protein, 155 aa, synthetic construct
MKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADI

SEQ ID NO: 250 - RNA, 465 nt, synthetic construct
  source 1..465: mol_type = other RNA
atgaaggatgctgttgaaataaatgacattactagcaaacttattgaagaaactcaagagttaaatgaagtagaagcagatttaataaaagatatggaaaaattaaaagaattagagaaagcattatcagaagattctaaagaaataatagatgcaaaagatgatacattagaaaaagttattgaagaggaacatgatataacgacgacgttggatgaagttgtagaattaaaagatgtcgaagaagacaagatcgaaaaagtatctgatttaaaagatcttgaagaagatatattaaaagaagtaaaagaaatcaaagaacttgaaagtgaaattttagaagattataaagaattaaaaactattgaaacagatattttagaagagaaaaaagaaatagaaaaagatcattttgaaaaattcgaagaagaagctgaagaaataaaagatcttgaagcagatata

SEQ ID NO: 251 - DNA, 465 bp, synthetic construct
  source 1..465: mol_type = other DNA
atgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatc

SEQ ID NO: 252 - skipped

SEQ ID NO: 253 - RNA, 465 nt, synthetic construct
  source 1..465: mol_type = other RNA
atgaaggatgccgttgagatcaacgacatcaccagcaagctgatcgaggaaacccaagagctgaacgaagtggaagccgacctgatcaaggacatggaaaaactgaaagagcttgagaaagccctgagcgaggacagcaaagagatcattgacgccaaggacgataccctcgaaaaagttatcgaggaagaacacgatatcaccaccacactggatgaggtggtcgagctgaaggacgttgaggaagataagatcgagaaagtgtccgacctgaaggatctggaagaagatatcctgaaagaagtgaaagaaatcaaagaactggaaagcgagatccttgaggactacaaagaactcaagaccatcgagacagacatcctcgaagaaaagaaagagatcgaaaaggaccacttcgagaagttcgaagaagaagccgaggaaatcaaggacctcgaagccgacatc

SEQ ID NO: 254 - protein, 9 aa, synthetic construct
KLIEETQEL

SEQ ID NO: 255 - protein, 9 aa, synthetic construct
VEADLIKDM

SEQ ID NO: 256 - protein, 9 aa, synthetic construct
EEHDITTTL

SEQ ID NO: 257 - protein, 9 aa, synthetic construct
TTLDEVVEL

SEQ ID NO: 258 - protein, 8 aa, synthetic construct
TLDEVVEL

SEQ ID NO: 259 - protein, 10 aa, synthetic construct
ELESEILEDY

SEQ ID NO: 260 - protein, 9 aa, synthetic construct
LESEILEDY

SEQ ID NO: 261 - protein, 11 aa, synthetic construct
KTIETDILEEK

SEQ ID NO: 262 - protein, 9 aa, synthetic construct
ETDILEEKK

SEQ ID NO: 263 - protein, 48 aa, synthetic construct
SKKKNKKGSGEPLIDVHDLISDMIKKEEELVEVNKRKSKYKLATSVLA

SEQ ID NO: 264 - RNA, 144 nt, synthetic construct
  source 1..144: mol_type = other RNA
agcaaaaaaaaaaataaaaaaggatcaggtgaaccattaatagatgtacacgatttaatatctgatatgatcaaaaaagaagaagaacttgttgaagttaacaaaagaaaatccaaatataaacttgccacttcagtacttgca

SEQ ID NO: 265 - DNA, 144 bp, synthetic construct
  source 1..144: mol_type = other DNA
tccaagaagaagaacaagaaaggctccggcgagcccctgatcgacgtgcacgatctgatctccgacatgatcaagaaagaggaagaactggtcgaagtcaacaagcggaagtccaagtacaagctggccacctctgtgctggct

SEQ ID NO: 266 - skipped

SEQ ID NO: 267 - RNA, 144 nt, synthetic construct
  source 1..144: mol_type = other RNA
tccaagaagaagaacaagaaaggctccggcgagcccctgatcgacgtgcacgatctgatctccgacatgatcaagaaagaggaagaactggtcgaagtcaacaagcggaagtccaagtacaagctggccacctctgtgctggct

SEQ ID NO: 268-286 - skipped (19 entries)

SEQ ID NO: 287 - protein, 574 aa, synthetic construct
MNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENIIDNNPQEPSPNPEEGKGENPNGFDLDENPENPPNPPNPPNPPNPPNPPNPDIPEQEPNIPEDSEKEVPSDVPKNPEDDREENFDIPKKPENKHDNQNNLPNDKSDRYIPYSPLSPKVLDNERKQSDPQSQDNNGNRHVPNSEDRETRPHGRNNENRSYNRKHNNTPKHPEREEHEKPDNNKKKAGSDNKYKIAGGIAGGLALLACAGLAYKFVVPGAATPYAGEPAPFDETLGEEDKDLDEPEQFRLPEENEWN

SEQ ID NO: 288-289 - skipped (2 entries)

SEQ ID NO: 290 - protein, 1162 aa, synthetic construct
MKHILYISFYFILVNLLIFHINGKIIKNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEQERLAKEKLQGQQSDLEQERLAKEKLQEQQSDLEQERLAKEKLQEQQSDLEQDRLAKEKLQEQQSDLEQERRAKEKLQEQQSDLERTKTSKEKLHEQQSDLEQERRAKEKLQEQQSDLEQERLAKEKLQEQQSDLEQERLAKEKLQEQQSDLEQERLAKEKLQEQQSDLEQERLAKEKLQEQQSDLEQERLAKEKLQEQQSDLEQDRLAKEKLQEQQSDLEQERLAKEKLQEQQSDLEQERRAKEKLQEQQSDLEQERRAKEKLQEQQSDLEQERRAKEKLQEQQSDLEQERRAKEKLQEQQSDLEQERLAKEKLQEQQSDLEQERRAKEKLQEQQSDLEQERLAKEKLQEQQSDLEQERRAKEKLQEQQSDLEQERRAKEKLQEQQSDLEQDRLAKEKLQEQQSDLEQERRAKEKLQEQQSDLERTKASTETLHEQQSDLEQERLAKEKLQEQQSDLEQERLAKEKLQEQQSDLERTKASTETLREQQSDLEQEKLAKEKLQGQQSDLEQERLAKEKLQGQQSDLEQERLAKEKLQGQQSDLEQERLAKEKLQGQQSDLEQERLAKEKLQERQSDLEQERLAKEKLQEQQSDLEQERLAKEKLQEQQSDLEQDRLAKEKLQEQQSDLEQERLAKEKLQGQHSDLERTKASKETLQEQQSDLEQERLAKEKLQEQQSDLEQERRAKEKLQEQQSDLEQERRAKEKLQEQQSDLEQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 291-292 - skipped (2 entries)

SEQ ID NO: 293 - protein, 165 aa, synthetic construct
ERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEE

SEQ ID NO: 294-295 - skipped (2 entries)

SEQ ID NO: 296 - protein, 132 aa, synthetic construct; identical to SEQ ID NO: 155

SEQ ID NO: 297-298 - skipped (2 entries)

SEQ ID NO: 299 - protein, 1558 aa, synthetic construct
MTNSNYKSNNKTYNENNNEQITTIFNRTNMNPIKKCHMREKINKYFFLIKILTCTILIWAVQYANNSDINKSWKKNTYVDKKLNKLFNRSLGESQVNGELASEEVKEKILDLLEEGNTLTESVDDNKNLEEAEDIKENILLSNIEEPKENIIDNLLNNIGQNSEKQESVSENVQVSDELFNELLNSVDVNGEVKENILEESQVNDDIFNSLVKSVQQEQQHNVEEKVEESVEENDEESVEENVEENVEENDDESVASSVEESIASSVDESIDSSIEENVAPTVEEIVAPTVEEIVAPSVVESVAPSVEESVEENVEESVAENVEESVAENVEESVAENVEESVAENVEESVAENVEESVAENVEEIVAPTVEESVAPTVEEIVAPSVEESVAPSVEEIVVPTVEESVAENVEEIVAPSVEEIVAPSVEEIVAPTVEESVAPTVEEIVAPSVEESVAPSVEEIVVPTVEESVAENVEESVAENVEEIVAPSVEEIVAPSVEEIVAPSVEEIVAPSVEEIVAPSVEEIVAPSVEEIVAPSVEEIVAPSVEEIVAPTVEEIVAPTVEEIVAPSVEEIVAPTVEESVAENVATNLSDNLLSNLLGGIETEEIKDSILNEIEEVKENVVTTILENVEETTAESVTTFSNILEEIQENTITNDTIEEKLEELHENVLSAALENTQSEEEKKEVIDVIEEVKEEVATTLIETVEQAEEESASTITEIFENLEENAVESNENVAENLEKLNETVFNTVLDKVEETVEISGESLENNEMDKAFFSEIFDNVKGIQENLLTGMFRSIETSIVIQSEEKVDLNENVVSSILDNIENMKEGLLNKLENISSTEGVQETVTEHVEQNVYVDVDVPAMKDQFLGILNEAGGLKEMFFNLEDVFKSESDVITVEEIKDEPVQKEVEKETVSIIEEMEENIVDVLEEEKEDLTDKMIDAVEESIEISSDSKEETESIKDKEKDVSLVVEEVQDNDMDESVEKVLELKNMEEELMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADILKEVSSLEVEEEKKLEEVHELKEEVEHIISGDAHIKGLEEDDLEEVDDLKGSILDMLKGDMELGDMDKESLEDVTAKLGERVESLKDVLSSALGMDEEQMKTRKKAQRPKLEEVLLKEEVKEEPKKKITKKKVRFDIKDKEPKDEIVEVEMKDEDIDEDIEEDVEEDIEEDKVEDIDEDIDEDIDEDIGEDKDEVIDLIVQKEKRIEKVKEKKKKLEKKVEEGVSGLKKHVDEVMKYVQKIDKEVDKEVSKALESKNDVTNVLKQNQDFFSKVKNFVKKYKVFAAPFISAVAAFASYVVGFFTFSLFSSCVTIASSTYLLSKVDKTINKNKERPFYSFVFDIFKNLKHYLQQMKEKFSKEKNNNVIEVTNKAEKKGNVQVTNKTEKTTKVDKNNKVPKKSRTQKSK

SEQ ID NO: 300-301 - skipped (2 entries)

SEQ ID NO: 302 - protein, 106 aa, synthetic construct
MKTIIIVTLFILILNTIIINPCTCVPNTLPIEYFDYETLKENVKIRDSNTKTQLIILSAVCASILLSLSSLWGIRIHYNHEMNKKEINNYELNSDLLKDADFYMVE

SEQ ID NO: 303-304 - skipped (2 entries)

SEQ ID NO: 305 - protein, 302 aa, synthetic construct
MWLCKRGLSVNDTTKCDVPCKDFYMLFLSNKKEKIKCGTFFGYIFLSKFMKLSISLLLLALIQNILLSNVSLISGSHLYKRNSRKFAEGYMKGSGSEKNVYLSNKNKEINMNQQSDNKMCDECDDMNQPGDVNKNDKTSNDQANSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTN

SEQ ID NO: 306-307 - skipped (2 entries)

SEQ ID NO: 308 - protein, 3597 aa, synthetic construct
MKLSVLYLVVSLYFFDRKIVCNKKNGKSKSNNHNYFNNTTKHIDKQVIYNLPENDNLFHVLKYLIHGENKNILYDVDESLYTKIENKEYITKEDLLRTLALIEKKNVDCNDNIKKVKIAKLLIDINDLLRTNYIILDDYKEINKNDLTKNGFLLKLQNSLNKRDMVNSKRVNQDIILRNQYLEKNYKLCSISSDMLIISTPRNLNISDTSIKEIKHPSSINIMNEIFCDNVISSNVSNISPLQLNKSIGLYKPLLDLDIKSRIDEYRKKIITYYQDSMNEIFYLIRDEFNEIKKKFYLYIHPSKMPEAKPNNDIFKKEDMEGNEIITYVSNKIENKENIKNKEEYILKSNNNNVIKISSKRTKKVINIPIKKKKLSNDIKENVNSSTKSDNNNIISNETVDACTKIESENGNKKKDDNVKNINRIYHIFLKKKNNEKKCDISKQTLYNNSFKENDVIKTFTHNDNILKKIIIIKDVSREKPTIKEIIHQQPLPVENSELYNISEHIQNKCNCCFYSIPLIHIYHPNGSMHCLSCSQYEKNVNRKCPIYEKYITFNYNDTCLLCVPSSYITNNCFCNLIHSKGLGEKTNDVPKKLNMMPHFSSIIDVYGNKNIDGYNNFENDNNLLVYSNNDEPILKKNHKERPNKKCSNRYKLKCPVGNGSSDMFSGQSISIFPIIGDDINRGHYENCPYYDEEDVMCYNTITNKKININNEDDNYEKILYDKDKIVYLNSSSFDTINIKIEGEDLNMNDRNEENDLLDKESIEYINEINEKEYYDSIEACKDYRYHTFEESIKNVNNRECYKNVYYSTVHKKEDNIDKKCSHDIESRNKRVNEYEDECENDNNNNNNNNDDDDGEERNYFLFDLRFIYKTGFFTKRKTIINSYLKTNKIDITYNKEKILYLFKNLFEKTFKDINYQIKESLVHMFPIVQNSEIQGFDIDFGKVKLHYATSINMFKYSVFDLINMKIYFISPDKTYILLEDIIREIREEKENSNNDGSRRKNKIINKYLHEMDITNIEWSTENSKTNTTNSESKTTTTRENTIVIKEKIGTHDEKKKKCDNTKNNRNNNDDNDDNDNDNDNNNDNNNDNNNNNNNNNNNNNNNNNNNNNNSNSNSCDNNNKRDNDSFEHVDIQKNDTITTNIDCNIIHNNTIDSSNKNRIKYNLNILKFLLKYKDLLSKEDINMISDKNNLVTINKNDKTNIGDEIIEITIRKNEKRNIENNCTYTNNSIINNIDNNISESININGGEGINAVIETVDKYLDKYFSVHNMPIYYMENMKISKNIIYVTSDVNSDLDMLKMSILGICNITNKSIDNFNFFLVRNEAELYDPNKCQKIPEHIKNVQDLYNYSKMLDMKIIIAPVYAEDIDIIPKSFDYLFKLYKDGDDEKGVTTDSRDNDCQQKGNVDTQERNKTENDTTRITLSNIKHNNIIKNNNKNNNNNNNNSHNNNHSYYNHELSLFNDAESNKPNDNTTTSNDKNYYFFNYDNKKSLILNDPENDENIEYLSEEISIYEDEHIYDEGNLYNSFVIYIDNPFKRINIENAPILKGYTLNLFINDIMNKLIHIPSTFINTQSYKKGMCVMSICYCFNYYSLNIYTNNIILLNFDKDVTLYEVIKEFKSRENEYLLIKNKMNEECQDVDNLSNIKLPHTIDLIDSKYIHLEFPLLYIDEHSNFFQDKITLMNVPENLTVYELCKSMKNILNESLISFNLSTINDIKIYTIRGRQWEMVLDNLLVFGLKINYEDIFTILIDNNLLTKNYYELLSNIIIDFRDNNMYIKKLDIYIDYIFQPYTIYNIPIYITPNNLKYILLQYTNTFLSNSLLDEFFFTYNKRHTTINVVPEVRNWEHVEDSDVYIYEENDINDKKKEQKRNISFIYVLNMLSTFYKIKFNSSKLLESVMGEIHQLCGKRFNENLWLDIKKSTKNEKDIEIYLENSSKKVLIKNVPLDLLVWKLINIIMRGSFNIPIEDKEKLYNLFMLVPKNKDKIKDKNVDYYFLSTPGYTVEEMLRVFEKKKKPKKKGKEKNSANDNNNNNNNDNNNDNYNDNYNNDNNINSNNNYGDMKQTYYKILLIKAYPQHLHGIQNNERFNEYLTFNLEALPSVIDVKNPTTCLSFYVNYKNENKMTHIINVPENISTDMLLKTILSDNFCRWKKLPNEEKMKFSLHLNKKEITNIKDYINNGDIAQLKTFILINNKKRRVQEKYIDLDNTTELGSNVNNISKEELKEIQLYDNVLREILNKNYIDYNNISLSGYTITVYVFDGNKYEPLTIYNVPLNITNKDIINFLLIKSNHINITKLIDEFLLLNEKLYNTYIVKNKNKNMLEEQNVLFIKKLNEEEEVFQLNESKFYLVHNPLFNPLDNLLQNIVHKKYDFKSEKIDIHSVGEKFEILTLNILINNDINNMHNNNVNNNYVNNNNINNNNINNNNINNNNINNNNINNNNFSFGKKKFVITVLNIPYNMYITEFLRYSIGYQRKWIYKFVLFYIIKGSEKYILNDGNDIIRHGKTVSEIFQICKSGDLCTLHIDINFNEADNNLENDIFNEEINAKEMKNVDVDSHADVDVDLDANVYLDANADVEGNINKFVNSLHNDNENRFIVLKELSKKIYTDDYENKKIDMSIICFEYNAGLYERNLFGSSRICNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNSINNMNNINNMNSINNINNINSINNINNCDNIDRIDNKVLKDYLCEEQNIITFIFCSHDNSIHDYLTSRMIYLPAIINYQTNYITFYSSINGFANNETTFHGFPNYLSPETMLTMLQYNFFHVIGKNYLKLYGSLYDKGTDEDIIYNTNDIRKSENKDIYNKNLIAMRGAIKFILRIKNKGKKKSLDTIQNLINNELNIACENVENDVDMEECNDFLSSLNNSSIFWKNGILGFMANSIVTRDNMNNSLILPYVDFEINEKMFLQNILYHINISPYLYKLFELTHIDKKCNTYKKKLKNSVAHIQCLLSYGFSIYFDLHYDSTFDKYKGFTLGDNSFDITQIHSFQNLHEQNFVDLILYNHDNTFITIKNVYRDMTVATFLNDLSKTRCHILGLTYNQISTYYKLTYEIYNETHDISPNLSIADVHNIVISFSKSSILLKIRKIDITNDISHNNNNNNNNDSDFIINSDNIDYINALPDLYLSPYPPVIDLSQNNFQVNIYMHSKFIPNDYSLRKDIVPHITINNVPSNVFILSLKELLKTIFIQHMEKSQTTTFQLNLYDFEVEFFILTFNRFTSKFHKMNSNVLSNLTISNFYSTYIHGGLFLNIDKIKIDIPNLLNDFIDHFSKVVIDFSHHNIVKQ

SEQ ID NO: 309-310 - skipped (2 entries)

SEQ ID NO: 311 - protein, 1964 aa, synthetic construct
MEKGSILSFIFFCSVVIFIRFIGYFFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSIISNNFLYVIRIIFLPFEKIYFSLKSLIMIKKMNMSSSYYYYYVNMFSLYKKNYNKYEEIFIHEQRVIYPNEYLKNEMLDKYRRVIRILSGQHDNPFIDSLLINPEKIEKDDLDVKQKKKKIIEELKKKKENTNTNTNTSTNTSANTNTSTNTSANTNTSTKESHILDESKLETFYRDELDKMGKEEIETYFKGNIDKKSLDEFHKILLEELNKMDKDELYEMYREELNRIEQEKIRNMNKEEINKTYKDEINNMNSDQVDKIHREELEKIEKEKINKMDKDEIDKIYREELDKMDRDAIYSMYIEDISNKNIKDLIKNEKETNKDKNKKKDIDINKKKKKDIDIDVDIDKDIHKDHVEELYGEVKNKLSKEELDRMDRDALYRVYLEELDRMNRDELYRVYLEELEKIDKEEKEKIHREKLHKIEKEKINKMDKDQIDKIYEEELNKMDSDEIQHVRRAILQDIQKEKIQNLELEEIDRLYKEELDRMDREARYEIPMRNLSRNEKDNIIHRNIKNESNQKNKKENVNVFIIHDNNDSNNNNNNNNRDVNNLNNKHTNNNYNENVEVELVVRNLDKDKGAKIEDIIDYFNKEIKKDKNVNVSNIVNFLNSKVGKDNTPIQHKKENQVDVVRKNIQIIQEDNIKNKGQKDNTEMLDNNKEITNIDIKNVDDIKNVGDIKSVGDIKSVDDINNVDGIKNVDGIKNVDGIKNVDGINNVGDINNAGDTNNAGDINNVGDINNSVDIYNVEHIDEAEKKPNLDNPKKFDWTQVFKDKVTEKIKNEEKFNNSKENIQNDIRDKEIHKDDRIKGITSREKNAEEINNNEKKDKFVYEFYTSNKKENIDKEEENNIDDKNIKIEIEPNYEINNNFEEENKNEINVIIDKEAKNNMDKDDSNNNNNIQKNNIIIKDNTNVSEEVHITESSKEIAEFFNNIIKNSNILDMCSKMNASDSEKGFICINGNNYIINPGTYHIINIKYPDYNNVRKKWYDSMDCISINNKDENNNNKEHNYYNKNDDDLYLKKSVEEFIPGFLSNINKVDDLARIFTPSFIQNDIFLNCIYKYRNDFDKNNNIYSFPMKIFLRKNSTKIKGCSFQIDEDPLLYKDYSEKESFLSNKIILNNSNRNTECVLHASNEIVGFQCGPPYKSYDNIQYRHLTNKSNDIQKNIFGIYSNNNSSYSHLFKNIFNNEHKLYNVGGYFQTEPINCFEFVNDNINVEDILPGAVPFPRFDLIHHDLDVNQTRYILLNETNQDKTISCTCNYFTEPNIVYTGKIIIKVEEEKIYKTKKLQTEFNDIINNKKEIYHEKKMNHIIKEKKDEDENDMSFNKNYVNNYNENFKINDINNFYNRNNHPNNNYHNDYHNNHSSKGNHTNKIHDTFLKNKYNISFNNLKFYNIKHENKNNQDVINYEYNVDDYHEVQDGQDESFKEEEDFIDFKENIINDNTNHNNTDLDDDKYNKYHNNNNNNNNNSSFKSIESNLDLQRSILQSGDTQQVVVINKSKNVNVLFPDKKKNHTHPNEEKRTFPKYISLVYKEKNKDNEKIDKTLTFIKEFYPPLLRGGKDSDPENKQTGVEINNGVEKKNDVQIKNDVEIKNDVEIKNDVEINDDVEIKNDVEIKNDVEIKNDVEINDDVEINDDVEINNGVEINDGVENKDNIHEGNNNLENDSFNEDTIEEPFENIFDFINEETSSNENSEIILDSADSIKRKLGHNFLDIISAGKFKIRHKEKKTKNKKKK

SEQ ID NO: 312-313 - skipped (2 entries)

SEQ ID NO: 314 - protein, 162 aa, synthetic construct
MKILSVFFLALFFIIFNKESLAEKTNKGTGSGVSSKKKNKKGSGEPLIDVHDLISDMIKKEEELVEVNKRKSKYKLATSVLAGLLGVVSTVLLGGVGLVLYNTEKGRHPFKIGSSDPADNANPDADSESNGEPNAGPQVTAQDVTPEQPQGDDNNLVSGTEH

SEQ ID NO: 315-316 - skipped (2 entries)

SEQ ID NO: 317 - protein, 287 aa, synthetic construct
MKVSYIFSFFLLFFVYKNTNTVVCDNGYGDLAATSALTTVIKDPISLTIKDIYEHGVKNPFTKIIHKLKKFIRYRKVLRWSRMWWVLLVREIVGDNTIEKKTEKALREIWDQCTIAVYNNTLNAVESKPLLFLHGILNECRNNFATKLRQDPSLIVAKIDQIIKSQIYRFWVSEPYLKIGRSHTLYTHITPDAVPQLPKECTLKHLSSYMEEKLKSMESKKNIESGKYEFDVDSSETDSTKDDGKPDDDDDDDDNFDDDDNFDDDTVEEEDASGDLFKNEKKDENKE

SEQ ID NO: 318-319 - skipped (2 entries)

SEQ ID NO: 320 - protein, 151 aa, synthetic construct
MCSTYSVEPLVYDSYEYVYLKPKKAGTSAVYYPLNISWKYVAKKRSVGCFGSRKKYTLTPEAYYYPYYYYYVYYYPSAARLVRTTRKEKVLKENNNKESEDENKQDNVGTEKKECDCSEKEKYIPTYVPLTESYYFPPSALYVPHYSVLVP

SEQ ID NO: 321-322 - skipped (2 entries)

SEQ ID NO: 323 - protein, 863 aa, synthetic construct
MSSILLFFVVFQYLLISFSGTSYKRFVVDGASIFLRNPYKITLGKSEKKGKVFSEFSEEEDSIVRRDTEKKKKWFGKGNDVNKKVKEKKNVILEEKQIGKEENNLVGQQNDSVGQQNDSVGQQNDSVGQQNDPVGQQNDSVGQQNDPVGQQNDPVGQQNDYVGQQNDSVGQQNDYVGQQNDYVGQQNDPVGQQNDSVGQQNDYVGQQNDPVGQQNDPVGQQNDPVGQQNDSVGQQNDYVGQQNDPVGQQNDYVGQQNDYVGQQNDPVGQQNDPVGQQNDPVEQEDNVGIVKKEKKEKIIVEKENEIGVIERENISGVEVINKDSEEDIYNSNVDKNFPADDNNRDEILVRRKKVSNKDSLFMNDIIRGENEDNDDDNEEEEEKVSKNLENVKLGELSKLQYIFGTNITDNDDDSEIRSESEIDDIDELDEEEYDWTEHVYNYKPTTYLLPGLGGSTLIAEYKNATIHSCSRYLLNSKPFRIWISLSRLLSIQSNIYCTFDTIRLKYDEKKNIYYNQPGVFIDVEKFGNLKGIEYLDYFNNTGIGITKYFNVVGQYFTSHGYVDGESIIGAPYDWRYPLSQQNYKILKEHIEYIYEKRNGTKVNLIGHSLGGLYLNFFLSRVVSKKWKQKHLSKIIFISTPFKGSVKTIRALIQSRKDFISFRITKLIKLSIPESMMKALGNSLGSLFDILPYREYYKRDQVVILINMSNTPIDEDHVQYLVTLCGIYKPECYRNRADVNLKVYTLKNWHELLDDKLKAKYENYKLYRERYYNKDHGVPIYCLYSTINKKETEYLLYFETQNTREEPTIYYGTGDGTVGTESLQACSNFYNTVLTHHFSDTSHVGILYTNETAKYIYDIVESPN

SEQ ID NO: 324-325 - skipped (2 entries)

SEQ ID NO: 326 - protein, 969 aa, synthetic construct
MKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

SEQ ID NO: 327-328 - skipped (2 entries)

SEQ ID NO: 329 - protein, 362 aa, synthetic construct
MIIPFHKKFISFFQIVLVVLLLCRSINCDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYSSSQGIYNIHTIYYANYE

SEQ ID NO: 330-331 - skipped (2 entries)

SEQ ID NO: 332 - protein, 1086 aa, synthetic construct
MFRIFFTLLIIILIKKTSAIDLIEGIFYEKNEIDKLTFSLDHRVRDNLKTDLILNNNGENDYAYLNKYVYTILNRDSTEKIKTFFSHNKDMKSCDYFISKEYNSSDKTNQICYKKTFCGVVIPNSEEIKTNKITNDKLYCAHFNSTHIIIYYISQPLLLEPHVVYEETFFEKGKNDQINCQGMYISLRSVHVHTHNAILQQETLTYIKNLCDGKNNCKFDFDSIKYENKSLTHYLFFINIQYQCISPLNLQENEMCDVYNDDTHKATCKYGFNKIELLKNVCEENYRCTQDICSVNQFCDGENETCTCKTSLLPSAKNNCEYNDLCTVLNCPENSTCEQIGNGKKAECKCENGKYYHNNKCYTKNDLELAIKIEPHKKEKFYKNNLYQGKALKPEYIFMQCENGFSIEVINAYVSCYRVSFNLNKLKYVTESLKKMCDGKTKCAYGNTIDPIDDLNHHNICNNFNTIFKYDYLCVFNNQNITSDKNSHLHSNIPSLYNSSILPDINKSKFHLISRNSRTNQYPHNNISMLEIQNEISSHNSNQFSTDPHTNSNNINNMNIKKVEIFRSRFSSKLQCQGGKINIDKAILKGGEGCNDLLLTNSLKSYCNDLSECDIGLIYHFDTYCINDQYLFVSYSCSNLCNKCHNNSTCYGNRFNYDCFCDNPYISKYGNKLCERPNDCESVLCSQNQVCQILPNDKLICQCEEGYKNVKGKCVPDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDKCVHNKKCSENSICVNVMNKEPICVCTYNYYKKDGVCLIQNPCLKDNGGCSRNSECTFKYSKINCTCKENYKNKDDSCVPNTNEYDESFTFQYNDDASIILGACGMIEFSYIYNQIIWKINNSKESYVFYYDYPTAGNIEVQIKNEIFHTIIYLKKKIGNSVIYDDFQVDHQTCIYENVFYYSNQN

SEQ ID NO: 333-334 - skipped (2 entries)

SEQ ID NO: 335 - protein, 791 aa, synthetic construct
MVDFNDLSVELKKTELIKEDLRNLSHIINNEFSYFCQNENKNVSFNNNISSYYNDDIFSKSTLNNLYTSWKLEDFSHFDFSSILDILKRNQYVMCSIYFLLIFSCIYFLTLLLYTKCIKTTLKKWFCRYCSENINENNSNHNEQRTVLQNVINKSCYFITYSSIICLLLFLLLSGITYMHYFIKTKKGIHSNICNIYTRLDKFLLNKCLDPKKVDTSCYSAEHILNDLSSILEEYKKVKQQAKDDTLLDENTPFPLLERYITTFNKLNVLKNNINKNNTTLENEYFHTYPALKGISETLTTIISEGNKNFGNARNVIKEVKSTIKYSFHTVDETIRNVFKDSVPKITGLITQAGKSIKGINNKYKIKERIPKYTNIILLTNIILLLPPFLILLGIIIFMIFILMGYIQKNNNFFIKLFGHFSAYFGLLTIIILSFGILFLSTSVIGGTSCILSERILKNELRFDILNNTLIDYCIKNESAPLIDDDITTSFVAKINSFDTGHIDHNINEYEKHFTILKESFFHKSLKFMDYIWIVIMKRENNTFLNRIRTEQVKKSLLITGIINENIKYENMEAIGIRSYLTTLNKIIFPENNGKICFNDIICEKENNTYNITENSKTTDQKYRNIRDGMDEHLRNDLDAIVQLFVYKARILKENIFDINDLDSNEKNKIGWSEYTPRNINGTQKKSIINTFLVNVIESINFSEIINFFDKMRDQFNVLKDLILLKIDTLTENTKCNKLVKELINVRKDYCNNVVLNLSTLSVYLIIFSITSFLLWYLFLFLWFYYNIKPS

SEQ ID NO: 336-337 - skipped (2 entries)

SEQ ID NO: 338 - protein, 622 aa, synthetic construct
MRKLYCVLLLSAFEFTYMINFGRGQNYWEHPYQNSDVYRPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSNTTFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYTYLSKNVVDNWEKVCPRKNLQNAKFGLWVDGNCEDIPHVNEFPAIDLFECNKLVFELSASDQPKQYEQHLTDYEKIKEGFKNKNASMIKSAFLPTGAFKADRYKSHGKGYNWGNYNTETQKCEIFNVKPTCLINNSSYIATTALSHPIEVENNFPCSLYKDEIMKEIERESKRIKLNDNDDEGNKKIIAPRIFISDDKDSLKCPCDPEMVSNSTCRFFVCKCVERRAEVTSNNEVVVKEEYKDEYADIPEHKPTYDKMKIIIASSAAVAVLATILMVYLYKRKGNAEKYDKMDEPQDYGKSNSRNDEMLDPEASFWGEEKRASHTTPVLMEKPYY

SEQ ID NO: 339-340 - skipped (2 entries)

SEQ ID NO: 341 - protein, 673 aa, synthetic construct
MNVLFLSYNICILFFVVCTLNFSTKCFSNGLLKNQNILNKSFDSITGRLLNETELEKNKDDNSKSETLLKEEKDEKDDVPTTSNDNLKNAHNNNEISSSTDPTNIINVNDKDNENSVDKKKDKKEKKHKKDKKEKKEKKDKKEKKDKKEKKHKKEKKHKKDKKKEENSEVMSLYKTGQHKPKNATEHGEENLYEEMVSEINNNAQGGLLLSSPYQYREQGGCGIISSVHETSNDTKDNDKENISEDKKEDHQQEEMLKTLDKKERKQKEKEMKEQEKIEKKKKKQEEKEKKKQEKERKKQEKKERKQKEKEMKKQKKIEKERKKKEEKEKKKKKHDKENEETMQQPDQTSEETNNEIMVPLPSPLTDVTTPEEHKEGEHKEEEHKEGEHKEGEHKEEEHKEEEHKKEEHKSKEHKSKGKKDKGKKDKGKHKKAKKEKVKKHVVKNVIEDEDKDGVEIINLEDKEACEEQHITVESRPLSQPQCKLIDEPEQLTLMDKSKVEEKNLSIQEQLIGTIGRVNVVPRRDNHKKKMAKIEEAELQKQKHVDKEEDKKEESKEVEEESKEVQEDEEEVEEDEEEEEEEEEEEEEEEEEEEEEEDEVEEDEDDAEEDEDDAEEDEDDAEEDDDDAEEDDDDAEEDDDEDEDEDEEEEEDEEEEEESEKKIKRNLRKNAKI

SEQ ID NO: 342-343 - skipped (2 entries)

SEQ ID NO: 344 - protein, 408 aa, synthetic construct
MLLFFAKLVVFTFFFWLLKYGKTRSYPKSGHKGHTKLNQPVVRTLADFNDMFANQKNTFNFLKHINHYKNEQDTNNTHTPNHDEYSHNLPKNHEESNANMNNHNSFNDKSVNKKEAFDQFLQTLLNNYEIMHKEDESKESNQHNYKEGPSYEDKKNMYKEILKGYYNVFFENYANDTESNVHNKPEEVHKHEEIHKHRKLHKHEEVHKPEEFHKPEEFHKHEKVHKHEEVHKPEEVHKHEENHKHEENHKPQMVGQAPPEKEIRQESRTLILGSFPQAGEILREDLWNKEDNKFSYALDPNDYASIEDKLLGSIFGYFKKNHDNLVKHLLQQINTYKHKYMELKEQYINEVMKLKKIYNKSIMVIFIASCISILGPVMLHMHQNNPEEFFATILSFSISLGLHNLLLT

SEQ ID NO: 345-346 - skipped (2 entries)

SEQ ID NO: 347 - protein, 2074 aa, synthetic construct
MMENKYPNELFCYINRYNINEIIENGEEKYVNEYDEDKNMSINHMNENDGICEYEIPFLLDYVDDSNKEDSEKNSLKSYLDDGASTILSKPDELENYNKQNENEFDENNNNKNNKIDQLKEKINIIIIPNKGVINNFEEILSMANRNDKNIEKKLNDRFYQICCKSIADINTHNLNKIKDLKKKKNNKGSLNIEHIDYGDIFLTIHDTLKSNNKIKGNNKTNLLHDSSYEIKKKTRRGTNIYKNPFHHRGSYLTSYENQKDIIYLNNLNNIMMDKYSNCSDSRKKEYSHFNSQEFSYDKYSMKDRMFLKNLYMKQNRLRDKRGKYHKLGDYQNIENYRKTGEHSFDCMNMSDIMHSNKMSHVNIMDHMIYKDNNNMSKLVDTINSREKDVKNYDDNFESYNNFFKNNNDEQHICLEYDDTYNLKDTVKNIIVEEEQCGKGVACICDKNEDVDDLFVSKKTNYSSNKKREDYEKVFLEDNLHLKQTPSKRTKINIIPDYYDNNRSNKSYKENEEDALFEVCGSLKNDDILYKDNKLNVINEDNIKEEDDKESVVHLDNDEDKKEEMYKDVYPNVLSCEKETIRRNEKYNKSLNSTSSFEKIDNPSEINVESKEDTEYFDLLIKKYEDTKINVYDNESLLLDLSNELREEMAKGDSNKNVNKVEDNDNKKENICHDNIMEDICHNNNVEDMYRNNNVEDMYRNNNVEDMYRNNNVEDMYRNNNVEDVCHNNNVEDVCHNNNVEDVCHNNNVEDVYHNNNVEDMYHDNNIEDVCHNNNVEDVCHNNNVEDHVNYDNEELNKKMDEMKEEKEERNEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLNLSDLNELNVDDINNNFYMSTPRKSIDERKDTECQTDFPLLDVSRNTNRTPRRKSVEVILVEKKLKKKKQKCMDKYTDANEDSNRRYPKRNRIKTLRYWIGERELTERNPYTGEIDVVGFSECKNLQDLSPHIIGPIEYKKIYLKNLNSNEHEENEDNNGDIIENNNGDVIENNNGDIIEDNNANEKNHNNLESEGKGIVYDDVNNLHVHTNSDNSAHSKKIKGAPSRFSNTNNGRKKRRRRKFINVVNYIKKKKKKKLIKSMDNMEVTDNFKNDMSDENKQSGDENKQSGDENKQSGDENKQSGDENKQTNNDIKQSDNDIKQSDDIYMNEDMNLFNDLNDNFDNNEYFINNGDKDSHAEEEMAIENIQSKSIEKDILNNEEQDNNNIFDIDNELIDMKDGNVDEMESDEKLKTFEKLESLKSTTHLNNTDNCDVNLSEQTNEINYDEEKKVNKKTNHEKMKKKKKKKKKKKKKKKKEKKQIDIMYKNLSRLNLNLLLPTKKKVKKSKNSFKKEEEKQKKKNKKVKKIKGINKGEKIKSNKKENKDNNNDSSTECVVEGEKGKDLHEFNKNGNLEDEQMDVDISMNISSINCESDNKNVSKEGEEEKKDIAENKEEVDKNKEEVYMDKHEMDLNNEEVYMDKNEMDLNNEEVYMDKHEMDLNNEEVYMDKHEMDLNNEEVYMDKHEMDLNKEEVYMDKHEMDLNNEEVDKENEYDENILSDNIIYNENNSFGNNKNSFFNNTSPLKTEIINEEENSLNEMKEDINEYVEMENKLDTEKIKDSEKIGGKIEVDNKMISPINRHNFYLTILEGMNKNFPRQWNKNNITLSKNQGQIYKGRKEKKRKRSYRNDEKLLDHSILNDINISDKMDERNELLESIKSNSTINNVLEIIKYDNRKKIKKNDTNKEIIKYDNFTSKYNNKSNDIQLNGGIYINKFKLSLDMPINKLAVSSNLGPPSSIGSTEIQPIQKNFNDFKMNINVYCIRMEPHEKYSSYSHKNNLVVYIDKGEKINIIINMSKTYEKGDFFYIPRFSNFQIINDSRCDCVLYVCPLI

SEQ ID NO: 348-349 - skipped (2 entries)

SEQ ID NO: 350 - protein, 182 aa, synthetic construct
MNALRRLPVICSFLVFLVFSNVLCFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 351-352 - skipped (2 entries)

SEQ ID NO: 353 - protein, 245 aa, synthetic construct
MKMKIPICFLIILVLLKCVLSYNLNNDLSKNNNFSLNTYVRKDDVEDDSKNEIVDNIQKMVDDFSDDIGFVKTSMREVLLDTEASLEEVSDHVVQNISKYSLTIEEKLNLFDGLLEEFIENNKGLISNLSKRQQKLKGDKIKKVCDLILKKLKKLENVNKLIKYKIILKYGNKDNKKEMIQTLKNEEGLSDDFKNNLSNYETEQNNDDIKEIELVNFISTNYDKFVVNLEDLNKELLKDLNMALS

SEQ ID NO: 354-355 - skipped (2 entries)

SEQ ID NO: 356 - protein, 842 aa, synthetic construct
MKLRILKKHYYVVFILLYLYDISCFKCIRLNNRSIYKNKYKNNVHIGTNENIRSIEKYSNVLCNSILCKNDKISSFINQRKNVDDDDESENDDMYESTTAGSSSETDNESDEEENDSSDNNNSDEEQIENSNNNNSDEEQNDSSSNDNNDEENEEQDDVMDNDQNDKKIKHSFNLANESKHTKEERVKEEKKLKIYDFINDKEKRLNFNGDQKDEDNEENDDKDENTLENRNIISKHTSVFPGLYFIGIGYNLLFGNPLGEADSLIDPGYRAQIYLMEWALSKEGIANDLSTLQPVNGWIRKENACSRVESITECSSISDYTKSLSAEAKVSGSYWGIASFSASTGYSSFLHEVTKRSKKTFLVKSNCVKYTIGLPPYIPWDKTTAYKNAVNELPAVFTGLDKESECPSDVYEENKTKSNCENVSLWMKFFDIYGTHIIYESQLGGKITKIINVSTSSIEQMKKNGVSVKAKIQAQFGFGSAGGSTDVNSSNSSANDEQSYDMNEQLIVIGGNPIKDVTKEENLFEWSKTVTNHPMPINIKLTPISDSFDSDDLKESYDKAIIYYSRLYGLSPHDTMQKDDKDIIKILTNADTVTKNSAPPINAQCPHGKVVMFGFSLKQNFWDNTNALKGYNIEVCEAGSNSCTSKQGSSNKYDTSYLYMECGDQPLPFSEQVISESTSTYNTVKCPNDYSILLGFGISSSSGRINSAEYVYSTPCIPGMKSCSLNMNNDNQKSYIYVLCVDTTIWSGVNNLSLVALDGAHGKVNRSKKYSDGELVGTCPLDGTVLTGFKVEFHTSSPYVQTPFEKCAKSLKACSVHGSGHAIGIQNFKSLFIYMLCKNNK

SEQ ID NO: 357-358 - skipped (2 entries)

SEQ ID NO: 359 - protein, 229 aa, synthetic construct
MKVSKLVLFAHIFFIINILCQYICLNASKVNKKGKIAEEKKRKNIKNIDKAIEEHNKRKKLIYYSLIASGAIASVAAILGLGYYGYKKSREDDLYYNKYLEYRNGEYNIKYQDGAIASTSEFYIEPEGINKINLNKPIIENKNNVDVSIKRYNNFVDIARLSIQKHFEHLSNDQKDSHVNNMEYMQKFVQGLQENRNISLSKYQENKAVMDLKYHLQKVYANYLSQEEN

SEQ ID NO: 360-361 - skipped (2 entries)

SEQ ID NO: 362 - protein, 108 aa, synthetic construct
MKVSRHTVLLNIILIVSLLGCVLTLNLFSANDDKRALKDIDRTLEKLLRKKQIIISTAAVALAITLGGLFGSLGYKSWKNKNKSKDKVNDGSDSEELDSSKEDKDKDE

SEQ ID NO: 363-364 - skipped (2 entries)

SEQ ID NO: 365 - protein, 526 aa, synthetic construct
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 366-367 - skipped (2 entries)

SEQ ID NO: 368 - protein, 378 aa, synthetic construct
MARNFECKKINSDDMTSSKKYSKNVGEKFNLISCTKLFALSMLFLICQNYENSPQSTSSHQEYQYNGLVLGNRILSELDQAENHTISYKTNNYEDSSVENPNQQTSDSSSLQTDEDKKKDDSDATSIGETSPTTETTSVEETVTIEDTESVEETESVEETPSTSTEETSSTGKKTYVDRIASILNPLINGEKKSTEKKSSEKKSSEKKSSDEQSSSDEQNSSDDQNSFDDQKLFEDIDNLINGIKSRYQEFSAKIKSPEFQNKCKSYMNTAKEMIEERRNCAMSFISRNLNALGIDKIFEDEFGGYALLGKMMLTKVFIDNMFIPDFLRNSSTIILTIVYFLIMMFIVGSYLDINQDTKTERRNTNESKLFNRTQPPM

SEQ ID NO: 369-370 - skipped (2 entries)

SEQ ID NO: 371 - protein, 327 aa, synthetic construct
MKKGNNGNFSMARNCECKKIDNDDMVSSTRYSNKFGEKFNLISCTKLFALGMLFLICKNCDNSTQTTSAYQEYQYNGLILGKSRILSELDQNKDQNLGYKTQSYEDSSTENASTSYMSTLKTDEESNTAGEDRKEDNNDAAFIGKKSTEKKPHIDRISSTLNPESTDRRKSSDEQKSSNDKNIFKDVDDLINGIKSRYQDITNKIKSPEFQNECKGYINVAKEMIEDHRNHAMRFVSRNLNSLGIDQIFNDEHGGYAFLAKIMLTKVFVDNLFVPNFLKNNSTILSTIVYFLIISFIVSNYLDATQNNERERKNLNKSNIFCRAKPM

SEQ ID NO: 372-373 - skipped (2 entries)

SEQ ID NO: 374 - protein, 406 aa, synthetic construct
MKKETKVDNGVIVPPTRHSQNWGKKLNLISCTKIFALIVLFMICQNGDNSTQSTSSYQEYQHNGLVLGKSRILSELDKTETHATDYKTQSNEDSSVEDPNSPTIITELASDSSKLKTDEESENEDTDKKKDDNDSTSIGETSSAGETTSAEEKLYTDIIDSILSSVVIDGKKIFEEKKESDGKNIFEKKPYDGKKLFEEKKPSDEKKTPGEKKSSNEKKTPGEKKSSNEKKSSSEQKTSSEQKPSSEQKPSSEQKPYDQKSFADDLDSIVNGIKSCYQEVANKVKSPEFQSACKEYVNTAKELFDEHKSSALNFVSSSMQNLGINQIFDDENFGGFATLGKLAVSKVMVDNLFIPDYLKNQSPIILTVVYFLILFMTFGTIYDILPNNRRRGNSNAPQTNYKPQPL

SEQ ID NO: 375-376 - skipped (2 entries)

SEQ ID NO: 377 - protein, 175 aa, synthetic construct
MKVPKVSALFLVFLLSVSFLPSSSLCDPNAANTKIPNIPNPTANAQSPTVSGNSIEQTAEEPNAATKVNNVPVPEKKNDAPVENKDIEKVNDQINAKSKTKPLLISAISAVVAIAIASALGYGIYTKKNSNTPNEDEPSIPSTDDDTGVSEESNEQPIPEELPIPETTENTETPQ

SEQ ID NO: 378-379 - skipped (2 entries)

SEQ ID NO: 380 - protein, 114 aa, synthetic construct
MKLAKALYFVAFLLAIKVLTPGSNNYVEANPPNSDKVANGGDRTLIKKIKDNKVAFISTIAATVALAIATTLGVMHYKKNAKENDSDDDIKIIPLSYPHKDPGKPINYEGNPFA

SEQ ID NO: 381 - DNA, 81 bp, unidentified
  source 1..81: mol_type = other DNA; note = Herpes simplex virus 1
atggggggggctgccgccaggttgggggccgtgattttgtttgtcgtcatagtgggcctccatggggtccgcagcaaatat

SEQ ID NO: 382 - protein, 27 aa, unidentified
  source 1..27: note = Herpes simplex virus 1
MGGAAARLGAVILFVVIVGLHGVRSKY

SEQ ID NO: 383 - RNA, 81 nt, unidentified
  source 1..81: mol_type = other RNA; note = Herpes simplex virus 1
atggggggggctgccgccaggttgggggccgtgattttgtttgtcgtcatagtgggcctccatggggtccgcagcaaatat

SEQ ID NO: 384 - DNA, 81 bp, unidentified
  source 1..81: mol_type = other DNA; note = Herpes simplex virus 1
atgggaggagccgccgccagactgggagccgtgatcctgttcgtggtgatcgtgggactgcatggagtgagaagcaagtac

SEQ ID NO: 385 - protein, 27 aa, unidentified
  source 1..27: note = Herpes simplex virus 1
MGGAAARLGAVILFVVIVGLHGVRSKY

SEQ ID NO: 386 - RNA, 81 nt, unidentified
  source 1..81: mol_type = other RNA; note = Herpes simplex virus 1
atgggaggagccgccgccagactgggagccgtgatcctgttcgtggtgatcgtgggactgcatggagtgagaagcaagtac

SEQ ID NO: 387 - DNA, 81 bp, unidentified
  source 1..81: mol_type = other DNA; note = Herpes simplex virus 1
atggggggggctgccgccaggttgggggccgtgattttgtttgtcgtcatagtgggcctccatggggtccgcggcaaatat

SEQ ID NO: 388 - protein, 27 aa, unidentified
  source 1..27: note = Herpes simplex virus 1
MGGAAARLGAVILFVVIVGLHGVRGKY

SEQ ID NO: 389 - RNA, 81 nt, unidentified
  source 1..81: mol_type = other RNA; note = Herpes simplex virus 1
atggggggggctgccgccaggttgggggccgtgattttgtttgtcgtcatagtgggcctccatggggtccgcggcaaatat

SEQ ID NO: 390 - DNA, 75 bp, unidentified
  source 1..75: mol_type = other DNA; note = Herpes simplex virus 2
atggggcgtttgacctccggcgtcgggacggcggccctgctagttgtcgcggtgggactccgcgtcgtctgcgcc

SEQ ID NO: 391 - protein, 25 aa, unidentified
  source 1..25: note = Herpes simplex virus 2
MGRLTSGVGTAALLVVAVGLRVVCA

SEQ ID NO: 392 - RNA, 75 nt, unidentified
  source 1..75: mol_type = other RNA; note = Herpes simplex virus 2
atggggcgtttgacctccggcgtcgggacggcggccctgctagttgtcgcggtgggactccgcgtcgtctgcgcc

SEQ ID NO: 393 - DNA, 84 bp, unidentified
  source 1..84: mol_type = other DNA; note = Herpes simplex virus 2
atggggcgtttgacctccggcgtcgggacggcggccctgctagttgtcgcggtgggactccgcgtcgtctgcgccaaatacgcc

SEQ ID NO: 394 - protein, 28 aa, unidentified
  source 1..28: note = Herpes simplex virus 2
MGRLTSGVGTAALLVVAVGLRVVCAKYA

SEQ ID NO: 395 - RNA, 84 nt, unidentified
  source 1..84: mol_type = other RNA; note = Herpes simplex virus 2
atggggcgtttgacctccggcgtcgggacggcggccctgctagttgtcgcggtgggactccgcgtcgtctgcgccaaatacgcc

SEQ ID NO: 396 - DNA, 54 bp, unidentified
  source 1..54: mol_type = other DNA; note = Plasmodium falciparum
atgatgagaaaattagctattttatctgtttcttcctttttatttgttgaggcc

SEQ ID NO: 397 - protein, 18 aa, unidentified
  source 1..18: note = Plasmodium falciparum
MMRKLAILSVSSFLFVEA

SEQ ID NO: 398 - RNA, 54 nt, unidentified
  source 1..54: mol_type = other RNA; note = Plasmodium falciparum
atgatgagaaaattagctattttatctgtttcttcctttttatttgttgaggcc

SEQ ID NO: 399 - DNA, 96 bp, unidentified
  source 1..96: mol_type = other DNA; note = Ebola virus
atgggtgttacaggaatcttgcagctgcctagagatcgattcaagaggacatcattctttctgtgggtgattatcctgttccaaagaacattttcc

SEQ ID NO: 400 - protein, 32 aa, unidentified
  source 1..32: note = Ebola virus
MGVTGILQLPRDRFKRTSFFLWVIILFQRTFS

SEQ ID NO: 401 - RNA, 96 nt, unidentified
  source 1..96: mol_type = other RNA; note = Ebola virus
atgggtgttacaggaatcttgcagctgcctagagatcgattcaagaggacatcattctttctgtgggtgattatcctgttccaaagaacattttcc

SEQ ID NO: 402 - DNA, 57 bp, Severe acute respiratory syndrome-related coronavirus
  source 1..57: mol_type = other DNA; note = SARS-CoV-2
atgtttgtgtttcttgtgctgctgcctcttgtgtcttctcagtgtgtgaatttgaca

SEQ ID NO: 403 - protein, 19 aa, Severe acute respiratory syndrome-related coronavirus
  source 1..19: note = SARS-CoV-2
MFVFLVLLPLVSSQCVNLT

SEQ ID NO: 404 - RNA, 56 nt, Severe acute respiratory syndrome-related coronavirus
  source 1..56: mol_type = other RNA; note = SARS-CoV-2
tgtttgtgtttcttgtgctgctgcctcttgtgtcttctcagtgtgtgaatttgaca

SEQ ID NO: 405 - DNA, 63 bp, Homo sapiens
  source 1..63: mol_type = other DNA
atggattggatttggagaatcctgttcctcgtgggagccgctacaggagcccactcccagatg

SEQ ID NO: 406 - protein, 21 aa, Homo sapiens
MDWIWRILFLVGAATGAHSQM

SEQ ID NO: 407 - RNA, 63 nt, Homo sapiens
  source 1..63: mol_type = other RNA
atggattggatttggagaatcctgttcctcgtgggagccgctacaggagcccactcccagatg

SEQ ID NO: 408 - protein, 20 aa, synthetic construct
METPAQLLFLLLLWLPDTTG

SEQ ID NO: 409 - protein, 18 aa, synthetic construct
MDWTWILFLVAAATRVHS

SEQ ID NO: 410 - protein, 24 aa, Japanese encephalitis virus
MLGSNSGQRVVFTILLLLVAPAYS

SEQ ID NO: 411 - protein, 17 aa, synthetic construct
MKCLLYLAFLFIGVNCA

SEQ ID NO: 412 - protein, 24 aa, synthetic construct
MCRGLSAVLILLVSLSAQLHVVVG

SEQ ID NO: 413 - DNA, 57 bp, Homo sapiens
  source 1..57: mol_type = other DNA
atggagttgggactgagctggattttccttttggctattttaaaaggtgtccagtgt

SEQ ID NO: 414 - protein, 19 aa, Homo sapiens
MELGLSWIFLLAILKGVQC

SEQ ID NO: 415 - RNA, 57 nt, Homo sapiens
  source 1..57: mol_type = other RNA
atggagttgggactgagctggattttccttttggctattttaaaaggtgtccagtgt

SEQ ID NO: 416 - DNA, 57 bp, Homo sapiens
  source 1..57: mol_type = other DNA
atggaactggggctccgctgggttttccttgttgctattttagaaggtgtccagtgt

SEQ ID NO: 417 - protein, 19 aa, Homo sapiens
MELGLRWVFLVAILEGVQC

SEQ ID NO: 418 - RNA, 57 nt, Homo sapiens
  source 1..57: mol_type = other RNA
atggaactggggctccgctgggttttccttgttgctattttagaaggtgtccagtgt

SEQ ID NO: 419 - DNA, 57 bp, Homo sapiens
  source 1..57: mol_type = other DNA
atgaaacacctgtggttcttcctcctgctggtggcagctcccagatgggtcctgtcc

SEQ ID NO: 420 - protein, 19 aa, Homo sapiens
MKHLWFFLLLVAAPRWVLS

SEQ ID NO: 421 - RNA, 57 nt, Homo sapiens
  source 1..57: mol_type = other RNA
atgaaacacctgtggttcttcctcctgctggtggcagctcccagatgggtcctgtcc

SEQ ID NO: 422 - DNA, 57 bp, Homo sapiens
  source 1..57: mol_type = other DNA
atggactggacctggaggatcctcttcttggtggcagcagcaacaggtgcccactcg

SEQ ID NO: 423 - protein, 19 aa, Homo sapiens
MDWTWRILFLVAAATGAHS

SEQ ID NO: 424 - RNA, 57 nt, Homo sapiens
  source 1..57: mol_type = other RNA
atggactggacctggaggatcctcttcttggtggcagcagcaacaggtgcccactcg

SEQ ID NO: 425 - DNA, 57 bp, Homo sapiens
  source 1..57: mol_type = other DNA
atggactggacctggaggttcctctttgtggtggcagcagctacaggtgtccagtcc

SEQ ID NO: 426 - protein, 19 aa, Homo sapiens
MDWTWRFLFVVAAATGVQS

SEQ ID NO: 427 - RNA, 57 nt, Homo sapiens
  source 1..57: mol_type = other RNA
atggactggacctggaggttcctctttgtggtggcagcagctacaggtgtccagtcc

SEQ ID NO: 428 - DNA, 57 bp, Homo sapiens
  source 1..57: mol_type = other DNA
atggagtttgggctgagctggctttttcttgtggcgattctaaaaggtgtccagtgt

SEQ ID NO: 429 - protein, 19 aa, Homo sapiens
MEFGLSWLFLVAILKGVQC

SEQ ID NO: 430 - RNA, 57 nt, Homo sapiens
  source 1..57: mol_type = other RNA
atggagtttgggctgagctggctttttcttgtggcgattctaaaaggtgtccagtgt

SEQ ID NO: 431 - DNA, 57 bp, Homo sapiens
  source 1..57: mol_type = other DNA
atggagtttgggctgagctgggttttcctcgttgctctttttagaggtgtccagtgt

SEQ ID NO: 432 - protein, 19 aa, Homo sapiens
MEFGLSWVFLVALFRGVQC

SEQ ID NO: 433 - RNA, 57 nt, Homo sapiens
  source 1..57: mol_type = other RNA
atggagtttgggctgagctgggttttcctcgttgctctttttagaggtgtccagtgt

SEQ ID NO: 434 - DNA, 78 bp, Homo sapiens
  source 1..78: mol_type = other DNA
atggacctcctgcacaagaacatgaaacacctgtggttcttcctcctcctggtggcagctcccagatgggtgctgtcc

SEQ ID NO: 435 - protein, 26 aa, Homo sapiens
MDLLHKNMKHLWFFLLLVAAPRWVLS

SEQ ID NO: 436 - RNA, 78 nt, Homo sapiens
  source 1..78: mol_type = other RNA
atggacctcctgcacaagaacatgaaacacctgtggttcttcctcctcctggtggcagctcccagatgggtgctgtcc

SEQ ID NO: 437 - DNA, 66 bp, Homo sapiens
  source 1..66: mol_type = other DNA
atggacatgagggtccctgctcagctcctggggctcctgctgctctggctctcaggtgccagatgt

SEQ ID NO: 438 - protein, 22 aa, Homo sapiens
MDMRVPAQLLGLLLLWLSGARC

SEQ ID NO: 439 - RNA, 66 nt, Homo sapiens
  source 1..66: mol_type = other RNA
atggacatgagggtccctgctcagctcctggggctcctgctgctctggctctcaggtgccagatgt

SEQ ID NO: 440 - DNA, 66 bp, Homo sapiens
  source 1..66: mol_type = other DNA
atgaaatacctattgcctacggcagccgctggattgttattactcgcggcccagccggccatggcc

SEQ ID NO: 441 - protein, 22 aa, Homo sapiens
MKYLLPTAAAGLLLLAAQPAMA

SEQ ID NO: 442 - RNA, 66 nt, Homo sapiens
  source 1..66: mol_type = other RNA
atgaaatacctattgcctacggcagccgctggattgttattactcgcggcccagccggccatggcc

SEQ ID NO: 443 - DNA, 60 bp, synthetic construct
  source 1..60: mol_type = other DNA
ttcaacgtggtcaacagcagcatcggcctgattatggtgctgtccttcctgttcctgaac

SEQ ID NO: 444 - protein, 20 aa, synthetic construct
FNVVNSSIGLIMVLSFLFLN

SEQ ID NO: 445 - RNA, 60 nt, synthetic construct
  source 1..60: mol_type = other RNA
ttcaacgtggtcaacagcagcatcggcctgattatggtgctgtccttcctgttcctgaac

SEQ ID NO: 446 - DNA, 123 bp, unidentified
  source 1..123: mol_type = other DNA; note = Herpes simplex virus 1
ggcctgatcgccggcgcggtgggcggcagtctcctggcagccctggtcatttgcggaattgtgtactggatgcgccgccacactcaaaaagccccaaagcgcatacgcctcccccacatccgg

SEQ ID NO: 447 - protein, 41 aa, unidentified
GLIAGAVGGSLLAALVICGIVYWMRRHTQKAPKRIRLPHIR

SEQ ID NO: 448 - RNA, 123 nt, unidentified
  source 1..123: mol_type = other RNA; note = Herpes simplex virus 1
ggcctgatcgccggcgcggtgggcggcagtctcctggcagccctggtcatttgcggaattgtgtactggatgcgccgccacactcaaaaagccccaaagcgcatacgcctcccccacatccgg

SEQ ID NO: 449 - DNA, 111 bp, synthetic construct
  source 1..111: mol_type = other DNA
cctaacaaaggcagcggcacaacctccggcaccacaagactgctgtctggccacacctgtttcaccctgactggcctgctgggcaccctggttacaatgggactgctgaca

SEQ ID NO: 450 - protein, 37 aa, synthetic construct
PNKGSGTTSGTTRLLSGHTCFTLTGLLGTLVTMGLLT

SEQ ID NO: 451 - RNA, 111 nt, synthetic construct
  source 1..111: mol_type = other RNA
cctaacaaaggcagcggcacaacctccggcaccacaagactgctgtctggccacacctgtttcaccctgactggcctgctgggcaccctggttacaatgggactgctgaca

SEQ ID NO: 452 - protein, 10 aa, synthetic construct
GGSGGGGSGG

SEQ ID NO: 453 - protein, 11 aa, synthetic construct
SGGGGSGGGGS

SEQ ID NO: 454 - protein, 6 aa, synthetic construct
GSGSGS

SEQ ID NO: 455 - protein, 9 aa, synthetic construct
GSPGSGSGS

SEQ ID NO: 456 - protein, 15 aa, synthetic construct
GGGGSGGGGSGGGGS

SEQ ID NO: 457 - protein, 10 aa, synthetic construct
GGSLGGGGSG

SEQ ID NO: 458 - skipped

SEQ ID NO: 459 - protein, 4 aa, synthetic construct
GGGS

SEQ ID NO: 460 - protein, 10 aa, synthetic construct
AGNRVRRSVG

SEQ ID NO: 461 - protein, 14 aa, synthetic construct
QVHFQPLPPAVVKL

SEQ ID NO: 462 - protein, 14 aa, synthetic construct
QFIKANSKFIGITE

SEQ ID NO: 463 - protein, 9 aa, unidentified
  source 1..9: note = Plasmodium sp.
MINAYLDKL

SEQ ID NO: 464 - DNA, 53 bp, synthetic construct
  source 1..53: mol_type = other DNA
agaataaactagtattcttctggtccccacagactcagagagaacccgccacc

SEQ ID NO: 465 - RNA, 53 nt, synthetic construct
  source 1..53: mol_type = other RNA
agaataaactagtattcttctggtccccacagactcagagagaacccgccacc

SEQ ID NO: 466 - DNA, 110 bp, synthetic construct
  source 1..110: mol_type = other DNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 467 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 468-469 - skipped (2 entries)

SEQ ID NO: 470 - DNA, 278 bp, synthetic construct
  source 1..278: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 471 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 472 - DNA, 47 bp, synthetic construct
  source 1..47: mol_type = other DNA
aactagtattcttctggtccccacagactcagagagaacccgccacc

SEQ ID NO: 473 - RNA, 47 nt, synthetic construct
  source 1..47: mol_type = other RNA
aactagtattcttctggtccccacagactcagagagaacccgccacc

SEQ ID NO: 474 - DNA, 278 bp, synthetic construct; identical to SEQ ID NO: 470
  source 1..278: mol_type = other DNA

SEQ ID NO: 475 - RNA, 278 nt, synthetic construct; identical to SEQ ID NO: 471
  source 1..278: mol_type = other RNA

SEQ ID NO: 476 - DNA, 110 bp, synthetic construct; identical to SEQ ID NO: 466
  source 1..110: mol_type = other DNA

SEQ ID NO: 477 - protein, 8 aa, synthetic construct
NANPNVDP

SEQ ID NO: 478 - RNA, 110 nt, synthetic construct; identical to SEQ ID NO: 467
  source 1..110: mol_type = other RNA

SEQ ID NO: 479 - protein, 55 aa, synthetic construct
IVGIVAGLAVLAVVVIGAVVATVMCRRKSSGGKGGSYSQAASSDSAQGSDVSLTA

SEQ ID NO: 480 - RNA, 12 nt, synthetic construct
  source 1..12: mol_type = other RNA
aaaaaaattttt

SEQ ID NO: 481 - protein, 15 aa, synthetic construct
MMRKLAILSVSSFLF

SEQ ID NO: 482 - protein, 15 aa, synthetic construct
ELNYDNAGTNLYNEL

SEQ ID NO: 483 - protein, 15 aa, synthetic construct
DNAGTNLYNELEMNY

SEQ ID NO: 484 - protein, 15 aa, synthetic construct
TNLYNELEMNYYGKQ

SEQ ID NO: 485 - protein, 15 aa, synthetic construct
NELEMNYYGKQENWY

SEQ ID NO: 486 - protein, 15 aa, synthetic construct
MNYYGKQENWYSLKK

SEQ ID NO: 487 - protein, 15 aa, synthetic construct
GKQENWYSLKKNSRS

SEQ ID NO: 488 - protein, 15 aa, synthetic construct
NWYSLKKNSRSLGEN

SEQ ID NO: 489 - protein, 15 aa, synthetic construct
LKKNSRSLGENDDGN

SEQ ID NO: 490 - protein, 15 aa, synthetic construct
SRSLGENDDGNNEDN

SEQ ID NO: 491 - protein, 15 aa, synthetic construct
GENDDGNNEDNEKLR

SEQ ID NO: 492 - protein, 15 aa, synthetic construct
DGNNEDNEKLRKPKH

SEQ ID NO: 493 - protein, 15 aa, synthetic construct
EDNEKLRKPKHKKLK

SEQ ID NO: 494 - protein, 14 aa, synthetic construct
KLRKPKHKKLKQPA

SEQ ID NO: 495 - protein, 15 aa, synthetic construct
SVSSFLFVEALFQEY

SEQ ID NO: 496 - protein, 15 aa, synthetic construct
FLFVEALFQEYQCYG

SEQ ID NO: 497 - protein, 15 aa, synthetic construct
EALFQEYQCYGSSSN

SEQ ID NO: 498 - protein, 15 aa, synthetic construct
QEYQCYGSSSNTRVL

SEQ ID NO: 499 - protein, 15 aa, synthetic construct
CYGSSSNTRVLNELN

SEQ ID NO: 500 - protein, 15 aa, synthetic construct
SSNTRVLNELNYDNA

SEQ ID NO: 501 - protein, 15 aa, synthetic construct
RVLNELNYDNAGTNL

SEQ ID NO: 502 - protein, 10 aa, synthetic construct
PKHKKLKQPA

SEQ ID NO: 503 - protein, 15 aa, synthetic construct
SKKKNKKGSGEPLID

SEQ ID NO: 504 - protein, 12 aa, synthetic construct
KSKYKLATSVLA

SEQ ID NO: 505 - protein, 15 aa, synthetic construct
NKKGSGEPLIDVHDL

SEQ ID NO: 506 - protein, 15 aa, synthetic construct
SGEPLIDVHDLISDM

SEQ ID NO: 507 - protein, 15 aa, synthetic construct
LIDVHDLISDMIKKE

SEQ ID NO: 508 - protein, 15 aa, synthetic construct
HDLISDMIKKEEELV

SEQ ID NO: 509 - protein, 15 aa, synthetic construct
SDMIKKEEELVEVNK

SEQ ID NO: 510 - protein, 15 aa, synthetic construct
KKEEELVEVNKRKSK

SEQ ID NO: 511 - protein, 15 aa, synthetic construct
ELVEVNKRKSKYKLA

SEQ ID NO: 512 - protein, 15 aa, synthetic construct
VNKRKSKYKLATSVL

SEQ ID NO: 513 - protein, 15 aa, synthetic construct
CNIPKNYTVGDVLRY

SEQ ID NO: 514 - protein, 15 aa, synthetic construct
LKYNNIIDLILYNTI

SEQ ID NO: 515 - protein, 15 aa, synthetic construct
LILYNTIIDIKENSF

SEQ ID NO: 516 - protein, 15 aa, synthetic construct
DIKENSFVKKDIPIY

SEQ ID NO: 517 - protein, 15 aa, synthetic construct
KKDIPIYVKKGNIFK

SEQ ID NO: 518 - protein, 15 aa, synthetic construct
KKGNIFKNVSLKYIP

SEQ ID NO: 519 - protein, 15 aa, synthetic construct
VSLKYIPINIKEDEL

SEQ ID NO: 520 - protein, 15 aa, synthetic construct
NIKEDELLTYLLHYL

SEQ ID NO: 521 - protein, 15 aa, synthetic construct
TYLLHYLTDNHEVVS

SEQ ID NO: 522 - protein, 15 aa, synthetic construct
DNHEVVSFIRDITCI

SEQ ID NO: 523 - protein, 15 aa, synthetic construct
VGDVLRYVKNKIIED

SEQ ID NO: 524 - protein, 15 aa, synthetic construct
VYPLCDSIYIFHEKK

SEQ ID NO: 525 - protein, 15 aa, synthetic construct
YIFHEKKQLSLIPSI

SEQ ID NO: 526 - protein, 15 aa, synthetic construct
LSLIPSISYHIQLKN

SEQ ID NO: 527 - protein, 15 aa, synthetic construct
YHIQLKNVYYISTVE

SEQ ID NO: 528 - protein, 15 aa, synthetic construct
YYISTVETYENFYNI

SEQ ID NO: 529 - protein, 15 aa, synthetic construct
YENFYNILGHMELNL

SEQ ID NO: 530 - protein, 15 aa, synthetic construct
GHMELNLNKFLHIQN

SEQ ID NO: 531 - protein, 15 aa, synthetic construct
KFLHIQNDGNNYKKK

SEQ ID NO: 532 - protein, 15 aa, synthetic construct
GNNYKKKEAYNNIYY

SEQ ID NO: 533 - protein, 15 aa, synthetic construct
AYNNIYYMNNVDELI

SEQ ID NO: 534 - protein, 15 aa, synthetic construct
KNKIIEDTNISMLND

SEQ ID NO: 535 - protein, 15 aa, synthetic construct
NNVDELIHIDVNIIF

SEQ ID NO: 536 - protein, 15 aa, synthetic construct
IDVNIIFPSIQKIIR

SEQ ID NO: 537 - protein, 15 aa, synthetic construct
SIQKIIRVKNLNVNI

SEQ ID NO: 538 - protein, 15 aa, synthetic construct
KNLNVNIKMEELLYK

SEQ ID NO: 539 - protein, 15 aa, synthetic construct
MEELLYKIFGCITNK

SEQ ID NO: 540 - protein, 15 aa, synthetic construct
FGCITNKIYKWGELF

SEQ ID NO: 541 - protein, 15 aa, synthetic construct
YKWGELFRNFVGKKE

SEQ ID NO: 542 - protein, 15 aa, synthetic construct
NFVGKKEKINIVNND

SEQ ID NO: 543 - protein, 15 aa, synthetic construct
INIVNNDIKNNSNNI

SEQ ID NO: 544 - protein, 14 aa, synthetic construct
KNNSNNINSINNMN

SEQ ID NO: 545 - protein, 15 aa, synthetic construct
NISMLNDLQLKIIHN

SEQ ID NO: 546 - protein, 15 aa, synthetic construct
KNYTVGDVLRYVKNK

SEQ ID NO: 547 - protein, 15 aa, synthetic construct
LRYVKNKIIEDTNIS

SEQ ID NO: 548 - protein, 15 aa, synthetic construct
IEDTNISMLNDLQLK

SEQ ID NO: 549 - protein, 15 aa, synthetic construct
LNDLQLKIIHNDNYI

SEQ ID NO: 550 - protein, 15 aa, synthetic construct
IHNDNYIDIPKDMNI

SEQ ID NO: 551 - protein, 15 aa, synthetic construct
IPKDMNIEYAINYLF

SEQ ID NO: 552 - protein, 15 aa, synthetic construct
YAINYLFIYTPFIVY

SEQ ID NO: 553 - protein, 15 aa, synthetic construct
YTPFIVYPSDDILKY

SEQ ID NO: 554 - protein, 15 aa, synthetic construct
SDDILKYNNIIDLIL

SEQ ID NO: 555 - protein, 15 aa, synthetic construct
NIIDLILYNTIIDIK

SEQ ID NO: 556 - protein, 15 aa, synthetic construct
QLKIIHNDNYIDIPK

SEQ ID NO: 557 - protein, 15 aa, synthetic construct
NTIIDIKENSFVKKD

SEQ ID NO: 558 - protein, 15 aa, synthetic construct
NSFVKKDIPIYVKKG

SEQ ID NO: 559 - protein, 15 aa, synthetic construct
PIYVKKGNIFKNVSL

SEQ ID NO: 560 - protein, 15 aa, synthetic construct
IFKNVSLKYIPINIK

SEQ ID NO: 561 - protein, 15 aa, synthetic construct
YIPINIKEDELLTYL

SEQ ID NO: 562 - protein, 15 aa, synthetic construct
DELLTYLLHYLTDNH

SEQ ID NO: 563 - protein, 15 aa, synthetic construct
HYLTDNHEVVSFIRD

SEQ ID NO: 564 - protein, 15 aa, synthetic construct
VVSFIRDITCICVYP

SEQ ID NO: 565 - protein, 15 aa, synthetic construct
TCICVYPLCDSIYIF

SEQ ID NO: 566 - protein, 15 aa, synthetic construct
CDSIYIFHEKKQLSL

SEQ ID NO: 567 - protein, 15 aa, synthetic construct
NYIDIPKDMNIEYAI

SEQ ID NO: 568 - protein, 15 aa, synthetic construct
EKKQLSLIPSISYHI

SEQ ID NO: 569 - protein, 15 aa, synthetic construct
PSISYHIQLKNVYYI

SEQ ID NO: 570 - protein, 15 aa, synthetic construct
LKNVYYISTVETYEN

SEQ ID NO: 571 - protein, 15 aa, synthetic construct
TVETYENFYNILGHM

SEQ ID NO: 572 - protein, 15 aa, synthetic construct
YNILGHMELNLNKFL

SEQ ID NO: 573 - protein, 15 aa, synthetic construct
LNLNKFLHIQNDGNN

SEQ ID NO: 574 - protein, 15 aa, synthetic construct
IQNDGNNYKKKEAYN

SEQ ID NO: 575 - protein, 15 aa, synthetic construct
KKKEAYNNIYYMNNV

SEQ ID NO: 576 - protein, 15 aa, synthetic construct
IYYMNNVDELIHIDV

SEQ ID NO: 577 - protein, 15 aa, synthetic construct
ELIHIDVNIIFPSIQ

SEQ ID NO: 578 - protein, 15 aa, synthetic construct
MNIEYAINYLFIYTP

SEQ ID NO: 579 - protein, 15 aa, synthetic construct
IIFPSIQKIIRVKNL

SEQ ID NO: 580 - protein, 15 aa, synthetic construct
IIRVKNLNVNIKMEE

SEQ ID NO: 581 - protein, 15 aa, synthetic construct
VNIKMEELLYKIFGC

SEQ ID NO: 582 - protein, 15 aa, synthetic construct
LYKIFGCITNKIYKW

SEQ ID NO: 583 - protein, 15 aa, synthetic construct
TNKIYKWGELFRNFV

SEQ ID NO: 584 - protein, 15 aa, synthetic construct
ELFRNFVGKKEKINI

SEQ ID NO: 585 - protein, 15 aa, synthetic construct
KKEKINIVNNDIKNN

SEQ ID NO: 586 - protein, 15 aa, synthetic construct
NNDIKNNSNNINSIN

SEQ ID NO: 587 - protein, 15 aa, synthetic construct
YLFIYTPFIVYPSDD

SEQ ID NO: 588 - protein, 15 aa, synthetic construct
IVYPSDDILKYNNII

SEQ ID NO: 589 - protein, 15 aa, synthetic construct
FCNRYMTEEPYNNIF

SEQ ID NO: 590 - protein, 15 aa, synthetic construct
RSALNIHVMSKYNML

SEQ ID NO: 591 - protein, 15 aa, synthetic construct
MSKYNMLNKYNKETN

SEQ ID NO: 592 - protein, 15 aa, synthetic construct
KYNKETNRLLKRNNN

SEQ ID NO: 593 - protein, 15 aa, synthetic construct
LLKRNNNVENRINNI

SEQ ID NO: 594 - protein, 15 aa, synthetic construct
ENRINNISNHYLCSG

SEQ ID NO: 595 - protein, 15 aa, synthetic construct
NHYLCSGFKKENRLF

SEQ ID NO: 596 - protein, 15 aa, synthetic construct
KKENRLFFLLFYKTI

SEQ ID NO: 597 - protein, 15 aa, synthetic construct
LLFYKTIKMMKLYIR

SEQ ID NO: 598 - protein, 15 aa, synthetic construct
MMKLYIRNLFMKYIK

SEQ ID NO: 599 - protein, 15 aa, synthetic construct
LFMKYIKIYYKTKHF

SEQ ID NO: 600 - protein, 15 aa, synthetic construct
EPYNNIFEIIKPENL

SEQ ID NO: 601 - protein, 15 aa, synthetic construct
YYKTKHFEKNIETNK

SEQ ID NO: 602 - protein, 15 aa, synthetic construct
KNIETNKKVVYVERD

SEQ ID NO: 603 - protein, 15 aa, synthetic construct
VVYVERDNLFDIERN

SEQ ID NO: 604 - protein, 15 aa, synthetic construct
LFDIERNNLFDILYM

SEQ ID NO: 605 - protein, 15 aa, synthetic construct
LFDILYMLKRIDSYV

SEQ ID NO: 606 - protein, 12 aa, synthetic construct
KRIDSYVKNIYS

SEQ ID NO: 607 - protein, 15 aa, synthetic construct
YMTEEPYNNIFEIIK

SEQ ID NO: 608 - protein, 15 aa, synthetic construct
NIFEIIKPENLYSSL

SEQ ID NO: 609 - protein, 15 aa, synthetic construct
ENLYSSLLLLSNEKE

SEQ ID NO: 610 - protein, 15 aa, synthetic construct
LLSNEKENDFPSSTN

SEQ ID NO: 611 - protein, 15 aa, synthetic construct
IIKPENLYSSLLLLS

SEQ ID NO: 612 - protein, 15 aa, synthetic construct
DFPSSTNCNGYMKCI

SEQ ID NO: 613 - protein, 15 aa, synthetic construct
NGYMKCIPFYNNVSE

SEQ ID NO: 614 - protein, 15 aa, synthetic construct
FYNNVSERWKRYNFI

SEQ ID NO: 615 - protein, 15 aa, synthetic construct
WKRYNFIQLYIIRSA

SEQ ID NO: 616 - protein, 15 aa, synthetic construct
LYIIRSALNIHVMSK

SEQ ID NO: 617 - protein, 15 aa, synthetic construct
NIHVMSKYNMLNKYN

SEQ ID NO: 618 - protein, 15 aa, synthetic construct
NMLNKYNKETNRLLK

SEQ ID NO: 619 - protein, 15 aa, synthetic construct
ETNRLLKRNNNVENR

SEQ ID NO: 620 - protein, 15 aa, synthetic construct
NNNVENRINNISNHY

SEQ ID NO: 621 - protein, 15 aa, synthetic construct
NNISNHYLCSGFKKE

SEQ ID NO: 622 - protein, 15 aa, synthetic construct
SSLLLLSNEKENDFP

SEQ ID NO: 623 - protein, 15 aa, synthetic construct
CSGFKKENRLFFLLF

SEQ ID NO: 624 - protein, 15 aa, synthetic construct
RLFFLLFYKTIKMMK

SEQ ID NO: 625 - protein, 15 aa, synthetic construct
KTIKMMKLYIRNLFM

SEQ ID NO: 626 - protein, 15 aa, synthetic construct
YIRNLFMKYIKIYYK

SEQ ID NO: 627 - protein, 15 aa, synthetic construct
YIKIYYKTKHFEKNI

SEQ ID NO: 628 - protein, 15 aa, synthetic construct
KHFEKNIETNKKVVY

SEQ ID NO: 629 - protein, 15 aa, synthetic construct
TNKKVVYVERDNLFD

SEQ ID NO: 630 - protein, 15 aa, synthetic construct
ERDNLFDIERNNLFD

SEQ ID NO: 631 - protein, 15 aa, synthetic construct
ERNNLFDILYMLKRI

SEQ ID NO: 632 - protein, 15 aa, synthetic construct
LYMLKRIDSYVKNIY

SEQ ID NO: 633 - protein, 15 aa, synthetic construct
EKENDFPSSTNCNGY

SEQ ID NO: 634 - protein, 15 aa, synthetic construct
STNCNGYMKCIPFYN

SEQ ID NO: 635 - protein, 15 aa, synthetic construct
KCIPFYNNVSERWKR

SEQ ID NO: 636 - protein, 15 aa, synthetic construct
VSERWKRYNFIQLYI

SEQ ID NO: 637 - protein, 15 aa, synthetic construct
NFIQLYIIRSALNIH

SEQ ID NO: 638 - protein, 15 aa, synthetic construct
QERRAKEKLQEQQRD

SEQ ID NO: 639 - protein, 15 aa, synthetic construct
LAEDLYGRLEIPAIE

SEQ ID NO: 640 - protein, 15 aa, synthetic construct
LYGRLEIPAIELPSE

SEQ ID NO: 641 - protein, 15 aa, synthetic construct
LEIPAIELPSENERG

SEQ ID NO: 642 - protein, 15 aa, synthetic construct
AIELPSENERGYYIP

SEQ ID NO: 643 - protein, 15 aa, synthetic construct
PSENERGYYIPHQSS

SEQ ID NO: 644 - protein, 15 aa, synthetic construct
ERGYYIPHQSSLPQD

SEQ ID NO: 645 - protein, 15 aa, synthetic construct
YIPHQSSLPQDNRGN

SEQ ID NO: 646 - protein, 15 aa, synthetic construct
QSSLPQDNRGNSRDS

SEQ ID NO: 647 - protein, 15 aa, synthetic construct
PQDNRGNSRDSKEIS

SEQ ID NO: 648 - protein, 15 aa, synthetic construct
RGNSRDSKEISIIEN

SEQ ID NO: 649 - protein, 15 aa, synthetic construct
AKEKLQEQQRDLEQR

SEQ ID NO: 650 - protein, 15 aa, synthetic construct
RDSKEISIIENTNRE

SEQ ID NO: 651 - protein, 15 aa, synthetic construct
EISIIENTNRESITT

SEQ ID NO: 652 - protein, 15 aa, synthetic construct
IENTNRESITTNVEG

SEQ ID NO: 653 - protein, 15 aa, synthetic construct
NRESITTNVEGRRDI

SEQ ID NO: 654 - protein, 15 aa, synthetic construct
ITTNVEGRRDIHKGH

SEQ ID NO: 655 - protein, 15 aa, synthetic construct
VEGRRDIHKGHLEEK

SEQ ID NO: 656 - protein, 15 aa, synthetic construct
RDIHKGHLEEKKDGS

SEQ ID NO: 657 - protein, 15 aa, synthetic construct
KGHLEEKKDGSIKPE

SEQ ID NO: 658 - protein, 15 aa, synthetic construct
EEKKDGSIKPEQKED

SEQ ID NO: 659 - protein, 15 aa, synthetic construct
DGSIKPEQKEDKSAD

SEQ ID NO: 660 - protein, 15 aa, synthetic construct
LQEQQRDLEQRKADT

SEQ ID NO: 661 - protein, 15 aa, synthetic construct
KPEQKEDKSADIQNH

SEQ ID NO: 662 - protein, 15 aa, synthetic construct
KEDKSADIQNHTLET

SEQ ID NO: 663 - protein, 15 aa, synthetic construct
SADIQNHTLETVNIS

SEQ ID NO: 664 - protein, 15 aa, synthetic construct
QNHTLETVNISDVND

SEQ ID NO: 665 - protein, 15 aa, synthetic construct
LETVNISDVNDFQIS

SEQ ID NO: 666 - protein, 15 aa, synthetic construct
NISDVNDFQISKYED

SEQ ID NO: 667 - protein, 15 aa, synthetic construct
VNDFQISKYEDEISA

SEQ ID NO: 668 - protein, 15 aa, synthetic construct
QISKYEDEISAEYDD

SEQ ID NO: 669 - protein, 15 aa, synthetic construct
YEDEISAEYDDSLID

SEQ ID NO: 670 - protein, 14 aa, synthetic construct
ISAEYDDSLIDEEE

SEQ ID NO: 671 - protein, 15 aa, synthetic construct
QRDLEQRKADTKKNL

SEQ ID NO: 672 - protein, 15 aa, synthetic construct
EQRKADTKKNLERKK

SEQ ID NO: 673 - protein, 15 aa, synthetic construct
ADTKKNLERKKEHGD

SEQ ID NO: 674 - protein, 15 aa, synthetic construct
KNLERKKEHGDVLAE

SEQ ID NO: 675 - protein, 15 aa, synthetic construct
RKKEHGDVLAEDLYG

SEQ ID NO: 676 - protein, 15 aa, synthetic construct
HGDVLAEDLYGRLEI

SEQ ID NO: 677 - protein, 15 aa, synthetic construct
DDEDLDEFKPIVQYD

SEQ ID NO: 678 - protein, 15 aa, synthetic construct
ENLDDLDEGIEKSSE

SEQ ID NO: 679 - protein, 15 aa, synthetic construct
DLDEGIEKSSEELSE

SEQ ID NO: 680 - protein, 15 aa, synthetic construct
GIEKSSEELSEEKIK

SEQ ID NO: 681 - protein, 15 aa, synthetic construct
SSEELSEEKIKKGKK

SEQ ID NO: 682 - protein, 15 aa, synthetic construct
LSEEKIKKGKKYEKT

SEQ ID NO: 683 - protein, 15 aa, synthetic construct
KIKKGKKYEKTKDNN

SEQ ID NO: 684 - protein, 15 aa, synthetic construct
GKKYEKTKDNNFKPN

SEQ ID NO: 685 - protein, 15 aa, synthetic construct
EKTKDNNFKPNDKSL

SEQ ID NO: 686 - protein, 15 aa, synthetic construct
DNNFKPNDKSLYDEH

SEQ ID NO: 687 - protein, 15 aa, synthetic construct
KPNDKSLYDEHIKKY

SEQ ID NO: 688 - protein, 15 aa, synthetic construct
LDEFKPIVQYDNFQD

SEQ ID NO: 689 - protein, 15 aa, synthetic construct
KSLYDEHIKKYKNDK

SEQ ID NO: 690 - protein, 15 aa, synthetic construct
DEHIKKYKNDKQVNK

SEQ ID NO: 691 - protein, 15 aa, synthetic construct
KKYKNDKQVNKEKEK

SEQ ID NO: 692 - protein, 15 aa, synthetic construct
NDKQVNKEKEKFIKS

SEQ ID NO: 693 - protein, 15 aa, synthetic construct
VNKEKEKFIKSLFHI

SEQ ID NO: 694 - protein, 15 aa, synthetic construct
KEKFIKSLFHIFDGD

SEQ ID NO: 695 - protein, 15 aa, synthetic construct
IKSLFHIFDGDNEIL

SEQ ID NO: 696 - protein, 15 aa, synthetic construct
FHIFDGDNEILQIVD

SEQ ID NO: 697 - protein, 15 aa, synthetic construct
DGDNEILQIVDELSE

SEQ ID NO: 698 - protein, 15 aa, synthetic construct
EILQIVDELSEDITK

SEQ ID NO: 699 - protein, 15 aa, synthetic construct
KPIVQYDNFQDEENI

SEQ ID NO: 700 - protein, 15 aa, synthetic construct
IVDELSEDITKYFMK

SEQ ID NO: 701 - protein, 12 aa, synthetic construct
LSEDITKYFMKL

SEQ ID NO: 702 - protein, 15 aa, synthetic construct
QYDNFQDEENIGIYK

SEQ ID NO: 703 - protein, 15 aa, synthetic construct
FQDEENIGIYKELED

SEQ ID NO: 704 - protein, 15 aa, synthetic construct
ENIGIYKELEDLIEK

SEQ ID NO: 705 - protein, 15 aa, synthetic construct
IYKELEDLIEKNENL

SEQ ID NO: 706 - protein, 15 aa, synthetic construct
LEDLIEKNENLDDLD

SEQ ID NO: 707 - protein, 15 aa, synthetic construct
IEKNENLDDLDEGIE

SEQ ID NO: 708 - protein, 15 aa, synthetic construct
MKDAVEINDITSKLI

SEQ ID NO: 709 - protein, 15 aa, synthetic construct
ELEKALSEDSKEIID

SEQ ID NO: 710 - protein, 15 aa, synthetic construct
ALSEDSKEIIDAKDD

SEQ ID NO: 711 - protein, 15 aa, synthetic construct
DSKEIIDAKDDTLEK

SEQ ID NO: 712 - protein, 15 aa, synthetic construct
IIDAKDDTLEKVIEE

SEQ ID NO: 713 - protein, 15 aa, synthetic construct
KDDTLEKVIEEEHDI

SEQ ID NO: 714 - protein, 15 aa, synthetic construct
LEKVIEEEHDITTTL

SEQ ID NO: 715 - protein, 15 aa, synthetic construct
IEEEHDITTTLDEVV

SEQ ID NO: 716 - protein, 15 aa, synthetic construct
HDITTTLDEVVELKD

SEQ ID NO: 717 - protein, 15 aa, synthetic construct
TTLDEVVELKDVEED

SEQ ID NO: 718 - protein, 15 aa, synthetic construct
EVVELKDVEEDKIEK

SEQ ID NO: 719 - protein, 15 aa, synthetic construct
VEINDITSKLIEETQ

SEQ ID NO: 720 - protein, 15 aa, synthetic construct
LKDVEEDKIEKVSDL

SEQ ID NO: 721 - protein, 15 aa, synthetic construct
EEDKIEKVSDLKDLE

SEQ ID NO: 722 - protein, 15 aa, synthetic construct
IEKVSDLKDLEEDIL

SEQ ID NO: 723 - protein, 15 aa, synthetic construct
SDLKDLEEDILKEVK

SEQ ID NO: 724 - protein, 15 aa, synthetic construct
DLEEDILKEVKEIKE

SEQ ID NO: 725 - protein, 15 aa, synthetic construct
DILKEVKEIKELESE

SEQ ID NO: 726 - protein, 15 aa, synthetic construct
EVKEIKELESEILED

SEQ ID NO: 727 - protein, 15 aa, synthetic construct
IKELESEILEDYKEL

SEQ ID NO: 728 - protein, 15 aa, synthetic construct
ESEILEDYKELKTIE

SEQ ID NO: 729 - protein, 15 aa, synthetic construct
LEDYKELKTIETDIL

SEQ ID NO: 730 - protein, 15 aa, synthetic construct
DITSKLIEETQELNE

SEQ ID NO: 731 - protein, 15 aa, synthetic construct
KELKTIETDILEEKK

SEQ ID NO: 732 - protein, 15 aa, synthetic construct
TIETDILEEKKEIEK

SEQ ID NO: 733 - protein, 15 aa, synthetic construct
DILEEKKEIEKDHFE

SEQ ID NO: 734 - protein, 15 aa, synthetic construct
EKKEIEKDHFEKFEE

SEQ ID NO: 735 - protein, 15 aa, synthetic construct
IEKDHFEKFEEEAEE

SEQ ID NO: 736 - protein, 15 aa, synthetic construct
HFEKFEEEAEEIKDL

SEQ ID NO: 737 - protein, 15 aa, synthetic construct
FEEEAEEIKDLEADI

SEQ ID NO: 738 - protein, 11 aa, synthetic construct
AEEIKDLEADI

SEQ ID NO: 739 - protein, 15 aa, synthetic construct
KLIEETQELNEVEAD

SEQ ID NO: 740 - protein, 15 aa, synthetic construct
ETQELNEVEADLIKD

SEQ ID NO: 741 - protein, 15 aa, synthetic construct
LNEVEADLIKDMEKL

SEQ ID NO: 742 - protein, 15 aa, synthetic construct
EADLIKDMEKLKELE

SEQ ID NO: 743 - protein, 15 aa, synthetic construct
IKDMEKLKELEKALS

SEQ ID NO: 744 - protein, 15 aa, synthetic construct
EKLKELEKALSEDSK

SEQ ID NO: 745 - protein, 8 aa, synthetic construct
GIRIHYNH

SEQ ID NO: 746 - protein, 15 aa, synthetic construct
ILNTIIINPCTCVPN

SEQ ID NO: 747 - protein, 15 aa, synthetic construct
PCTCVPNTLPIEYFD

SEQ ID NO: 748 - protein, 15 aa, synthetic construct
LPIEYFDYETLKENV

SEQ ID NO: 749 - protein, 15 aa, synthetic construct
ETLKENVKIRDSNTK

SEQ ID NO: 750 - protein, 15 aa, synthetic construct
IRDSNTKTQLIILSA

SEQ ID NO: 751 - protein, 15 aa, synthetic construct
CASILLSLSSLWGIR

SEQ ID NO: 752 - protein, 12 aa, synthetic construct
SSLWGIRIHYNH

SEQ ID NO: 753 - protein, 15 aa, synthetic construct
IIINPCTCVPNTLPI

SEQ ID NO: 754 - protein, 15 aa, synthetic construct
VPNTLPIEYFDYETL

SEQ ID NO: 755 - protein, 15 aa, synthetic construct
YFDYETLKENVKIRD

SEQ ID NO: 756 - protein, 15 aa, synthetic construct
ENVKIRDSNTKTQLI

SEQ ID NO: 757 - protein, 15 aa, synthetic construct
NTKTQLIILSAVCAS

SEQ ID NO: 758 - protein, 15 aa, synthetic construct
LLSLSSLWGIRIHYN

SEQ ID NO: 759 - protein, 15 aa, synthetic construct
NSSDSDCEPLPFGLK

SEQ ID NO: 760 - protein, 15 aa, synthetic construct
GKLERKDMYLIWHYS

SEQ ID NO: 761 - protein, 15 aa, synthetic construct
RKDMYLIWHYSHSLL

SEQ ID NO: 762 - protein, 15 aa, synthetic construct
YLIWHYSHSLLRDKF

SEQ ID NO: 763 - protein, 15 aa, synthetic construct
HYSHSLLRDKFNKMK

SEQ ID NO: 764 - protein, 15 aa, synthetic construct
SLLRDKFNKMKSSLW

SEQ ID NO: 765 - protein, 15 aa, synthetic construct
DKFNKMKSSLWSICG

SEQ ID NO: 766 - protein, 15 aa, synthetic construct
KMKSSLWSICGKLAH

SEQ ID NO: 767 - protein, 15 aa, synthetic construct
SLWSICGKLAHEHKL

SEQ ID NO: 768 - protein, 15 aa, synthetic construct
ICGKLAHEHKLPFKI

SEQ ID NO: 769 - protein, 15 aa, synthetic construct
LAHEHKLPFKIKMKK

SEQ ID NO: 770 - protein, 15 aa, synthetic construct
SDCEPLPFGLKPSDL

SEQ ID NO: 771 - protein, 15 aa, synthetic construct
HKLPFKIKMKKWWKC

SEQ ID NO: 772 - protein, 15 aa, synthetic construct
FKIKMKKWWKCCGHV

SEQ ID NO: 773 - protein, 15 aa, synthetic construct
MKKWWKCCGHVTDEL

SEQ ID NO: 774 - protein, 15 aa, synthetic construct
WKCCGHVTDELLIKE

SEQ ID NO: 775 - protein, 15 aa, synthetic construct
GHVTDELLIKEHDDY

SEQ ID NO: 776 - protein, 15 aa, synthetic construct
DELLIKEHDDYNSIY

SEQ ID NO: 777 - protein, 15 aa, synthetic construct
IKEHDDYNSIYNYIN

SEQ ID NO: 778 - protein, 15 aa, synthetic construct
DDYNSIYNYINNESS

SEQ ID NO: 779 - protein, 15 aa, synthetic construct
SIYNYINNESSSREQ

SEQ ID NO: 780 - protein, 15 aa, synthetic construct
YINNESSSREQFLIF

SEQ ID NO: 781 - protein, 15 aa, synthetic construct
PLPFGLKPSDLNRKV

SEQ ID NO: 782 - protein, 15 aa, synthetic construct
ESSSREQFLIFLNMI

SEQ ID NO: 783 - protein, 15 aa, synthetic construct
REQFLIFLNMIKHSW

SEQ ID NO: 784 - protein, 15 aa, synthetic construct
LIFLNMIKHSWTTFT

SEQ ID NO: 785 - protein, 15 aa, synthetic construct
NMIKHSWTTFTMETF

SEQ ID NO: 786 - protein, 15 aa, synthetic construct
HSWTTFTMETFIKCK

SEQ ID NO: 787 - protein, 15 aa, synthetic construct
TFTMETFIKCKISLE

SEQ ID NO: 788 - protein, 15 aa, synthetic construct
ETFIKCKISLENNMR

SEQ ID NO: 789 - protein, 15 aa, synthetic construct
KCKISLENNMRNVTN

SEQ ID NO: 790 - protein, 11 aa, synthetic construct
SLENNMRNVTN

SEQ ID NO: 791 - protein, 15 aa, synthetic construct
GLKPSDLNRKVTEED

SEQ ID NO: 792 - protein, 15 aa, synthetic construct
SDLNRKVTEEDLERM

SEQ ID NO: 793 - protein, 15 aa, synthetic construct
RKVTEEDLERMIIEL

SEQ ID NO: 794 - protein, 15 aa, synthetic construct
EEDLERMIIELPGKL

SEQ ID NO: 795 - protein, 15 aa, synthetic construct
ERMIIELPGKLERKD

SEQ ID NO: 796 - protein, 15 aa, synthetic construct
IELPGKLERKDMYLI

SEQ ID NO: 797 - protein, 7 aa, synthetic construct
NMRNVTN

SEQ ID NO: 798 - protein, 15 aa, synthetic construct
NHLGNVKYLVIVFLI

SEQ ID NO: 799 - protein, 15 aa, synthetic construct
YREEVCNDEVDLYLL

SEQ ID NO: 800 - protein, 15 aa, synthetic construct
VCNDEVDLYLLMDCS

SEQ ID NO: 801 - protein, 15 aa, synthetic construct
EVDLYLLMDCSGSIR

SEQ ID NO: 802 - protein, 15 aa, synthetic construct
YLLMDCSGSIRRHNW

SEQ ID NO: 803 - protein, 15 aa, synthetic construct
DCSGSIRRHNWVNHA

SEQ ID NO: 804 - protein, 15 aa, synthetic construct
SIRRHNWVNHAVPLA

SEQ ID NO: 805 - protein, 15 aa, synthetic construct
HNWVNHAVPLAMKLI

SEQ ID NO: 806 - protein, 15 aa, synthetic construct
NHAVPLAMKLIQQLN

SEQ ID NO: 807 - protein, 15 aa, synthetic construct
PLAMKLIQQLNLNDN

SEQ ID NO: 808 - protein, 15 aa, synthetic construct
KLIQQLNLNDNAIHL

SEQ ID NO: 809 - protein, 15 aa, synthetic construct
NVKYLVIVFLIFFDL

SEQ ID NO: 810 - protein, 15 aa, synthetic construct
QLNLNDNAIHLYASV

SEQ ID NO: 811 - protein, 15 aa, synthetic construct
NDNAIHLYASVFSNN

SEQ ID NO: 812 - protein, 15 aa, synthetic construct
IHLYASVFSNNAREI

SEQ ID NO: 813 - protein, 15 aa, synthetic construct
ASVFSNNAREIIRLH

SEQ ID NO: 814 - protein, 15 aa, synthetic construct
SNNAREIIRLHSDAS

SEQ ID NO: 815 - protein, 15 aa, synthetic construct
REIIRLHSDASKNKE

SEQ ID NO: 816 - protein, 15 aa, synthetic construct
RLHSDASKNKEKALI

SEQ ID NO: 817 - protein, 15 aa, synthetic construct
DASKNKEKALIIIKS

SEQ ID NO: 818 - protein, 15 aa, synthetic construct
NKEKALIIIKSLLST

SEQ ID NO: 819 - protein, 15 aa, synthetic construct
ALIIIKSLLSTNLPY

SEQ ID NO: 820 - protein, 15 aa, synthetic construct
IKSLLSTNLPYGKTN

SEQ ID NO: 821 - protein, 15 aa, synthetic construct
LSTNLPYGKTNLTDA

SEQ ID NO: 822 - protein, 15 aa, synthetic construct
LPYGKTNLTDALLQV

SEQ ID NO: 823 - protein, 15 aa, synthetic construct
KTNLTDALLQVRKHL

SEQ ID NO: 824 - protein, 15 aa, synthetic construct
TDALLQVRKHLNDRI

SEQ ID NO: 825 - protein, 15 aa, synthetic construct
LQVRKHLNDRINREN

SEQ ID NO: 826 - protein, 15 aa, synthetic construct
KHLNDRINRENANQL

SEQ ID NO: 827 - protein, 15 aa, synthetic construct
DRINRENANQLVVIL

SEQ ID NO: 828 - protein, 15 aa, synthetic construct
RENANQLVVILTDGI

SEQ ID NO: 829 - protein, 15 aa, synthetic construct
NQLVVILTDGIPDSI

SEQ ID NO: 830 - protein, 15 aa, synthetic construct
FLIFFDLFLVNGRDV

SEQ ID NO: 831 - protein, 15 aa, synthetic construct
VILTDGIPDSIQDSL

SEQ ID NO: 832 - protein, 15 aa, synthetic construct
DGIPDSIQDSLKESR

SEQ ID NO: 833 - protein, 15 aa, synthetic construct
DSIQDSLKESRKLSD

SEQ ID NO: 834 - protein, 15 aa, synthetic construct
DSLKESRKLSDRGVK

SEQ ID NO: 835 - protein, 15 aa, synthetic construct
ESRKLSDRGVKIAVF

SEQ ID NO: 836 - protein, 15 aa, synthetic construct
LSDRGVKIAVFGIGQ

SEQ ID NO: 837 - protein, 15 aa, synthetic construct
GVKIAVFGIGQGINV

SEQ ID NO: 838 - protein, 15 aa, synthetic construct
AVFGIGQGINVAFNR

SEQ ID NO: 839 - protein, 15 aa, synthetic construct
IGQGINVAFNRFLVG

SEQ ID NO: 840 - protein, 12 aa, synthetic construct
INVAFNRFLVGC

SEQ ID NO: 841 - protein, 15 aa, synthetic construct
FDLFLVNGRDVQNNI

SEQ ID NO: 842 - protein, 15 aa, synthetic construct
LVNGRDVQNNIVDEI

SEQ ID NO: 843 - protein, 15 aa, synthetic construct
RDVQNNIVDEIKYRE

SEQ ID NO: 844 - protein, 15 aa, synthetic construct
NNIVDEIKYREEVCN

SEQ ID NO: 845 - protein, 15 aa, synthetic construct
DEIKYREEVCNDEVD

SEQ ID NO: 846 - protein, 15 aa, synthetic construct
LNASKVNKKGKIAEE

SEQ ID NO: 847 - protein, 12 aa, synthetic construct
SGAIASVAAILG

SEQ ID NO: 848 - protein, 15 aa, synthetic construct
KVNKKGKIAEEKKRK

SEQ ID NO: 849 - protein, 15 aa, synthetic construct
KGKIAEEKKRKNIKN

SEQ ID NO: 850 - protein, 15 aa, synthetic construct
AEEKKRKNIKNIDKA

SEQ ID NO: 851 - protein, 15 aa, synthetic construct
KRKNIKNIDKAIEEH

SEQ ID NO: 852 - protein, 15 aa, synthetic construct
IKNIDKAIEEHNKRK

SEQ ID NO: 853 - protein, 15 aa, synthetic construct
DKAIEEHNKRKKLIY

SEQ ID NO: 854 - protein, 15 aa, synthetic construct
EEHNKRKKLIYYSLI

SEQ ID NO: 855 - protein, 15 aa, synthetic construct
KRKKLIYYSLIASGA

SEQ ID NO: 856 - protein, 15 aa, synthetic construct
MKVSRHTVLLNIILI

SEQ ID NO: 857 - protein, 15 aa, synthetic construct
LGYKSWKNKNKSKDK

SEQ ID NO: 858 - protein, 9 aa, synthetic construct
KNKSKDKVN

SEQ ID NO: 859 - protein, 15 aa, synthetic construct
RHTVLLNIILIVSLL

SEQ ID NO: 860 - protein, 15 aa, synthetic construct
CVLTLNLFSANDDKR

SEQ ID NO: 861 - protein, 15 aa, synthetic construct
SANDDKRALKDIDRT

SEQ ID NO: 862 - protein, 15 aa, synthetic construct
LKDIDRTLEKLLRKK

SEQ ID NO: 863 - protein, 15 aa, synthetic construct
EKLLRKKQIIISTAA

SEQ ID NO: 864 - protein, 15 aa, synthetic construct
ALAITLGGLFGSLGY

SEQ ID NO: 865 - protein, 15 aa, synthetic construct
LFGSLGYKSWKNKNK

SEQ ID NO: 866 - protein, 13 aa, synthetic construct
SWKNKNKSKDKVN

SEQ ID NO: 867 - protein, 15 aa, synthetic construct
LNLFSANDDKRALKD

SEQ ID NO: 868 - protein, 15 aa, synthetic construct
DKRALKDIDRTLEKL

SEQ ID NO: 869 - protein, 15 aa, synthetic construct
DRTLEKLLRKKQIII

SEQ ID NO: 870 - protein, 15 aa, synthetic construct
RKKQIIISTAAVALA

SEQ ID NO: 871 - protein, 15 aa, synthetic construct
TAAVALAITLGGLFG

SEQ ID NO: 872 - protein, 15 aa, synthetic construct
TLGGLFGSLGYKSWK

SEQ ID NO: 873 - protein, 17 aa, synthetic construct
PSDKHIKEYLNKIQNSL

SEQ ID NO: 874 - protein, 24 aa, synthetic construct
GIQVRIKPGSANKPKDELDYANDI

SEQ ID NO: 875 - protein, 48 aa, synthetic construct
STEWSPCSVTCGNGIQVRIKPGSANKPKDELDYANDIEKKICKMEKCS

SEQ ID NO: 876 - protein, 105 aa, synthetic construct
KTIIIVTLFILILNTIIINPCTCVPNTLPIEYFDYETLKENVKIRDSNTKTQLIILSAVCASILLSLSSLWGIRIHYNHEMNKKEINNYELNSDLLKDADFYMVE

SEQ ID NO: 877 - protein, 144 aa, synthetic construct
AGCAAAAAAAAAAATAAAAAAGGATCAGGTGAACCATTAATAGATGTACACGATTTAATATCTGATATGATCAAAAAAGAAGAAGAACTTGTTGAAGTTAACAAAAGAAAATCCAAATATAAACTTGCCACTTCAGTACTTGCA

(19) United States
(12) Patent Application Publication
(10) Pub. No.: US 2026/0256898 A1
(43) Pub. Date: Sep. 3, 2026

(54) COMPOSITIONS FOR DELIVERY OF LIVER STAGE ANTIGENS AND RELATED METHODS

(71) Applicant: BioNTech SE, Mainz (DE)

(72) Inventors: Asaf Poran, Stoneham, MA (US); Daniel Abram Rothenberg, Melrose, MA (US); Theresa Addona, Needham, MA (US); Raquel Mariska Furtado, Arlington, MA (US); Ugur Sahin, Mainz (DE); Thomas Ziegenhals, Undenheim (DE)

(21) Appl. No.: 19/114,293

(22) PCT Filed: Sep. 22, 2023
(86) PCT No.: PCT/US2023/074956

§ 371 Date: Mar. 21, 2025

Related U.S. Application Data
(60) Provisional application No. 63/515,331, filed on Jul. 24, 2023.

(30) Foreign Application Priority Data
Sep. 23, 2022 (WO) PCT/US2022/044625

(51) Int. Cl. A61K 39/015; A61K 39/00; A61P 33/06; C07K 14/005; C07K 14/445

(52) U.S. Cl. CPC A61K 39/015; A61P 33/06; C07K 14/005; C07K 14/445; A61K 2039/53; A61K 2039/55555; A61K 2039/572; C07K 2319/03; C12N 2710/16622; C12N 2710/16634

(57) ABSTRACT

The present disclosure provides compositions (e.g., pharmaceutical compositions) for delivery of Plasmodium protein antigens and related technologies (e.g., components thereof and/or methods relating thereto). Among other things, the present disclosure provides polyribonucleotides encoding Plasmodium protein antigens.

### BACKGROUND

Malaria is a mosquito-borne infectious disease caused by protozoan parasites of the Plasmodium genus. According to the World Health Organization, an estimated 3.4 billion people in 92 countries are at risk of being infected with the malaria parasite and developing disease.

### SUMMARY

The present disclosure provides pharmaceutical compositions (e.g., immunogenic compositions, e.g., vaccines) for delivering particular Plasmodium antigens (e.g., Plasmodium T-cell antigens) to a subject (e.g., a patient) and related technologies (e.g., methods). Plasmodium antigens may also be referred to as “malaria antigens” or “malarial antigens” herein. In particular, the present disclosure provides malaria vaccine compositions and related technologies (e.g., methods). The present disclosure includes the unexpected discovery that antigens disclosed herein and fragments thereof, are particularly advantageous for use in preventing or treating malaria, e.g., in antigen constructs and/or vaccines as further disclosed herein.

In some embodiments, the present disclosure provides a polyribonucleotide encoding a polypeptide, wherein the polypeptide comprises one or more one or more Plasmodium T-cell antigens. In some embodiments, the one or more Plasmodium T-cell antigens comprises at least 2 and at most 10 Plasmodium T-cell antigens. In some embodiments, the encoded polypeptide comprises at least 25 amino acids and at most 1100 amino acids. In some embodiments, the encoded polypeptide comprises at least 25 amino acids and at most 500 amino acids.

In some embodiments, a polyribonucleotide disclosed herein encodes one or more Plasmodium T cell antigens comprising two or more of:
(i) an antigenic Plasmodium CSP polypeptide fragment;
(ii) an antigenic Plasmodium LSA-1 (a) polypeptide fragment;
(iii) an antigenic Plasmodium LSA-1 (b) polypeptide fragment;
(iv) an antigenic Plasmodium TRAP polypeptide fragment;
(v) an antigenic Plasmodium LSAP2 polypeptide fragment;
(vi) an antigenic Plasmodium UIS3 polypeptide fragment;
(vii) an antigenic Plasmodium ETRAMP10.3 polypeptide fragment;
(viii) an antigenic Plasmodium LISP-1 polypeptide fragment;
(ix) an antigenic Plasmodium LISP-2 polypeptide fragment; and
(x) an antigenic Plasmodium LSA-3 polypeptide fragment.

In some embodiments, a polyribonucleotide disclosed herein encodes one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment; and
(v) an antigenic Plasmodium LSAP2 polypeptide fragment.

In some embodiments, a polyribonucleotide encodes an amino acid sequence with at least 85% sequence identity to an amino acid sequence according to SEQ ID NO: 15.

In some embodiments, a polyribonucleotide encodes a polypeptide comprising one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-3 polypeptide fragment;
(vii) an antigenic Plasmodium LSA-1 (a) polypeptide fragment; and
(viii) an antigenic Plasmodium LSA-1 (b) polypeptide fragment.

In some embodiments, a polyribonucleotide encodes a polypeptide comprising or consisting of an amino acid sequence with at least 85% sequence identity to an amino acid sequence according to SEQ ID NO: 18.

In some embodiments, a polyribonucleotide encodes one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment;
(viii) an antigenic Plasmodium LISP-2 polypeptide fragment; and
(ix) an antigenic Plasmodium LISP-1 polypeptide fragment.

In some embodiments, a polyribonucleotide encodes a polypeptide comprising or consisting of an amino acid sequence with at least 85% sequence identity to an amino acid sequence according to SEQ ID NO: 24.

In some embodiments, a polyribonucleotide encodes one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment; and
(viii) an antigenic Plasmodium LISP-1 polypeptide fragment.

In some embodiments, a polyribonucleotide encodes a polypeptide that comprises or consists of an amino acid sequence with at least 85% sequence identity to an amino acid sequence according to SEQ ID NO: 27.

In some embodiments, a polyribonucleotide encodes a polypeptide that comprises one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LISP-2 polypeptide fragment; and
(vii) an antigenic Plasmodium LISP-1 polypeptide fragment.

In some embodiments, a polyribonucleotide encodes a polypeptide that comprises or consists of an amino acid sequence with at least 85% sequence identity to an amino acid sequence according to SEQ ID NO: 30.

In some embodiments, a polyribonucleotide encodes a polypeptide that encodes one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (b) polypeptide fragment; and
(vii) an antigenic Plasmodium LISP-1 polypeptide fragment.

In some embodiments, a polyribonucleotide encodes a polypeptide that comprises or consists of an amino acid sequence with at least 85% sequence identity to an amino acid sequence according to SEQ ID NO: 33.

In some embodiments, a polyribonucleotide encodes one or more Plasmodium T cell antigens comprising:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment;
(viii) an antigenic Plasmodium LISP-2 polypeptide fragment;
(ix) an antigenic Plasmodium LISP-1 polypeptide fragment; and
(x) an antigenic Plasmodium LSA-3 polypeptide fragment.

In some embodiments, a polyribonucleotide encodes a polypeptide that comprises or consists of an amino acid sequence with at least 85% sequence identity to an amino acid sequence according to SEQ ID NO: 36.

In some embodiments, a polyribonucleotide encodes a polypeptide that comprises or consists of one or more Plasmodium T cell antigens comprising:
(i) an antigenic Plasmodium LSA-1 (a) polypeptide fragment;
(ii) an antigenic Plasmodium LSA-1 (b) polypeptide fragment;
(iii) an antigenic Plasmodium LISP-2 polypeptide fragment;
(iv) an antigenic Plasmodium LISP-1 polypeptide fragment; and
(v) an antigenic Plasmodium LSA-3 polypeptide fragment.

In some embodiments, a polyribonucleotide encodes a polypeptide that comprises or consists of an amino acid sequence with at least 85% sequence identity to an amino acid sequence according to SEQ ID NO: 48.

In some embodiments, a polyribonucleotide encodes a polypeptide that comprises one or more Plasmodium T cell antigens comprising:
(i) an antigenic Plasmodium LSA-1 (a) polypeptide fragment;
(ii) an antigenic Plasmodium LSA-1 (b) polypeptide fragment;
(iii) an antigenic Plasmodium LISP-2 polypeptide fragment; and
(iv) an antigenic Plasmodium LISP-1 polypeptide fragment.

In some embodiments, a polyribonucleotide encodes a polypeptide that comprises or consists of an amino acid sequence with at least 85% sequence identity to an amino acid sequence according to SEQ ID NO: 45.

In some embodiments, a polyribonucleotide encodes one or more Plasmodium T cell antigens comprising an antigenic Plasmodium CSP polypeptide fragment, wherein the antigenic Plasmodium CSP polypeptide fragment comprises a Plasmodium CSP N-terminal region. In some embodiments, the antigenic Plasmodium CSP polypeptide fragment further comprises a Plasmodium CSP N-terminal end region. In some embodiments, the antigenic Plasmodium CSP polypeptide fragment further comprises a Plasmodium CSP junction region.

In some embodiments, an antigenic Plasmodium CSP polypeptide fragment comprises or consists of an amino acid sequence with at least 85% sequence identity to an amino acid sequence according to SEQ ID NO: 133.

In some embodiments, one or more Plasmodium T cell antigens do not comprise an antigenic Plasmodium berghei CSP polypeptide fragment.

In some embodiments, one or more Plasmodium T cell antigens comprise the antigenic Plasmodium LSA-1 (a) polypeptide fragment, wherein the antigenic Plasmodium LSA-1 (a) polypeptide fragment comprises or consists of an amino acid sequence with at least 85% sequence identity to an amino acid sequence according to SEQ ID NO: 144.

In some embodiments, one or more Plasmodium T cell antigens comprise the antigenic Plasmodium LSA-1 (b) polypeptide fragment, wherein the antigenic Plasmodium LSA-1 (b) polypeptide fragment comprises or consists of an amino acid sequence with at least 85% sequence identity to an amino acid sequence according to SEQ ID NO: 155.

In some embodiments, one or more Plasmodium T cell antigens comprise the antigenic Plasmodium TRAP polypeptide fragment, wherein the antigenic Plasmodium TRAP polypeptide fragment comprises or consists of an amino acid sequence with at least 85% sequence identity to an amino acid sequence according to SEQ ID NO: 171.

In some embodiments, one or more Plasmodium T cell antigens comprise the antigenic Plasmodium LSAP2 polypeptide fragment, wherein the antigenic Plasmodium LSAP2 polypeptide fragment comprises or consists of an amino acid sequence with at least 85% sequence identity to an amino acid sequence according to SEQ ID NO: 198.

In some embodiments, one or more Plasmodium T cell antigens comprise the antigenic Plasmodium UIS3 polypeptide fragment, wherein the antigenic Plasmodium UIS3 polypeptide fragment comprises or consists of an amino acid sequence with at least 85% sequence identity to an amino acid sequence according to SEQ ID NO: 212.

In some embodiments, one or more Plasmodium T cell antigens comprise the antigenic Plasmodium ETRAMP10.3 polypeptide fragment, wherein the antigenic Plasmodium ETRAMP10.3 polypeptide fragment comprises or consists of an amino acid sequence with at least 85% sequence identity to an amino acid sequence according to SEQ ID NO: 219.

In some embodiments, one or more Plasmodium T cell antigens comprise the antigenic Plasmodium LISP-1 polypeptide fragment, wherein the antigenic Plasmodium LISP-1 polypeptide fragment comprises or consists of an amino acid sequence with at least 85% sequence identity to an amino acid sequence according to SEQ ID NO: 229.

In some embodiments, one or more Plasmodium T cell antigens comprise the antigenic Plasmodium LISP-2 polypeptide fragment, wherein the antigenic Plasmodium LISP-2 polypeptide fragment comprises or consists of an amino acid sequence with at least 85% sequence identity to an amino acid sequence according to SEQ ID NO: 238.

In some embodiments, one or more Plasmodium T cell antigens comprise the antigenic Plasmodium LSA-3 polypeptide fragment, wherein the antigenic Plasmodium LSA-3 polypeptide fragment comprises or consists of an amino acid sequence with at least 85% sequence identity to an amino acid sequence according to SEQ ID NO: 249.

In some embodiments, one or more Plasmodium T cell antigens each comprise one or more T cell epitopes.

In some embodiments, a polyribonucleotide encodes a polypeptide that does not comprise an antigenic fragment of a bacterial polypeptide. In some embodiments, an encoded polypeptide does not comprise an antigenic bacillus Calmette-Guérin (BCG) polypeptide fragment, optionally wherein the antigenic BCG polypeptide fragment comprises an amino acid sequence according to SEQ ID NO: 461. In some embodiments, an encoded polypeptide does not comprise an antigenic tetanus toxin (TT) polypeptide fragment, optionally wherein the antigenic TT polypeptide fragment comprises an amino acid sequence according to SEQ ID NO: 462.

In some embodiments, one or more Plasmodium T cell antigens do not comprise an antigenic Plasmodium sporozoite threonine-asparagine-rich protein (STARP) polypeptide fragment, optionally wherein the antigenic Plasmodium STARP polypeptide fragment comprises an amino acid sequence according to SEQ ID NO: 463.

In some embodiments, a polyribonucleotide encodes a polypeptide that further comprises an MHC class I trafficking signal (MITD). In some embodiments, wherein the MITD comprises or consists of an amino acid sequence according to SEQ ID NO: 479.

In some embodiments, a polyribonucleotide encodes a polypeptide that comprises a secretory signal.

In some embodiments, a secretory signal comprises or consists a Plasmodium secretory signal. In some embodiments, a Plasmodium secretory signal comprises or consists of a Plasmodium CSP secretory signal. In some embodiments, a Plasmodium CSP secretory signal comprises or consists of an amino acid sequence according to SEQ ID NO: 397.

In some embodiments, a secretory signal comprises or consists of a heterologous secretory signal. In some embodiments, a heterologous secretory signal comprises or consists of a non-human secretory signal.

In some embodiments, a heterologous secretory signal comprises or consists of a viral secretory signal.

In some embodiments, a viral secretory signal comprises or consists of an HSV secretory signal. In some embodiments, an HSV secretory signal comprises or consists of an HSV-1 or HSV-2 secretory signal. In some embodiments, an HSV secretory signal comprises or consists of an HSV glycoprotein D (gD) secretory signal. In some embodiments, an HSV gD secretory signal comprises or consists of an amino acid sequence according to SEQ ID NO: 382. In some embodiments, an HSV gD secretory signal comprises or consists of an amino acid sequence according to SEQ ID NO: 388.

In some embodiments, a secretory signal comprises or consists of an Ebola virus secretory signal. In some embodiments, an Ebola virus secretory signal comprises or consists of an Ebola virus spike glycoprotein (SGP) secretory signal. In some embodiments, an Ebola virus SGP secretory signal comprises or consists of an amino acid sequence according to SEQ ID NO: 400.

In some embodiments, a secretory signal is located at the N-terminus of the polypeptide.

In some embodiments, a polypeptide comprises a transmembrane region.

In some embodiments, a transmembrane region comprises or consists of a Plasmodium

transmembrane region. In some embodiments, a Plasmodium transmembrane region comprises or consists of a Plasmodium CSP glycosylphosphatidylinositol (GPI) anchor region.

In some embodiments, a Plasmodium CSP GPI anchor region comprises or consists of an amino acid sequence according to SEQ ID NO: 444.

In some embodiments, a transmembrane region comprises or consists of a heterologous transmembrane region. In some embodiments, a heterologous transmembrane region does not comprise a hemagglutin transmembrane region. In some embodiments, a heterologous transmembrane region comprises or consists of a non-human transmembrane region. In some embodiments, a heterologous transmembrane region comprises or consists of a viral transmembrane region.

In some embodiments, a heterologous transmembrane region comprises or consists of an HSV transmembrane region. In some embodiments, a HSV transmembrane region comprises or consists of an HSV-1 or HSV-2 transmembrane region. In some embodiments, a HSV transmembrane region comprises or consists of an HSV gD transmembrane region. In some embodiments, a HSV gD transmembrane region comprises or consists of an amino acid sequence according to SEQ ID NO: 447.

In some embodiments, a transmembrane region comprises or consists of a human transmembrane region. In some embodiments, a human transmembrane region comprises or consists of a human decay accelerating factor glycosylphosphatidylinositol (hDAF-GPI) anchor region. In some embodiments, an hDAF-GPI anchor region comprises or consists of an amino acid sequence according to SEQ ID NO: 450.

In some embodiments, a polypeptide does not comprise a secretory signal.

In some embodiments, a polypeptide does not comprise a transmembrane region.

In some embodiments, a polypeptide comprises one or more linkers. In some embodiments, one or more linkers comprise or consist of an amino acid sequence according to SEQ ID NO: 452. In some embodiments, one or more linkers comprise or consist of an amino acid sequence according to SEQ ID NO: 459. In some embodiments, one or more linkers comprise or consist of an amino acid sequence according to SEQ ID NO: 456. In some embodiments, one or more linkers comprise or consist of an amino acid sequence according to SEQ ID NO: 460.

In some embodiments, a polypeptide comprises a linker between two Plasmodium T-cell antigens.

In some embodiments, one or more Plasmodium T cell antigens are one or more P. falciparum T cell antigens. In some embodiments, one or more P. falciparum T cell antigens are from P. falciparum isolate 3D7. In some embodiments, one or more Plasmodium T-cell antigens are from a Plasmodium species capable of infecting a human.

In some embodiments, each of the one or more Plasmodium T-cell antigens comprise at least 21 amino acids.

In some embodiments, a polyribonucleotide is an isolated polyribonucleotide.

In some embodiments, a polyribonucleotide is an engineered polyribonucleotide.

In some embodiments, a polyribonucleotide is a codon-optimized polyribonucleotide.

In some embodiments, provided herein is an RNA construct comprising in 5′ to 3′ order:
(i) a 5′ UTR that comprises or consists of a modified human alpha-globin 5′-UTR;
(ii) a polyribonucleotide of any one of claims 1-82;
(iii) a 3′ UTR that comprises or consists of a first sequence from the amino terminal enhancer of split (AES) messenger RNA and a second sequence from the mitochondrial encoded 12S ribosomal RNA; and
(iv) a polyA tail sequence.

In some embodiments, a 5′ UTR comprises or consists of a ribonucleic acid sequence according to SEQ ID NO: 465.

In some embodiments, a 3′ UTR comprises or consists of a ribonucleic acid sequence according to SEQ ID NO: 471.

In some embodiments, a polyA tail sequence is a split polyA tail sequence.

In some embodiments, a split polyA tail sequence comprises or consists of a ribonucleic acid sequence according to SEQ ID NO: 467.

In some embodiments, a RNA construct comprises a 5′ cap.

In some embodiments, a RNA construct comprises a cap proximal sequence comprising positions +1, +2, +3, +4, and +5 of the polyribonucleotide.

In some embodiments, a RNA construct comprises a 5′ cap comprising or consisting of m7(3′OMeG)(5′)ppp(5′)(2′OMeA1)pG2, wherein A1 is position +1 of the polyribonucleotide, and G₂ is position +2 of the polyribonucleotide. In some embodiments, a RNA construct further comprises a cap proximal sequence comprising A1 and G₂ of the Cap1 structure, and a sequence comprising: A3A4Us (SEQ ID NO: 480) at positions +3, +4 and +5 respectively of the polyribonucleotide.

In some embodiments, disclosed herein is a composition comprising one or more polyribonucleotides (e.g., one or more polyribonucleotides disclosed herein).

In some embodiments, disclosed herein is a composition comprising one or more RNA constructs (e.g., one or more RNA constructs disclosed herein).

In some embodiments, a composition disclosed herein comprises lipid nanoparticles, polyplexes (PLX), lipidated polyplexes (LPLX), or liposomes. In some embodiments, one or more polyribonucleotides are fully or partially encapsulated within the lipid nanoparticles, polyplexes (PLX), lipidated polyplexes (LPLX), or liposomes.

In some embodiments, a composition disclosed herein further comprises lipid nanoparticles, wherein the one or more polyribonucleotides are encapsulated within the lipid nanoparticles. In some embodiments, lipid nanoparticles target liver cells. In some embodiments, lipid nanoparticles target secondary lymphoid organ cells. In some embodiments, the lipid nanoparticles are cationic lipid nanoparticles.

In some embodiments, lipid nanoparticles each comprise:
(a) a polymer-conjugated lipid;
(b) a cationically ionizable lipid; and
(c) one or more neutral lipids.

In some embodiments, a polymer-conjugated lipid comprises a PEG-conjugated lipid. In some embodiments, a polymer-conjugated lipid comprises 2-[(polyethylene glycol)-2000]-N,N-ditetradecylacetamide.

In some embodiments, one or more neutral lipids comprise 1,2-Distearoyl-sn-glycero-3-phosphocholine (DPSC).

In some embodiments, one or more neutral lipids comprise cholesterol.

In some embodiments, a cationically ionizable lipid comprises [(4-Hydroxybutyl) azanediyl]di(hexane-6,1-diyl)bis(2-hexyldecanoate).

In some embodiments, lipid nanoparticles have an average diameter of about 50-150 nm.

In some embodiments, the present disclosure provides a pharmaceutical composition comprising a composition (e.g., a composition disclosed herein) and at least one pharmaceutically acceptable excipient. In some embodiments, a pharmaceutical comprises a cryoprotectant, optionally wherein the cryoprotectant is sucrose. In some embodiments, a pharmaceutical comprises an aqueous buffered solution, optionally wherein the aqueous buffered solution comprises one or more of Tris base, Tris HCl, NaCl, KCl, Na₂HPO₄, and KH₂PO₄.

In some embodiments, the present disclosure provides a combination comprising:
(i) a first pharmaceutical composition comprising a first polyribonucleotide, wherein the first polyribonucleotide encodes a first polypeptide, and the first polypeptide comprises a one or more Plasmodium T-cell antigens; and
(ii) a second pharmaceutical composition comprising a second polyribonucleotide, wherein the second polyribonucleotide encodes a second polypeptide, and the second polypeptide comprises one or more Plasmodium antigenic polypeptide regions or portions thereof.

In some embodiment, a combination comprises a first pharmaceutical composition comprising a polyribonucleotide disclosed herein.

In some embodiments, a combination disclosed herein comprises a second polyribonucleotide encoding a polypeptide that comprises one or more Plasmodium antigenic polypeptide regions or portions thereof and comprises one or more Plasmodium CSP regions or portions thereof.

In some embodiments, a combination comprises:
(i) a first pharmaceutical composition comprising a polyribonucleotide encoding a first polypeptide, wherein the first polypeptide comprises a one or more Plasmodium T-cell antigens, and wherein the one or more Plasmodium T-cell antigens comprises a Plasmodium N-terminal region or portion thereof, but not a Plasmodium C-terminal region or portion thereof; and
(ii) a second pharmaceutical composition comprising a polyribonucleotide encoding a second polypeptide, wherein the second polypeptide comprises one or more Plasmodium CSP polypeptide regions or portions thereof, and wherein the one or more Plasmodium CSP polypeptide regions or portions thereof comprises a Plasmodium CSP C-terminal region or portion thereof, but not a Plasmodium CSP N-terminal region or portion thereof.

In some embodiments, combinations disclosed herein comprise a first pharmaceutical composition and a second pharmaceutical composition, wherein the first and second pharmaceutical compositions are not in the same composition.

In some embodiments, disclosed herein is a combination comprising:
(i) a first pharmaceutical composition comprising a first polyribonucleotide; and
(ii) a second pharmaceutical composition comprising a second polyribonucleotide, wherein the second polyribonucleotide.

In some embodiments, the present disclosure provides a method comprising administering a polyribonucleotide (e.g., a polyribonucleotide disclosed herein) to a subject.

In some embodiments, the present disclosure provides a method comprising administering an RNA construct (e.g., an RNA construct disclosed herein) to a subject.

In some embodiments, the present disclosure provides a method comprising administering a composition (e.g., a composition disclosed herein) to a subject.

In some embodiments, the present disclosure provides a method comprising administering one or more doses of a pharmaceutical composition (e.g., a pharmaceutical composition disclosed herein) to a subject.

In some embodiments, the present disclosure provides a pharmaceutical composition for use in the treatment of a malaria infection, wherein the method comprises administering one or more doses of the pharmaceutical composition to a subject.

In some embodiments, the present disclosure provides a pharmaceutical composition for use in the prevention of a malaria infection comprising administering one or more doses of the pharmaceutical composition to a subject.

In some embodiments, a method disclosed herein, or a pharmaceutical composition for use disclosed herein comprises administering two or more doses of the pharmaceutical composition to a subject.

In some embodiments, a method comprises administering three or more doses of a pharmaceutical composition disclosed herein to a subject. In some embodiments, a pharmaceutical composition for use comprises administering three or more doses of a pharmaceutical composition disclosed herein to a subject. In some embodiments, the second of the three or more doses is administered to the subject at least 4 weeks after the first of the three or more doses is administered to the subject. In some embodiments, the third of the three or more doses is administered to the subject at least 4 weeks after the second of the three or more doses is administered to the subject.

In some embodiments, a method comprises administering a fourth dose of a pharmaceutical composition disclosed herein to a subject. In some embodiments, a pharmaceutical composition for use comprises administering a fourth dose of a pharmaceutical composition disclosed herein to a subject. In some embodiments, the fourth dose is administered to a subject at least one year after the third of the three or more doses is administered to the subject.

In some embodiments, a method comprises administering a combination (e.g., a combination disclosed herein). In some embodiments, a method comprises administering a combination comprising a first pharmaceutical composition and a second pharmaceutical composition. In some embodiments, the first and the second pharmaceutical composition are administered on the same day. In some embodiments, the first and second pharmaceutical compositions are administered on different days. In some embodiments, the first and second pharmaceutical compositions are administered to a subject at different locations on the subject's body.

In some embodiments, the present disclosure provides a method of treating a malaria infection.

In some embodiments, the present disclosure provides a method of preventing a malaria infection.

In some embodiments, a subject has or is at risk of developing a malaria infection. In some embodiments, a subject is human.

In some embodiments, administration of a pharmaceutical composition disclosed herein, a combination disclosed herein, or a polyribonucleotide disclosed herein induces an anti-malaria immune response in a subject. In some embodiments, an anti-malaria immune response in the subject comprises an adaptive immune response. In some embodiments, an anti-malaria immune response comprises a T-cell response. In some embodiments, a T-cell response is or comprises a CD4+ T cell response, a CD8+ T cell response, and/or B-cell response. In some embodiments, an anti-malaria immune system response comprises the production of antibodies directed against the one or more Plasmodium antigens.

In some embodiments, the present disclosure provides a use of a pharmaceutical composition (e.g., a pharmaceutical composition described herein) in the treatment of a malaria infection.

In some embodiments, the present disclosure provides a use of a pharmaceutical composition (e.g., a pharmaceutical composition described herein) in the prevention of a malaria infection.

In some embodiments, the present disclosure provides a use of a pharmaceutical composition (e.g., a pharmaceutical composition disclosed herein), in inducing an anti-malaria immune response in a subject.

In some embodiments, the present disclosure provides a polypeptide encoded by a polyribonucleotide described herein.

In some embodiments, the present disclosure provides a polypeptide encoded by an RNA construct described herein.

In some embodiments, the present disclosure provides a host cell comprising a polyribonucleotide (e.g., a polyribonucleotide described herein).

In some embodiments, the present disclosure comprises a host cell (e.g., a host cell comprising a polyribonucleotide disclosed herein, an RNA construct described herein, and/or a polypeptide disclosed herein).

### BRIEF DESCRIPTION OF THE DRAWING

FIG. 1 presents an exemplary workflow for identification, selection and/or characterization of antigens (e.g., Plasmodium proteins, including particular variants, and/or epitopes thereof, in particular T cell epitopes) for use in accordance with the present disclosure.

FIGS. 2A-2K show immunological characterization of eleven Plasmodium proteins (specifically, CSP, TRAP, EXP1, UIS3, ETRAMP10.3, LISP-1, LISP-2, LSA-1, LSA-3, LSAP1, and LSAP2), and also depict fragments selected for inclusion in an antigen (e.g., a string construct antigen) for use in accordance with the present disclosure.

FIG. 2L shows antigenic fragments of Plasmodium polypeptides encoded by exemplary RNA constructs described herein.

FIG. 2M shows antigenic fragments of Plasmodium protein LSA-3.

FIG. 3 presents a schematic representation of exemplary Plasmodium T cell string polypeptide constructs containing antigens, as described herein.

FIGS. 4A-4F depict activation of T-cells, as assessed by secretion of IFN-γ. FIG. 4A shows an exemplary study design including dosing and peptide string construct design. FIGS. 4B-4D show an assessment of IFN-γ secretion using isolated splenocytes (from mice immunized with different T cell peptide string constructs) incubated with construct specific antigen peptide pools (15mers, 11aa overlap across antigen). FIG. 4E depicts a comparison of isolated splenocytes (from mice in group 2 and 3, and splenocytes isolated from mice in group 4) response to specific antigen peptide pools. FIG. 4F depicts a comparison of isolated splenocytes (from mice in group 2 and splenocytes isolated from mice in group 1) response to specific antigen peptide pools.

FIGS. 5A-5I depict activation of T-cells, as assessed by secretion of IFN-γ. FIG. 5A shows an exemplary study design including dosing and peptide string construct design. FIGS. 5B-5I show an assessment of IFN-γ secretion using isolated splenocytes (from mice immunized with different T cell peptide string constructs) incubated with construct specific antigen peptide pools (15mers, 11aa overlap across antigen).

FIGS. 6A-6B depict assessment of activation of T-cells, as assessed by secretion of IFN-γ using isolated splenocytes (from mice immunized with T cell peptide string constructs individually or with T cell strings constructs in combination).

FIGS. 7A-7B depict assessment of activation of T-cells, as assessed by secretion of IFN-γ using isolated splenocytes (from mice immunized with shorter T cell peptide string constructs or a longer T cell peptide string with the same antigenic content).

FIG. 8 depicts transfection of a combination including RNA constructs 55 and 57 into cells to generate detectable protein product. Relative protein expression 24 h after co-transfecting 2.5 μg each drug product into HEK293T cell lines are shown.

### DEFINITIONS

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, provided compounds show one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by 13C-or 14C-enriched carbon are within the scope of this disclosure.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent,” may refer to a physical entity. In some embodiments, an agent may be characterized by a particular feature and/or effect. For example, as used herein, the term “therapeutic agent” refers to a physical entity has a therapeutic effect and/or elicits a desired biological and/or pharmacological effect. In some embodiments, an agent may be a compound, molecule, or entity of any chemical class including, for example, a small molecule, polypeptide, nucleic acid, saccharide, lipid, metal, or a combination or complex thereof.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy-and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antigen: The term “antigen”, as used herein, refers to an agent that elicits an immune response; and/or (ii) an agent that binds to a T cell receptor (e.g., when presented by an MHC molecule) or to an antibody.

Anti-malaria immune response. The term “anti-malaria immune response”, as used herein, refers to an immune response directed to one or more antigens derived from Plasmodium.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

C-terminal domain: The term “C-terminal domain”, as used herein, refers to a region of a CSP polypeptide that corresponds to amino acids 273-397 of wild-type CSP sequence of Plasmodium falciparum (isolate 3D7)(SEQ ID NO:1).

C-terminal region: The term “C-terminal region”, as used herein, refers to a region of a CSP polypeptide that corresponds to amino acids 273-375 of wild-type CSP sequence (SEQ ID NO:1). In some embodiments, a serine follows immediately after the C-terminal region. In some embodiments, a serine and a valine follow immediately after the C-terminal region.

Central domain: The term “central domain”, as used herein, refers to a region of a CSP polypeptide that corresponds to amino acids 105-272 of wild-type CSP sequence (SEQ ID NO:1).

Combination therapy. As used herein, the term “combination therapy” refers to those situations in which a subject is simultaneously exposed to two or more therapeutic regimens (e.g., two or more therapeutic agents (e.g., two or more antibody agents)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, administration of combination therapy may involve administration of one or more agent(s) or modality (ies) to a subject receiving the other agent(s) or modality (ies) in the combination. For clarity, combination therapy does not require that individual agents be administered together in a single composition (or even necessarily at the same time), although in some embodiments, two or more agents, or active moieties thereof, may be administered together in a combination composition.

Comparable. As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Dosing regimen: Those skilled in the art will appreciate that the term “dosing regimen” (or “therapeutic regimen”) may be used to refer to a set of unit doses (typically more than one) that are administered individually to a subject, typically separated by periods of time. In some embodiments, a given therapeutic agent has a recommended dosing regimen, which may involve one or more doses.

Encode: As used herein, the term “encode” or “encoding” refers to sequence information of a first molecule that guides production of a second molecule having a defined sequence of nucleotides (e.g., a polyribonucleotide) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule encodes a polypeptide if transcription and translation of RNA corresponding to that gene produces the polypeptide in a cell or other biological system. In some embodiments, a coding region of a polyribonucleotide encoding a target antigen refers to a coding strand, the nucleotide sequence of which is identical to the polyribonucleotide sequence of such a target antigen. In some embodiments, a coding region of a polyribonucleotide encoding a target antigen refers to a non-coding strand of such a target antigen, which may be used as a template for transcription of a gene or cDNA.

Expression. As used herein, the term “expression” of a nucleic acid sequence refers to the generation of a gene product from the nucleic acid sequence. In some embodiments, a gene product can be a transcript, e.g., a polyribonucleotide as provided herein. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Heterologous: As used herein, the term “heterologous”, with respect to secretory signal or transmembrane region, refers to a secretory signal or transmembrane region from a virus or an organism other than Plasmodium.

Homology. As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided composition (e.g., a pharmaceutical composition) may be “increased” relative to that obtained with a comparable reference composition. Alternatively or additionally, in some embodiments, an assessed value achieved in a subject may be “increased” relative to that obtained in the same subject under different conditions (e.g., prior to or after an event; or presence or absence of an event such as administration of a composition (e.g., a pharmaceutical composition) as described herein, or in a different, comparable subject (e.g., in a comparable subject that differs from the subject of interest in prior exposure to a condition, e.g., absence of administration of a composition (e.g., a pharmaceutical composition) as described herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

In order: As used herein with reference to a polynucleotide or polyribonucleotide, “in order” refers to the order of features from 5′ to 3′ along the polynucleotide or polyribonucleotide. As used herein with reference to a polypeptide, “in order” refers to the order of features moving from the N-terminal-most of the features to the C-terminal-most of the features along the polypeptide. “In order” does not mean that no additional features can be present among the listed features. For example, if Features A, B, and C of a polynucleotide are described herein as being “in order, Feature A, Feature B, and Feature C,” this description does not exclude, e.g., Feature D being located between Features A and B.

Isolated: The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Junction region: The term “junction region”, as used herein, refers to a region of a CSP polypeptide that corresponds to amino acids 93-104 of wild-type CSP sequence (SEQ ID NO:1). Junction region variant. The term “junction region variant”, as used herein, refers to a junction region that comprises one or more substitution mutation as compared to amino acids 93-104 of wild-type CSP sequence (SEQ ID NO:1).

Linker. As used herein, the term “linker” refers to a portion of a polypeptide that connects different regions, portions, or antigens to one another.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

Major repeat region: As used herein, the term “major repeat region” refers to a region of a CSP polypeptide that corresponds to amino acids 129-272 of wild-type CSP sequence (SEQ ID NO:1) and contains 35 repeats of the amino acid sequence NANP (SEQ ID NO: 108). The 35 repeats of the amino acid sequence NANP (SEQ ID NO: 108) are separated into two contiguous stretches, the first stretch containing 17 repeats of the amino acid sequence NANP (SEQ ID NO: 108) and second stretch containing 18 repeats of the amino acid sequence NANP (SEQ ID NO: 108) which flank an amino acid sequence of NVDP (SEQ ID NO: 105). A portion of the major repeat region contains at least the amino acid sequence NPNA (SEQ ID NO: 104). Preferably a portion of the major repeat region contains at least the amino acid sequences NANPNA (SEQ ID NO: 114) and NPNANP (SEQ ID NO: 111). As used herein, “repeat” in reference to sequence A refers to sequence A being present once, and “one or more repeats” of sequence A refers to sequence A being present one or more times.

Merozoite stage specific Plasmodium antigen: As used herein, the term “merozoite stage specific Plasmodium antigen” refers to an antigen that is expressed during the merozoite stage of the Plasmodium life cycle.

Minor repeat region: As used herein, the term “minor repeat region” refers to a region of a CSP polypeptide that corresponds to amino acids 105-128 of wild-type CSP sequence (SEQ ID NO:1) and contains 3 repeats of the amino acid sequence NANPNVDP (SEQ ID NO: 477). A minor repeat region does not contain the amino acid sequence NPNA (SEQ ID NO: 104), and does not contain the amino acid sequence NANPNA (SEQ ID NO: 114) or NPNANP (SEQ ID NO: 111). As used herein, “repeat” in reference to sequence A refers to sequence A being present once, and three repeats of sequence A refers to sequence A being present three times.

N-terminal domain: As used herein, the term “N-terminal domain” refers to a region of a CSP polypeptide that corresponds to amino acids 19-92 of wild-type CSP sequence (SEQ ID NO:1).

N-terminal end region. As used herein, the term “N-terminal end region” refers to a region of a CSP polypeptide that corresponds to amino acids 81-92 of wild-type CSP sequence (SEQ ID NO:1).

N-terminal region: As used herein, the term “N-terminal region” refers to a region of a CSP polypeptide that corresponds to amino acids 19-80 of wild-type CSP sequence (SEQ ID NO:1).

RNA lipid nanoparticle: As used herein, the term “RNA lipid nanoparticle” refers to a nanoparticle comprising at least one lipid and RNA molecule(s), e.g., one or more polyribonucleotides as provided herein. In some embodiments, an RNA lipid nanoparticle comprises at least one cationic amino lipid. In some embodiments, an RNA lipid nanoparticle comprises at least one cationic amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the average particle diameter. In some embodiments, RNA lipid nanoparticles may be prepared by mixing lipids with RNA molecules described herein.

Neutralization. As used herein, the term “neutralization” refers to an event in which binding agents such as antibodies bind to a biological active site of a parasite such as a receptor binding protein, thereby inhibiting the parasitic infection of cells. In some embodiments, the term “neutralization” refers to an event in which binding agents eliminate or significantly reduce ability of infecting cells.

Nucleic acid/Polynucleotide. As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutically effective amount: The term “pharmaceutically effective amount” or “therapeutically effective amount” refers to the amount which achieves a desired reaction or a desired effect alone or together with further doses. In the case of the treatment of a particular disease (e.g., malaria), a desired reaction in some embodiments relates to inhibition of the course of the disease (e.g., malaria). In some embodiments, such inhibition may comprise slowing down the progress of a disease (e.g., malaria) and/or interrupting or reversing the progress of the disease (e.g., malaria). In some embodiments, a desired reaction in a treatment of a disease (e.g., malaria) may be or comprise delay or prevention of the onset of a disease (e.g., malaria) or a condition (e.g., a malaria associated condition). An effective amount of a composition (e.g., a pharmaceutical composition) described herein will depend, for example, on disease (e.g., malaria) or a condition (e.g., a malaria associated condition) to be treated, the severity of such a disease (e.g., malaria) or a condition (e.g., a malaria associated condition), individual parameters of the patient, including, e.g., age, physiological condition, size and weight, the duration of treatment, the type of an accompanying therapy (if present), the specific route of administration and similar factors. Accordingly, doses of a composition (e.g., a pharmaceutical composition) described herein may depend on various of such parameters. In the case that a reaction in a patient is insufficient with an initial dose, higher doses (or effectively higher doses achieved by a different, more localized route of administration) may be used.

Polypeptide. As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide. In some embodiments, a polypeptide is a Plasmodium T cell string polypeptide construct described herein. A Plasmodium T cell string polypeptide construct is a polypeptide that includes one or more T-cell antigens from one or more Plasmodium proteins, or one or more portions thereof. In some embodiments, a Plasmodium T cell string polypeptide construct additionally includes one or more additional amino acid sequences, such as a secretory signal (e.g., a heterologous secretory signal), a transmembrane region (e.g., a heterologous transmembrane region), a trafficking signal, and/or a linker, as described herein.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time. In some embodiments, prevention refers to reducing the risk of developing clinical malaria.

Reference. As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA) or Polyribonucleotide. As used herein, the term “ribonucleic acid,” “RNA,” or “polyribonucleotide” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is a mRNA. In some embodiments, where an RNA is a mRNA, a RNA typically comprises at its 3′ end a poly(A) region. In some embodiments, where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods). In some embodiments, a polyribonucleotide encodes a polypeptide, which is preferably is a Plasmodium T cell string polypeptide construct.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Secretory signal: As used herein, the term “secretory signal” refers to an amino acid sequence motif that targets associated polypeptides for translocation to a secretory pathway.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In preferred embodiments, a subject is a human subject. In some embodiments, a subject is suffering from a disease, disorder, or condition (e.g., malaria and/or a malaria-associated condition). In some embodiments, a subject is susceptible to a disease, disorder, or condition (e.g., malaria and/or a malaria-associated condition). In some embodiments, a subject displays one or more symptoms or characteristics of a disease, disorder, or condition (e.g., malaria and/or a malaria-associated condition). In some embodiments, a subject displays one or more non-specific symptoms of a disease, disorder, or condition (e.g., malaria and/or a malaria-associated condition). In some embodiments, a subject does not display any symptom or characteristic of a disease, disorder, or condition (e.g., malaria and/or a malaria-associated condition). In some embodiments, a subject is someone with one or more features characteristic of susceptibility to or risk of a disease, disorder, or condition (e.g., malaria and/or a malaria-associated condition). In some embodiments, a subject is a patient. In some embodiments, a subject is an individual to whom diagnosis and/or therapy is and/or has been administered.

Suffering from: An individual who is “suffering from” a disease, disorder, and/or condition (e.g., malaria and/or a malaria-associated condition) has been diagnosed with and/or displays one or more symptoms of a disease, disorder, and/or condition.

Susceptible to. An individual who is “susceptible to” a disease, disorder, and/or condition (e.g., malaria and/or a malaria-associated condition) is one who has a higher risk of developing the disease, disorder, and/or condition (e.g., malaria and/or a malaria-associated condition) than does a member of the general public. In some embodiments, an individual who is susceptible to a disease, disorder and/or condition (e.g., malaria and/or a malaria-associated condition) may not have been diagnosed with the disease, disorder, and/or condition (e.g., malaria and/or a malaria-associated condition). In some embodiments, an individual who is susceptible to a disease, disorder, and/or condition (e.g., malaria and/or a malaria-associated condition) may exhibit symptoms of the disease, disorder, and/or condition (e.g., malaria and/or a malaria-associated condition). In some embodiments, an individual who is susceptible to a disease, disorder, and/or condition (e.g., malaria and/or a malaria-associated condition) may not exhibit symptoms of the disease, disorder, and/or condition (e.g., malaria and/or a malaria-associated condition). In some embodiments, an individual who is susceptible to a disease, disorder, and/or condition (e.g., malaria and/or a malaria-associated condition) will develop the disease, disorder, and/or condition (e.g., malaria and/or a malaria-associated condition). In some embodiments, an individual who is susceptible to a disease, disorder, and/or condition (e.g., malaria and/or a malaria-associated condition) will not develop the disease, disorder, and/or condition (e.g., malaria and/or a malaria-associated condition).

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition (e.g., malaria and/or a malaria-associated condition). In some embodiments, a therapeutic agent or therapy is a medical intervention that can be performed to alleviate, relieve, inhibit, present, delay onset of, reduce severity of, and/or reduce incidence of one or more symptoms or features of a disease, disorder, and/or condition.

Transmembrane region: As used herein, the term “transmembrane region” refers to a region of a polypeptide that spans a biological membrane, such as the plasma membrane of a cell.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition (e.g., malaria and/or a malaria-associated condition). Treatment may be administered to a subject who does not exhibit signs of a disease, disorder, and/or condition (e.g., malaria and/or a malaria-associated condition). In some embodiments, treatment may be administered to a subject who exhibits only early signs of the disease, disorder, and/or condition (e.g., malaria and/or a malaria-associated condition), for example for the purpose of decreasing the risk of developing pathology associated with the disease, disorder, and/or condition. In some embodiments, treatment may be administered to a subject at a later-stage of disease, disorder, and/or condition (e.g., malaria and/or a malaria-associated condition).

Variant: As used herein, the term “variant” refers to a molecule that shows significant structural (e.g., primary or secondary) identity with a reference molecule but differs structurally from the reference molecule. For example, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone).

### DETAILED DESCRIPTION OF CERTAIN EMBODIMENTS

### I. Malaria

Malaria is a mosquito-borne infectious disease caused by single-celled eukaryotic Plasmodium parasites that are transmitted by the bite of Anopheles spp. mosquitoes (Phillips, M., et al. Malaria. Nat Rev Dis Primers 3, 17050 (2017), which is incorporated herein by reference in its entirety). Mosquitoes that transmit malaria must have been infected through a previous blood meal taken from an infected subject (e.g., a human). When a mosquito bites an infected subject a small amount of blood is taken in containing Malaria parasites. The infected mosquito can then subsequently bite a non-infected subject, infecting the subject.

Malaria remains one of the most serious infectious diseases, causing approximately 200 million clinical cases and 500,000-600,000 deaths annually. Although significant effort has been invested in developing therapeutic treatments for malaria, many malaria parasites have developed resistance to available therapeutics. According to Malaria Eradication Research Agenda Initiative, malaria eradication will only be achievable through effective vaccination.

In 2015, the European Medicines Agency gave a positive review to a malaria vaccine candidate known as “RTS,S”, a milestone in malaria vaccine development. In 2019, the World Health Organization launched pilot programs that provide RTS,S to children at least 5 months of age in parts of three sub-Saharan African countries. RTS,S/AS01 is an adjuvanted protein subunit vaccine that consists of a portion of the major repeat region and the C-terminus of CSP from Plasmodium falciparum fused to the Hepatitis B surface antigen (HBsAg). The vaccine is a mix of this PICSP-HBsAg compound with HBsAg that forms virus-like particles (RTS,S/AS01; Mosquirix™). RTS,S is administered according to a regimen that requires four doses: an initial 3-dose schedule given at least 1 month apart, and a 4th dose 15-18 months after dose 3 (see, for example, Vandoolaeghe & Schuerman Expert Rev Vaccines. 15:1481, 2016; PATH_MVI_RTSS_Fact Sheet_042019, each of which is incorporated herein by reference in its entirety). Reports indicate that RTS,S protects approximately 30% to 50% of children from clinical disease over 18 months. RTS,S has been reported to induce protective antibody and CD4+ T-cell responses, but only negligible CD8+ T cell responses (see, for example, Moris et al. Hum Vaccin Immunother 14:17, 2018, which is incorporated herein by reference in its entirety). Phase III studies of RTS,S delivered as a three-dose series with a booster after 1 yr (year) showed moderate vaccine efficacy in children aged 5 to 17 months preventing 36% of clinical malaria cases over the full study period with a median follow-up of 4 yrs, with a range of 20% in high to 66% in low transmission settings. Furthermore, published literature suggests that protection wanes over time including reports of potential negative efficacy after 5 yrs in children with high malaria exposure (Olotu et al. 2016, N. Engl. J. Med. 374:2519-29, which is incorporated herein by reference in its entirety). Thus, an effective malaria vaccine remains an unmet medical need of critical importance for global health.

### Lifecycle

During a blood meal, infected mosquitos inject, along with their anticoagulating saliva, sporozoites known as the liver stage of Plasmodium spp. Sporozoites journey through the skin to the lymphatics and into hepatocytes of the liver. This journey happens very quickly; it can be complete within only a few minutes (Sinnis et al., Parasitol Int. 2007 September; 56 (3): 171-8, which is herein incorporated by reference in its entirety). This is a time known to be a bottle kneck of Malaria infection most favorable for therapeutic intervention, as only a small number (thought to be a few hundred at maximum) of sporozoites are injected by the mosquito, with only fraction of that number establishing infection in the liver and developing into mature live-stage parasites (Flores-Garcia et al., mBio. 2018 Nov. 20; 9 (6): e02194-18, which is herein incorporated by reference in its entirety). Thus, a subject whose immune system is primed to clear sporozoites before they enter hepatocytes can efficiently clear an infection.

One particular challenge associated with clearing a malarial infection during this bottle neck is that the most abundant and immunogenic protein on the sporozoite surface, the circumsporozoite protein (CSP), is only exposed to the immune system in small quantities and for short duration of time due to the variably low inoculum from the mosquito and the kinetics of hepatocyte infection after inoculation. After liver infection is established, the parasite differentiates into a stage which no longer expresses CSP and instead has a different mosaic of surface antigens. Furthermore, due to the density and close proximity of neighboring CSPs on the surface of the parasite coupled with the bi-valency of antibodies, binding of antibodies to CSP can produce a phenomenon referred to as CSP precipitation reaction, whereby antibodies can crosslink neighboring CSP and cause them to precipitate and shed from the parasite surface, leaving a trail of precipitated antibody bound CSP that the parasite can replace through its normal CSP translocation process (Livingstone et al., Sci Rep 11, 5318 (2021); Steward et al., J Protozool. 1991 July-August; 38 (4): 411-21, which are herein incorporated by reference in their entirety).

When moving from an inoculation site in the skin to the liver, sporozoites traverse host cells (Mota et al., Science 2001 Jan. 5;291 (5501): 141-4, which is herein incorporated by reference in its entirety). Sporozoites traverse different types of host cells at the dermis, including fibroblasts and phagocytes (Amino et al., Cell Host Microbe. 2008 Feb. 14;3 (2): 88-96, which is herein incorporated by reference in its entirety), and the liver sinusoidal barrier, containing liver endothelial cellsand Kupffer cells (Frevert et al., PLOS Biol 3 (6): e192. 2005, which is herein incorporated by reference in its entirety) and sinusoidal endothelial cells (Tavares et al., J Exp Med 2013 May 6;210 (5): 905-15, which is herein incorporated by reference in its entirety), in order to gain access to hepatocytes. Sporozoites preferentially traverse cells with low-sulfated heparin sulfate proteoglycans (HSPGs) but preferentially invade cells with high-sulfated HSPGs (Coppi et al., Cell Host & Microbe 2, 316-327, November 2007, which is herein incorporated by reference in its entirety).

Cell traversal was first observed as non-phagocytic entry of P. berghei sporozoites into macrophages followed by “escape” from these cells (Vanderberg et al., J. Euk. Microbiol. 37:528-536, 1990, which is herein incorporated by reference in its entirety). The biochemical, biophysical, and stepwise processes of traversal are still being explored. However, it has been suggested by electron microscopy that host cell rupture occurs upon entry and exit from the host cell (Mota et al., 2001; Tavares et al., 2013, which is herein incorporated by reference in its entirety). It has also been shown that P. yoelii sporozoites can enter hepatocytes via a transient vacuole and that host membrane rupture occurs upon cell exit rather than cell entry (Risco-Castillo et al., Cell Host Microbe 2015 Nov. 11; 18 (5): 593-603, which is herein incorporated by reference in its entirety).

Sporozoites also traverse hepatocytes before establishing a productive hepatocyte infection (Mota et al., 2001, which is herein incorporated by reference in its entirety). Several possibilities emerged as to why this occurs. The first hypothesis suggested that migration through hepatocytes primes parasites for invasion by activating apical exocytosis (Mota et al., Nat Med 2002 November; 8 (11): 1318-22, which is herein incorporated by reference in its entirety). The second theory suggested that traversal releases hepatocyte growth factor (HGF), making neighboring hepatocytes more susceptible to infection (Carrolo et al., Nat Med. 2003 November;9 (11): 1363-9, which is herein incorporated by reference in its entirety). Lastly, other studies suggest that it takes some time for sporozoites to switch off the machinery for traversal and activate invasion machinery (Amino et al., 2008, Coppi et al., 2007, which are herein incorporated by reference in their entirety), and that traversal primarily functions to penetrate cell barriers and avoid phagocytosis en route to the liver (Amino et al., 2008, Coppi et al., 2007, Tavares et al., 2013, which are herein incorporated by reference in their entirety).

Although it has been shown that sporozoites traverse human cells (Behet et al., Malar J 2014 Apr. 5; 13:136; Cha et al., J Exp Med 2015 Aug. 24; 212 (9): 1391-403; Dumoulin et al., PLOS One 2015 Jun. 12;10 (6): e0129623; van Schaijk et al., PLOS ONE, 3 (10). e3549 2008, which are herein incorporated by reference in their entirety), the molecular basis for the traversal process is largely unstudied. Antibodies against circumsporozoite protein (CSP) impair traversal (Dumoulin et al., 2015, which is herein incorporated by reference in its entirety), but this is likely due to inhibition of motility rather than a direct effect (Cha et al., J Exp Med 2016 Sep. 19; 213 (10): 2099-112, which is herein incorporated by reference in its entirety). Furthermore, antibodies induced by chloroquine prophylaxis with sporozoites interfere with cell traversal, and these may also target CSP (Behet et al., 2014). Recently it was shown that glyceraldehyde 3-phosphate dehydrogenase (GAPDH) on the parasite surface interacts with CD68 on Kupffer cells during traversal (Cha et al., 2015, Cha et al., 2016, which are herein incorporated by reference in their entirety).

In rodent malaria parasites such as P. berghei, two sporozoite microneme proteins have been identified that appear to be essential for cell traversal (sporozoite microneme protein essential for cell traversal [SPECT1; Ishino et al., PLOS Biol., 2 (2004), pp. 77-84] and SPECT2 [Ishino et al., Cell. Microbiol., 7 (2005), pp. 199-208], also called perforin-like protein 1 [PLP1] [Kaiser et al., Mol. Biochem. Parasitol., 133 (2004), pp. 15-26], which are herein incorporated by reference in their entirety). Even though genetic disruption of SPECT1 or SPECT2 rendered sporozoites unable to traverse murine cells, they still invaded hepatocytes in vitro (Ishino et al., 2004, Ishino et al., 2005, which are herein incorporated by reference in their entirety). When injected into rodents, sporozoites lacking SPECT1 or SPECT2 were impaired for liver infection, but a small number of sporozoites could still establish liver infection that resulted in subsequent patency. However, depletion of Kupffer cells allowed mutants to establish liver infection at levels comparable with wild-type parasites (Ishino et al., 2004, Ishino et al., 2005, which are herein incorporated by reference in their entirety). This data suggests that traversal by rodent-infecting sporozoites is important for navigating through the sinusoidal layer, but not for hepatocyte invasion, malarial exoerythrocytic forms development, or growth within erythrocytes (Ishino et al., 2004, Ishino et al., 2005, which are herein incorporated by reference in their entirety).

The ortholog of SPECT2 in P. yoelii, PLP1, has been shown to play a role in cell traversal. Although this protein is not required for hepatocyte entry, it plays a role in egress from transient vacuoles during traversal (Risco-Castillo et al., 2015, which are herein incorporated by reference in their entirety). Thus, sporozoites that infect rodents can traverse host cells by generating a vacuole at the entry step and use a perforin-like protein (e.g., SPECT2/PLP1) to escape from this compartment and/or a host cell, during cell exit.

Once sporozoites have invaded liver cells, they differentiate into merozoites, a replicative form of the parasite capable of lysing hepatocytes after multiple rounds of replication. Within a few days, a few hundred sporozoites can become hundreds of thousands of merozoites. When infected liver cells rupture, they release the merozoites into the bloodstream, where they invade red blood cells and begin the asexual reproductive stage, which is the symptomatic stage of the disease. Within a small number of days, millions of merozoites can be present in blood.

Malaria symptoms typically develop 4-8 days after initial red blood cell invasion. Replication cycle of merozoites within the red blood cells continues for 36-72 hours, until hemolysis, releasing the merozoites for another round of red blood cell infection. Thus, in synchronous infections (infections that originate from a single infectious bite), fever occurs every 36-72 hours, when infected red blood cells lyse and release endotoxins en masse.

Plasmodium spp. parasites gain entry into red blood cells through specific ligand-receptor interactions mediated by proteins on the surface of the parasite that interact with receptors on the host erythrocyte (mature red blood cell) or reticulocyte (immature red blood cell), whereas P. falciparum can invade and replicate in erythrocytes and reticulocytes, P. vivax and other species predominantly invade reticulocytes, which are less abundant than erythrocytes. Most of the erythrocyte-binding proteins or reticulocyte-binding proteins that have been associated with invasion are redundant or are expressed as a family of variant forms; however, for P. falciparum, two essential red blood cell receptors (basigin and complement decay-accelerating factor (also known as CD55)) have been identified.

Plasmodium vivax and Plasmodium ovale can also enter a dormant state in the liver, the hypnozoite.

Merozoites released from red blood cells can invade other red blood cells and continue to replicate, or in some cases, they differentiate into male or female gametocytes. Gametocytes concentrate in skin capillaries and are then taken up by the mosquito vector in another blood meal. In the gut of the mosquito, each male gametocyte produces eight microgametes after three rounds of mitosis; the female gametocyte matures into a macrogamete. Male microgametes are motile forms with flagellae and seek the female macrogamete. The male and female gametocytes fuse, forming a diploid zygote, which elongates into an ookinete; this motile form secretes a chitinase in order to enter the peritrophic membrane and traverse the midgut epithelium to the basal lateral side of the midgut, establishing itself in the basal lamina as an oocyst Oocysts mature over 14-15 days, undergoing cycles of replication to form sporozoites that are ultimately liberated into the hemocoel, an environment rich in sugars and subtrates beneficial to the parasite's survival. Thousands of sporozoites can form from a single oocyst and become randomly distributed throughout the hemocoel. These sporozoites are motile and rapidly destroy the hemolymph, with only approximately 20% successfully invading the salivary gland. Following invasion of the salivary gland, sporozoites are re-programmed via an unknown mechanism to prepare for liver invasion. Evidence of this reprogramming has been demonstrated by the inability of midgut sporzoites (directly from oocysts) to invade hepatocytes, and also by the fact that sporzoites which have successfully invaded a salivary gland are unable to do re-invade another salivary gland if presented one. Salivary gland sporozoites alter mosquito behavior and salivary gland function, as less saliva is produced resulting in an increase in mosquito probing behavior, increasing the chances of transmission to a human host via a mosquito bite.

Some drugs that prevent Plasmodium spp. invasion or proliferation in the liver have prophylactic activity, drugs that block the red blood cell stage are required for the treatment of the symptomatic phase of the disease, and compounds that inhibit the formation of gametocytes or their development in the mosquito (including drugs that kill mosquitoes feeding on blood) are transmission-blocking agents (Phillips, et al. Malaria. Nat Rev Dis Primers 3, 17050 (2017), which is incorporated herein by reference in its entirety).

### B. Genome

Since completion of the first sequence of P. falciparum 3D7 genome in 2002, genomic research on malaria parasites has rapidly advanced. Except for a short diploid phase after fertilization in the mosquito midgut, Plasmodium parasites are haploid throughout their life cycle. The genomes of different species range from 20 to 35 megabases, contain 14 chromosomes, a circular plastid genome of approximately 35 kilobases, and multiple copies of a 6 kilobase mitochondrial DNA. Comparison of genomes from different species showed that homologous genes are often found in synthetic blocks arranged in different orders among different chromosomes.

The adenine-thymine (AT) content of Plasmodium spp. can also be very different, e.g., ~80% AT in P. falciparum, P. reichenowi, and P. gallinaceum, ~75% AT in rodent malaria parasites; and ~60% AT in P. vivax, P. knowlesi, and P. cynomolgi. AT content is often higher in introns and intergenic noncoding regions than in protein-coding exons, with an average of 80.6% AT for the whole P. falciparum genome versus 86.5% for noncoding sequences. The high AT content of P. falciparum reflects large numbers of low-complexity regions, simple sequence repeats, and microsatellites, as well as a highly skewed codon usage bias. Polymorphisms of AT-rich repeats provide abundant markers for linkage mapping of drug resistance genes and for tracing the evolution and structure of parasite populations.

Malaria parasite genomes carry multigene families that serve important roles in parasite interactions with their hosts, including, for example, antigenic variation, signaling, protein trafficking, and adhesion. Among the gene families, genes encoding P. falciparum erythrocyte membrane protein 1 (PfEMP1) have been studied most extensively. Each individual P. falciparum parasite carries a unique set of 50 to 150 copies of the var gene in its genome, where switches of gene expression can produce antigenic variation. PfEMP1 plays an important role in the pathogenesis of clinical developments such as in cerebral and placental malaria, in which it mediates the cytoadherence of infected red blood cells (iRBCs; infected erythrocytes) in the deep tissues. Different PfEMP1 molecules bind to various host molecules, including α2-macroglobulin, CD36, chondroitin sulfate A (CSA), complement 1q, CR1, E-selectins and P-selectins, endothelial protein C receptor (EPCR), heparan sulfate, ICAM1, IgM, IgG, PECAM1, thrombospondin (TSP), and VCAM1. Such binding leads to activation of various host inflammatory responses. Hemoglobinopathies, including the hemoglobin C and hemoglobin S trait conditions, interfere with PfEMP1 display in knob structures of the iRBCs. This poor display of PfEMP1 on the host cell surface offers protection against malaria by reducing the cytoadherence and activation of inflammatory processes that promote the development of severe disease.

Members of the large Plasmodium interspersed repeat (pir) multigene family are named differently by parasite species, for example, yir in P. yoelii, bir in P. berghei, vir in P. vivax. Several P. falciparum gene families (stevor, rif, and P/MC-2TM) are classified with pir by their similar gene structures, which characteristically include a short first exon, a long second exon, and a third exon encoding a transmembrane domain. In a recent study, the pir genes from P. chabaudi (cir) were shown to be expressed in different cellular locations, within and on the surface of iRBCs, and in merozoites. Malaria parasites devote large portions of their genomes to gene families that ensure evasion of host immune defenses and protection of molecular processes essential to infection. These families emphasize the importance of research on their roles in parasite-host interactions and virulence, despite the difficulties inherent to their investigation.

An additional, exemplary polymorphic gene family comprises a group of 14 genes encoding proteins with six cysteines (6-Cys). These proteins often localize on the parasite surface interacting with host proteins and are expressed at different parasite developmental stages. 6-Cys proteins also demonstrate diverse functions and have been shown to play roles in, for example, parasite fertilization, mating interactions, evasion of immune responses, and invasion of hepatocytes. The proteins expressed in asexual stages are generally polymorphic and/or under selection, suggesting that they could be targets of the host immune response; however, their functions in parasite development remain largely unknown.

Plasmodium genomes can be highly polymorphic. Early studies demonstrated polymorphisms involving tens to hundreds of kilobases and that the chromosome structure in P. falciparum is largely conserved in central regions but extensively polymorphic is both length and sequence near the telomeres. Much of the subtelomeric variation was explained by recombination within blocks of repetitive sequences and families of genes.

The frequency of simple sequence repeats (microsatellites) in P. falciparum is estimated to be approximately one polymorphic microsatellite per kb DNA. Without wishing to be bound by any one theory, this high rate may reflect the AT-rich nature of the genome. Microsatellites seem to be less frequent in other Plasmodium species that have genomes with lower AT contents. In addition to the highly polymorphic and repetitive structure of Plasmodium genomes, there are also large numbers of Single Nucleotide Polymorphisms (SNPs) and Copy Number Variations (CNVs)(Su et al., Plasmodium Genomics and Genetics: New Insights into Malaria Pathogenesis, Drug Resistance, Epidemiology, and Evolution. Clin Microbiol Rev. 2019 Jul. 31;32 (4), which is incorporated herein by reference in its entirety).

### C. Plasmodium Proteins

Plasmodium parasites are known to express various proteins at different stages of their lifecycles. Exemplary Plasmodium proteins are described below, and exemplary amino acid sequences are provided in Table 2.

Circumsporozoite protein (CSP) is a multifunctional protein that is involved in Plasmodium life cycle, as it is required for the formation of sporozoites in the mosquito midgut, the release of sporozoites from the oocyst, invasion of salivary glands, attachment of sporozoites to hepatocytes in the liver, and sporozoite invasion of hepatocytes (see, e.g., Zhao et al. (2016) PLOS ONE 11 (8): e0161607). CSP is present in all Plasmodium species, and although variation exists in the amino acid sequence across species, the overall domain structure of a central repeat region and nonrepeat flanking regions is well conserved (see, e.g., Zhao et al. (2016) PLOS ONE 11 (8): e0161607; Wahl et al. (2022) J. Exp. Med. 219: e20201313, which are herein incorporated by reference in their entirety). CSP sequences are known (see, e.g., UniProt accession numbers A0A2L1CF52, A0A2L, 1CF88, C6FGZ3, C6FH2,7 C6FHG7, M1V060, M1VOA3, M1VOBO, M1VOC4, M1VOE0, M1V914, M1VFN9, M1VKZ2, P02893, Q5EIJ9, Q5EIK2, Q5EIK8, Q5EIL3, Q5EIL5, Q5EIL8, Q5R2L2, Q7K740, Q819G₅, Q8I9J3, Q819J4), and Table 1 includes exemplary sequences for CSP P. falciparum isolates from Asia, South America and Africa.

TABLE 1
Exemplary Sequences for CSP P. falciparum isolates from Asia, South America and Africa

| Accession number | Country | References |
|---|---|---|
| AB121015.1 | 7G8 (Brazil) | Tanabe, K., et al., Genetic distance in housekeeping genes |
| | | between Plasmodium falciparum and Plasmodium reichenowi |
| | | and within P. falciparum. J. Mol. Evol. 59 (5), 687-694 (2004), |
| | | which is incorporated herein by reference. |
| AB121017.1 | Dd2 (Indochina) | Tanabe (2004) |
| AB121018.1 | HB3 (Honduras) | Tanabe (2004) |
| AB121020.1 | MAD20 (PNG) | Tanabe (2004) |
| AB121021.1 | RO-33 (Ghana) | Tanabe (2004) |
| AJ269946.1 | K1 (Thailand) | de Stricker, K., et al., Sequence variation in the non-repeat |
| | | region of the Plasmodium falciparum glutamate rich protein |
| | | (GLURP) from Brazil, Senegalese and Burmese field isolates |
| | | and from laboratory strains. (Unpublished), which is |
| | | incorporated herein by reference. |
| M15505.1 | Wellcome (West | Lockyer M J, Schwarz R T. Strain variation in the |
| | African) | circumsporozoite protein gene of Plasmodium falciparum. Mol |
| | | Biochem Parasitol. 2; 22(1): 101-8 (1987), which is |
| | | incorporated herein by reference. |
| M22982.1 | NF54 (Netherlands) | Caspers P, Gentz R, Matile H, Pink J R, Sinigaglia FThe |
| | | circumsporozoite protein gene from NF54, a Plasmodium |
| | | falciparum isolate used in malaria vaccine trials. Mol Biochem |
| | | Parasitol. 35(2): 185-9. (1989), which is incorporated herein by |
| | | reference. |
| AF540441.1, | India | Escalante A A, Grebert H M, Isea R, Goldman I F, Basco |
| AF540442.1, | | L, Magris M, Biswas S, Kariuki S and Lal A A. A study of genetic |
| AF540472.1, | | diversity in the gene encoding the circumsporozoite protein |
| AF540481.1 to | | (CSP) of Plasmodium falciparum from different transmission |
| AF540488.1 | | areas--XVI. Asembo Bay Cohort Project. Mol Biochem |
| | | Parasitol125(1-2): 83-90 (2002), which is incorporated herein |
| | | by reference. |
| AJ269943.1 | India | de Stricker, K. (Unpublished) |
| DQ521663.1 to | Iran | Zakeri S, et al., Restricted T-cell epitope diversity in the |
| DQ521752.1 | | circumsporozoite protein from Plasmodium falciparum |
| | | populations prevalent in Iran. Am J Trop Med |
| | | Hyg.; 76(6): 1046-51 (2007), which is incorporated herein by |
| | | reference. |
| AJ269945.1, | Myanmar | de Stricker, K (Unpublished) |
| AJ269955.1- | | |
| AJ269960.1 | | |
| DQ193595.1 | Myanmar | Jalloh A, van Thien H, Ferreira M U, Ohashi J, Matsuoka |
| | | H, Kanbe T, Kikuchi A, Kawamoto F. Sequence variation in the |
| | | T-cell epitopes of the Plasmodium falciparum circumsporozoite |
| | | protein among field isolates is temporally stable: a 5-year |
| | | longitudinal study in southern Vietnam. J Clin Microbiol |
| | | 44(4): 1229-35 (2006), which is incorporated herein by |
| | | reference in its entirety. |
| FJ232142.1- | Thailand | Putaporntip C, Jongwutiwes S, Hughes A L. Natural selection |
| FJ232166.1, | | maintains a stable polymorphism at the circumsporozoite |
| FJ232168.1- | | protein locus of Plasmodium falciparum in a low endemic area. |
| FJ232364.1 | | Infect Genet Evol. 9(4): 567-73 (2009), which is incorporated |
| | | herein by reference in its entirety. |
| GQ890702.1- | Thailand | Jongwutiwes S, Putaporntip C, Hughes A L. Bottleneck effects |
| GQ890789.1 | | on vaccine-candidate antigen diversity of malaria parasites in |
| | | Thailand. Vaccine. 19; 28(18): 3112-7. (2010), which is |
| | | incorporated herein by reference in its entirety. |
| M19752.1 | Thailand | del Portillo H A, Nussenzweig R S, Enea V. Circumsporozoite |
| | | gene of a Plasmodium falciparum strain from Thailand. Mol |
| | | Biochem Parasitol. 24(3): 289-94. (1987), which is |
| | | incorporated herein by reference in its entirety. |
| M83149.1, | Thailand | Jongwutiwes S, Tanabe K, Hughes M K, Kanbara H, Hughes A L. |
| M83150.1-M83174.1 | | Allelic variation in the circumsporozoite protein of Plasmodium |
| | | falciparum from Thai field isolates. Am J Trop Med Hyg; |
| | | 51(5): 659-68 (1994), which is incorporated herein by |
| | | reference in its entirety. |
| DQ193573.1- | Vietnam | Jalloh (2006) |
| DQ193594.1 | | |
| DQ193593.1- | Indonesia | Jalloh (2006) |
| DQ193594.1 | | |
| AB116602.1- | Vanuatu | Tanabe (2004) |
| AB116607.1 | | |
| AF540458.1- | Venezuela | Escalante (2002). |
| AF540460.1, | | |
| AF540464.1, | | |
| AF540466.1, | | |
| AF540469.1- | | |
| AF540471.1, | | |
| AF540478.1- | | |
| AF540479.1 | | |
| AJ269941.1, | Brazil | de Stricker (Unpublished) |
| AJ269971.1- | | |
| AJ269978.1 | | |
| K02194.1 | Brazil | Dame J B, Williams J L et al. Structure of the gene encoding the |
| | | immunodominant surface antigen on the sporozoite of the |
| | | human malaria parasite Plasmodium falciparum. |
| | | Science. 10; 225(4662): 593-1984), which is incorporated herein |
| | | by reference in its entirety. |
| AF540443.1, | Asembo Bay | Escalante (2002). |
| AF540447.1- | | |
| AF540448.1, | | |
| AF540450.1- | | |
| AF540451.1, | | |
| AF540455.1- | | |
| AF540457.1, | | |
| AF540461.1- | | |
| AF540463.1, | | |
| AF540465.1, | | |
| AF540467.1, | | |
| AF540473.1 = | | |
| AF540477.1 | | |
| AF181833.1- | Gambia | Alloueche A, Silveira H, Conway D J, Bojang K, Doherty |
| AF181835.1, | | T, Cohen J, Pinder M, Greenwood B M. High-throughput |
| AY878598.1- | | sequence typing of T-cell epitope polymorphisms in |
| AY878641.1 | | Plasmodium falciparum circumsporozoite protein. Mol Biochem |
| | | Parasitol. 5; 106(2): 273-82 (2000), which is incorporated |
| | | herein by reference in its entirety. |
| AY878598.1 | Gambia | Weedall G D, Preston B M, Thomas A W, Sutherland C J, Conway |
| | | D J. Differential evidence of natural selection on two leading |
| | | sporozoite stage malaria vaccine candidate antigens. Int J |
| | | Parasitol. 37(1): 77-85 (2007), which is incorporated herein by |
| | | reference in its entirety. |
| AJ269948.1, | Senegal | de Stricker (Unpublished) |
| AJ269961.1- | | |
| AJ269970.1 | | |
| GQ119637.1- | Sierra Leone | Jalloh A, Jalloh M, Matsuoka H. T-cell epitope polymorphisms |
| GQ119678.1 | | of the Plasmodium falciparum circumsporozoite protein among |
| | | field isolates from Sierra Leone: age-dependent haplotype |
| | | distribution. Malar J. 5; 8: 120. (2009), which is incorporated |
| | | herein by reference in its entirety. |
| AF540444.1- | Cameroon | Escalante (2002). |
| AF540446.1, | | |
| AF540449.1, | | |
| AF540452.1- | | |
| AF540454.1, | | |
| AF540468.1, | | |
| AF540480.1 | | |

Exemplary CSP amino acid sequence is provided in SEQ ID NO: 1.

RH5 is found in Plasmodium falciparum (P. falciparum) and not found in the other species of Plasmodium that infect humans. RH5 orthologues are also found in other species belonging to the Lavarenia subgenus, which includes parasites that infect chimpanzees and gorillas, indicating a unique role in P. falciparum invasion of human erythrocytes. See, e.g., Ragotte, et al. Trends Parasitol. 36 (6) 2020, which is incorporated herein by reference in its entirety. RH5 is expressed during the mature schizont stages and can complex with Cysteine-rich Protective Antigen (CyRPA) and RH5-interacting Protein (Ripr) to form an elongated protein trimer on the merozoite surface that binds to erythrocyte surface protein basigin. See, e.g., Ragotte Trends Parasitol 2020 June; 36 (6): 545-559, which is herein incorporated by reference in its entirety).

In humans, RH5 binding to basigin plays an essential role in invasion, acting downstream of membrane deformation. Binding of RH5 to basigin is required for the induction of a spike in calcium within the erythrocyte, which is blocked when merozoites attempt to invade in the presence of anti-RH5, anti-Ripr, or anti-basigin antibodies or soluble basigin. See, e.g., Ragotte (2020).

RH5 is a 63 kDa protein expressed during the mature schizont stage. It is processed and cleaved to a 45 kDa form which is shed by the parasite. The structure of P/RH5 reveals a kite-like architecture formed from the coming together of two three-helical bundles. See, e.g., Ragotte (2020).

RH5 sequences are known (see, e.g., UniProt accession numbers A0A159SK44, A0A159SK99, A0A159SKS8, A0A159SKW8, A0A159SL23, A0A159SL78, A0A159SL96, A0A159SLM7, A0A159SMC8, A0A159SMR9, A0A161FQTO, A0A1B1UZE2, A0A1B1UZE4, A0A1B1UZE5, A0A346RCI1, A0A346RCJO, A0A346RCJ2, A0A346RCJ3, A0A346RCJ4, A0A346RCK4, A0A346RCK5, A0A346RCK6, A0A346RCK9, B2L3N7, Q8IFM5), and exemplary RH5 amino acid sequence is provided in SEQ ID NO: 365.

P113 is a glycosylphosphatidylinositol (GPI)-linked protein that interacts directly with the N terminus of unprocessed RH5, providing a mechanism by which the RH5 invasion complex is tethered to the merozoite surface. See, e.g., Ragotte (2020). P113 orthologues are found in all Plasmodium species sequenced thus far, suggestive of a common and conserved function(s)(Bullen et al. (2022) Molecular Microbiology 117:1245-1262, which is herein incorporated by reference in its entirety). Despite this, in rodent model of malaria, P. berghei, p113 knockout parasites were viable indicating the protein was not essential for asexual blood stage growth and invasion. The knockout parasites do, however, display defects in natural sporozoite transmission, leading to delayed patency in infected mice (Offeddu et al. (2014) Mol. Biochem. Parasitology 193:101-109, which is herein incorporated by reference in its entirety).

Plasmodium P113 sequences are known (see, e.g., Uniprot accession number Q8ILP3). Exemplary P113 amino acid sequence is provided in SEQ ID NO: 326.

Cysteine-Rich Protective Antigen (CyRPA) is a 43 kDa protein with a predicted N-terminal secretion signal. CyRPA is part of a multi-protein complex, including RH5 and Ripr, important for triggering Ca²⁺ release and establishment of tight junctions. P/CyRPA is highly conserved, with only a single SNP above 5% prevalence, is essential for invasion (as conditional knockdown causes the loss of invasion activity), and has poor sero-reactivity from natural exposure (See, e.g., Ragotte (2020)).

Plasmodium CyRPA sequences are known (see, e.g., Uniprot accession number A0A2S1Q7P0, A0A2S1Q7P5, A0A2S1Q7Q4, Q8IFM8). Exemplary CyRPA amino acid sequence is provided in SEQ ID NO: 329.

RH5-interacting Protein (Ripr) is an approximately 120 kDa protein and localized to micronemes during the schizont stage of the P. falciparum life cycle. The full-length 120 kDa protein is processed into two fragments of similar size, an N-terminal fragment (including EGF domains 1 and 2) and a C-terminal fragment (including EGF domains 3-10). Ripr colocalizes with RH5 and CyRPA during parasite invasion at the junction between merozoites and erythrocyte. Parasites with conditional knockouts of P/Ripr induce membrane deformation, but cannot complete invasion (See, e.g., Ragotte (2020)).

Plasmodium Ripr sequences are known (see, e.g., UniProt accession numbers A0A193PDI9, A0A193PDK3, A0A193PDK8, A0A193PDL3, A0A193PDL9, A0A193PDP4, A0A193PDQ8, A0A193PE01, A0A193PE05, A0A193PE07, 097302, A0A193PE17). Exemplary Ripr amino acid sequence is provided in SEQ ID NO: 332.

E140 is found in every Plasmodium species for which genomic sequence is available, and is well conserved, with amino acid identity ranging from 34-92% among species. See, e.g., Smith, et al. PLOS one 15.5 (2020): e0232234; http://doi: 10.1371/journal.pone.023223; and U.S. Patent Publication No. US 2019/0117752; which are incorporated herein by reference in their entirety. E140 is also highly conserved (95-99%) in P. falciparum strains isolated from different locations around the world, and exhibits a low mutation frequency. E140 is expressed at different life stages of malaria parasites (specifically, E140 has been detected in sporozoites, liver, and blood stage parasites).

Protein structure algorithms predict that the E140 protein has five transmembrane domains, presumable spanning a parasite or host-derived membrane. E140 displays distinct patterns of protein expression in mature sporozoites, late liver, and late schizont stages. It traffics to the anterior and posterior ends of the sporozoite, the parasitophorous vacuole space of the late liver stage and around developing merozoites in the late schizont stage. It is also known to be expressed in mature salivary gland sporozoites as well as oocyst-derived sporozoites and oocysts.

E140 sequences are known (see, e.g., UniProt accession numbers A0A650D649, A0A650D653, A0A650D672, A0A650D687, A0A650D690, A0A650D694, A0A650D6A3, A0A650D6B8, A0A650D6L3, A0A650D6L7, Q8I299), and exemplary E140 amino acid sequence is provided in SEQ ID NO: 335.

CelTOS is required for sporozoite traversal through Kupfer cells during the liver invasion process. CelTOS forms a pore from within the cell, allowing for sporozoite egress into the liver. Antibody epitopes have been characterized from immunized mice and infected human populations (Pf and Pv). In mouse studies, immunization with CelTOS has been shown to provide protection and against challenge. Vaccination with CelTOS may generate antibodies that can bind the extracellular domain of the pore-forming complex, blocking complete formation of the pore and preventing sporozoite traversal into the liver. See, e.g., Jimah et al., Elife 2016 Dec. 1; 5: e20621. doi: 10.7554/eLife.20621, which is incorporated herein by reference in its entirety.

Plasmodium CelTOS sequences are known (see, e.g., Uniprot accession number M1ETJ8, Q53UB7, A0A2R4QLA5, A0A2R4QLI0, A0A2R4QLI5, A0A2R4QLJ1, A0A2R4QLJ4, M1ETJ8, Q53UB8, Q8I5P1). Exemplary CelTOS amino acid sequence is provided in SEQ ID NO: 350.

SPECT1 and SPECT2 (the latter also sometimes referred to as perforin-like protein 1 (PLP1)) are essential Plasmodium proteins that may play a role in cell traversal. See Yang et al., Cell Rep. 2017 Mar. 28; 18 (13): 3105-3116. doi: 10.1016/j.celrep.2017.03.017, which is incorporated herein by reference in its entirety. Targeted disruption of P. falciparum SPECT1 or SPECT2 has been shown to reduce infectivity of sporozoites in liver-stage development in humanized mice. However, mechanisms of cell traversal of these two proteins are yet to be defined in P. falciparum. See Yang et al.

SPECT1 and SPECT2 are considered attractive pre-erythrocytic immune targets due to the key role they are thought to play in the crossing of the malaria parasite across the dermis and the liver sinusoidal wall, prior to invasion of hepatocytes. Recombinant P. falciparum SPECT2 has been shown to cause lysis of red blood cells in a Ca²⁺-dependent manner, as has the MACPF/CDC domain of P/SPECT2. PISPECT2 has also been implicated in the Ca²⁺-dependent egress of P. falciparum merozoites from red blood cells.

Plasmodium SPECT1 and SPECT2 sequences are known (see, e.g., UniProt accession numbers Q8IDR4 and Q9U0J9), and exemplary amino acid sequence is provided in SEQ ID NO: 353 and SEQ ID NO: 356, respectively.

Exported protein 1 (EXP1) is a single pass transmembrane protein with an N-terminal signal peptide expressed during intraerythrocytic stage and liver stage (see, e.g., Spielmann et al., Int J Med Microbiol. 2012 October; 302 (4-5): 179-86, which is herein incorporated by reference in its entirety). EXP1 was shown to initially localize to dense granules in merozoites and then be transported to parasitophorous vacuolar membrane (PVM) after invasion (see, e.g., Iriko et al., Parasitol Int. 2018 October; 67 (5): 637-639, which is herein incorporated by reference in its entirety). Once localized to the PVM, EXP1 forms homo-oligomers with a N-terminus that is exposed to the parasitophorous vacuolar lumen and a C-terminus that is exposed to the red blood cell cytosol (see, e.g., Mesén-Ramírez et al., PLOS Biol. 2019 Sep. 30;17 (9): e3000473, which is herein incorporated by reference in its entirety).

EXP1 has been demonstrated to possess glutathione S-transferase (GST) activity that may protect Plasmodium from oxidative damage (see, e.g., Mesén-Ramírez et al., PLOS Biol 17 (9) 2019 Sep. 30; 17 (9): e3000473, which is herein incorporated by reference in its entirety). Recently, it was demonstrated that EXP1 is important for Plasmodium survival by maintaining correct localization of EXP2, a nutrient-permeable channel in the PVM (see, e.g., Mesén-Ramírez et al., 2020).

P. falciparum EXP1 polypeptide sequences are known (see, e.g., UniProt accession number Q8IIF0, W7JTD3, Q25840, Q548U2, Q5VKK2, Q5VKK5, Q5WRH8, Q6V9G₄, Q6V9G₆, Q6V9G₉, Q6V9H1, Q6V9H2, Q9U590, P04923, P04926). Exemplary EXP1 amino acid sequence is provided in SEQ ID NO: 314.

Upregulated in infective sporozoites gene 3 (UIS3) is a membrane-bound protein localized to sporozoite parasitophorous vacuolar membrane (PVM) in infected hepatocytes. UIS3 was shown to interact with liver fatty acid-binding protein (L-FABP) and be involved in fatty acid and/or lipid import during phases of Plasmodium growth (see, e.g., Sharma et al., J Biol Chem. 2008 Aug. 29; 283 (35): 24077-24088; Mikolajczak et al., Int J Parasitol. 2007 April;37 (5): 483-9, which are herein incorporated by reference in their entirety).

After sporozoite invasion of host liver cells, there is synthesis of vital Plasmodium structural features (e.g., parasitophorous vacuolar membrane). During hepatocytic stages, the Plasmodium relies on host fatty acids for rapid synthesis of its membranes (see, e.g., Sharma et al., J Biol Chem. 2008 Aug. 29; 283 (35): 24077-24088, which is herein incorporated by reference in its entirety). UIS3 insertion in the PVM provides Plasmodium a method to import essential fatty acids and/or lipids during rapid sporozoites growth phases (see, e.g., Sharma et al., 2008).

Immunization with UIS3-deficient Plasmodium berghei sporozoites protected against malaria in rodent malaria model (see, e.g., Mueller et al., Nature. 2005 Jan. 13; 433 (7022): 164-7, which is herein incorporated by reference in its entirety). UIS3-deficient Plasmodium berghei can start the transformation process in the liver; however, they show severe defects during transformation into trophozoites (see, e.g., Mueller et al., 2005). UIS3-deficient Plasmodium berghei are also unable to develop into mature liver schizonts and therefore abort malaria infection within the liver itself (see, e.g., Mueller et al., 2005). Further, it was previously demonstrated that UIS3 derived from Plasmodium berghei and UIS3 derived from Plasmodium falciparum exhibited a low (i.e. 34%) amino acid sequence identity (see, e.g., Mueller et al., 2005).

Plasmodium UIS3 sequences are known (see, e.g., UniProt accession number A0A509ARS3, A0A1C6YLP3, Q8IEU1, A0A384KLI1, A0A1G₄H423, A0A077YB01, Q9NFU4). Exemplary UIS3 amino acid sequence is provided in SEQ ID NO: 359.

Upregulated in infective sporozoites gene 4 (UIS4) contains a single transmembrane domain and localizes to secretory organelles of sporozoites and to the parasitophorous vacuole membrane (PVM) of liver stages. UIS4 is not expressed in blood stages or early sporozoites that are produced in oocysts (see, e.g., Mackellar et al., Eukaryot Cell. 2010 May; 9 (5): 784-794, which is herein incorporated by reference in its entirety).

Deletion of UIS4 gene is associated with arrest of early liver stage development (see, e.g., Vaughan and Kappe, Cold Spring Harb Perspect Med. 2017 Jun. 1; 7 (6): α025486, which is herein incorporated by reference in its entirety). Recently, UIS4 was demonstrated to be involved in Plasmodium berghei survival by eluding host actin structures deployed as part of host cytosolic defense (see, e.g., Bana et al., iScience. 2022 Apr. 22;25 (5): 104281. doi: 10.1016/j.isci.2022.104281. eCollection 2022 May 20, which is herein incorporated by reference in its entirety). P. falciparum has an ortholog to UIS4 named ETRAMP10.3 which is not able serve as a functional compliment to P. yoelii UIS4, indicating it likely serves a different function in P. falciparum's life cycle (see Mackellar et al., Eukaryot. Cell 9:784-94 (2010), which is herein incorporated by reference in its entirety).

Plasmodium falciparum early transcribed membrane protein 10.3 (ETRAMP10.3) is an approximately 10 kDa protein and member of the early transcribed membrane proteins multigene family, a family which is conserved across Plasmodium species and includes proteins located in the parasitophorous vacuole. Several ETRAMP proteins are specific to P. falciparum and not found in Plasmodium species that infect other organisms. ETRAMP10.3 is one example, which is expressed in both liver and blood stage P. falciparum parasites. ETRAMP10.3 transcription has been found to peak during the transition from ring to trophozoite stages of P. falciparum blood stage infection in a human host. ETRAMP10.3 localizes to the parasitophorous vacuole and is exported to a host erythrocyte during blood stage infection. Although ETRAMP10.3 is sometimes referred to as Upregulated in Infectious Sporozoites gene 4 (UIS4), ETRAMP10.3 is understood to be an ortholog of UIS4 on the basis of synteny and structural similarity. However, ETRAMP10.3 is not a functional ortholog of UIS4 and may play a different biological role. Although the biological function of ETRAMP10.3 has not yet been completely resolved, localization to vesicular structures in the host erythrocyte suggests a role in host-parasite interaction or in remodeling of infected erythrocyte. ETRAMP10.3 appears to play a key role in the Plasmodium life cycle. When ETRAMP10.3 is deleted, the deletion can lead to the disruption of liver-stage development in mice and asexual blood stage progression.

Although the terms “UIS4” and “ETRAMP10.3” in the literature are sometimes used to refer to different proteins, in context of the present disclosure, the terms “UIS4” and “ETRAMP10.3” interchangeably to refer to ETRAMP10.3.

Plasmodium ETRAMP10.3 sequences are known (see, e.g., UniProt accession number Q8IJM9, which is incorporated herein by reference in its entirety). An exemplary ETRAMP10.3 amino acid sequence is provided in SEQ ID NO: 362.

Liver specific protein 1 (LISP-1) is expressed during Plasmodium development in hepatocytes and localized to the parasitophorous vacuolar membrane (PVM)(see, e.g., Ishino et al., Cell Microbiol. 2009 September; 11 (9): 1329-1339). LISP-1 was shown to be expressed at high levels during late liver stages development and to be involved in PVM breakdown and subsequent merozoite release (see, e.g., Ishino et al., Cell Microbiol. 2009 September; 11 (9): 1329-1339, which is herein incorporated by reference in its entirety).

Intracellular Plasmodium deficient in LISP-1 develop into hepatic merozoites and display normal infectivity to erythrocytes (see, e.g., Ishino et al., Cell Microbiol. 2009 September; 11 (9): 1329-1339, which is herein incorporated by reference in its entirety). However, LISP1-deficient liver-stage Plasmodium do not rupture PVM and remain trapped inside hepatocytes (see, e.g., Ishino et al., 2009).

Plasmodium LISP-1 sequences are known (see, e.g., UniProt accession number A0A2I0C2X6, Q8ILR5). Exemplary LISP-1 amino acid sequence is provided in SEQ ID NO: 308.

Liver specific protein 2 (LISP-2) contains a modified 6-cys domain and is expressed during Plasmodium development in hepatocytes (see, e.g., Orito et al., Mol Microbiol. 2013 January; 87 (1): 66-79, which is herein incorporated by reference in its entirety). LISP-2 was shown to be expressed by liver stages Plasmodium, exported to hepatocytes, and be distributed throughout the host cell, including the nucleus (see, e.g., Orito et al., 2013).

Intracellular Plasmodium deficient in LISP2 do not mature effectively during merozoites development (see, e.g., Orito et al., 2013).

Plasmodium LISP-2 sequences are known (see, e.g., UniProt accession number A0A2I0BZR4, Q8I1X6, Q9U0D4). Exemplary LISP-2 amino acid sequence is provided in SEQ ID NO: 311.

Thrombospondin-related adhesion protein (TRAP) contains an N-terminal domain that is commonly referred to as von Willebrand factor A domain, although it is most similar to an integrin I domain because it contains a metal ion-dependent adhesion site (MIDAS) with a bound Mg²⁺ ion that is required for sporozoite motility in vitro and infection in vivo (see, e.g., Lu et al., PLOS One. 2020; 15 (1): e0216260, which is herein incorporated by reference in its entirety). The I domain is inserted in an extensible β-ribbon and followed by a thrombospondin repeat (TSR) domain, a proline-rich segment at the C-terminus, a single-pass transmembrane domain, and a cytoplasmic domain (see, e.g., Lu et al., 2020). Sequence analysis of the proline-rich segment revealed the presence of SH3-domain binding PxxP motifs in Plasmodium TRAPs (Akhouri et al., Malar J. 2008 Apr. 22; 7:63. doi: 10.1186/1475-2875-7-63, which is herein incorporated by reference in its entirety).

TRAP is stored in the micronemes and becomes surface exposed at the sporozoite anterior tip when parasite comes in contact with host cells (Akhouri et al., Malar J. 2008 Apr. 22; 7:63. doi: 10.1186/1475-2875-7-63, which is herein incorporated by reference in its entirety). TRAP also plays an important role in liver cell invasion of sporozoites by helping sporozoites in gliding motility and in recognition of host receptors on the mosquito salivary gland and hepatocytes (Akhouri et al., Malar J. 2008 Apr. 22; 7:63. doi: 10.1186/1475-2875-7-63, which is herein incorporated by reference in its entirety).

Plasmodium TRAP sequences are known (see, e.g., UniProt accession numbers A0A5Q2EXK8, A0A5Q2EZD7, A0A5Q2F1F6, A0A5Q2F2B8, A0A5Q2F2H6, A0A5Q2F4G₉, O76110, P16893, Q01507, Q26020, Q76NM2, W8VNB6), and exemplary TRAP amino acid sequence is provided in SEQ ID NO: 287.

Liver-stage-associated protein (LSAP-1) has been shown to be found mainly at the periphery of the intracellular hepatic parasite throughout its development, but not in blood stage parasites and possibly in minor quantities in salivary gland sporozoites (see, e.g., Siau et al., PLOS Pathog. 2008 Aug. 8;4 (8): e1000121, which is herein incorporated by reference in its entirety). LSAP-1 is among the most abundant transcripts in the salivary gland transcriptome but has not been detected in proteomic surveys of sporozoites. Rather, expression has only been detected only in liver stages (see, e.g., Siau et al., 2008).

Plasmodium LSAP-1 sequences are known (see, e.g., UniProt accession number Q81632, W7JR53). Exemplary LSAP-1 amino acid sequence is provided in SEQ ID NO: 302.

Like LSAP-1, LSAP-2 is also among the most abundant transcripts in the salivary gland transcriptome but has not been detected in proteomic surveys of sporozoites. LSAP-2 has shown some efficacy as a vaccine when combined with other antigens. See, e.g., Halbroth et al., Infect Immun. 2020 Jan. 22; 88 (2): e00573-19. doi: 10.1128/IAI.00573-19. Print 2020 Jan. 22, which is incorporated herein by reference in its entirety.

Plasmodium LSAP-2 sequences are known (see, e.g., UniProt accession number Q81632, W7JR53). Exemplary LSAP-2 amino acid sequence is provided in SEQ ID NO: 305.

Liver-Stage Antigen 1 (LSA-1) is expressed after Plasmodium have invaded hepatocytes and antigen accumulates in the parasitophorous vacuole (see, e.g., Tucker, K. et al., 2016, ‘Pre-Erythrocytic Vaccine Candidates in Malaria’, in A. J. Rodriguez-Morales (ed.), Current Topics in Malaria, IntechOpen, London. 10.5772/65592, which is herein incorporated by reference in its entirety). The function of LSA-1 remains currently not known (see, e.g., Tucker, K. et al., 2016).

LSA-1 is a 230 kDa preerythrocytic stage protein containing a large central region consisting of over eighty 17 amino acid residue repeat units flanked by highly conserved C-and N-terminal regions (Richie, T. L. and Parekh, F. K. (2009) Malaria. In Vaccines for Biodefense and Emerging and Neglected Diseases (Barrett, A. D. T. and Stanberry L. R., eds), pp. 1309-1364, Elsevier, which is herein incorporated by reference in its entirety). LSA1 is expressed only by liver stage Plasmodium and not by sporozoites (Richie, T. L. and Parekh, F. K. (2009) Malaria, which is herein incorporated by reference in its entirety). In Vaccines for Biodefense and Emerging and Neglected Diseases (Barrett, A. D. T. and Stanberry L. R., eds, pp. 1309-1364, Elsevier, which is herein incorporated by reference in its entirety). The repeat region results in significant variation of the protein between strains of Plasmodium falciparum (see, e.g., Tucker, K. et al., 2016).

Plasmodium LSA-1 sequences are known (see, e.g., UniProt accession number Q25886, Q25887, Q25893, Q26028, Q9GTX5, 096125). Exemplary LSA-1 amino acid sequence is provided in SEQ ID NO: 290.

Liver stage antigen 3 (LSA-3) is a 200-kDa protein that is composed of three nonrepeating regions (NR-A, NR-B, and NR-C) flanking two short repeat regions and one long repeat region (see, e.g., Tucker, K. et al., 2016). The nonrepeat regions are well conserved across geographically diverse strains of Plasmodium falciparum (see, e.g., Tucker, K. et al., 2016). The most significant variation is in the repeating regions due to organization and number of repeating subunits rather than composition of the repeating regions (see, e.g., Tucker, K. et al., 2016).

Recently, in vitro data has shown that antibodies against LSA-3 (in particular, the C-terminal portion of LSA-3) may provide some protection (see, e.g., Morita et al, Sci Rep. 2017 Apr. 5; 7:46086. doi: 10.1038/srep46086, which is herein incorporated by reference in its entirety).

Plasmodium LSA-3 sequences are known (see, e.g., UniProt accession number C7DU21, C7DU22, C7DU23, C7DU24, C7DU25, C7DU26, C7DU27, C7DU28, C7DU29, C7DU32, C7DU33, C7DU34, C7DU36, C7DU37, C7DU38, C7DU39, C7DU40, Q8I042, Q8I0A5, Q8I0D0, Q8IFR1, Q8IFR2, Q8IFR3, Q8IFR4, Q8IFR5, Q8IFR6, Q8IFR7, Q8IFR8, Q8IFR9, Q8IFSO, Q8IFS1, Q8IFS2, Q8IFS3, Q8IFS4, Q8IFS5, Q8IFS6, Q8IFS7, Q8IFS8, Q8IFS9, Q8IFTO, Q8IFT1, Q8IFT2, Q8IFT3, Q8IFT4, Q9UON9, Q9UOP0, A0A2I0BVD6, AOPFM9, 096275). Exemplary LSA-3 amino acid sequence is provided in SEQ ID NO: 299.

Glutamic acid-rich protein (GARP) is a 80kDA protein which derives its name from its glutamic rich amino acid sequence which comprises 24% of all its residues. GARP is predominantly expressed in ring stages and trophozoites and has been shown to be a non-essential gene in cell culture but highly immunogenic in animal models (Hon et al., Trends Parasitol. 2020 August; 36 (8): 653-655, which is herein incorporated by reference in its entirety). Although GARP is non-essential in cell culture, its localization to the periphery of infected erythrocytes may indicate a role in the sequestration of infected erythrocytes. GARP's involvement in sequestration has been proposed to occur by way of binding with a chloride/bicarbonate anion exchanger (Lau et al., PLOS Pathog. 10, e1004135. 2014, which is herein incorporated by reference in its entirety). Antibodies against GARP have been proposed to serve as signatures of protection against severe malaria and have shown efficacy in experimental trials in monkeys. See, e.g., Hon et al, Trends in Paras 2020 August; 36 (8): 653-655. doi: 10.1016/j.pt.2020.05.012 and Laue et al, Plos Path. 2014 10, e1004135, which are herein incorporated by reference in their entirety. GARP sequences are known (see, e.g., UniProt accession number, Q9GTW3, Q9UON1), and exemplary GARP amino acid sequence is provided in SEQ ID NO: 341.

Parasite-infected erythrocyte specific protein 2 (PIESP2)(see, e.g., UniProt accession number Q81488) is a highly immunogenic protein first expressed in the trophozoite stage and believed to be important for the clinical progression of cerebral malaria. Although this protein is predominantly found within erythrocytes, it has been shown to be present on the surface of erythrocytes, allowing them to adhere to endothelial cells in the vasculature of the brain. Antibodies against PIESP2 have been shown to prevent vascular adherence of Plasmodium and could prove valuable in preventing the preventing inflammatory response in the brain and impairment of the blood-brain barrier during cerebral malaria progression (see, e.g., Liu et al, Int J Biol Macromol. 2021 Apr. 30; 177:535-547. doi: 10.1016/j.ijbiomac.2021.02.145, which is herein incorporated by reference in its entirety). PIESP2 sequences are known (see, e.g., UniProt accession number Q8I488), and exemplary PIESP2 amino acid sequence is provided in SEQ ID NO: 344.

Shizont egress antigen-1 (SEA1) is a large 244 kDA protein lacking transmembrane domains or known targeting signals. The function of SEA1 is not known; however, it has been shown to be effective in rodent vaccine studies and has even been proposed as a target of protective antibodies found in children. SEA1 received its name after it was reported that antibodies agasint this protein inhibited egress of Plasmodium merizoites. SEA1 localizes closely to centromers during nuclear division, implicating its role in the essential process of replication. To date, various studies have proposed a role for SEA1 in egress, but also in mitotic division of nuclei during replication. (see, e.g., Perrin et al, mBio. 2021 Mar. 9;12 (2): e03377-20. doi: 10.1128/mBio.03377-20, which is herein incorporated by reference in its entirety). SEA1 sequences are known (see, e.g., UniProt accession number A0A143ZXM2), and exemplary SEA1 amino acid sequence is provided in SEQ ID NO: 347.

### D. Embodiments of Malarial Sequences

An exemplary full length CSP polypeptide amino sequence from Plasmidum falciparum isolate 3D7 corresponds to SEQ ID NO:1, and includes the following: a secretory signal (amino acids 1-18); an N-terminal domain (amino acids 19-104); a junction region (amino acids 93-104), a central domain (amino acids 105-272); and a C-terminal domain (amino acids 273-397). In exemplary SEQ ID NO:1, the N-terminal domain includes an N-terminal region (amino acids 19-80); an N-terminal end region (amino acids 81-92); and a junction region (amino acids 93-104). In exemplary SEQ ID NO:1, the junction region includes an R1 region (amino acids 93-97) and amino acids ADGNPDP (SEQ ID NO: 93) at positions 98-104. In exemplary SEQ ID NO:1, the central domain includes a minor repeat region (amino acids 105-128) and a major repeat region (amino acids 129-272). In exemplary SEQ ID NO:1, the minor repeat region includes three repeats of the amino acid sequence NANPNVDP (SEQ ID NO:477). In exemplary SEQ ID NO:1, the major repeat region includes 35 repeats of the amino acid sequence NANP (SEQ ID NO: 108), wherein 35 repeats of the amino acid sequence NANP are separated into two contiguous stretches, and wherein one stretch includes 17 repeats of the amino acid sequence NANP and one includes 18 repeats of the amino acid sequence NANP which flank an amino acid sequence of NVDP (SEQ ID NO: 105). The major repeat region includes the amino acid sequences NPNANP (SEQ ID NO: 111) and NANPNA (SEQ ID NO: 114). In exemplary SEQ ID NO:1, the C-terminal domain includes a C-terminal region (amino acids 273-375) and a transmembrane domain (amino acids 376-397). In exemplary SEQ ID NO:1, the C-terminal region includes a Th2R region (amino acids 314-327) and a Th3R region (amino acids 352-363).

TABLE 2
Exemplary amino acid sequences

| SEQ ID | |
|---|---|
| NO: | Protein |
| 1 | CSP |
| 287 | TRAP |
| 290 | LSA-1 |
| 293 | LSA-1a |
| 296 | LSA-1b |
| 299 | LSA-3 |
| 302 | LSAP1 |
| | (3D7) |
| 305 | LSAP2 |
| | (3D7) |
| 308 | LISP1 |
| | (3D7) |
| 311 | LISP2 |
| | (3D7) |
| 314 | EXP1 |
| | (3D7) |
| 317 | EXP2 |
| | (3D7) |
| 320 | SPELD |
| | (3D7) |
| 323 | PL |
| | (3D7) |
| 326 | P113 |
| | (3D7) |
| 329 | CyRPA |
| | (3D7) |
| 332 | RIPR |
| | (3D7) |
| 335 | E140 |
| 338 | AMA-1 |
| 341 | GARP |
| 344 | PIESP2 |
| 347 | SEA-1/SEP-1 |
| 350 | celTOS Pf |
| | (3D7) |
| 353 | SPECT1 |
| | (3D7) |
| 356 | SPECT2 |
| 359 | UIS3 |
| 362 | ETRAMP10.3 |
| 365 | RH5 |
| 368 | IBIS1 |
| 371 | IBIS1 |
| 374 | IBIS1 |
| 377 | PY02667 |
| 380 | PY03652 |

### II. Plasmodium T Cell String Polypeptide Constructs

The present disclosure, among other things, utilizes RNA technologies as a modality to express one or more Plasmodium T cell string polypeptide construct (also referred to as “malarial T-cell string polypeptide constructs” or “malarial T-cell peptide string constructs”) that includes one or more T-cell antigens from one or more Plasmodium proteins, or one or more portions thereof, (e.g., one or more antigenic fragments thereof) described herein. Plasmodium T-cell string polypeptide constructs as described herein can include one or more T-cell antigens from one or more Plasmodium polypeptide, or one or more portions thereof, (e.g., one or more antigenic fragments thereof) as described herein. In some embodiments, a Plasmodium T-cell string polypeptide construct as described herein comprises one or more Plasmodium liver stage antigens that elicit a T cell response. As is understood in the art, a “T-cell antigen” as described herein can induce a T cell response in a subject or model system. In some embodiments, a Plasmodium T-cell string polypeptide construct that targets the liver stage of a Plasmodium infection includes polypeptides or antigenic portions thereof that are expected to be both of relatively high abundance in infected hepatocytes and elicit T cell response(s). Polyribonucleotides as described herein encoding Plasmodium T-cell string polypeptide constructs, as well as Plasmodium T-cell string polypeptide constructs described herein, are designed to deliver a polypeptide to a subject, and in turn, for protein degradation and processing for presentation within the subject so that the subject raises an immune response (e.g., T cell response(s)). Methods to determine the presence of a T-cell response are well known in the art and described in the examples. In a preferred embodiment, Plasmodium T-cell string polypeptide constructs as described herein include more than one T-cell antigen and/or epitope from Plasmodium liver stage polypeptides or one or more portions thereof. In some preferred embodiments, one or more Plasmodium liver stage polypeptides or antigenic portions thereof comprises between 2 to 20 liver stage polypeptides or antigenic portions thereof (e.g., antigenic portions that induce a T cell response). In some embodiments, a Plasmodium T-cell string polypeptide construct comprises 2, 3, 4, 5, 6, 7, 8, 9, 10, 11, 12, 13, 14, 15, 16, 17, 18, 19 or 20, preferably 4, 5, 6, 7, 8, 9, 10, 11 or 12 different liver stage polypeptides or antigenic portions thereof (e.g., which each are capable of eliciting a T-cell response).

For example, in some embodiments, a Plasmodium T cell string polypeptide construct includes one or more Plasmodium T-cell antigens from CSP, LSA-1 (a), LSA-1 (b), TRAP, LSAP2, UIS3, ETRAMP10.3, LISP-1, LISP-2, LSA-3, EXP1, LSAP1 and/or polypeptide regions or portions thereof (e.g., one or more antigenic fragments thereof). In some embodiments, a Plasmodium T cell string polypeptide construct comprises between about 25 amino acids and about 1200 amino acids, e.g., between about 25 amino acids and about 1100 amino acids, e.g., between about 25 amino acids and about 1000 amino acids, e.g., between about 25 amino acids and about 750 amino acids, e.g., between about 25 amino acids and about 500 amino acids. In some embodiments, a Plasmodium T cell string polypeptide construct comprises about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, or about 1200 amino acids. In some embodiments, a Plasmodium T cell string polypeptide construct additionally includes one or more additional amino acid sequences, such as a secretory signal (e.g., a heterologous secretory signal), a transmembrane region (e.g., a heterologous transmembrane region), a trafficking signal, and/or a linker, as described herein.

### A Selection of T-cell Antigens

In some embodiments, a T-cell antigen utilized in a Plasmodium T cell string polypeptide construct described herein includes Plasmodium protein sequences identified and/or characterized by one or more of:
HLA-I or HLA-II binding (e.g., to HLA allele(s) present in a relevant population);
HLA ligandomics data confirmed by mass spectrometry;
Relatively high expression;
Sequence conservation;
Expression during the early liver stage of parasite lifecycle;
Localization to the parasitophorous vaculous membrane;
Serum reactivity;
Immunogenicity (e.g., presence of one or more B-cell and/or T-cell epitopes; evidence of ability to
induce sterile protection in model systems including, e.g., humans, non-human primates, and/or mice); and
Absence of sequences 8 amino acids and greater that overlap with human proteome unless 6 or more amino acids are from a linker sequence.

In some embodiments, such characteristics are experimentally or computationally assessed. In some embodiments, such characteristics are assessed by consultation with published reports.

For example, in some embodiments, HLA-I and/or HLA-II binding is experimentally assessed; in some embodiments, it is predicted. In some embodiments, predicted HLA-I or HLA-II binding is assessed using an algorithm such as neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMHCIIpan is utilized to predict or characterize likelihood of MHC binding for peptides as described herein. In some embodiments, an MHC-peptide presentation prediction algorithm or MHC-peptide presentation predictor is or comprises RECON® (Real-time Epitope Computation for ONcology), which offers high quality MHC-peptide presentation prediction based on expression, processing and binding capabilities. See, for example, Abelin et al., Immunity 21:315, 2017; Abelin et al., Immunity 15:766, 2019, each of which is incorporated herein by reference in its entirety.

In some embodiments, HLA binding and/or ligandomics assessments can consider the geographic region of subjects to be immunized. For example, in some embodiments, HLA allelic diversity can be considered. In some embodiments, T cell antigens comprise peptides (e.g., epitopes) expected or determined, when considered together, to bind to a significant percentage (e.g., at least 60%, 65%, 70%, 75%, 80%, 85%, 90%, 95%, or more) of HLA alleles expected or known to be present in a relevant region or population. In some embodiments, T cell antigens comprise peptides expected or determined, when considered together, to bind to the most prevalent (e.g., the 1, 2, 3, 4, 5, 6, 7, 8, 9, or 10 most prevalent, or at least 1, 2, 3, 4, or 5 of the 10 most prevalent, etc.) HLA alleles expected or known to be present in a relevant region or population).

In some embodiments, expression level is experimentally determined (e.g., in a model system or in infected humans). In some embodiments, expression level is a reported level (e.g., in a published or presented report). In some embodiments, expression level is assessed as RNA (e.g., via RNASeq). In some embodiments (and typically preferably), expression levels is assessed as protein.

In some embodiments, sequence conservation is assessed, for example, using publicly available sequence evaluation software (such as, for example, multiple sequence alignment programs MAFFT, Clustal Omega, etc.). In some embodiments, sequence conservation is determined by consultation with published resources (e.g., sequences). In some embodiments, sequence conservation includes consideration of currently or recently detected strains (e.g., in an active outbreak).

In some embodiments, surface exposure is assessed by reference to publicly available database and/or software. In some embodiments, surface exposure is assessed by reference to publicly available data, e.g., as described in Swearingen et al., “Interrogating the Plasmodium Sporozoite Surface: Identification of Surface-Exposed Proteins and Demonstration of Glycosylation on CSP and TRAP by Mass Spectrometry-Based Proteomics” PLOS Pathog (2016), the content of which is incorporated herein by reference for the purposes described herein.

In some embodiments, serum reactivity is assessed by contacting serum samples from infected individuals with polypeptides including sequences of interest (e.g., as may be displayed via, for example, phage display or peptide array, etc.; see, for example, Whittemore et al PlosOne, 2016, which is incorporated herein by reference in its entirety). In some embodiments, serum reactivity is assessed by consultation with literature reports and or database data indicating serum-recognized sequences.

In some embodiments, assessment of immunoreactivity and/or of presence of an epitope may be or comprise consultation with the Immune Epitope Database (IEDB) which those skilled in the art will be aware is a freely available resource funded by NIAID that catalogs experimental data on antibody and T cell epitopes (see iedb.org).

In some embodiments, ability to induce sterile protection is assessed, for example, as described in one or more of Schofield et al. “γ Interferon, CD8+ T cells and antibodies required for immunity to malaria sporozoites” Nature 330, 664-666 (1987); Weiss et al. (1988). “CD8+ T cells (cytotoxic/suppressors) are required for protection in mice immunized with malaria sporozoites” Proc. Natl. Acad. Sci. U.S.A. 85, 573-576; Romero et al. “Cloned cytotoxic T cells recognize an epitope in the circumsporozoite protein and protect against malaria.” Nature 341, 323-326 (1989); Rodrigues et al. (1991) “CD8+ cytolytic T cell clones derived against the Plasmodium yoelii circumsporozoite protein protect against malaria.” Int. Immunol. 3, 579-585; Chakravarty et al. “CD8+T lymphocytes protective against malaria liver stages are primed in skin-draining lymph nodes.” Nat Med. 2007 September;13 (9): 1035-41. Epub 2007 Aug. 19., each of which is incorporated herein by reference in its entirety).

In some embodiments, T cell antigens are characterized by dendritic cell presentation which, in turn may be indicative of HLA binding and/or of immunogenicity. Without wishing to be bound by any particular theory, it is proposed that dendritic cell presentation, e.g., in peripheral lymph nodes, may induce CD8+ T cells that migrate to the liver and, for example, may eliminate parasite-infected hepatocytes. See, for example, Chakravarty et al. “CD8+T lymphocytes protective against malaria liver stages are primed in skin-draining lymph nodes.” Nat Med. 2007 September; 13 (9): 1035-41. Epub 2007 Aug. 19, the entire content of which is incorporated herein by reference for the purposes described herein.

### B. Exemplary T-cell Antigens

In some embodiments, a Plasmodium T cell string polypeptide construct described herein includes one or more one or more Plasmodium T-cell antigens. In some embodiments, a Plasmodium T cell string polypeptide construct described herein includes one or more Plasmodium T-cell antigens from a Plasmodium protein selected from CSP, LSA-1 (a), LSA-1 (b), TRAP, LSAP2, UIS3, IS4, LISP-1, LISP-2, LSA-3, EXP1, and LSAP1.

In some embodiments, a Plasmodium T cell string polypeptide construct described herein includes 2 to about 20 Plasmodium T-cell antigens, (e.g., about 2 to about 15, about 2 to about 10, about 2 to about 9, about 2 to about 8, about 2 to about 7, about 2 to about 6, or about 2 to about 5 Plasmodium T-cell antigens). In some embodiments, a Plasmodium T cell string polypeptide construct described herein includes about 2, 3, 4, 5, 6, 7, 8, 9, 10, 11, 12, 13, 14, or 15 Plasmodium T-cell antigens. In some embodiments, a Plasmodium T cell string polypeptide construct described herein includes four Plasmodium T cell antigens. In some embodiments, a Plasmodium T cell string polypeptide construct described herein includes five Plasmodium T cell antigens. In some embodiments, a Plasmodium T cell string polypeptide construct described herein includes six Plasmodium T cell antigens. In some embodiments, a Plasmodium T cell string polypeptide construct described herein includes seven Plasmodium T cell antigens. In some embodiments, a Plasmodium T cell string polypeptide construct described herein includes eight Plasmodium T cell antigens. In some embodiments, a Plasmodium T cell string polypeptide construct described herein includes nine Plasmodium T cell antigens. In some embodiments, a Plasmodium T cell string polypeptide construct described herein includes ten Plasmodium T cell antigens. In some embodiments, a malaria T-cell peptide string construct described herein comprises only one or more immunogenic portions of the Plasmodium T-cell antigens included.

In some embodiments, a Plasmodium T cell string polypeptide construct described herein includes a CSP polypeptide, e.g., Plasmodium CSP, e.g., P. falciparum CSP, preferably from Plasmodium falciparum isolate 3D7. In some embodiments, a Plasmodium T cell string polypeptide construct described herein includes a CSP polypeptide comprising or consisting of the amino acid sequence of SEQ ID NO:1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO:1. In some embodiments, a Plasmodium T cell string polypeptide construct described herein includes an antigenic Plasmodium CSP polypeptide fragment. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a LSA-1 (a) polypeptide, e.g., Plasmodium LSA-1 (a) polypeptide. In some embodiments, a Plasmodium T cell string polypeptide construct described herein includes a LSA-1 (a) polypeptide comprising or consisting of the amino acid sequence of SEQ ID NO: 293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide. In some embodiments, a Plasmodium T cell string polypeptide construct described herein includes a LSA-1 (b) polypeptide comprising or consisting of the amino acid sequence of SEQ ID NO: 296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a TRAP polypeptide, e.g., Plasmodium TRAP polypeptide. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSAP1 polypeptide, e.g., Plasmodium LSAP1 polypeptide. In some embodiments, a Plasmodium T cell string polypeptide construct described herein includes a LSAP1 polypeptide comprising or consisting of the amino acid sequence of SEQ ID NO:302. In some embodiments, a Plasmodium T cell string polypeptide construct described herein includes a LSAP1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 302. In some embodiments, a Plasmodium T cell string polypeptide construct described herein includes an antigenic Plasmodium LSAP1 polypeptide fragment. In some embodiments, an antigenic Plasmodium LSAP1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 192. In some embodiments, an antigenic Plasmodium LSAP1 polypeptide fragment comprises or consists of the amino acid according to SEQ ID NO: 192.

In some embodiments, a Plasmodium T cell string polypeptide construct described herein includes a LSAP2 polypeptide, e.g., Plasmodium LSAP2 polypeptide. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes an ETRAMP10.3 polypeptide, e.g., Plasmodium ETRAMP10.3 polypeptide. In some embodiments, a Plasmodium T cell string polypeptide construct described herein includes a ETRAMP10.3 polypeptide comprising or consisting of the amino acid sequence of SEQ ID NO: 362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid SEQ ID NO: 234, 235, or 236.

In some embodiments, a Plasmodium T cell string polypeptide construct described herein includes a LISP-2 polypeptide, e.g., Plasmodium LISP-2 polypeptide. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 247.

In some embodiments, a Plasmodium T cell string polypeptide construct described herein includes a LSA-3 polypeptide, e.g., Plasmodium LSA-3 polypeptide. In some embodiments, a Plasmodium T cell string polypeptide construct described herein includes a LSA-3 polypeptide comprising or consisting of the amino acid sequence of SEQ ID NO: 299. In some embodiments, a Plasmodium T cell string polypeptide construct described herein includes a LSA-3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 299. In some embodiments, a Plasmodium T cell string polypeptide construct described herein includes an antigenic Plasmodium LSA-3 polypeptide fragment. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 249. In some embodiments, an antigenic Plasmodium LSA-3 polypeptide fragment comprises or consists of the amino acid according to SEQ ID NO: 249. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 254, 255, 256, 257, 258, 259, 260, 261, or 262. In some embodiments, an antigenic Plasmodium LSA-3 polypeptide fragment comprises or consists of the amino acid sequence according to SEQ ID NO: 254, 255, 256, 257, 258, 259, 260, 261, or 262.

In some embodiments, a Plasmodium T cell string polypeptide construct described herein includes a EXP1 polypeptide, e.g., Plasmodium EXP1 polypeptide. In some embodiments, a Plasmodium T cell string polypeptide construct described herein includes a EXP1 polypeptide comprising or consisting of the amino acid sequence of SEQ ID NO: 314. In some embodiments, a Plasmodium T cell string polypeptide construct described herein includes a EXP1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 314. In some embodiments, a Plasmodium T cell string polypeptide construct described herein includes an antigenic Plasmodium EXP1 polypeptide fragment. In some embodiments, an antigenic Plasmodium EXP1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 263. In some embodiments, an antigenic Plasmodium EXP1 polypeptide fragment comprises or consists of the amino acid according to SEQ ID NO: 263.

### C. Trafficking Signals

In some embodiments, a Plasmodium T cell string polypeptide construct described herein includes a trafficking signal. For example, in some embodiments, a trafficking signal is an MHC class I trafficking signal (MITD). In some embodiments, the MITD comprises or consists of an amino acid sequence according to SEQ ID NO: 479.

### D. Secretory Signals

In some embodiments, a Plasmodium T cell string polypeptide construct described herein includes a secretory signal, e.g., that is functional in mammalian cells. In some embodiments, a secretory signal comprises or consists of a Plasmodium secretory signal. In some embodiments, a Plasmodium secretory signal comprises or consists of a Plasmodium CSP secretory signal.

In some embodiments, a utilized secretory signal is a heterologous secretory signal. In some embodiments, a heterologous secretory signal comprises or consists of a non-human secretory signal. In some embodiments, a heterologous secretory signal comprises or consists of a viral secretory signal. In some embodiments, a viral secretory signal comprises or consists of an HSV secretory signal (e.g., an HSV-1 or HSV-2 secretory signal). In some embodiments, an HSV secretory signal comprises or consists of an HSV glycoprotein D (gD) secretory signal. In some embodiments, a secretory signal comprises or consists of an Ebola virus secretory signal. In some embodiments, an Ebola virus secretory signal comprises or consists of an Ebola virus spike glycoprotein (SGP) secretory signal.

In some embodiments, a secretory signal is characterized by a length of about 15 to 30 amino acids.

In many embodiments, a secretory signal is positioned at the N-terminus of a Plasmodium T cell string polypeptide construct described herein. In some embodiments, a secretory signal preferably allows transport of a Plasmodium T cell string polypeptide construct with which it is associated into a defined cellular compartment, preferably a cell surface, endoplasmic reticulum (ER) or endosomal-lysosomal compartment.

In some embodiments, a secretory signal is selected from an S1S2 secretory signal (aa 1-19), an immunoglobulin secretory signal (aa 1-22), a human SPARC secretory signal, a human insulin isoform 1 secretory signal, a human albumin secretory signal, etc. Those skilled in the art will be aware of other secretory signal such as, for example, as disclosed in WO2017/081082 (e.g., SEQ ID NOs: 1-1115 and 1728, or fragments variants thereof), which is herein incorporated by reference in its entirety. In some embodiments, a Plasmodium T cell string polypeptide construct described herein does not comprise a secretory signal.

In some embodiments, a secretory signal is one listed in Table 3, or a secretory signal having 1, 2, 3, 4, or 5 amino acid differences relative thereto. In some embodiments, a signal sequence is selected from those included in the Table 3 below and/or those encoded by the sequences in Table 4 below.

TABLE 3
Exemplary secretory signals

| SEQ | |
|---|---|
| ID NO: | Signal |
| 382 | HSV-1 gD SP |
| 391 | HSV-2 gD SP |
| 394 | HSV-2 |
| 397 | Csp (isolate 3D7) |
| 388 | HSV-1 gD SP |
| 400 | Ebola spike glycoprotein GP |
| 403 | SARS-CoV-2-S |
| 406 | human Ig heavy chain signal |
| | peptide (huSec) |
| 408 | HuIgGk signal peptide |
| 409 | IgE heavy chain epsilon-1signal |
| | peptide |
| 410 | Japanese encephalitis PRM signal |
| | sequence |
| 411 | VSVg protein signal sequence |
| 412 | TRIO |
| 414 | human Ig heavy chain signal peptide |
| 417 | human Ig heavy chain signal peptide |
| 420 | human Ig heavy chain signal peptide |
| 423 | human Ig heavy chain signal peptide |
| 426 | human Ig heavy chain signal peptide |
| 429 | human Ig heavy chain signal peptide |
| 432 | human Ig heavy chain signal peptide |
| 435 | human Ig heavy chain signal peptide |
| 438 | human Ig kappa chain signal peptide |
| 441 | human Ig kappa chain signal peptide |

TABLE 4
Exemplary polynucleotide sequences encoding secretory signals

| SEQ | |
|---|---|
| ID NO: | Signal |
| 381 | HSV-1 gD SP wild-type |
| 384 | HSV-1 gD SP |
| | Opt10 nt sequence |
| 402 | SARS-CoV-2-S |
| 405 | human Ig heavy chain signal |
| | peptide (huSec) |
| 413 | human Ig heavy chain signal |
| | peptide |
| 416 | human Ig heavy chain signal |
| | peptide |
| 419 | human Ig heavy chain signal |
| | peptide |
| 422 | human Ig heavy chain signal |
| | peptide |
| 425 | human Ig heavy chain signal |
| | peptide |
| 428 | human Ig heavy chain signal |
| | peptide |
| 431 | human Ig heavy chain signal |
| | peptide |
| 434 | human Ig heavy chain signal |
| | peptide |
| 437 | human Ig kappa chain signal |
| | peptide |
| 440 | human Ig kappa chain signal |
| | peptide |

### E. Transmembrane Regions

In some embodiments, a Plasmodium T cell string polypeptide construct described herein includes a transmembrane region. In some embodiments, a transmembrane region comprises or consists of a Plasmodium transmembrane region. In some embodiments, a utilized transmembrane region is one that is normally associated with CSP in nature. In some embodiments, a Plasmodium transmembrane region comprises or consists of a Plasmodium CSP glycosylphosphatidylinositol (GPI) anchor region, e.g., amino acids 374-397 of SEQ ID NO: 1. In some embodiments, a utilized transmembrane region is a heterologous transmembrane region.

In some embodiments, a transmembrane region is located at the N-terminus of a Plasmodium T cell string polypeptide construct. In some embodiments, a transmembrane region is located at the C-terminus of a Plasmodium T cell string polypeptide construct. In some embodiments, a transmembrane region is not located at the N-terminus or C-terminus of a Plasmodium T cell string polypeptide construct.

Transmembrane regions are known in the art, any of which can be utilized in a Plasmodium T cell string polypeptide construct described herein. In some embodiments, a transmembrane region comprises or is a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor.

In some embodiments, a heterologous transmembrane region does not comprise a hemagglutin transmembrane region. In some embodiments, a heterologous transmembrane region comprises or consists of a non-human transmembrane region. In some embodiments, a heterologous transmembrane region comprises or consists of a viral transmembrane region. In some embodiments, a heterologous transmembrane region comprises or consists of an HSV transmembrane region, e.g., an HSV-1 or HSV-2 transmembrane region. In some embodiments, an HSV transmembrane region comprises or consists of an HSV gD transmembrane region, e.g., comprising or consisting of an amino acid sequence according to SEQ ID NO: 447.

In some embodiments, a heterologous transmembrane region comprises or consists of a human transmembrane region. In some embodiments, a human transmembrane region comprises or consists of a human decay accelerating factor glycosylphosphatidylinositol (hDAF-GPI) anchor region. In some embodiments, an hDAF-GPI anchor region comprises or consists of an amino acid sequence according to SEQ ID NO: 450.

In some embodiments, a Plasmodium T cell string polypeptide construct described herein does not comprise a transmembrane region.

### F. Linkers

In some embodiments, a Plasmodium T cell string polypeptide construct described herein includes one or more linkers. In some embodiments, a linker is or comprises 2, 3, 4, 5, 6, 7, 8, 9, 10 or more amino acids. In some embodiments, a linker is or comprises no more than about 30, 25, 20, 15, 10 or fewer amino acids. A linker can include any amino acid sequence and is not limited to any particular amino acids. In some embodiments, a linker comprises one or more glycine (G) amino acids. In some embodiments, a linker comprises one or more serine(S) amino acids. In some embodiments, a linker includes amino acids selected based on a cleavage predictor to generate highly-cleavable linkers.

In some embodiments, a linker is or comprises S-G₄-S-G₄-S. In some embodiments, a linker is or comprises an amino acid sequence according to SEQ ID NO: 455. In some embodiments, a linker is or comprises an amino acid sequence according to SEQ ID NO: 452. In some embodiments, a linker is one according to any one of SEQ ID NOS: 453, 455, 452, 458 (GGS), 459 (GGGS), 456, 460, 454, or 457. In some embodiments, a linker is or comprises a sequence as set forth in WO2017/081082, which is incorporated herein by reference in its entirety (see SEQ ID NOs: 1509-1565, or a fragment or variant thereof).

In some embodiments, a Plasmodium T cell string polypeptide construct described herein comprises a linker between two Plasmodium T-cell antigens.

### G. Embodiments of Plasmodium T Cell String Polypeptide Constructs

In some embodiments, a Plasmodium T cell string polypeptide construct described herein includes two or more of: (i) an antigenic Plasmodium CSP polypeptide fragment; (ii) an antigenic Plasmodium LSA-1 (a) polypeptide fragment; (iii) an antigenic Plasmodium LSA-1 (b) polypeptide fragment; (iv) an antigenic Plasmodium TRAP polypeptide fragment; (v) an antigenic Plasmodium LSAP2 polypeptide fragment; (vi) an antigenic Plasmodium UIS3 polypeptide fragment; (vii) an antigenic Plasmodium ETRAMP10.3 polypeptide fragment; (viii) an antigenic Plasmodium LISP-1 polypeptide fragment; (ix) an antigenic Plasmodium LISP-2 polypeptide fragment; and (x) an antigenic Plasmodium LSA-3 polypeptide fragment.

In some embodiments, a Plasmodium T cell string polypeptide construct described herein includes one or more Plasmodium polypeptides or portions thereof from Plasmodium falciparum. In some embodiments, one or more Plasmodium polypeptides or portions thereof are one or more P. falciparum T cell antigens. In some embodiments, one or more P. falciparum T cell antigens are from P. falciparum isolate 3D7.

In some embodiments, a Plasmodium T cell string polypeptide construct describes herein does not include one or more Plasmodium polypeptides or portions thereof from Plasmodium berghei (e.g., antigenic Plasmodium berghei CSP polypeptide fragments).

In some embodiments, a Plasmodium T cell string polypeptide construct describes herein does not include an antigenic fragment of a bacterial polypeptide. In some embodiments, a Plasmodium T cell string polypeptide construct describes herein does not include an antigenic bacillus Calmette-Guérin (BCG) polypeptide fragment. In some embodiments, an antigenic BCG polypeptide fragment comprises an amino acid sequence according to SEQ ID NO: 461. In some embodiments, a Plasmodium T cell string polypeptide construct describes herein does not include an antigenic tetanus toxin (TT) polypeptide fragment. In some embodiments, an antigenic TT polypeptide fragment comprises an amino acid sequence according to SEQ ID NO: 462.

In some embodiments, a Plasmodium T cell string polypeptide construct describes herein does not include an antigenic Plasmodium sporozoite threonine-asparagine-rich protein (STARP) polypeptide fragment. In some embodiments, an antigenic Plasmodium STARP polypeptide fragment comprises an amino acid sequence according to SEQ ID NO: 463.

In some embodiments, a Plasmodium T cell string polypeptide construct described herein includes one or more Plasmodium polypeptide regions or portions thereof (e.g., antigenic fragments) as described above. Exemplary combinations are described below.

Constructs including CSP, TRAP, LSA-1 (a), LSA-1 (b), LSA-3, LSAP2

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSA-3 polypeptide (or one or more antigenic Plasmodium LSA-3 polypeptide fragments), and a Plasmodium LSAP2 polypeptide (or one or more antigenic Plasmodium LSAP2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO:293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSA-3 polypeptide, e.g., Plasmodium LSA-3 polypeptide e.g., P. falciparum LSA-3 polypeptide, preferably from Plasmodium falciparum isolate 3D7. In some embodiments, a Plasmodium T cell string polypeptide construct described herein includes a LSA-3 polypeptide comprising or consisting of the amino acid sequence of SEQ ID NO: 299. In some embodiments, a Plasmodium T cell string polypeptide construct described herein includes a LSA-3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 299. In some embodiments, a Plasmodium T cell string polypeptide construct described herein includes an antigenic Plasmodium LSA-3 polypeptide fragment. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 249. In some embodiments, an antigenic Plasmodium LSA-3 polypeptide fragment comprises or consists of the amino acid according to SEQ ID NO: 249. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 254, 255, 256, 257, 258, 259, 260, 261, or 262. In some embodiments, an antigenic Plasmodium LSA-3 polypeptide fragment comprises or consists of the amino acid sequence according to SEQ ID NO: 254, 255, 256, 257, 258, 259, 260, 261, or 262.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium LSA-1 (a) polypeptide fragment; (iv) an antigenic Plasmodium LSA-1 (b) polypeptide fragment; (v) an antigenic Plasmodium LSA-3 polypeptide fragment; and (vi) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium LSA-1 (a) polypeptide fragment; (iv) an antigenic Plasmodium LSA-1 (b) polypeptide fragment; (v) an antigenic Plasmodium LSA-3 polypeptide fragment; and (vi) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO:3. In some embodiments, a Plasmodium T cell string polypeptide construct described herein includes the amino acid sequence of SEQ ID NO: 3. In some embodiments, a Plasmodium T cell string polypeptide construct described herein includes in order: (i) an antigenic Plasmodium LSA-1 (a) polypeptide fragment; (ii) an antigenic Plasmodium LSA-1 (b) polypeptide fragment; (iii) an antigenic Plasmodium LSAP2 polypeptide fragment; (iv) an antigenic Plasmodium CSP polypeptide fragment; (v) an antigenic Plasmodium LSA-3 polypeptide fragment; and (vi) an antigenic Plasmodium TRAP polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 6. In some embodiments, a Plasmodium T cell string polypeptide construct described herein includes the amino acid sequence of SEQ ID NO: 6.

### Constructs Including LSAP1, EXP1, UIS3, ETRAMP10.3, LISP-1, LISP-2

In some embodiments, a Plasmodium T cell string polypeptide construct described herein includes a Plasmodium LSAP1 polypeptide (or one or more antigenic Plasmodium LSAP1 polypeptide fragments), a Plasmodium EXP1 polypeptide (or one or more antigenic Plasmodium EXP1 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments) wherein the Plasmodium is preferably Plasmodium falciparum and more preferably Plasmodium falciparum isolate 3D7.

In some embodiments, a Plasmodium T cell string polypeptide construct described herein includes a LSAP1 polypeptide, e.g., Plasmodium LSAP1 polypeptide e.g., P. falciparum LSAP1 polypeptide, preferably from Plasmodium falciparum isolate 3D7. In some embodiments, a Plasmodium T cell string polypeptide construct described herein includes a LSAP1 polypeptide comprising or consisting of the amino acid sequence of SEQ ID NO: 302. In some embodiments, a Plasmodium T cell string polypeptide construct described herein includes a LSAP1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 302. In some embodiments, a Plasmodium T cell string polypeptide construct described herein includes an antigenic Plasmodium LSAP1 polypeptide fragment. In some embodiments, an antigenic Plasmodium LSAP1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 192. In some embodiments, an antigenic Plasmodium LSAP1 polypeptide fragment comprises or consists of the amino acid according to SEQ ID NO: 192.

In some embodiments, a Plasmodium T cell string polypeptide construct described herein includes a EXP1 polypeptide, e.g., Plasmodium EXP1 polypeptide, e.g., P. falciparum EXP1 polypeptide, preferably from Plasmodium falciparum isolate 3D7. In some embodiments, a Plasmodium T cell string polypeptide construct described herein includes a EXP1 polypeptide comprising or consisting of the amino acid sequence of SEQ ID NO: 314. In some embodiments, a Plasmodium T cell string polypeptide construct described herein includes a EXP1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 314. In some embodiments, a Plasmodium T cell string polypeptide construct described herein includes an antigenic Plasmodium EXP1 polypeptide fragment. In some embodiments, an antigenic Plasmodium EXP1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 263. In some embodiments, an antigenic Plasmodium EXP1 polypeptide fragment comprises or consists of the amino acid according to SEQ ID NO: 263.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 247.

In some embodiments, a Plasmodium T cell string polypeptide construct described herein includes: (i) an antigenic Plasmodium LSAP1 polypeptide fragment; (ii) an antigenic Plasmodium EXP1 polypeptide fragment; (iii) an antigenic Plasmodium UIS3 polypeptide fragment; (iv) an antigenic Plasmodium ETRAMP10.3 polypeptide fragment; (v) an antigenic Plasmodium LISP-1 polypeptide fragment; and (vi) an antigenic Plasmodium LISP-2 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium EXP1 polypeptide fragment; (ii) an antigenic Plasmodium UIS3 polypeptide fragment; (iii) an antigenic Plasmodium ETRAMP10.3 polypeptide fragment; (iv) an antigenic Plasmodium LSAP1 polypeptide fragment; (v) an antigenic Plasmodium LISP-2 polypeptide fragment; and (vi) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 9. In some embodiments, a Plasmodium T cell string polypeptide construct described herein includes the amino acid sequence of SEQ ID NO: 9. In some embodiments, a Plasmodium T cell string polypeptide construct described herein includes in order: (i) an antigenic Plasmodium UIS3 polypeptide fragment; (ii) an antigenic Plasmodium LSAP1 polypeptide fragment; (iii) an antigenic Plasmodium LISP-1 polypeptide fragment; (iv) an antigenic Plasmodium EXP1 polypeptide fragment; (v) an antigenic Plasmodium ETRAMP10.3 polypeptide fragment; and (vi) an antigenic Plasmodium LISP-2 polypeptide fragment. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 12. In some embodiments, a Plasmodium T cell string polypeptide construct described herein includes the amino acid sequence of SEQ ID NO: 12.

### Constructs Including CSP, TRAP, LSAP2, UIS3, ETRAMP10.3

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), and a Plasmodium ETRAMP10.3 polypeptide (or one or more antigenic Plasmodium ETRAMP10.3 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; and (v) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic

Plasmodium UIS3 polypeptide fragment; (iv) an antigenic Plasmodium ETRAMP10.3 polypeptide fragment; and (v) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 15. In some embodiments, a Plasmodium T cell string polypeptide construct described herein includes the amino acid sequence of SEQ ID NO: 15.

In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; and (v) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 57. In some embodiments, a Plasmodium T cell string polypeptide construct described herein includes the amino acid sequence of SEQ ID NO: 57.

### Constructs Including CSP, TRAP, LSA-1 (a), LSA-1 (b), LSA-3, LSAP2, UIS3, ETRAMP10.3

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSA-3 polypeptide (or one or more antigenic Plasmodium LSA-3 polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), and a Plasmodium ETRAMP10.3 polypeptide (or one or more antigenic Plasmodium ETRAMP10.3 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO: 293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSA-3 polypeptide, e.g., Plasmodium LSA-3 polypeptide e.g., P. falciparum LSA-3 polypeptide, preferably from Plasmodium falciparum isolate 3D7. In some embodiments, a Plasmodium T cell string polypeptide construct described herein includes a LSA-3 polypeptide comprising or consisting of the amino acid sequence of SEQ ID NO: 299. In some embodiments, a Plasmodium T cell string polypeptide construct described herein includes a LSA-3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 299. In some embodiments, a Plasmodium T cell string polypeptide construct described herein includes an antigenic Plasmodium LSA-3 polypeptide fragment. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 249. In some embodiments, an antigenic Plasmodium LSA-3 polypeptide fragment comprises or consists of the amino acid according to SEQ ID NO: 249. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 254, 255, 256, 257, 258, 259, 260, 261, or 262. In some embodiments, an antigenic Plasmodium LSA-3 polypeptide fragment comprises or consists of the amino acid sequence according to SEQ ID NO: 254, 255, 256, 257, 258, 259, 260, 261 or 262.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO: 362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-3 polypeptide fragment; (vii) an antigenic Plasmodium LSA-1 (a) polypeptide fragment; and (viii) an antigenic Plasmodium LSA-1 (b) polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-3 polypeptide fragment; (vii) an antigenic Plasmodium LSA-1 (a) polypeptide fragment; and (viii) an antigenic Plasmodium LSA-1 (b) polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 18. In some embodiments, a Plasmodium T cell string polypeptide construct described herein includes the amino acid sequence of SEQ ID NO: 18.

### Constructs Including CSP, TRAP, LSA-1 (a), LSA-1 (b), LSAP2, UIS3, ETRAMP10.3, LISP-1, LISP-2

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184), 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO:293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 246.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (a) polypeptide fragment; (vii) an antigenic Plasmodium LSA-1 (b) polypeptide fragment; (viii) an antigenic Plasmodium LISP-2 polypeptide fragment; and (ix) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (a) polypeptide fragment; (vii) an antigenic Plasmodium LSA-1 (b) polypeptide fragment; (viii) an antigenic Plasmodium LISP-2 polypeptide fragment; and (ix) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 24 In some embodiments, a Plasmodium T cell string polypeptide construct described herein includes the amino acid sequence of SEQ ID NO: 24.

### Constructs Including CSP, TRAP, LSA-1 (a), LSA-1 (b), LSAP2, UIS3, ETRAMP10.3, LISP-1

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), and a Plasmodium LISP-1 polypeptide (or one or more antigenic Plasmodium LISP-1 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO:293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (a) polypeptide fragment; (vii) an antigenic Plasmodium LSA-1 (b) polypeptide fragment; and (viii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (a) polypeptide fragment; (vii) an antigenic Plasmodium LSA-1 (b) polypeptide fragment; and (viii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 27. In some embodiments, a Plasmodium T cell string polypeptide construct described herein includes the amino acid sequence of SEQ ID NO: 27.

### Constructs Including CSP, TRAP, LSAP2, UIS3, ETRAMP10.3, LISP-1, LISP-2

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 247.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LISP-2 polypeptide fragment; and (vii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LISP-2 polypeptide fragment; and (vii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 30. In some embodiments, a Plasmodium T cell string polypeptide construct described herein includes the amino acid sequence of SEQ ID NO: 30.

### Constructs Including CSP, TRAP, LSA-1 (b), LSAP2, UIS3, ETRAMP10.3, LISP-1

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSA-1 (b) polypeptide (or one or more antigenic Plasmodium LSA-1 (b) polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), and a Plasmodium LISP-1 polypeptide (or one or more antigenic Plasmodium LISP-1 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (b) polypeptide fragment; and (vii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (b) polypeptide fragment; and (vii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 33. In some embodiments, a Plasmodium T cell string polypeptide construct described herein includes the amino acid sequence of SEQ ID NO: 33.

### Constructs Including CSP, TRAP, LSA-1 (a), LSA-1 (b), LSA-3, LSAP2, UIS3, ETRAMP10.3, LISP-1, LISP-2

In some embodiments, a Plasmodium T cell string polypeptide construct described herein includes a CSP polypeptide (or one or more antigenic Plasmodium CSP polypeptide fragments), a Plasmodium TRAP polypeptide (or one or more antigenic Plasmodium TRAP polypeptide fragments), a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSA-3 polypeptide (or one or more antigenic Plasmodium LSA-3 polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a CSP polypeptide, e.g., Plasmodium CSP polypeptide, e.g., P. falciparum CSP polypeptide, preferably from Plasmodium falciparum isolate 3D7. In some embodiments, a Plasmodium T cell string polypeptide construct described herein includes a CSP polypeptide comprising or consisting of the amino acid sequence of SEQ ID NO: 1. In some embodiments, a Plasmodium T cell string polypeptide construct described herein includes a CSP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 1. In some embodiments, a Plasmodium T cell string polypeptide construct described herein includes one or more antigenic Plasmodium CSP polypeptide fragments, preferably from Plasmodium falciparum isolate 3D7. In some embodiments, an antigenic Plasmodium CSP polypeptide fragment comprises a Plasmodium CSP N-terminal region. In some embodiments, an antigenic Plasmodium CSP polypeptide fragment comprises a Plasmodium CSP N-terminal domain. In some embodiments, an antigenic Plasmodium CSP polypeptide fragment comprises a Plasmodium CSP N-terminal region and junction region. In some embodiments, an antigenic Plasmodium CSP polypeptide fragment comprises a Plasmodium CSP N-terminal end region and junction region. In some embodiments, an antigenic Plasmodium CSP polypeptide fragment comprises a Plasmodium CSP N-terminal domain and junction region. In some embodiments, an antigenic Plasmodium CSP polypeptide fragment containing at least some part of the N-terminal domain does not contain a C-terminal region. In some embodiments, an antigenic Plasmodium CSP polypeptide fragment does not contain a N-terminal domain. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 133. In some embodiments, an antigenic Plasmodium CSP polypeptide fragment comprises or consists of the amino acid according to SEQ ID NO: 133. In some embodiments, an antigenic Plasmodium CSP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 138, 139, 140, 141, or 142. In some embodiments, an antigenic Plasmodium CSP polypeptide fragment comprises or consists of the amino acid sequence according to SEQ ID NO: 138, 139, 140, 141, or 142.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO:293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSA-3 polypeptide, e.g., Plasmodium LSA-3 polypeptide e.g., P. falciparum LSA-3 polypeptide, preferably from Plasmodium falciparum isolate 3D7. In some embodiments, a Plasmodium T cell string polypeptide construct described herein includes a LSA-3 polypeptide comprising or consisting of the amino acid sequence of SEQ ID NO: 299. In some embodiments, a Plasmodium T cell string polypeptide construct described herein includes a LSA-3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 299. In some embodiments, a Plasmodium T cell string polypeptide construct described herein includes an antigenic Plasmodium LSA-3 polypeptide fragment. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 249. In some embodiments, an antigenic Plasmodium LSA-3 polypeptide fragment comprises or consists of the amino acid according to SEQ ID NO: 249. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 254, 255, 256, 257, 258, 259, 260, 261, or 262. In some embodiments, an antigenic Plasmodium LSA-3 polypeptide fragment comprises or consists of the amino acid sequence according to SEQ ID NO: 254, 255, 256, 257, 258, 259, 260, 261, or 162.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 245, 246, or 247.

In some embodiments, a Plasmodium T cell string polypeptide construct described herein includes: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (a) polypeptide fragment; (vii) an antigenic Plasmodium LSA-1 (b) polypeptide fragment; (viii) an antigenic Plasmodium LISP-2 polypeptide fragment; (ix) an antigenic Plasmodium LISP-1 polypeptide fragment; and (x) an antigenic Plasmodium LSA-3 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium CSP polypeptide fragment; (ii) an antigenic Plasmodium TRAP polypeptide fragment; (iii) an antigenic Plasmodium UIS3 polypeptide fragment; (iv) an antigenic Plasmodium ETRAMP10.3 polypeptide fragment; (v) an antigenic Plasmodium LSAP2 polypeptide fragment; (vi) an antigenic Plasmodium LSA-1 (a) polypeptide fragment; (vii) an antigenic Plasmodium LSA-1 (b) polypeptide fragment; (viii) an antigenic Plasmodium LISP-2 polypeptide fragment; (ix) an antigenic Plasmodium LISP-1 polypeptide fragment; and (x) an antigenic Plasmodium LSA-3 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 36. In some embodiments, a Plasmodium T cell string polypeptide construct described herein includes the amino acid sequence of SEQ ID NO: 36.

### Constructs Including LSA-1 (a), LSA-1 (b), LISP-1, LISP-2

In some embodiments, a Plasmodium T cell string polypeptide construct described herein includes a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO:293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 247.

In some embodiments, a Plasmodium T cell string polypeptide construct described herein includes: (i) an antigenic Plasmodium LSA-1 (a) polypeptide fragment; (ii) an antigenic Plasmodium LSA-1 (b) polypeptide fragment; (iii) an antigenic Plasmodium LISP-2 polypeptide fragment; and (iv) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium LSA-1 (a) polypeptide fragment; (ii) an antigenic Plasmodium LSA-1 (b) polypeptide fragment; (iii) an antigenic Plasmodium LISP-2 polypeptide fragment; and (iv) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 45. In some embodiments, a Plasmodium T cell string polypeptide construct described herein includes the amino acid sequence of SEQ ID NO: 45.

### Constructs Including LSA-1 (a), LSA-1 (b), LSA-3, LISP-1, LISP-2

In some embodiments, a Plasmodium T cell string polypeptide construct described herein includes a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSA-3 polypeptide (or one or more antigenic Plasmodium LSA-3 polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO: 293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSA-3 polypeptide, e.g., Plasmodium LSA-3 polypeptide e.g., P. falciparum LSA-3 polypeptide, preferably from Plasmodium falciparum isolate 3D7. In some embodiments, a Plasmodium T cell string polypeptide construct described herein includes a LSA-3 polypeptide comprising or consisting of the amino acid sequence of SEQ ID NO: 299. In some embodiments, a Plasmodium T cell string polypeptide construct described herein includes a LSA-3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 299. In some embodiments, a Plasmodium T cell string polypeptide construct described herein includes an antigenic Plasmodium LSA-3 polypeptide fragment. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 249. In some embodiments, an antigenic Plasmodium LSA-3 polypeptide fragment comprises or consists of the amino acid according to SEQ ID NO: 249. In some embodiments, an antigenic Plasmodium LSA-3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 254, 255, 256, 257, 258, 259, 260, 261, or 262. In some embodiments, an antigenic Plasmodium LSA-3 polypeptide fragment comprises or consists of the amino acid sequence according to SEQ ID NO: 254, 255, 256, 257, 258, 259, 260, 261, or 262.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 247.

In some embodiments, a Plasmodium T cell string polypeptide construct described herein includes: (i) an antigenic Plasmodium LSA-1 (a) polypeptide fragment; (ii) an antigenic Plasmodium LSA-1 (b) polypeptide fragment; (iii) an antigenic Plasmodium LISP-2 polypeptide fragment; (iv) an antigenic Plasmodium LISP-1 polypeptide fragment; and (v) an antigenic Plasmodium LSA-3 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium LSA-1 (a) polypeptide fragment; (ii) an antigenic Plasmodium LSA-1 (b) polypeptide fragment; (iii) an antigenic Plasmodium LISP-2 polypeptide fragment; (iv) an antigenic Plasmodium LISP-1 polypeptide fragment; and (v) an antigenic Plasmodium LSA-3 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 48. In some embodiments, a Plasmodium T cell string polypeptide construct described herein includes the amino acid sequence of SEQ ID NO: 48.

### Constructs Including TRAP, LSA-1 (a), LSA-1 (b), LSAP2, UIS3, ETRAMP10.3, LISP-1, LISP-2

In some embodiments, a Plasmodium T cell string polypeptide construct described herein includes a Plasmodium TRAP polypeptide (or one or more antigenic Plasmodium TRAP polypeptide fragments), a Plasmodium LSA-1 (a) polypeptide (or one or more antigenic Plasmodium LSA-1 (a) polypeptide fragments), a Plasmodium LSA-1 (b) polypeptide (or one or more antigenic Plasmodium LSA-1 (b) polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), a Plasmodium ETRAMP10.3 polypeptide (or one or more antigenic Plasmodium ETRAMP10.3 polypeptide fragments), a Plasmodium LISP-1 polypeptide (or one or more antigenic Plasmodium LISP-1 polypeptide fragments), and a Plasmodium LISP-2 polypeptide (or one or more antigenic Plasmodium LISP-2 polypeptide fragments), wherein the Plasmodium is preferably Plasmodium falciparum and more preferably Plasmodium falciparum isolate 3D7.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186, 187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSA-1 (a) polypeptide, e.g., Plasmodium LSA-1 (a) polypeptide e.g., P. falciparum LSA-1 (a) polypeptide, preferably from Plasmodium falciparum isolate 3D7. In some embodiments, a Plasmodium T cell string polypeptide construct described herein includes a LSA-1 (a) polypeptide comprising or consisting of the amino acid sequence of SEQ ID NO:293. In some embodiments, a Plasmodium T cell string polypeptide construct described herein includes a LSA-1 (a) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 293. In some embodiments, a Plasmodium T cell string polypeptide construct described herein includes an antigenic Plasmodium LSA-1 (a) polypeptide fragment. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 144. In some embodiments, an antigenic Plasmodium LSA-1 (a) polypeptide fragment comprises or consists of the amino acid according to SEQ ID NO: 144. In some embodiments, an antigenic Plasmodium LSA-1 (a) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 149, 150, 151, 152, or 153. In some embodiments, an antigenic Plasmodium LSA-1 (a) polypeptide fragment comprises or consists of the amino acid sequence according to SEQ ID NO: 149, 150, 151, 152, or 153.

In some embodiments, a Plasmodium T cell string polypeptide construct described herein includes a LSA-1 (b) polypeptide, e.g., Plasmodium LSA-1 (b) polypeptide e.g., P. falciparum LSA-1 (b) polypeptide, preferably from Plasmodium falciparum isolate 3D7. In some embodiments, a Plasmodium T cell string polypeptide construct described herein includes a LSA-1 (b) polypeptide comprising or consisting of the amino acid sequence of SEQ ID NO:296. In some embodiments, a Plasmodium T cell string polypeptide construct described herein includes a LSA-1 (b) polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 296. In some embodiments, a Plasmodium T cell string polypeptide construct described herein includes an antigenic Plasmodium LSA-1 (b) polypeptide fragment. In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 155. In some embodiments, an antigenic Plasmodium LSA-1 (b) polypeptide fragment comprises or consists of the amino acid according to SEQ ID NO: 155 In some embodiments, an antigenic Plasmodium LSA-1 (b) polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 160, 161, 162, 163, 164, 165, 166, 167, 168, or 169. In some embodiments, an antigenic Plasmodium LSA-1 (b) polypeptide fragment comprises or consists of the amino acid sequence according to SEQ ID NO: 160, 161, 162, 163, 164, 165, 166, 167, 168, or 169.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203, 204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes a LISP-1 polypeptide, e.g., Plasmodium LISP-1 polypeptide e.g., P. falciparum LISP-1 polypeptide, preferably from Plasmodium falciparum isolate 3D7. In some embodiments, a Plasmodium T cell string polypeptide construct described herein includes a LISP-1 polypeptide comprising or consisting of the amino acid sequence of SEQ ID NO: 308. In some embodiments, a Plasmodium T cell string polypeptide construct described herein includes a LISP-1 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 308. In some embodiments, a Plasmodium T cell string polypeptide construct described herein includes an antigenic Plasmodium LISP-1 polypeptide fragment. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 229. In some embodiments, an antigenic Plasmodium LISP-1 polypeptide fragment comprises or consists of the amino acid according to SEQ ID NO: 229. In some embodiments, an antigenic Plasmodium LISP-1 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 234, 235, or 236. In some embodiments, an antigenic Plasmodium LISP-1 polypeptide fragment comprises or consists of the amino acid sequence according to SEQ ID NO: 234, 235, or 236.

In some embodiments, a Plasmodium T cell string polypeptide construct described herein includes a LISP-2 polypeptide, e.g., Plasmodium LISP-2 polypeptide e.g., P. falciparum LISP-2 polypeptide, preferably from Plasmodium falciparum isolate 3D7. In some embodiments, a Plasmodium T cell string polypeptide construct described herein includes a LISP-2 polypeptide comprising or consisting of the amino acid sequence of SEQ ID NO: 311. In some embodiments, a Plasmodium T cell string polypeptide construct described herein includes a LISP-2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 311. In some embodiments, a Plasmodium T cell string polypeptide construct described herein includes an antigenic Plasmodium LISP-2 polypeptide fragment. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 238. In some embodiments, an antigenic Plasmodium LISP-2 polypeptide fragment comprises or consists of the amino acid according to SEQ ID NO: 238. In some embodiments, an antigenic Plasmodium LISP-2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 243, 244, 245, 246, or 247. In some embodiments, an antigenic Plasmodium LISP-2 polypeptide fragment comprises or consists of the amino acid sequence according to SEQ ID NO: 243, 244, 245, 246, or 247.

In some embodiments, a Plasmodium T cell string polypeptide construct described herein includes: (i) an antigenic Plasmodium TRAP polypeptide fragment; (ii) an antigenic Plasmodium UIS3 polypeptide fragment; (iii) an antigenic Plasmodium ETRAMP10.3 polypeptide fragment; (iv) an antigenic Plasmodium LSAP2 polypeptide fragment; (v) an antigenic Plasmodium LSA-1 (a) polypeptide fragment; (vi) an antigenic Plasmodium LSA-1 (b) polypeptide fragment; (vii) an antigenic Plasmodium LISP-2 polypeptide fragment; and (viii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium TRAP polypeptide fragment; (ii) an antigenic Plasmodium UIS3 polypeptide fragment; (iii) an antigenic Plasmodium ETRAMP10.3 polypeptide fragment; (iv) an antigenic Plasmodium LSAP2 polypeptide fragment; (v) an antigenic Plasmodium LSA-1 (a) polypeptide fragment; (vi) an antigenic Plasmodium LSA-1 (b) polypeptide fragment; (vii) an antigenic Plasmodium LISP-2 polypeptide fragment; and (viii) an antigenic Plasmodium LISP-1 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 51. In some embodiments, a Plasmodium T cell string polypeptide construct described herein includes the amino acid sequence of SEQ ID NO: 51.

### Constructs Including TRAP, LSAP2, UIS3, ETRAMP10.3

In some embodiments, a Plasmodium T cell string polypeptide construct described herein includes a Plasmodium TRAP polypeptide (or one or more antigenic Plasmodium TRAP polypeptide fragments), a Plasmodium LSAP2 polypeptide (or one or more antigenic Plasmodium LSAP2 polypeptide fragments), a Plasmodium UIS3 polypeptide (or one or more antigenic Plasmodium UIS3 polypeptide fragments), and a Plasmodium ETRAMP10.3 polypeptide (or one or more antigenic Plasmodium ETRAMP10.3 polypeptide fragments), wherein the Plasmodium is preferably Plasmodium falciparum and more preferably Plasmodium falciparum isolate 3D7.

In some embodiments, a Plasmodium T cell string polypeptide construct described herein includes a TRAP polypeptide, e.g., Plasmodium TRAP polypeptide e.g., P. falciparum TRAP polypeptide, preferably from Plasmodium falciparum isolate 3D7. In some embodiments, a Plasmodium T cell string polypeptide construct described herein includes a TRAP polypeptide comprising or consisting of the amino acid sequence of SEQ ID NO: 287. In some embodiments, a Plasmodium T cell string polypeptide construct described herein includes a TRAP polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 287. In some embodiments, a Plasmodium T cell string polypeptide construct described herein includes an antigenic Plasmodium TRAP polypeptide fragment. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 171. In some embodiments, an antigenic Plasmodium TRAP polypeptide fragment comprises or consists of the amino acid according to SEQ ID NO: 171. In some embodiments, an antigenic Plasmodium TRAP polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 176, 177, 178, 179, 180, 181, 182, 183, 184, 185, 186,187, 188, 189, or 190. In some embodiments, an antigenic Plasmodium TRAP polypeptide fragment comprises or consists of the amino acid sequence according to SEQ ID NO: 176, 177, 178, 179, 180, 181, 182, 183, 184, 185, 186, 187, 188, 189, or 190.

In some embodiments, a Plasmodium T cell string polypeptide construct described herein includes a LSAP2 polypeptide, e.g., Plasmodium LSAP2 polypeptide e.g., P. falciparum LSAP2 polypeptide, preferably from Plasmodium falciparum isolate 3D7. In some embodiments, a Plasmodium T cell string polypeptide construct described herein includes a LSAP2 polypeptide comprising or consisting of the amino acid sequence of SEQ ID NO: 305. In some embodiments, a Plasmodium T cell string polypeptide construct described herein includes a LSAP2 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 305. In some embodiments, a Plasmodium T cell string polypeptide construct described herein includes an antigenic Plasmodium LSAP2 polypeptide fragment. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 198. In some embodiments, an antigenic Plasmodium LSAP2 polypeptide fragment comprises or consists of the amino acid according to SEQ ID NO: 198. In some embodiments, an antigenic Plasmodium LSAP2 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 203,204, 205, 206, 207, 208, 209, or 210. In some embodiments, an antigenic Plasmodium LSAP2 polypeptide fragment comprises or consists of the amino acid sequence according to SEQ ID NO: 203, 204, 205, 206, 207, 208, 209, or 210.

In some embodiments, a Plasmodium T cell string polypeptide construct described herein includes a UIS3 polypeptide, e.g., Plasmodium UIS3 polypeptide e.g., P. falciparum UIS3 polypeptide, preferably from Plasmodium falciparum isolate 3D7. In some embodiments, a Plasmodium T cell string polypeptide construct described herein includes a UIS3 polypeptide comprising or consisting of the amino acid sequence of SEQ ID NO: 359. In some embodiments, a Plasmodium T cell string polypeptide construct described herein includes a UIS3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 359. In some embodiments, a Plasmodium T cell string polypeptide construct described herein includes an antigenic Plasmodium UIS3 polypeptide fragment. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 212. In some embodiments, an antigenic Plasmodium UIS3 polypeptide fragment comprises or consists of the amino acid according to SEQ ID NO: 212. In some embodiments, an antigenic Plasmodium UIS3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 217. In some embodiments, an antigenic Plasmodium UIS3 polypeptide fragment comprises or consists of the amino acid sequence according to SEQ ID NO: 217.

In some embodiments, a Plasmodium T cell string polypeptide construct described herein includes a ETRAMP10.3 polypeptide, e.g., Plasmodium ETRAMP10.3 polypeptide e.g., P. falciparum ETRAMP10.3 polypeptide, preferably from Plasmodium falciparum isolate 3D7. In some embodiments, a Plasmodium T cell string polypeptide construct described herein includes a ETRAMP10.3 polypeptide comprising or consisting of the amino acid sequence of SEQ ID NO:362. In some embodiments, a Plasmodium T cell string polypeptide construct described herein includes a ETRAMP10.3 polypeptide comprising or consisting of an amino acid sequence having at least 85% (e.g., at least 90%, 91%, 92%, 93%, 94%, 95%, 96%, 97%, 98%, or 99%) sequence identity to an amino acid sequence according to SEQ ID NO: 362. In some embodiments, a Plasmodium T cell string polypeptide construct described herein includes an antigenic Plasmodium ETRAMP10.3 polypeptide fragment. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to an amino acid sequence according to SEQ ID NO: 219. In some embodiments, an antigenic Plasmodium ETRAMP10.3 polypeptide fragment comprises or consists of the amino acid according to SEQ ID NO: 219. In some embodiments, an antigenic Plasmodium ETRAMP10.3 polypeptide fragment comprises or consists of an amino acid sequence with at least 85% (e.g., at least 90%, 91%, 92%, 93%, 94%, 95%, 96%, 97%, 98%, or 99%) sequence identity to SEQ ID NO: 224, 225, 226, or 227. In some embodiments, an antigenic Plasmodium ETRAMP10.3 polypeptide fragment comprises or consists of the amino acid sequence according to SEQ ID NO: 224, 225, 226, or 227.

In some embodiments, a Plasmodium T cell string polypeptide construct described herein includes: (i) an antigenic Plasmodium TRAP polypeptide fragment; (ii) an antigenic Plasmodium UIS3 polypeptide fragment; (iii) an antigenic Plasmodium ETRAMP10.3 polypeptide fragment; and (iv) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes in order: (i) an antigenic Plasmodium TRAP polypeptide fragment; (ii) an antigenic Plasmodium UIS3 polypeptide fragment; (iii) an antigenic Plasmodium ETRAMP10.3 polypeptide fragment; and (iv) an antigenic Plasmodium LSAP2 polypeptide fragment, wherein the Plasmodium is preferably Plasmodium falciparum and more preferably Plasmodium falciparum isolate 3D7. In some embodiments, a Plasmodium T cell string polypeptide construct described herein includes an amino acid sequence with at least 85% (e.g., at least 90%, 91%, 92%, 93%, 94%, 95%, 96%, 97%, 98%, or 99%) sequence identity to an amino acid sequence according to SEQ ID NO: 54. In some embodiments, a Plasmodium T cell string polypeptide construct described herein includes the amino acid sequence of SEQ ID NO: 54.

### H. Exemplary Construct Sequences

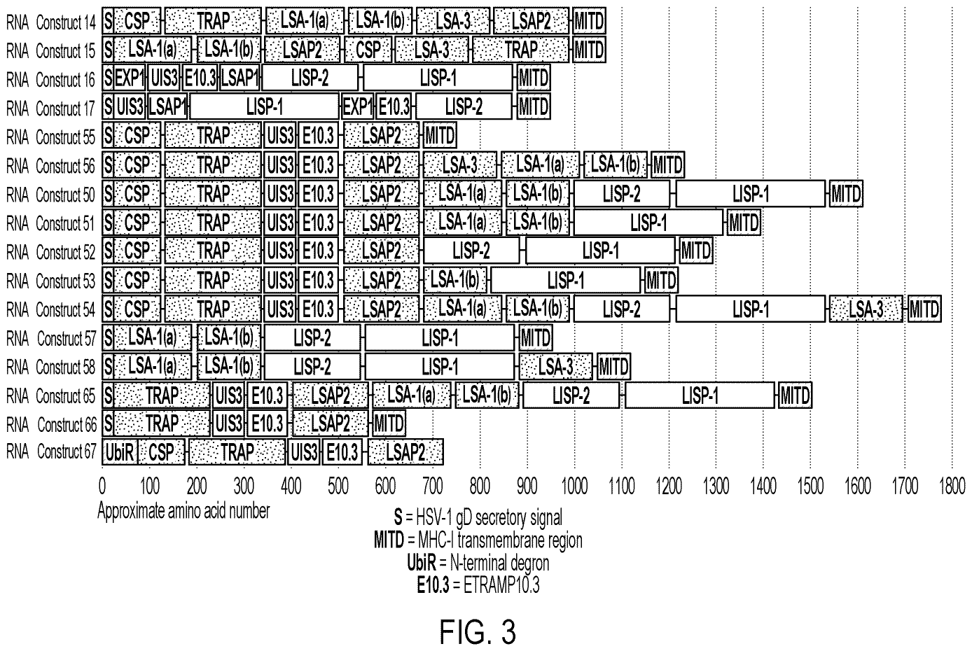

In some embodiments, a Plasmodium T cell string polypeptide construct described herein has an amino acid sequence provided in Table 5, and/or is encoded by a nucleotide sequence provided in Table 6A or 6B. Exemplary Plasmodium T cell string polypeptide constructs are also shown schematically in FIG. 3.

TABLE 5
Amino Acid Sequences for exemplary Constructs

| SEQ ID | |
|---|---|
| NO. | Construct |
| 3 | RNA Construct |
| | 14 |
| 6 | RNA Construct |
| | 15 |
| 9 | RNA Construct |
| | 16 |
| 12 | RNA Construct |
| | 17 |
| 15 | RNA Construct |
| | 48 |
| 18 | RNA Construct |
| | 49 |
| 21 | RNA Construct |
| | 3C |
| 24 | RNA Construct |
| | 50 |
| 27 | RNA Construct |
| | 51 |
| 30 | RNA Construct |
| | 52 |
| 33 | RNA Construct |
| | 53 |
| 36 | RNA Construct |
| | 54 |
| 39 | RNA Construct |
| | 55 |
| 42 | RNA Construct |
| | 56 |
| 45 | RNA Construct |
| | 57 |
| 48 | RNA Construct |
| | 58 |
| 51 | RNA Construct |
| | 65 |
| 54 | RNA Construct |
| | 66 |
| 57 | RNA Construct |
| | 67 |

TABLE 6A
DNA Sequences For exemplary Constructs

| SEQ ID | |
|---|---|
| NO. | Construct |
| 2 | RNA Construct |
| | 14 |
| 5 | RNA Construct |
| | 15 |
| 8 | RNA Construct |
| | 16 |
| 11 | RNA Construct |
| | 17 |
| 14 | RNA Construct |
| | 48 |
| 17 | RNA Construct |
| | 49 |
| 20 | RNA Construct |
| | 3C |
| 23 | RNA Construct |
| | 50 |
| 26 | RNA Construct |
| | 51 |
| 29 | RNA Construct |
| | 52 |
| 32 | RNA Construct |
| | 53 |
| 35 | RNA Construct |
| | 54 |
| 38 | RNA Construct |
| | 55 |
| 41 | RNA Construct |
| | 56 |
| 44 | RNA Construct |
| | 57 |
| 47 | RNA Construct |
| | 58 |
| 50 | RNA Construct |
| | 65/MAS4h |
| 53 | RNA Construct |
| | 66 |
| 56 | RNA Construct |
| | 67 |

TABLE 6B
RNA Sequences For exemplary Constructs

| SEQ ID | |
|---|---|
| NO. | Construct |
| 4 | RNA |
| | Construct 14 |
| 7 | RNA |
| | Construct 15 |
| 10 | RNA |
| | Construct 16 |
| 13 | RNA |
| | Construct 17 |
| 16 | RNA |
| | Construct 48 |
| 19 | RNA |
| | Construct 49 |
| 22 | RNA |
| | Construct 30 |
| 25 | RNA |
| | Construct 50 |
| 28 | RNA |
| | Construct 51 |
| 31 | RNA |
| | Construct 52 |
| 34 | RNA |
| | Construct 53 |
| 37 | RNA |
| | Construct 54 |
| 40 | RNA |
| | Construct 55 |
| 43 | RNA |
| | Construct 56 |
| 46 | RNA |
| | Construct 57 |
| 49 | RNA |
| | Construct 58 |
| 52 | RNA |
| | Construct 65 |
| 55 | RNA |
| | Construct 66 |
| 58 | RNA |
| | Construct 67 |

### III. Polyribonucleotides

### A. Exemplary Polyribonucleotides Features

Polyribonucleotides described herein encode one or more Plasmodium T cell string polypeptide constructs described herein. In some embodiments, polyribonucleotides described herein can comprise a nucleotide sequence that encodes a 5′UTR of interest and/or a 3′ UTR of interest. In some embodiments, polynucleotides described herein can comprise a nucleotide sequence that encodes a polyA tail. In some embodiments, polyribonucleotides described herein may comprise a 5′ cap, which may be incorporated during transcription, or joined to a polyribonucleotide post-transcription.

### 1. 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH)(e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550, which is herein incorporated by reference in its entirety, reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Zust et al. (2011) Nature Immunology, 12:137-143, which are herein incorporated by reference in their entirety.

RNA capping is well researched and is described, e.g., in Decroly E et al. (2012) Nature Reviews 10:51-65; and in Ramanathan A. et al., (2016) Nucleic Acids Res; 44 (16): 7511-7526, the entire contents of each of which is hereby incorporated by reference. For example, in some embodiments, a 5′-cap structure which may be suitable in the context of the present invention is a cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

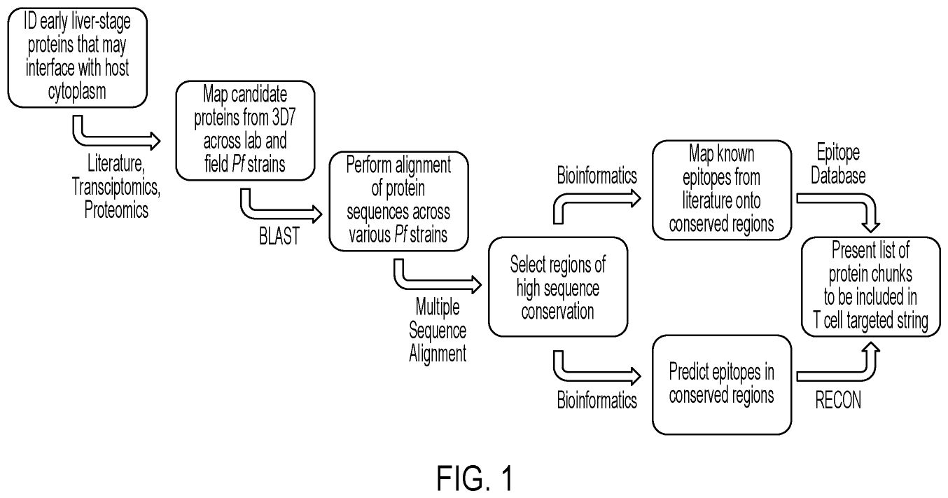

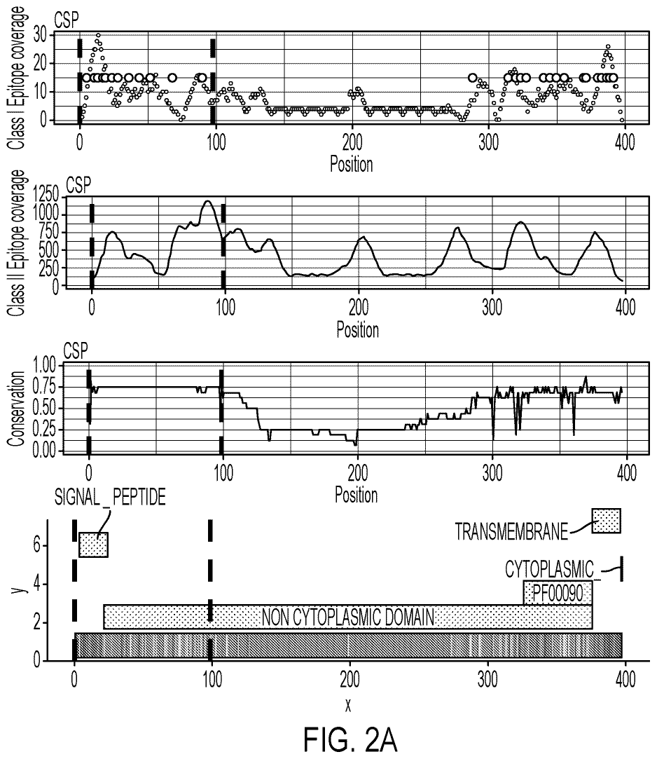

In some embodiments, a utilized 5′ caps is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m^{27,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position of the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, wherein each of N₁ and N₂ is as defined and described herein.

In some embodiments, a cap2 structure comprises a guanosine nucleoside methylated at the 7-position of guanine ((m⁷)G) and optionally methylated at the 2′ or 3′ position of the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., (m₇)Gpp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and G* comprises a structure of formula (I):

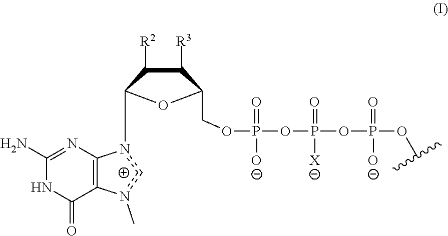

or a salt thereof,
wherein
   each R² and R³ is —OH or —OCH₃; and
   X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m^{27,2′-O})GppSpN₁, or (m₂^{7,3′-O})GppSpN₁), wherein N₁ is as defined and described herein. In some embodiments, the 5′ cap is a dinucleotide cap0 structure (e.g., (m⁷)GpppN₁, (m^{27,2′-O})GpppN₁, (m₂^{7,3′-O})GpppN₁, (m⁷)GppSpN₁, (m^{27,2-O})GppSpN₁, or (m₂^{7,3′-O})GppSpN₁), wherein N₁ is G. In some embodiments, the 5′ cap is a dinucleotide cap0 structure (e.g., (m⁷)GpppN₁, (m^{27,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is A, U, or C. In some embodiments, the 5′ cap is a dinucleotide cap1 structure (e.g., (m⁷)Gppp(m²′-O)N₁, (m^{27,2′-O})Gppp(m^{2′-O})N₁, (m₂^{7,3′-O})Gppp(m^{2′-O})N₁, (m⁷)GppSp(m^{2′-O})N₁, (m^{27,2′-O})GppSp(m^{2′-O})N₁, or (m₂^{7,3′-O})GppSp(m^{2′-O})N₁), wherein N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m^{27,2′-O})GppSpG(“beta-S-ARCA”). In some embodiments, the 5′ cap is (m⁷)GpppG(“Ecap0”), having a structure:

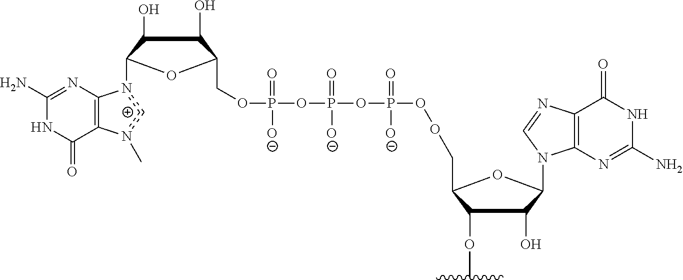

or a salt thereof.

In some embodiments, the 5′ cap is (m⁷)Gppp(m^{2′-O})G (“Ecap1”), having a structure:

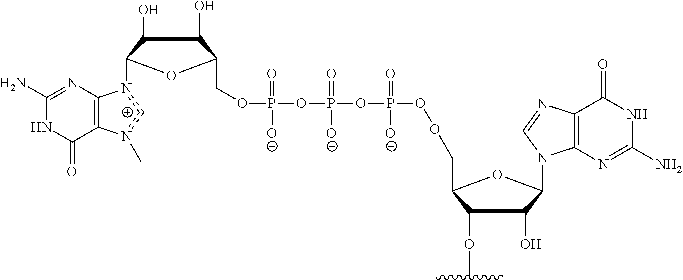

or a salt thereof.

In some embodiments, the 5′ cap is (m₂^{7,3′-O})GpppG (“ARCA” or “D1”), having a structure:

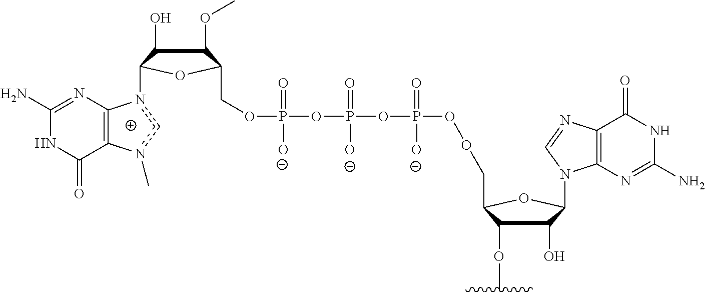

or a salt thereof.

In some embodiments, the 5′ cap is (m₂^{7,2′-O})GppSpG (“beta-S-ARCA”), having a structure:

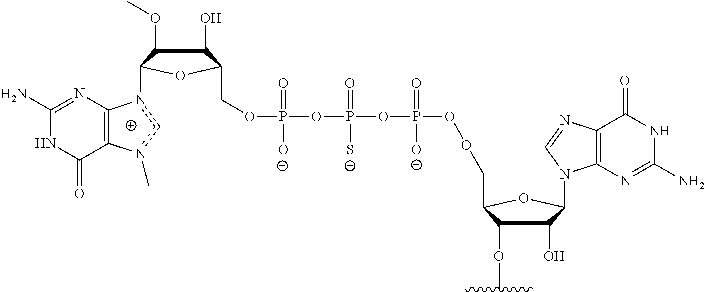

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a dinucleotide cap G*N₁pN₂, wherein N₁ and N₂ are as defined above and herein, and G* comprises a structure of formula (I):

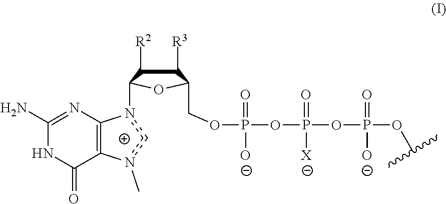

or a salt thereof, wherein R², R³, and X are as defined and described herein.

In some embodiments, the 5′ cap is a trinucleotide cap0 structure (e.g. (m⁷)GpppN₁pN₂, (m^{27,2′-O})GpppN₁pN₂, or (m₂^{7,3′-O})GpppN₁pN₂), wherein N₁ and N₂ are as defined and described herein). In some embodiments, the 5′ cap is a trinucleotide cap1 structure (e.g., (m⁷)Gppp(m^{2′-O})N₁pN₂, (m₂^{7,2′-O})Gppp(m²′-Q)N₁pN₂, (m₂^{7,3′-O})Gppp(m^{2′-O})N₁pN₂), wherein N₁ and N₂ are as defined and described herein. In some embodiments, the 5′ cap is a trinucleotide cap2 structure (e.g., (m⁷)Gppp(m^{2′-O})N₁p(m^{2′-O})N₂, (m^{27,2′O})Gppp(m^{2′-O})N₁p(m^{2′-O})N₂, (m₂^{7,3′-O})Gpp(m^{2′-O})N₁p(m^{2′-O})N₂), wherein N₁ and N₂ are as defined and described herein. In some embodiments, the 5′ cap is selected from the group consisting of (m₂^{7,3′-O})Gppp(m^{2′-O})ApG (“CleanCap AG”, “CC413”), (m₂^{7,3′O})Gppp(m^{2′-O})GpG (“CleanCap GG”), (m⁷)Gppp(m^{2′-O}) ApG, (m⁷)Gppp(m^{2′-O})GpG, (m₂^{7,3′-O})Gppp(m₂^{6,2′-O})ApG, and (m⁷)Gppp(m^{2′O})ApU.

In some embodiments, the 5′ cap is (m₂^{7,3′-O})Gppp(m^{2′-O}) ApG (“CleanCap AG”, “CC413”), having a structure:

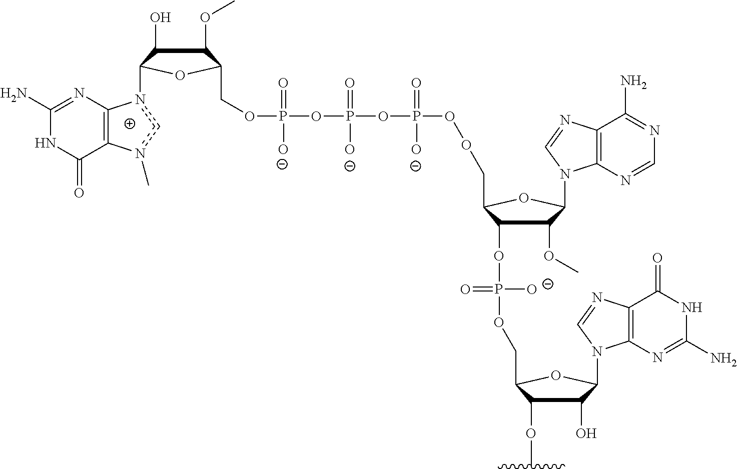

or a salt thereof.

In some embodiments, the 5′ cap is (m₂^{7,3′-O})Gppp(m^{2′-O})GpG (“CleanCap GG”), having a structure:

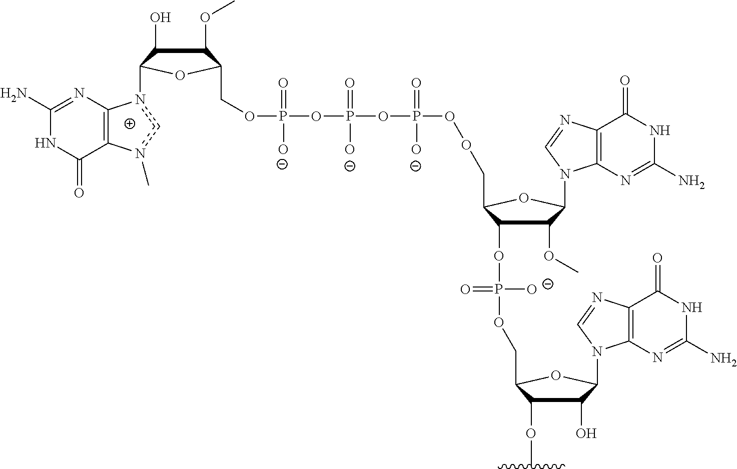

or a salt thereof.

In some embodiments, the 5′ cap is (m⁷)Gppp(m^{2′-O}) ApG, having a structure:

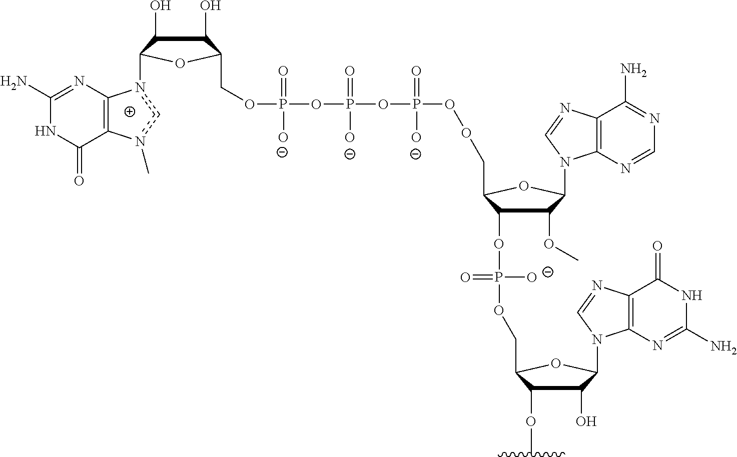

or a salt thereof.

In some embodiments, the 5′ cap is (m⁷)Gppp(m^{2′-O})GpG, having a structure:

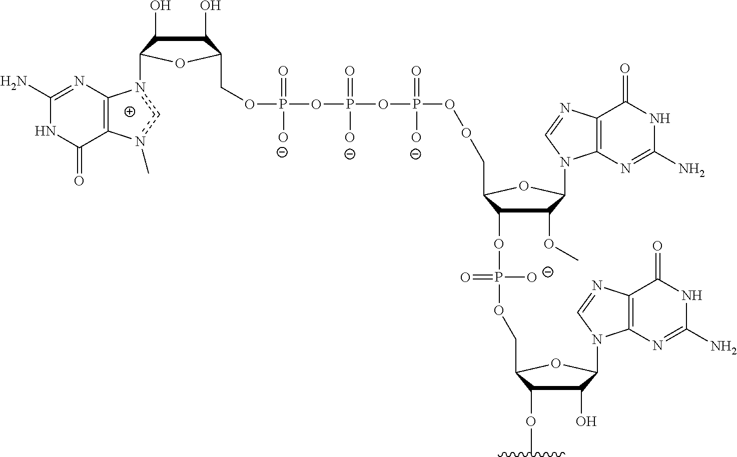

or a salt thereof.

In some embodiments, the 5′ cap is (m₂^{7,3′-O})Gpp(m₂^{6,2′-O}) ApG, having a structure:

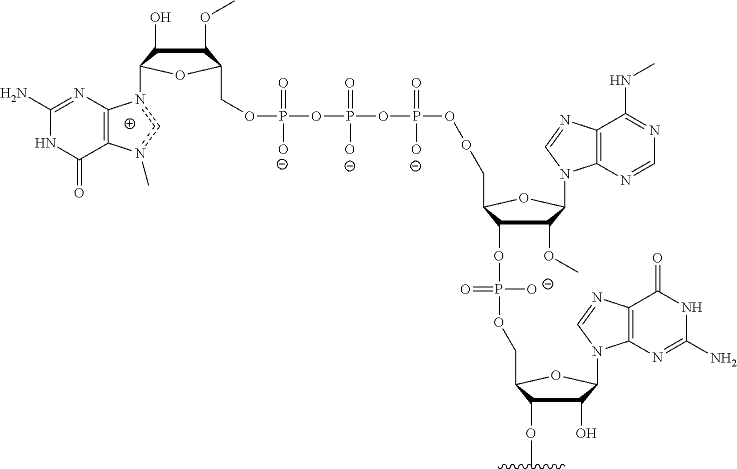

or a salt thereof.

In some embodiments, the 5′ cap is (m⁷)Gppp(m^{2′-O})ApU, having a structure:

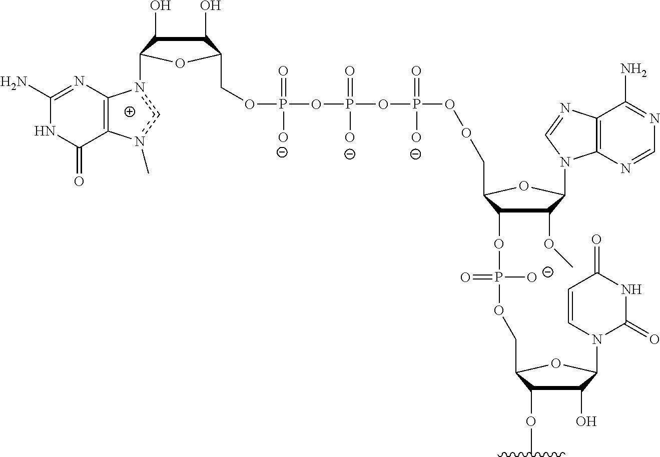

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N₁pN₂pN₃, wherein N₁, N₂, and N₃ are as defined above and herein, and G* comprises a structure of formula (I):

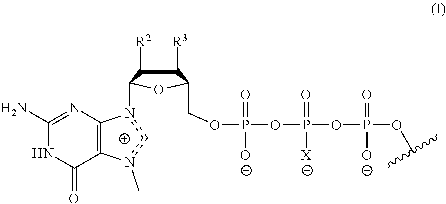

or a salt thereof, wherein R², R³, and X are as defined and described herein.

In some embodiments, the 5′ cap is a tetranucleotide cap0 structure (e.g. (m⁷)GpppN₁pN₂pN₃, (m₂^{7,2′O})GpppN₁pN₂PN₃, or (m₂^{7,3′-O})GpppN₁N₂pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂PN₃, (m^{27,2′-O})Gppp(m^{2′-O})N₁pN₂PN₃, (m₂^{7,3′-O})Gppp(m^{2′-O})N₁pN₂N₃), wherein N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂PN₃, (m^{27,2′-O})Gppp(m^{2′-O})N₁p(m^{2′-O})N₂PN₃, (m₂^{7,3′O})Gppp(m^{2′-O})N₁p(m^{2′-O})N₂pN₃), wherein N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′O})GpG, having a structure:

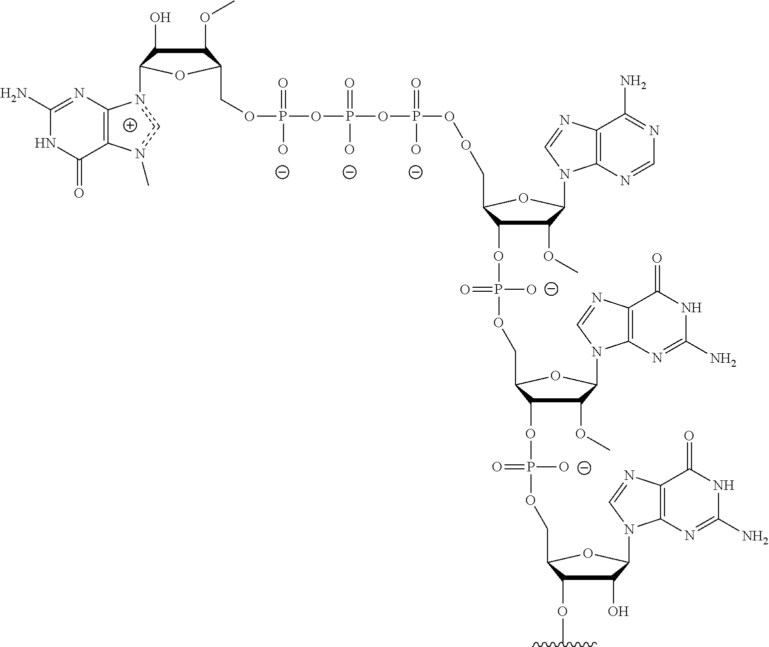

or a salt thereof.

In some embodiments, the 5′ cap is (m₂^{7,3′-O})Gppp(m^{2′-O})Gp(m^{2′-O})GpC, having a structure:

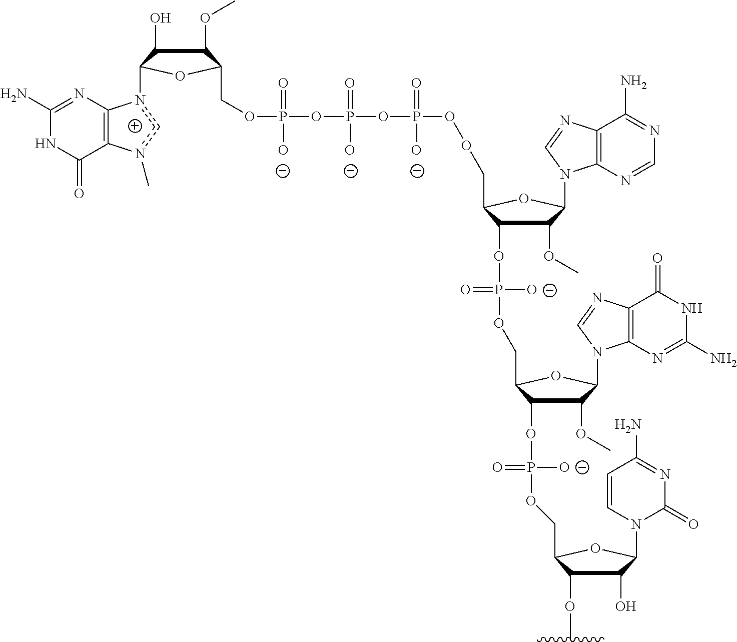

or a salt thereof.

In some embodiments, the 5′ cap is (m⁷)Gppp(m^{2′-O})Ap(m^{2′-O})UpA, having a structure:

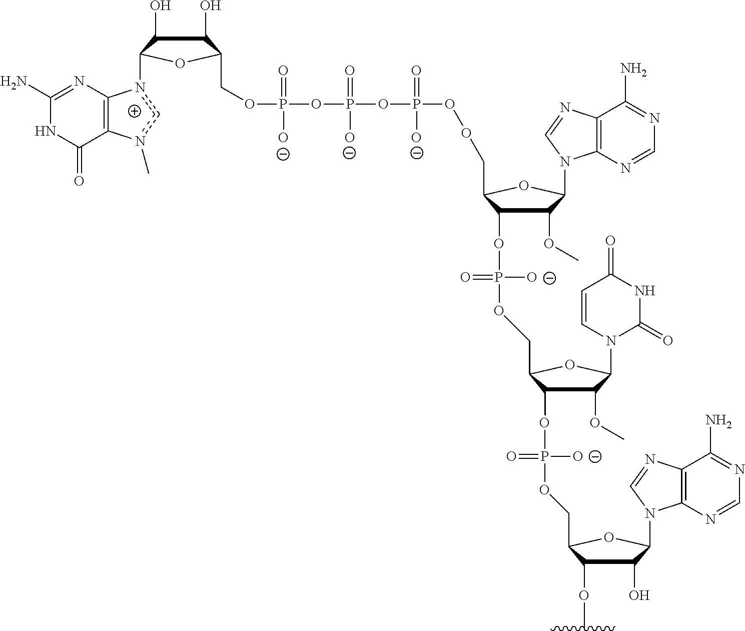

or a salt thereof.

In some embodiments, the 5′ cap is (m⁷)Gppp(m^{2′-O})Ap(m^{2′-O})GpG, having a structure:

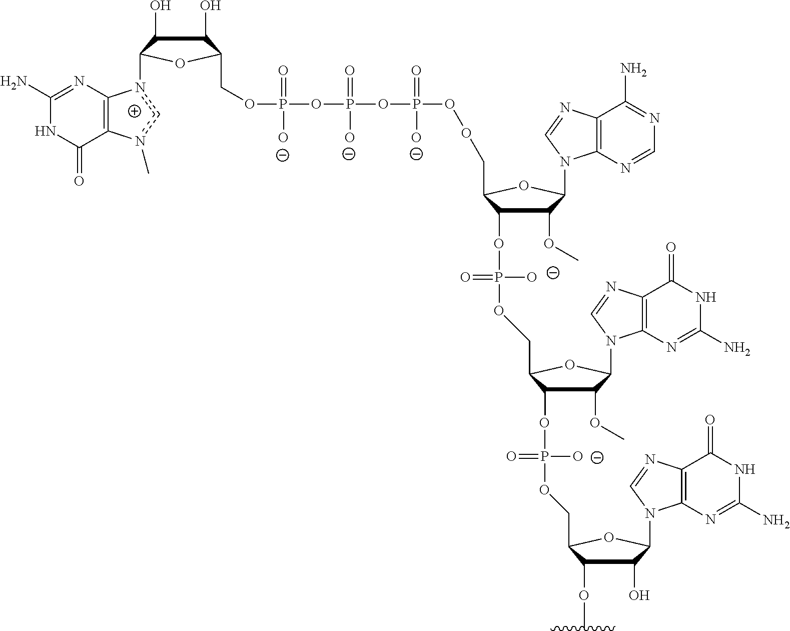

or a salt thereof.

### 2. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide +1 (N₁) of an RNA polynucleotide. In some embodiments, a cap structure comprises an m⁷ Guanosine cap and nucleotide +2 (N₂) of an RNA polynucleotide. In some embodiments, a cap structure comprises an m⁷ Guanosine cap and nucleotides +1 and +2 (N₁ and N₂) of an RNA polynucleotide. In some embodiments, a cap structure comprises an m⁷ Guanosine cap and nucleotides +1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

### 3. 5′ UTR

In some embodiments, a nucleic acid (e.g., DNA, RNA) utilized in accordance with the present disclosure comprises a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-June 5′ UTR or a fragment thereof. In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence according to SEQ ID NO: 464. In some embodiments, an RNA disclosed herein comprises a 5′ UTR having the sequence according to SEQ ID NO: 464.

### 4. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a polyA tail comprises a specific number of Adenosines, such as about 50 or more, about 60 or more, about 70 or more, about 80 or more, about 90 or more, about 100 or more, about 120, or about 150 or about 200. In some embodiments a polyA tail of a string construct may comprise 200 A residues or less. In some embodiments, a polyA tail of a string construct may comprise about 200 A residues. In some embodiments, a polyA tail of a string construct may comprise 180 A residues or less. In some embodiments, a polyA tail of a string construct may comprise about 180 A residues. In some embodiments, a polyA tail may comprise 150 residues or less.

In some embodiments, RNA comprises a poly(A) sequence comprising the nucleotide sequence of SEQ ID NO: 466, or a nucleotide sequence having at least 99%, 98%, 97%, 96%, 95%, 90%, 85%, or 80% identity to the nucleotide sequence of SEQ ID NO: 466. In some embodiments, a poly(A) tail comprises a plurality of A residues interrupted by a linker. In some embodiments, a linker comprises the nucleotide sequence of SEQ ID NO: 468.

### 5. 3′ UTR

In some embodiments, an RNA utilized in accordance with the present disclosure comprises a 3′-UTR. As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” does preferably not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a XhoI site.

In some embodiments, an RNA construct comprises an F element. In some embodiments, a F element sequence is a 3′-UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of SEQ ID NO: 470. In some embodiments, an RNA disclosed herein comprises a 3′ UTR with the sequence of SEQ ID NO: 470.

In some embodiments, a 3′UTR is an FI element as described in WO2017/060314, which is herein incorporated by reference in its entirety.

### B. RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or portion thereof. An saRNA has multiple ORFs.

In some embodiments, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

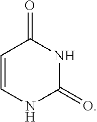

The term “uridine,” as used herein, describes one of the nucleosides that can occur in RNA. The structure of uridine is:

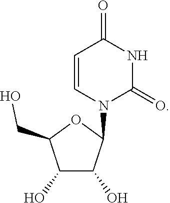 [SMILES: O=c1ccn([C@@H]2O[C@H](CO)[C@@H](O)[C@H]2O)c(=O)[nH]1]

UTP (uridine 5′-triphosphate) has the following structure:

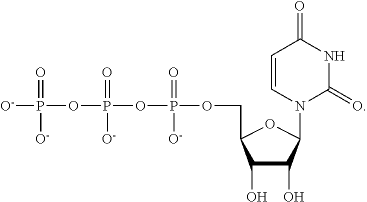

Pseudo-UTP (pseudouridine 5′-triphosphate) has the following structure:

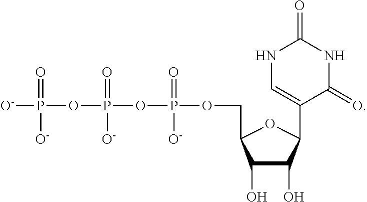

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

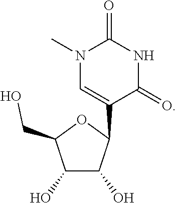 [SMILES: Cn1cc([C@@H]2O[C@H](CO)[C@@H](O)[C@H]2O)c(=O)[nH]c1=O]

N1-methyl-pseudo-UTP has the following structure:

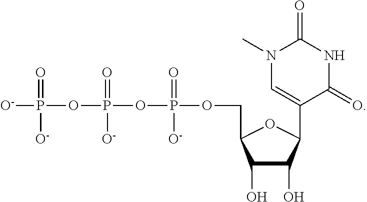

Another exemplary modified nucleoside is 5-methyl-uridine (m5U), which has the structure:

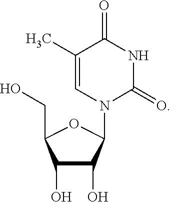

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3ψ), 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′—OH-ara-uridine, 5-(2-carbomethoxyvinyl) uridine, 5-[3-(1-E-propenylamino) uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124, which is herein incorporated by reference in its entirety). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a Plasmodium T cell string polypeptide construct described herein. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### C. Codon Optimization and GC Enrichment

As used herein, the term “codon-optimized” refers to alteration of codons in a coding region of a nucleic acid molecule (e.g., a polyribonucleotide) to reflect the typical codon usage of a host organism (e.g., a subject receiving a nucleic acid molecule (e.g., a polyribonucleotide)) without preferably altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a polyribonucleotide) may differ from wild type sequences encoding the relevant antigen or fragment or epitope thereof, even when the amino acid sequence of the antigen or fragment or epitope thereof is wild type.

In some embodiments, strategies for codon optimization for expression in a relevant subject (e.g., a human), and even, in some cases, for expression in a particular cell or tissue.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide) of the present disclosure is codon optimized, wherein the codons in the polynucleotide (e.g., the polyribonucleotide) are adapted to human codon usage (herein referred to as “human codon optimized polynucleotide”). Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 7. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 7). Accordingly, in some embodiments, such a procedure (as exemplified for Ala) is applied for each amino acid encoded by the coding sequence of a polynucleotide to obtain sequences adapted to human codon usage.

TABLE 7
Human codon usage table with frequencies
indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gin | CAG* | 0.73 |
| Cys | TGC* | 0.58 | Gin | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Tor | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0 42 |
| Lou | CTC | 0.20 | Tyr | TAC* | 0.58 |
| Met | ATG* | 1 | Stop | TGA* | 0 61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an mRNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); mRNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLOS ONE 6 (3): e17596; Rabb et al., “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content that is increased compared to a wild type coding sequence for a malarial construct described herein, or a portion thereof. In some embodiments, guanosine/cytidine (G/C) content of a coding region is modified relative to a wild type coding sequence for a malarial construct described herein, but the amino acid sequence encoded by the polyribonucleotide not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a polyribonucleotide, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a polyribonucleotide described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a polyribonucleotide described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of a polyribonucleotide may incorporate one or more elements established to contribute to stability and/or translation efficiency of the polyribonucleotide; exemplary such elements are described, for example, in PCT/EP2006/009448 incorporated herein by reference. In some embodiments, to increase expression of a polyribonucleotide used according to the present disclosure, a polyribonucleotide may be modified within the coding region, i.e., the sequence encoding the expressed peptide or protein, without altering the sequence of the expressed peptide or protein, for example so as to increase the GC-content to increase mRNA stability and/or to perform a codon optimization and, thus, enhance translation in cells.

D. Embodiments of polyribonucleotides encoding Plasmodium T cell string polypeptide constructs

In the following, exemplary embodiments of polyribonucleotides encoding Plasmodium T cell string polypeptide constructs are described, wherein certain terms used when describing elements thereof have the following meanings:
cap: 5′-cap structure, e.g., selected from the group consisting of m27,2′OG(5′)ppSp(5′)G (in particular its D1 diastereomer), m27,3′OG(5′)ppp(5′)G, and m₂^{7,3′-O}Gppp (m12′-O) ApG.
hAg-Kozak: 5′-UTR sequence of the human alpha-globin mRNA with an optimized ‘Kozak sequence’ to increase translational efficiency.
sec: Sequences encoding a secretory signal.
Antigen: Sequences encoding one or more Plasmodium polypeptides or portions thereof (e.g., one or more Plasmodium T-cell antigens from CSP, LSA-1 (a), LSA-1 (b), TRAP, LSAP2, UIS3, IS4, LISP-1, LISP-2, LSA-3, EXP1, LSAP1 and/or polypeptide regions or portions thereof (e.g., one or more antigenc fragments thereof)), e.g., from Plasmodium falciparum, preferably Plasmodium falciparum isolate 3D7.
MITD: Sequences encoding a trafficking signal.
Linker: Sequences coding for peptide linkers.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In some embodiments, a polyribonucleotide encoding a Plasmodium T cell string polypeptide construct described herein has one of the following structures:
cap-hAg-Kozak-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-MITD-FI-A30L70; and
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-Linker-Antigen-MITD-FI-A30L70.

In some embodiments, hAg-Kozak comprises the nucleotide sequence of SEQ ID NO: 473. In some embodiments, FI comprises the nucleotide sequence of SEQ ID NO: 475. In some embodiments, A30L70 comprises the nucleotide sequence of SEQ ID NO: 478.

In some embodiments, a utilized secretory signal is a heterologous secretory signal. In some embodiments, a heterologous secretory signal comprises or consists of a non-human secretory signal. In some embodiments, a heterologous secretory signal comprises or consists of a viral secretory signal. In some embodiments, a viral secretory signal comprises or consists of an HSV secretory signal (e.g., an HSV-1 or HSV-2 secretory signal). In some embodiments, an HSV secretory signal comprises or consists of an HSV glycoprotein D (gD) secretory signal. In some embodiments, the secretory signal is an HSV glycoprotein D (gD) secretory signal and has an amino acid sequence according to SEQ ID NO: 382, SEQ ID NO: 388). In some embodiments, a secretory signal comprises or consists of an Ebola virus secretory signal. In some embodiments, an Ebola virus secretory signal comprises or consists of an Ebola virus spike glycoprotein (SGP) secretory signal. In some embodiments, the secretory signal is an Ebola SGP secretory signal and has an amino acid sequence according to SEQ ID NO: 400.

In some embodiments, the MITD is an MHC class I trafficking signal. In some embodiments, the MITD comprises or consists of an amino acid sequence according to SEQ ID NO: 479.

In some embodiments, the linker is selected from an amino acid sequence as defined in SEQ ID NOs: 452-460, 468, and 469. In some embodiments, a linker has the amino acid sequence of SEQ ID NO: 452. In some embodiments, a linker has the amino acid sequence GGGS (SEQ ID NO: 459). In some embodiments, a linker has the amino acid sequence of SEQ ID NO: 456). In some embodiments, a linker has the amino acid sequence of SEQ ID NO: 460).

In some embodiments, the sequence encoding a Plasmodium T cell string polypeptide construct described herein comprises a modified nucleoside replacing (partially or completely, preferably completely) uridine, wherein the modified nucleoside is selected from the group consisting of pseudouridine (ψ), N1-methyl-pseudouridine (m1ψ), and 5-methyl-uridine.

In some embodiments, the sequence encoding a Plasmodium T cell string polypeptide construct described herein is codon-optimized.

In some embodiments, the G/C content of the sequence encoding a Plasmodium T cell string polypeptide construct described herein is increased compared to the wild type coding sequence.

In some embodiments, the RNA (in particular, mRNA) described herein comprises:
a 5′ UTR comprising the nucleotide sequence of SEQ ID NO: 465, or a nucleotide sequence having at least 99%, 98%, 97%, 96%, 95%, 90%, 85%, or 80% identity to the nucleotide sequence of SEQ ID NO: 465;
a 3′ UTR comprising the nucleotide sequence of SEQ ID NO: 471, or a nucleotide sequence having at least 99%, 98%, 97%, 96%, 95%, 90%, 85%, or 80% identity to the nucleotide sequence of SEQ ID NO: 471; and
a poly-A sequence comprising the nucleotide sequence of SEQ ID NO: 467.

In some embodiments, the RNA (in particular, mRNA) described herein comprises:
m27,3′-OGppp(m12′-O) ApG as capping structure at the 5′-end of the mRNA;
a 5′ UTR comprising the nucleotide sequence of SEQ ID NO: 465, or a nucleotide sequence having at least 99%, 98%, 97%, 96%, 95%, 90%, 85%, or 80% identity to the nucleotide sequence of SEQ ID NO: 465;
a 3′ UTR comprising the nucleotide sequence of SEQ ID NO: 471, or a nucleotide sequence having at least 99%, 98%, 97%, 96%, 95%, 90%, 85%, or 80% identity to the nucleotide sequence of SEQ ID NO: 471; and
a poly-A sequence comprising the nucleotide sequence of SEQ ID NO: 467.

In some embodiments, the RNA is unmodified. In some embodiments, the RNA is modified. In some embodiments, the RNA comprises N1-methyl-pseudouridine (m1ψ) in place of at least one uridine (e.g., in place of each uridine).

In some embodiments, the RNA (in particular, mRNA) described herein comprises:
m27,3′-OGppp(m12′-O) ApG as capping structure at the 5′-end of the mRNA;
a 5′ UTR comprising the nucleotide sequence of SEQ ID NO: 465, or a nucleotide sequence having at least 99%, 98%, 97%, 96%, 95%, 90%, 85%, or 80% identity to the nucleotide sequence of SEQ ID NO: 465;
a 3′ UTR comprising the nucleotide sequence of SEQ ID NO: 471, or a nucleotide sequence having at least 99%, 98%, 97%, 96%, 95%, 90%, 85%, or 80% identity to the nucleotide sequence of SEQ ID NO: 471; and
a poly-A sequence comprising the nucleotide sequence of SEQ ID NO: 467; and
N1-methyl-pseudouridine (m1ψ) in place of at least one uridine (e.g., in place of each uridine).

In some embodiments, a Plasmodium T cell string polypeptide construct described herein includes one or more Plasmodium polypeptides or portions thereof from Plasmodium falciparum.

In some embodiments, RNA (in particular, mRNA) described herein (e.g., contained in the compositions/formulations of the present disclosure and/or used in the methods of the present disclosure) may be presented as a product containing the vaccine RNA as active substance and other ingredients comprising: ALC-0315 ((4-hydroxybutyl) azanediyl)bis(hexane-6,1-diyl)bis(2-hexyldecanoate), ALC-0159 (2-[(polyethylene glycol)-2000]-N,N-ditetradecylacetamide), 1,2-Distearoyl-sn-glycero-3-phosphocholine (DSPC), and cholesterol.

In some embodiments, the RNA (in particular, mRNA) described herein is formulated or is to be formulated as a liquid, a solid, or a combination thereof.

In some embodiments, the RNA (in particular, mRNA) described herein is formulated or is to be formulated for injection.

In some embodiments, the RNA (in particular, mRNA) described herein is formulated or is to be formulated for intramuscular administration.

In some embodiments, the RNA (in particular, mRNA) described herein is formulated or is to be formulated as a composition, e.g., a pharmaceutical composition.

In some embodiments, the composition comprises a cationically ionizable lipid.

In some embodiments, the composition comprises a cationically ionizable lipid and one or more additional lipids. In some embodiments, the one or more additional lipids are selected from polymer-conjugated lipids, neutral lipids, and combinations thereof. In some embodiments, the neutral lipids include phospholipids, steroid lipids, and combinations thereof. In some embodiments, the one or more additional lipids are a combination of a polymer-conjugated lipid, a phospholipid, and a steroid lipid.

In some embodiments, the composition comprises a cationically ionizable lipid; a polymer-conjugated lipid which is a PEG-conjugated lipid; cholesterol; and a phospholipid. In some embodiments, the phospholipid is DSPC. In some embodiments, the phospholipid is DOPE.

In some embodiments, the composition comprises a cationically ionizable lipid; a polymer-conjugated lipid which is 2-[(polyethylene glycol)-2000]-N,N-ditetradecylacetamide; cholesterol; and a phospholipid. In some embodiments, the phospholipid is DSPC. In some embodiments, the phospholipid is DOPE.

In some embodiments, the composition comprises a cationically ionizable lipid which is ((4-hydroxybutyl) azanediyl)bis(hexane-6,1-diyl)bis(2-hexyldecanoate); a polymer-conjugated lipid which is 2-[(polyethylene glycol)-2000]-N,N-ditetradecylacetamide; cholesterol; and a phospholipid. In some embodiments, the phospholipid is DSPC. In some embodiments, the phospholipid is DOPE. In some embodiments, at least a portion of (i) the RNA, (ii) the cationically ionizable lipid, and if present, (iii) the one or more additional lipids is present in particles. In some embodiments, the particles are nanoparticles, such as lipid nanoparticles (LNPs).

In some embodiments, the composition, in particular the pharmaceutical composition, is a vaccine.

In some embodiments, the composition, in particular the pharmaceutical composition, further comprises one or more pharmaceutically acceptable carriers, diluents and/or excipients.

In some embodiments, the RNA and/or the composition, in particular the pharmaceutical composition, is/are a component of a kit.

In some embodiments, the kit further comprises instructions for use of the RNA for inducing an immune response against Plasmodium falciparum in a subject.

In some embodiments, the kit further comprises instructions for use of the RNA for therapeutically or prophylactically treating a Plasmodium falciparum infection in a subject.

In some embodiments, the subject is a human.

In some embodiments, the RNA (in particular, mRNA), e.g., RNA encoding a Plasmodium T cell string polypeptide construct, described in the present disclosure is non-immunogenic. RNA encoding an immunostimulant may be administered according to the present disclosure to provide an adjuvant effect. The RNA encoding an immunostimulant may be standard RNA or non-immunogenic RNA.

### E. Combinations of Polyribonucleotides

The present disclosure, among other things, utilizes RNA technologies as a modality to express two or more polypeptide constructs. As described further herein, in some embodiments, a polypeptide construct can be a Plasmodium T cell string polypeptide construct. In some embodiments, a Plasmodium T cell string polypeptide construct includes one or more T-cell antigens from one or more Plasmodium proteins, or one or more portions thereof, (e.g., one or more antigenic fragments thereof).

In some embodiments, two or more polypeptide constructs include a first polypeptide construct and a second polypeptide construct. In some embodiments, a first polypeptide construct is a Plasmodium T cell string polypeptide construct. In some embodiments, such a Plasmodium T cell string polypeptide construct comprises one or more Plasmodium T-cell antigens from CSP, LSA-1 (a), LSA-1 (b), TRAP, LSAP2, UIS3, IS4, LISP-1, LISP-2, LSA-3, EXP1, LSAP1 and/or polypeptide regions or portions thereof (e.g., one or more antigenic fragments thereof). In some embodiments, a Plasmodium T cell string polypeptide construct additionally includes one or more additional amino acid sequences, such as a secretory signal (e.g., a heterologous secretory signal), a transmembrane region (e.g., a heterologous transmembrane region), a trafficking signal, and/or a linker, as described herein.

In some embodiments, a second polypeptide construct is a Plasmodium polypeptide construct. As described further herein, in some embodiments, a Plasmodium polypeptide construct includes one or more Plasmodium proteins, or one or more portions thereof (e.g., immunogenic fragments of a Plasmodium polypeptide). In some embodiments, one or more Plasmodium proteins, or one or more portions thereof (e.g., immunogenic fragments of a Plasmodium polypeptide) comprise or consist of one or more Plasmodium CSP polypeptide regions or portions thereof (e.g., immunogenic fragments of Plasmodium CSP). In some embodiments, a second polypeptide construct includes one or more Plasmodium polypeptide regions or portions thereof (e.g., immunogenic fragments of a Plasmodium polypeptide), wherein the one or more Plasmodium polypeptide regions or portions thereof are different than one or more T-cell antigens from one or more Plasmodium proteins, or one or more portions thereof, (e.g., one or more antigenic fragments thereof), which are included in a first polypeptide construct. While the one or more Plasmodium polypeptide regions or portions thereof are different than one or more T-cell antigens from one or more Plasmodium proteins, or one or more portions thereof included in a first polypeptide construct, such that the second polypeptide construct is not identical to the first polypeptide construct, the present disclosure contemplates that there may be some Plasmodium polypeptide regions or portions thereof that are common to the first and second polypeptide constructs. For example, if a first polypeptide construct comprises a Plasmodium CSP major repeat region portion, a second polypeptide construct can comprise, among other Plasmodium polypeptide regions or portions thereof, a Plasmodium CSP major repeat region portion. In some embodiments, a second polypeptide construct additionally includes one or more additional amino acid sequences, such as a secretory signal (e.g., a heterologous secretory signal), a transmembrane region (e.g., a heterologous transmembrane region), a helper antigen, a multimerization region, and/or a linker, as described herein.

In certain embodiments, the present disclosure provides a combination comprising two or more polypeptide constructs as described herein. For example, in some embodiments, a combination comprises (i) a first polypeptide that comprises one or more Plasmodium T-cell antigens, as described herein; and (ii) a second polypeptide that comprises one or more Plasmodium proteins, or one or more portions thereof (e.g., Plasmodium antigenic polypeptide regions or portions thereof). In some embodiments, a combination comprises (i) a first polypeptide that comprises one or more Plasmodium T-cell antigens, as described herein; and (ii) a second polypeptide that comprises one or more Plasmodium CSP regions or portions thereof. In some embodiments, a combination comprises (i) a first polypeptide that comprises a one or more Plasmodium T-cell antigens, wherein the one or more Plasmodium T-cell antigens comprises a Plasmodium N-terminal region or portion thereof, but not a Plasmodium C-terminal region or portion thereof; and (ii) a second polypeptide that comprises one or more Plasmodium CSP polypeptide regions or portions thereof, wherein the one or more Plasmodium CSP polypeptide regions or portions thereof comprises a Plasmodium CSP C-terminal region or portion thereof, but not a Plasmodium CSP N-terminal region or portion thereof.

In accordance with the above, the present disclosure provides a combination comprising two or more polyribonucleotides that each encode a polypeptide construct. For instance, in some embodiments, a combination as provided herein can include (i) a first polyribonucleotide that encodes a first polypeptide, wherein the first polypeptide comprises a one or more Plasmodium T-cell antigens, as described herein; and (ii) a second polyribonucleotide that a second polypeptide, wherein the second polypeptide comprises one or more Plasmodium proteins, or one or more portions thereof (e.g., Plasmodium antigenic polypeptide regions or portions thereof). In some embodiments, a combination as provided herein can include (i) a first polyribonucleotide that encodes a first polypeptide, wherein the first polypeptide comprises a one or more Plasmodium T-cell antigens, as described herein; and (ii) a second polyribonucleotide that a second polypeptide, wherein the second polypeptide comprises one or more Plasmodium CSP regions or portions thereof. In some embodiments, a combination comprises (i) a first polyribonucleotide encoding a first polypeptide, wherein the first polypeptide comprises a one or more Plasmodium T-cell antigens, and wherein the one or more Plasmodium T-cell antigens comprises a Plasmodium N-terminal region or portion thereof, but not a Plasmodium C-terminal region or portion thereof; and (ii) a second polyribonucleotide encoding a second polypeptide, wherein the second polypeptide comprises one or more Plasmodium CSP polypeptide regions or portions thereof, and wherein the one or more Plasmodium CSP polypeptide regions or portions thereof comprises a Plasmodium CSP C-terminal region or portion thereof, but not a Plasmodium CSP N-terminal region or portion thereof.

In some embodiments, a first polyribonucleotide, as described herein, encode a polypeptide construct described herein has one of the following structures:
cap-hAg-Kozak-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-Linker-Antigen-FI-A30L70;
cap-hAg-Kozak-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-Antigen-Linker-Antigen-Linker-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-MITD-FI-A30L70;
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-MITD-FI-A30L70; and
cap-hAg-Kozak-sec-Antigen-Linker-Antigen-Linker-Antigen-Linker-Antigen-Linker-Antigen-Linker-Antigen-Linker-Antigen-Linker-Antigen-Linker-Antigen-Linker-Antigen-MITD-FI-A30L70.

In the above, certain terms used when describing elements thereof have the following meanings: cap: 5′-cap structure, e.g., selected from the group consisting of m27,2′OG(5′)ppSp(5′)G (in particular its D1 diastereomer), m27,3′OG(5′)ppp(5′)G, and m27,3′-OGppp(m12′-O) ApG.
hAg-Kozak: 5′-UTR sequence of the human alpha-globin mRNA with an optimized ‘Kozak sequence’ to increase translational efficiency.
sec: Sequences encoding a secretory signal.
Antigen: Sequences encoding one or more Plasmodium polypeptides or portions thereof (e.g., one or more Plasmodium T-cell antigens from CSP, LSA-1 (a), LSA-1 (b), TRAP, LSAP2, UIS3, IS4, LISP-1, LISP-2, LSA-3, EXP1, LSAP1 and/or polypeptide regions or portions thereof (e.g., one or more antigenc fragments thereof)), e.g., from Plasmodium falciparum, preferably Plasmodium falciparum isolate 3D7. MITD: Sequences encoding a trafficking signal.
Linker: Sequences coding for peptide linkers.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells. In some embodiments, hAg-Kozak comprises the nucleotide sequence of SEQ ID NO: 473. In some embodiments, FI comprises the nucleotide sequence of SEQ ID NO: 475. In some embodiments, A30L70 comprises the nucleotide sequence of SEQ ID NO: 478.

In some embodiments, a utilized secretory signal is a heterologous secretory signal. In some embodiments, a heterologous secretory signal comprises or consists of a non-human secretory signal. In some embodiments, a heterologous secretory signal comprises or consists of a viral secretory signal. In some embodiments, a viral secretory signal comprises or consists of an HSV secretory signal (e.g., an HSV-1 or HSV-2 secretory signal). In some embodiments, an HSV secretory signal comprises or consists of an HSV glycoprotein D (gD) secretory signal. In some embodiments, the secretory signal is an HSV glycoprotein D (gD) secretory signal and has an amino acid sequence according to SEQ ID NO: 382 or SEQ ID NO: 388. In some embodiments, a secretory signal comprises or consists of an Ebola virus secretory signal. In some embodiments, an Ebola virus secretory signal comprises or consists of an Ebola virus spike glycoprotein (SGP) secretory signal. In some embodiments, the secretory signal is an Ebola SGP secretory signal and has an amino acid sequence according to SEQ ID NO: 400.

In some embodiments, the MITD is an MHC class I trafficking signal. In some embodiments, the MITD comprises or consists of an amino acid sequence according to SEQ ID NO: 479.

In some embodiments, the different elements (e.g., sec, Antigen) of a first polypeptide may be linked by one or more linkers, e.g., a linker selected from an amino acid sequence as defined in SEQ ID NOS: 452-460, 468, and 469. In some embodiments, a linker has the amino acid sequence of SEQ ID NO: 452. In some embodiments, a linker has the amino acid sequence GGGS (SEQ ID NO: 459). In some embodiments, a linker has the amino acid sequence of SEQ ID NO: 456. In some embodiments, a linker has the amino acid sequence of SEQ ID NO: 460.

In some embodiments, a Plasmodium T cell string polypeptide construct described herein includes one or more Plasmodium polypeptides or portions thereof from Plasmodium falciparum. In some embodiments, one or more Plasmodium polypeptides or portions thereof are one or more P. falciparum T cell antigens. In some embodiments, one or more P. falciparum T cell antigens are from P. falciparum isolate 3D7.

In some embodiments, a Plasmodium T cell string polypeptide construct describes herein does not include one or more Plasmodium polypeptides or portions thereof from Plasmodium berghei (e.g., antigenic Plasmodium berghei CSP polypeptide fragments).

In some embodiments, a Plasmodium T cell string polypeptide construct describes herein does not include an antigenic fragment of a bacterial polypeptide. In some embodiments, a Plasmodium T cell string polypeptide construct describes herein does not include an antigenic bacillus Calmette-Guérin (BCG) polypeptide fragment. In some embodiments, an antigenic BCG polypeptide fragment comprises an amino acid sequence according to SEQ ID NO: 461. In some embodiments, a Plasmodium T cell string polypeptide construct describes herein does not include an antigenic tetanus toxin (TT) polypeptide fragment. In some embodiments, an antigenic TT polypeptide fragment comprises an amino acid sequence according to SEQ ID NO: 462.

In some embodiments, a Plasmodium T cell string polypeptide construct describes herein does not include an antigenic Plasmodium sporozoite threonine-asparagine-rich protein (STARP) polypeptide fragment. In some embodiments, an antigenic Plasmodium STARP polypeptide fragment comprises an amino acid sequence according to SEQ ID NO: 463.

In certain embodiments, the present disclosure provides a combination comprising two or more polypeptide constructs as described herein. For example, in some embodiments, a combination comprises (i) a first polypeptide comprising an antigenic Plasmodium CSP polypeptide fragment, an antigenic Plasmodium TRAP polypeptide fragment, an antigenic Plasmodium UIS3 polypeptide fragment, an antigenic Plasmodium ETRAMP10.3 polypeptide fragment, and an antigenic Plasmodium LSAP2 polypeptide fragment, and (ii) a second polypeptide comprising an antigenic Plasmodium LSA-1 (a) polypeptide fragment, an antigenic Plasmodium LSA-1 (b) polypeptide fragment, an antigenic Plasmodium LISP-2 polypeptide fragment, and an antigenic Plasmodium LISP-1 polypeptide fragment. In some embodiments, a first polypeptide comprises comprises or consists of an amino acid sequence with at least 85%, at least 90%, at least 95%, at least 96%, at least 97%, at least 98%, at least 99%, or 100% sequence identity to an amino acid sequence according to SEQ ID NO: 15. In some embodiments, a first polypeptide comprises comprises or consists of an amino acid sequence with 100% sequence identity to an amino acid sequence according to SEQ ID NO: 15. In some embodiments, a second polypeptide comprises comprises or consists of an amino acid sequence with at least 85%, at least 90%, at least 95%, at least 96%, at least 97%, at least 98%, at least 99%, or 100% sequence identity to an amino acid sequence according to SEQ ID NO: 48. In some embodiments, a second polypeptide comprises comprises or consists of an amino acid sequence with 100% sequence identity to an amino acid sequence according to SEQ ID NO: 48.

In accordance with the above, the present disclosure provides a combination comprising two or more polyribonucleotides that each encode a polypeptide construct. For instance, in some embodiments, a combination as provided herein can include (i) a first polyribonucleotide that encodes a first polypeptide, wherein the first polypeptide comprises an antigenic Plasmodium CSP polypeptide fragment, an antigenic Plasmodium TRAP polypeptide fragment, an antigenic Plasmodium UIS3 polypeptide fragment, an antigenic Plasmodium ETRAMP10.3 polypeptide fragment, and an antigenic Plasmodium LSAP2 polypeptide fragment, and (ii) a second polyribonucleotide that encodes a second polypeptide, wherein the second polypeptide comprises an antigenic Plasmodium LSA-1 (a) polypeptide fragment, an antigenic Plasmodium LSA-1 (b) polypeptide fragment, an antigenic Plasmodium LISP-2 polypeptide fragment, and an antigenic Plasmodium LISP-1 polypeptide fragment. In some embodiments, a first polypeptide comprises comprises or consists of an amino acid sequence with at least 85%, at least 90%, at least 95%, at least 96%, at least 97%, at least 98%, at least 99%, or 100% sequence identity to an amino acid sequence according to SEQ ID NO: 15. In some embodiments, a first polypeptide comprises comprises or consists of an amino acid sequence with 100% sequence identity to an amino acid sequence according to SEQ ID NO: 15. In some embodiments, a second polypeptide comprises comprises or consists of an amino acid sequence with at least 85%, at least 90%, at least 95%, at least 96%, at least 97%, at least 98%, at least 99%, or 100% sequence identity to an amino acid sequence according to SEQ ID NO: 48. In some embodiments, a second polypeptide comprises comprises or consists of an amino acid sequence with 100% sequence identity to an amino acid sequence according to SEQ ID NO: 48.

In some embodiments, a first and/or a second polyribonucleotide described herein is unmodified. In some embodiments, a first and/or a second polyribonucleotide described herein is modified. In some embodiments, a first and/or a second polyribonucleotide described herein comprises N1-methyl-pseudouridine (m1ψ) in place of at least one uridine (e.g., in place of each uridine). In some embodiments, a first and/or a second polyribonucleotide described herein comprises a modified nucleoside replacing (partially or completely, preferably completely) uridine, wherein the modified nucleoside is selected from the group consisting of pseudouridine (ψ), N1-methyl-pseudouridine (m1ψ), and 5-methyl-uridine.

In some embodiments, a first and/or a second polyribonucleotide described herein is codon-optimized.

In some embodiments, the G/C content of a first and/or a second polyribonucleotide described herein is increased compared to the wild type coding sequence.

In some embodiments, a first and/or a second polyribonucleotide described herein comprises:
a 5′ UTR comprising the nucleotide sequence of SEQ ID NO: 465, or a nucleotide sequence having at least 99%, 98%, 97%, 96%, 95%, 90%, 85%, or 80% identity to the nucleotide sequence of SEQ ID NO: 465;
a 3′ UTR comprising the nucleotide sequence of SEQ ID NO: 471, or a nucleotide sequence having at least 99%, 98%, 97%, 96%, 95%, 90%, 85%, or 80% identity to the nucleotide sequence of SEQ ID NO: 471; and
a poly-A sequence comprising the nucleotide sequence of SEQ ID NO: 467.

In some embodiments, a first and/or a second polyribonucleotide described herein comprises: m27,3′-OGppp(m12′-O) ApG as capping structure at the 5′-end of the polyribonucleotide;
a 5′ UTR comprising the nucleotide sequence of SEQ ID NO: 465, or a nucleotide sequence having at least 99%, 98%, 97%, 96%, 95%, 90%, 85%, or 80% identity to the nucleotide sequence of SEQ ID NO: 465;
a 3′ UTR comprising the nucleotide sequence of SEQ ID NO: 471, or a nucleotide sequence having at least 99%, 98%, 97%, 96%, 95%, 90%, 85%, or 80% identity to the nucleotide sequence of SEQ ID NO: 471; and
a poly-A sequence comprising the nucleotide sequence of SEQ ID NO: 467.

In some embodiments, a first and/or a second polyribonucleotide described herein comprises: m27,3′-OGppp(m12′-O) ApG as capping structure at the 5′-end of the mRNA;
a 5′ UTR comprising the nucleotide sequence of SEQ ID NO: 465, or a nucleotide sequence having at least 99%, 98%, 97%, 96%, 95%, 90%, 85%, or 80% identity to the nucleotide sequence of SEQ ID NO: 465;
a 3′ UTR comprising the nucleotide sequence of SEQ ID NO: 471, or a nucleotide sequence having at least 99%, 98%, 97%, 96%, 95%, 90%, 85%, or 80% identity to the nucleotide sequence of SEQ ID NO: 471;
a poly-A sequence comprising the nucleotide sequence of SEQ ID NO: 467; and
N1-methyl-pseudouridine (m1ψ) in place of at least one uridine (e.g., in place of each uridine).

In some embodiments, a combination described above can be administered in a pharmaceutical composition as described herein. In some embodiments, two or more polyribonucleotides of a combination as described above can be administered in separate pharmaceutical compositions as described herein. For example, in some embodiments, a combination provided herein comprises (i) a first pharmaceutical composition comprising a first polyribonucleotide, wherein the first polyribonucleotide encodes a first polypeptide, and the first polypeptide comprises a one or more Plasmodium T-cell antigens; and (ii) a second pharmaceutical composition comprising a second polyribonucleotide, wherein the second polyribonucleotide encodes a second polypeptide, and the second polypeptide comprises one or more Plasmodium antigenic polypeptide regions or portions thereof. In some embodiments, a combination provided herein comprises (i) a first pharmaceutical composition comprising a first polyribonucleotide, wherein the first polyribonucleotide encodes a first polypeptide, and the first polypeptide comprises a one or more Plasmodium T-cell antigens; and (ii) a second pharmaceutical composition comprising a second polyribonucleotide, wherein the second polyribonucleotide encodes a second polypeptide, and the second polypeptide comprises one or more Plasmodium CSP regions or portions thereof. In some embodiments, a combination provided herein comprises (i) a first pharmaceutical composition comprising a polyribonucleotide encoding a first polypeptide, wherein the first polypeptide comprises a one or more Plasmodium T-cell antigens, and wherein the one or more Plasmodium T-cell antigens comprises a Plasmodium N-terminal region or portion thereof, but not a Plasmodium C-terminal region or portion thereof; and (ii) a second pharmaceutical composition comprising a polyribonucleotide encoding a second polypeptide, wherein the second polypeptide comprises one or more Plasmodium CSP polypeptide regions or portions thereof, and wherein the one or more Plasmodium CSP polypeptide regions or portions thereof comprises a Plasmodium CSP C-terminal region or portion thereof, but not a Plasmodium CSP N-terminal region or portion thereof.

In some embodiments, a combination as provided herein can include (i) a first pharmaceutical composition comprising a first polyribonucleotide, wherein the first polyribonucleotide encodes a first polypeptide, and the first polypeptide comprises an antigenic Plasmodium CSP polypeptide fragment, an antigenic Plasmodium TRAP polypeptide fragment, an antigenic Plasmodium UIS3 polypeptide fragment, an antigenic Plasmodium ETRAMP10.3 polypeptide fragment, and an antigenic Plasmodium LSAP2 polypeptide fragment, and (ii) a second pharmaceutical composition comprising a second polyribonucleotide, wherein the second polyribonucleotide encodes a second polypeptide, and the second polypeptide comprises an antigenic Plasmodium LSA-1 (a) polypeptide fragment, an antigenic Plasmodium LSA-1 (b) polypeptide fragment, an antigenic Plasmodium LISP-2 polypeptide fragment, and an antigenic Plasmodium LISP-1 polypeptide fragment. In some embodiments, a first polypeptide comprises comprises or consists of an amino acid sequence with at least 85%, at least 90%, at least 95%, at least 96%, at least 97%, at least 98%, at least 99%, or 100% sequence identity to an amino acid sequence according to SEQ ID NO: 15. In some embodiments, a first polypeptide comprises comprises or consists of an amino acid sequence with 100% sequence identity to an amino acid sequence according to SEQ ID NO: 15. In some embodiments, a second polypeptide comprises comprises or consists of an amino acid sequence with at least 85%, at least 90%, at least 95%, at least 96%, at least 97%, at least 98%, at least 99%, or 100% sequence identity to an amino acid sequence according to SEQ ID NO: 48. In some embodiments, a second polypeptide comprises comprises or consists of an amino acid sequence with 100% sequence identity to an amino acid sequence according to SEQ ID NO: 48.

In some embodiments, a first or a second polyribonucleotide as described herein (e.g., contained in the compositions/formulations of the present disclosure and/or used in the methods of the present disclosure) may be presented as a product containing the first or the second polyribonucleotide as described herein as active substance and other ingredients comprising: ALC-0315 ((4-hydroxybutyl) azanediyl)bis(hexane-6,1-diyl)bis(2-hexyldecanoate), ALC-0159 (2-[(polyethylene glycol)-2000]-N,N-ditetradecylacetamide), 1,2-Distearoyl-sn-glycero-3-phosphocholine (DSPC), and cholesterol.

In some embodiments, a first or a second polyribonucleotide as described herein is formulated or is to be formulated as a liquid, a solid, or a combination thereof.

In some embodiments, a first or a second polyribonucleotide as described herein is formulated or is to be formulated for injection.

In some embodiments, a first or a second polyribonucleotide as described herein is formulated or is to be formulated for intramuscular administration.

In some embodiments, a first or a second polyribonucleotide as described herein is formulated or is to be formulated as a composition, e.g., a pharmaceutical composition.

In some embodiments, a composition comprises a cationically ionizable lipid.

In some embodiments, a composition comprises a cationically ionizable lipid and one or more additional lipids. In some embodiments, one or more additional lipids are selected from polymer-conjugated lipids, neutral lipids, and combinations thereof. In some embodiments, neutral lipids include phospholipids, steroid lipids, and combinations thereof. In some embodiments, one or more additional lipids are a combination of a polymer-conjugated lipid, a phospholipid, and a steroid lipid.

In some embodiments, a composition comprises a cationically ionizable lipid; a polymer-conjugated lipid which is a PEG-conjugated lipid; cholesterol; and a phospholipid. In some embodiments, a phospholipid is DSPC. In some embodiments, a phospholipid is DOPE.

In some embodiments, a composition comprises a cationically ionizable lipid; a polymer-conjugated lipid which is 2-[(polyethylene glycol)-2000]-N,N-ditetradecylacetamide; cholesterol; and a phospholipid. In some embodiments, a phospholipid is DSPC. In some embodiments, a phospholipid is DOPE.

In some embodiments, a composition comprises a cationically ionizable lipid which is ((4-hydroxybutyl) azanediyl)bis(hexane-6,1-diyl)bis(2-hexyldecanoate); a polymer-conjugated lipid which is 2-[(polyethylene glycol)-2000]-N,N-ditetradecylacetamide; cholesterol; and a phospholipid. In some embodiments, a phospholipid is DSPC. In some embodiments, a phospholipid is DOPE. In some embodiments, at least a portion of (i) a first or a second polyribonucleotide as described herein, (ii) a cationically ionizable lipid, and if present, (iii) one or more additional lipids is present in particles. In some embodiments, particles are nanoparticles, such as lipid nanoparticles (LNPs).

In some embodiments, a composition, in particular the pharmaceutical composition, is a vaccine.

In some embodiments, a composition, in particular the pharmaceutical composition, further comprises one or more pharmaceutically acceptable carriers, diluents and/or excipients.

### IV. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid compositions, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells (e.g., liver cells). In some embodiments, such lipid nanoparticles may be particularly useful to deliver polyribonucleotides when polyribonucleotides are intravenously or intramuscularly administered to a subject.

### A. Lipid Compositions

### 1. Lipids and Lipid-Like Materials

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of a polar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

A “lipid-like material” is a substance that is structurally and/or functionally related to a lipid but may not be considered a lipid in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

Specific examples of amphiphilic compounds that may be included in an amphiphilic layer include, but are not limited to, phospholipids, aminolipids and sphingolipids.

Generally, lipids may be divided into eight categories: fatty acids, glycerolipids, glycerophospholipids, sphingolipids, saccharolipids, polyketides (derived from condensation of ketoacyl subunits), sterols and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

Glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are members of a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterols, such as cholesterol and its derivatives, or tocopherol and its derivatives, are important components of membrane lipids, along with the glycerophospholipids and sphingomyelins.

Saccharolipids are compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

Lipids and lipid-like materials may be cationic, anionic or neutral. Neutral lipids or lipid-like materials exist in an uncharged or neutral zwitterionic form at a selected pH.

In some embodiments, suitable lipids or lipid-like materials for use in the present disclosure include those described in WO2020/128031 and US20200163878, the entire contents of each of which are incorporated herein by reference for the purposes described herein.

### 2. Cationic or Cationically Ionizable Lipids or Lipid-Like Materials

In some embodiments cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

Cationic lipids or lipid-like materials are characterized in that they have a net positive charge (e.g., at a relevant pH). Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

In some embodiments, a cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N-(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 2-[({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N₁-[2-((1S)-1-[(3-aminopropyl)amino]-4-[di(3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy) heptadecanedioate (L319), N-Dodecyl-3-((2-dodecylcarbamoyl-ethyl)-{2-[(2-dodecylcarbamoyl-ethyl)-2-[{(2-dodecylcarbamoyl-ethyl)-[2-(2-dodecylcarbamoyl-ethylamino)-ethyl]-amino}-ethylamino) propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the present disclosure include those described in WO2020/128031 and US20200163878, the entire contents of each of which are incorporated herein by reference for the purposes described herein. Further suitable cationic lipids for use in the present disclosure include those described in WO2010/053572 (including CI 2-200 described at paragraph) and WO2012/170930, both of which are incorporated herein by reference for the purposes described herein. Additional suitable cationic lipids for use in the present disclosure include HGT4003, HGT5000, HGTS001, HGT5001, HGT5002 (see US20150140070A1, which is herein incorporated by reference in its entirety).

In some embodiments, formulations that are useful for pharmaceutical compositions (e.g., immunogenic compositions, e.g., vaccines) compositions as described herein can comprise at least one cationic lipid. Representative cationic lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In some embodiments, amino or cationic lipids useful in accordance with the present disclosure have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, a protonatable lipid has a pKa of the protonatable group in the range of about 4 to about 11, e.g., a pKa of about 5 to about 7.

In some embodiments, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of total lipid present in a lipid composition utilized in accordance with the present disclosure.

### 3. Additional Lipids or Lipid-Like Materials

In some embodiments, formulations utilized in accordance with the present disclosure may comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. In some embodiments, optimizing a formulation of nucleic acid particles by addition of other hydrophobic moieties, such as cholesterol and lipids, in addition to an ionizable/cationic lipid or lipid-like material may, for example, enhance particle stability and efficacy of nucleic acid delivery.

In some embodiments, a lipid or lipid-like material may be incorporated which may or may not affect the overall charge of particles. In certain embodiments, such lipid or lipid-like material is a non-cationic lipid or lipid-like material.

In some embodiments, a non-cationic lipid may comprise, e.g., one or more anionic lipids and/or neutral lipids. An “anionic lipid” is negatively charged (e.g., at a selected pH).

A “neutral lipid” exists either in an uncharged or neutral zwitterionic form (e.g., at a selected pH). In some embodiments, a formulation comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific exemplary phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain embodiments, a formulation utilized in accordance with the present disclosure includes DSPC or DSPC and cholesterol.

In certain embodiments, formulations utilized in accordance with the present disclosure include both a cationic lipid and an additional (non-cationic) lipid.

In some embodiments, formulations herein include a polymer conjugated lipid such as a pegylated lipid. “Pegylated lipids” comprise both a lipid portion and a polyethylene glycol portion. Pegylated lipids are known in the art.

Without wishing to be bound by theory, the amount of (total) cationic lipid compared to the amount of other lipid(s) in formulation may affect important characteristics, such as charge, particle size, stability, tissue selectivity, and bioactivity of the nucleic acid. In some embodiments, the molar ratio of the at least one cationic lipid to the at least one additional lipid is from about 10:0 to about 1:9, about 4:1 to about 1:2, or about 3:1 to about 1:1.

In some embodiments, a non-cationic lipid, in particular a neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in a formulation.

### 4. Lipoplex Particles

In certain embodiments of the present disclosure, the RNA described herein may be present in RNA lipoplex particles.

An “RNA lipoplex particle” contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

In some embodiments, RNA lipoplex particles have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### 5. Lipid Nanoparticles (LNPs)

In some embodiments, nucleic acid such as RNA described herein is administered in the form of lipid nanoparticles (LNPs). In some embodiments, LNPs may comprise any lipid capable of forming a particle to which the one or more nucleic acid molecules are attached, or in which the one or more nucleic acid molecules are encapsulated.

In some embodiments, an LNP comprises one or more cationic lipids, and one or more stabilizing lipids. Stabilizing lipids include neutral lipids and pegylated lipids.

In some embodiments, an LNP comprises a cationic lipid, a neutral lipid, a sterol, a polymer conjugated lipid; and an RNA, encapsulated within or associated with the lipid nanoparticle.

In some embodiments, a neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, a sterol is cholesterol.

In some embodiments, a polymer conjugated lipid is a pegylated lipid. In some embodiments, a pegylated lipid has the following structure:

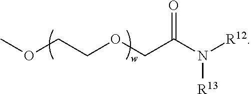

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is DMG-PEG 2000, e.g., having the following structure:

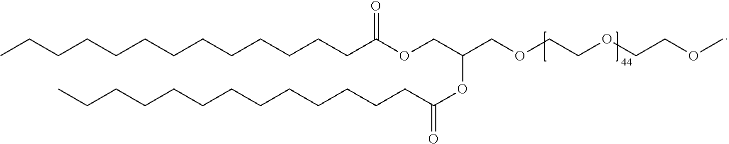

In some embodiments, a cationic lipid component of LNPs has the structure of Formula (III):

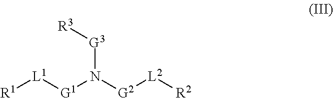

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(═O)—, —NRaC(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G₁ and G₂ are each independently unsubstituted C₁-C₁₂ alkylene or C₁-C₁₂ alkenylene;
G₃ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

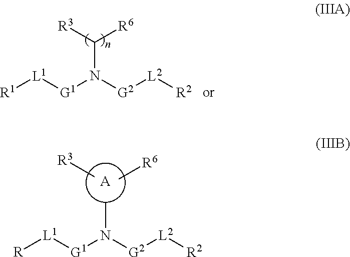

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

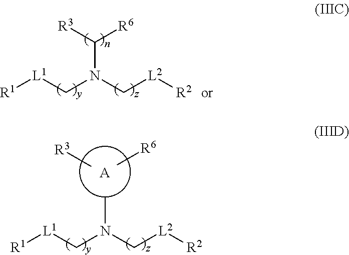

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

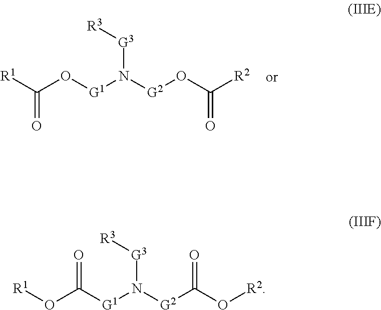

In some of the foregoing embodiments of Formula (III), the lipid has one of the following structures (IIIG), (IIIH), (IIII), or (IIIJ):

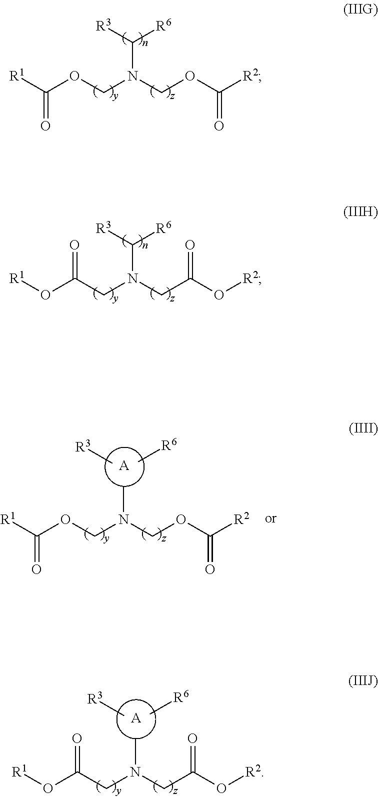

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G₃ is unsubstituted. In other embodiments, G₃ is substituted. In various different embodiments, G₃ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

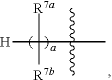

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈alkyl. For example, in some embodiments, C₁-C₈alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

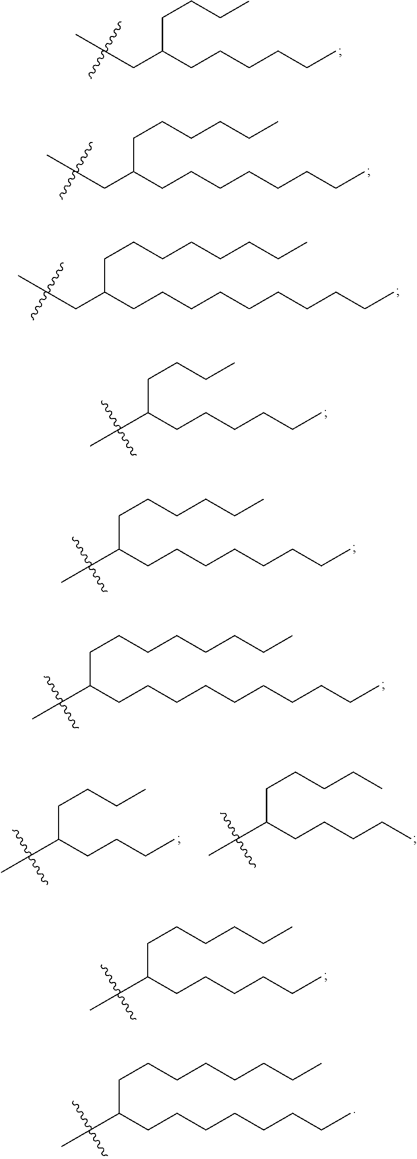

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in in Table 8 below.

TABLE 8
Exemplary Compounds of Formula (III)

| No. | Structure |
|---|---|
| III-1 | 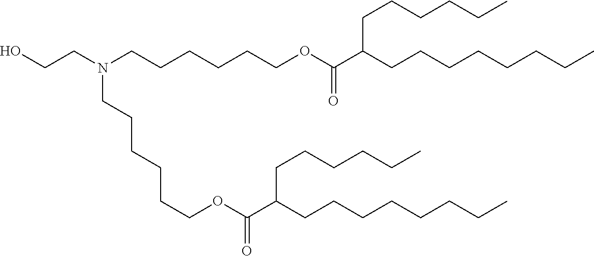 [SMILES: CCCCCCCCC(CCCCCC)C(=O)OCCCCCCN(CCO)CCCCCCOC(=O)C(CCCCCC)CCCCCCCC] |
| III-2 | 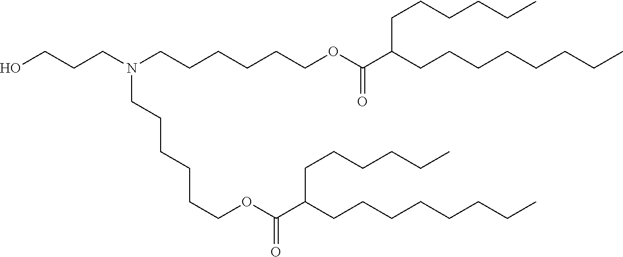 [SMILES: CCCCCCCCC(CCCCCC)C(=O)OCCCCCCN(CCCO)CCCCCCOC(=O)C(CCCCCC)CCCCCCCC] |
| III-3 | 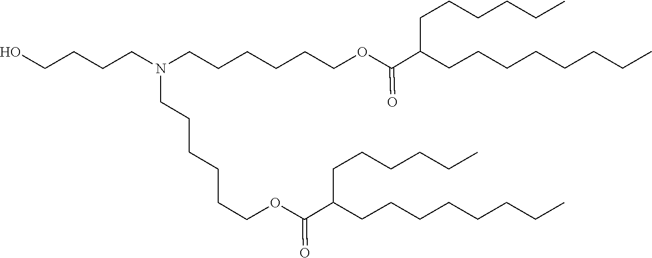 [SMILES: CCCCCCCCC(CCCCCC)C(=O)OCCCCCCN(CCCCO)CCCCCCOC(=O)C(CCCCCC)CCCCCCCC] |
| III-4 | 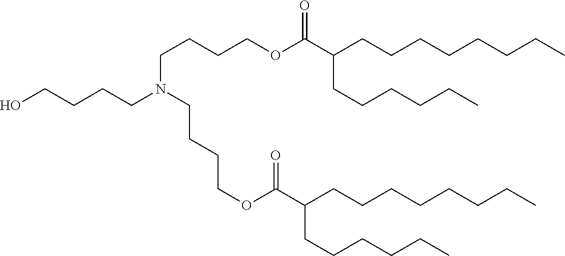 [SMILES: CCCCCCCCC(CCCCCC)C(=O)OCCCCN(CCCCO)CCCCOC(=O)C(CCCCCC)CCCCCCCC] |
| III-5 | 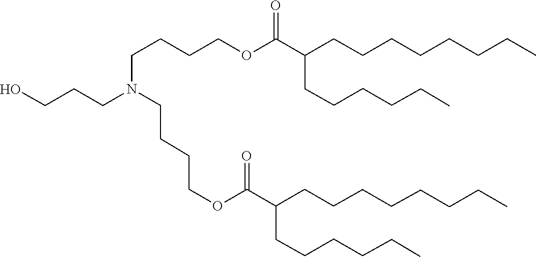 [SMILES: CCCCCCCCC(CCCCCC)C(=O)OCCCCN(CCCO)CCCCOC(=O)C(CCCCCC)CCCCCCCC] |
| III-6 | 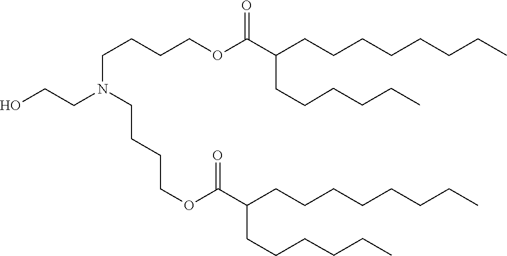 [SMILES: CCCCCCCCC(CCCCCC)C(=O)OCCCCN(CCO)CCCCOC(=O)C(CCCCCC)CCCCCCCC] |
| III-7 | 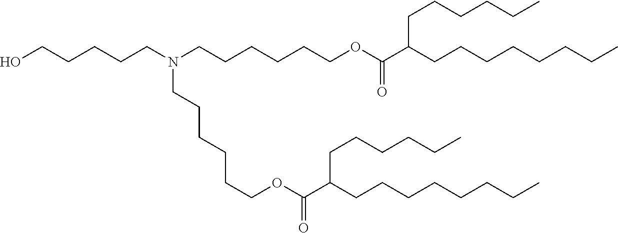 [SMILES: CCCCCCCCC(CCCCCC)C(=O)OCCCCCCN(CCCCCO)CCCCCCOC(=O)C(CCCCCC)CCCCCCCC] |
| III-8 | 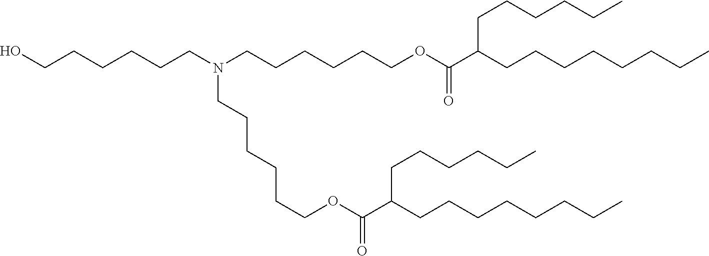 [SMILES: CCCCCCCCC(CCCCCC)C(=O)OCCCCCCN(CCCCCCO)CCCCCCOC(=O)C(CCCCCC)CCCCCCCC] |
| III-9 | 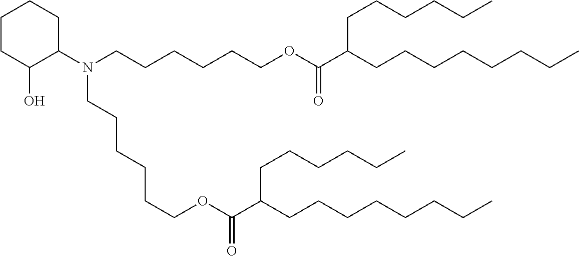 [SMILES: CCCCCCCCC(CCCCCC)C(=O)OCCCCCCN(CCCCCCOC(=O)C(CCCCCC)CCCCCCCC)C1CCCCC1O] |
| III-10 | 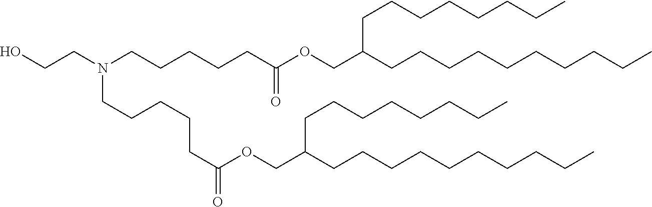 [SMILES: CCCCCCCCCCC(CCCCCCCC)COC(=O)CCCCCN(CCO)CCCCCC(=O)OCC(CCCCCCCC)CCCCCCCCCC] |
| III-11 | 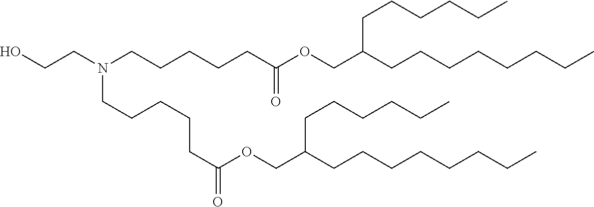 [SMILES: CCCCCCCCC(CCCCCC)COC(=O)CCCCCN(CCO)CCCCCC(=O)OCC(CCCCCC)CCCCCCCC] |
| III-12 | 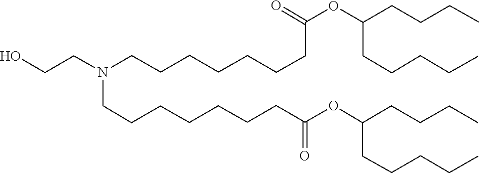 [SMILES: CCCCCC(CCCC)OC(=O)CCCCCCCN(CCO)CCCCCCCC(=O)OC(CCCC)CCCCC] |
| III-13 | 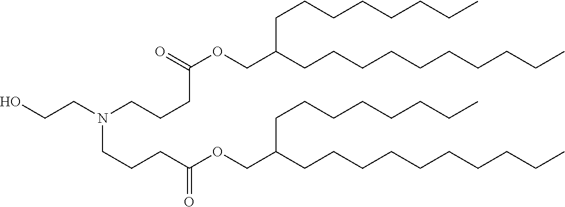 [SMILES: CCCCCCCCCCC(CCCCCCCC)COC(=O)CCCN(CCO)CCCC(=O)OCC(CCCCCCCC)CCCCCCCCCC] |
| III-14 | 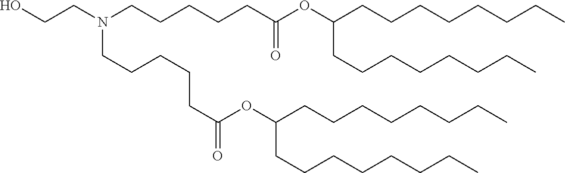 [SMILES: CCCCCCCCC(CCCCCCCC)OC(=O)CCCCCN(CCO)CCCCCC(=O)OC(CCCCCCCC)CCCCCCCC] |
| III-15 | 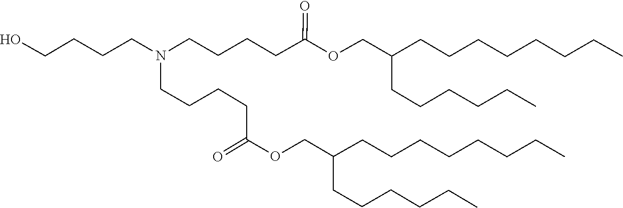 [SMILES: CCCCCCCCC(CCCCCC)COC(=O)CCCCN(CCCCO)CCCCC(=O)OCC(CCCCCC)CCCCCCCC] |
| III-16 | 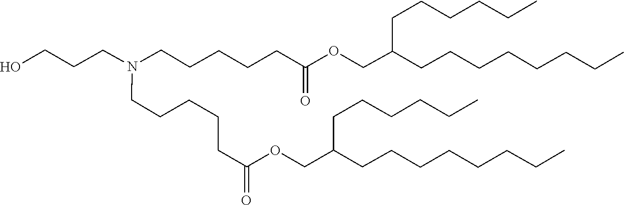 [SMILES: CCCCCCCCC(CCCCCC)COC(=O)CCCCCN(CCCO)CCCCCC(=O)OCC(CCCCCC)CCCCCCCC] |
| III-17 | 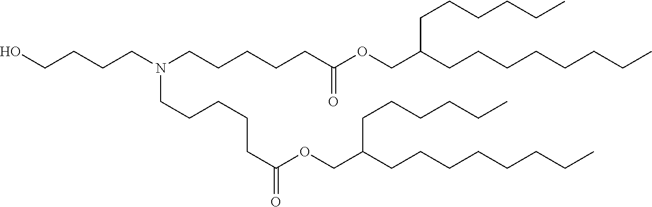 [SMILES: CCCCCCCCC(CCCCCC)COC(=O)CCCCCN(CCCCO)CCCCCC(=O)OCC(CCCCCC)CCCCCCCC] |
| III-18 | 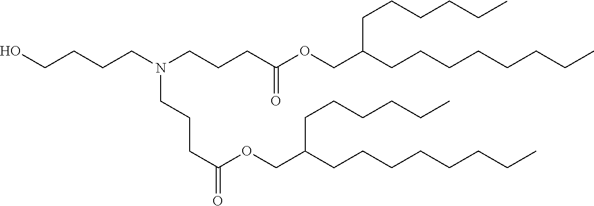 [SMILES: CCCCCCCCC(CCCCCC)COC(=O)CCCN(CCCCO)CCCC(=O)OCC(CCCCCC)CCCCCCCC] |
| III-19 | 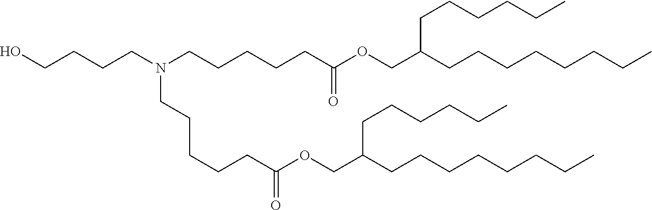 [SMILES: CCCCCCCCC(CCCCCC)COC(=O)CCCCCN(CCCCO)CCCCCC(=O)OCC(CCCCCC)CCCCCCCC] |
| III-20 | 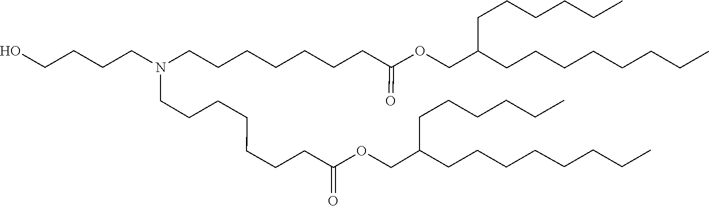 [SMILES: CCCCCCCCC(CCCCCC)COC(=O)CCCCCCCN(CCCCO)CCCCCCCC(=O)OCC(CCCCCC)CCCCCCCC] |
| III-21 | 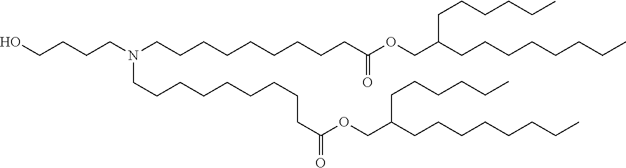 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 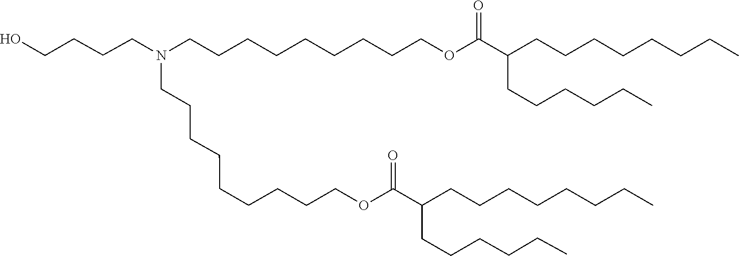 [SMILES: CCCCCCCCC(CCCCCC)C(=O)OCCCCCCCCCN(CCCCO)CCCCCCCCCOC(=O)C(CCCCCC)CCCCCCCC] |
| III-23 | 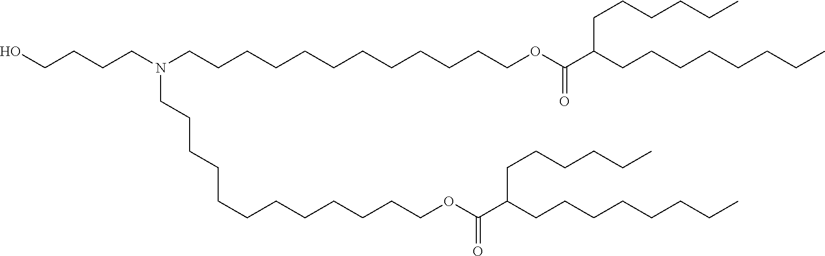 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 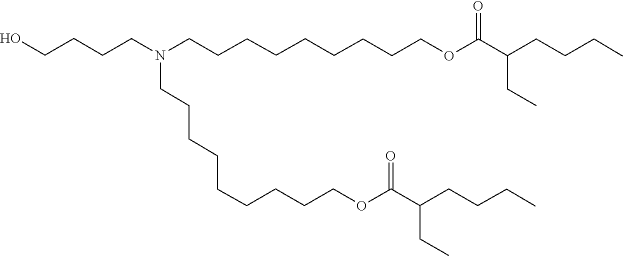 [SMILES: CCCCC(CC)C(=O)OCCCCCCCCCN(CCCCO)CCCCCCCCCOC(=O)C(CC)CCCC] |
| III-25 | 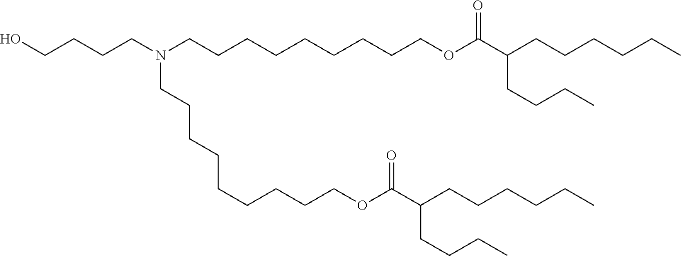 [SMILES: CCCCCCC(CCCC)C(=O)OCCCCCCCCCN(CCCCO)CCCCCCCCCOC(=O)C(CCCC)CCCCCC] |
| III-26 | 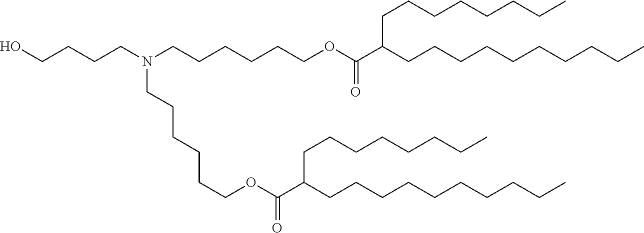 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 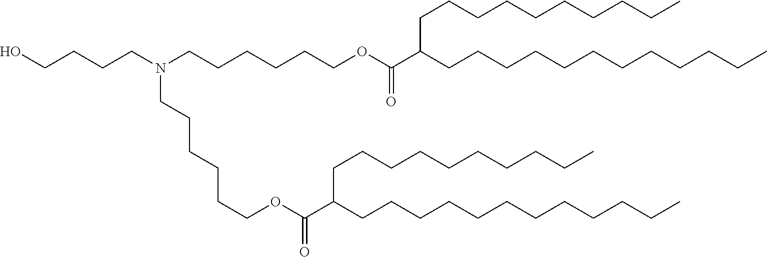 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 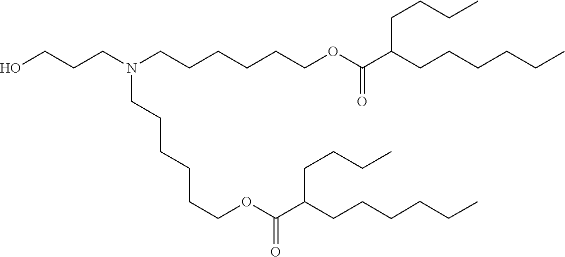 [SMILES: CCCCCCC(CCCC)C(=O)OCCCCCCN(CCCO)CCCCCCOC(=O)C(CCCC)CCCCCC] |
| III-29 | 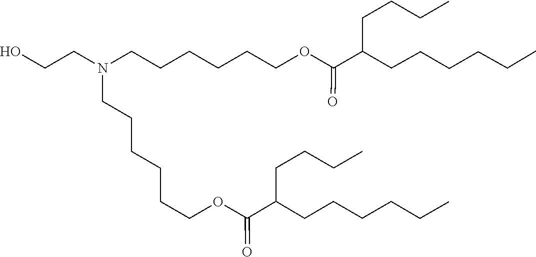 [SMILES: CCCCCCC(CCCC)C(=O)OCCCCCCN(CCO)CCCCCCOC(=O)C(CCCC)CCCCCC] |
| III-30 | 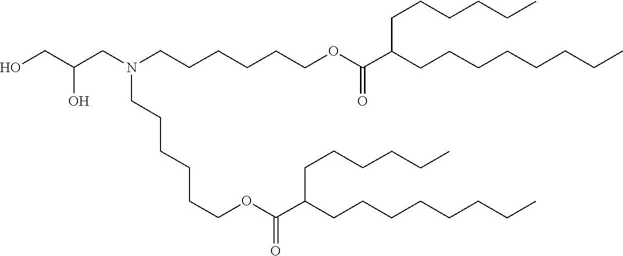 [SMILES: CCCCCCCCC(CCCCCC)C(=O)OCCCCCCN(CCCCCCOC(=O)C(CCCCCC)CCCCCCCC)CC(O)CO] |
| III-31 | 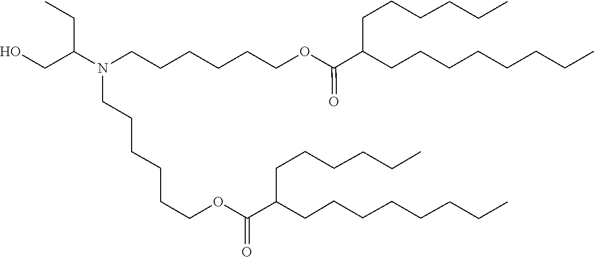 [SMILES: CCCCCCCCC(CCCCCC)C(=O)OCCCCCCN(CCCCCCOC(=O)C(CCCCCC)CCCCCCCC)C(CC)CO] |
| III-32 | 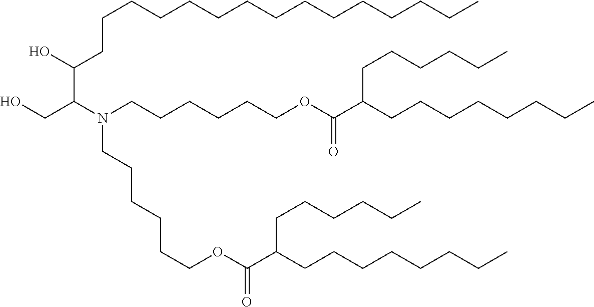 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 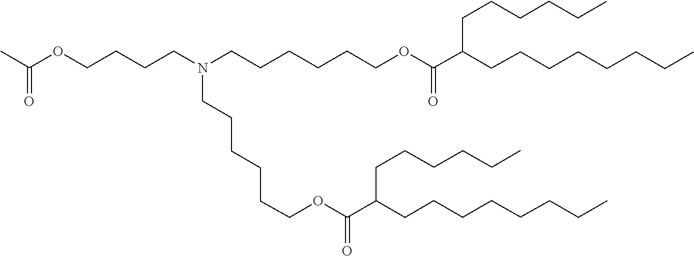 [SMILES: CCCCCCCCC(CCCCCC)C(=O)OCCCCCCN(CCCCCCOC(=O)C(CCCCCC)CCCCCCCC)CCCCOC(C)=O] |
| III-34 | 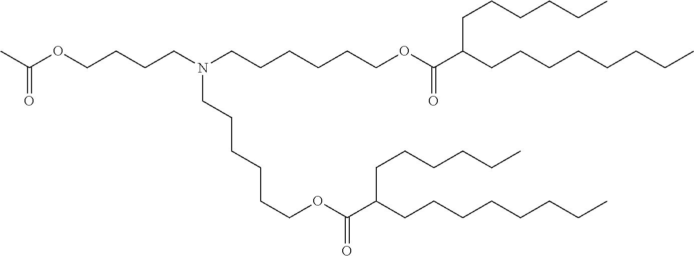 [SMILES: CCCCCCCCC(CCCCCC)C(=O)OCCCCCCN(CCCCCCOC(=O)C(CCCCCC)CCCCCCCC)CCCCOC(C)=O] |
| III-35 | 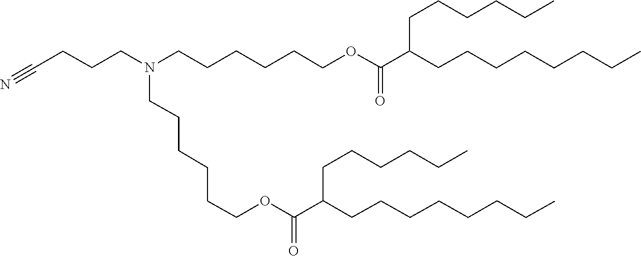 [SMILES: CCCCCCCCC(CCCCCC)C(=O)OCCCCCCN(CCCC#N)CCCCCCOC(=O)C(CCCCCC)CCCCCCCC] |
| III-36 | 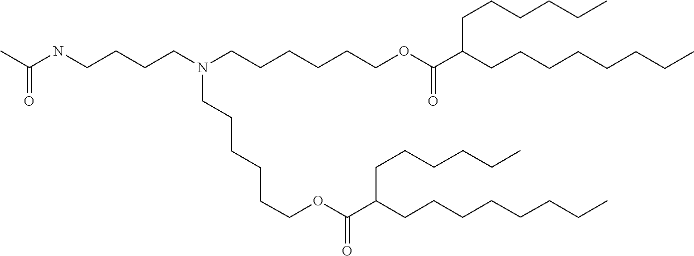 [SMILES: CCCCCCCCC(CCCCCC)C(=O)OCCCCCCN(CCCCCCOC(=O)C(CCCCCC)CCCCCCCC)CCCCNC(C)=O] |

In various different embodiments, a cationic lipid has one of the structures set forth in Table 9 below.

TABLE 9
Exemplary Cationic Lipid Structures

| No. | Structure |
|---|---|
| A | 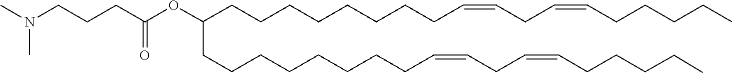 [SMILES: CCCCC/C=C\C/C=C\CCCCCCCCC(CCCCCCCC/C=C\C/C=C\CCCCC)OC(=O)CCCN(C)C] |
| B | 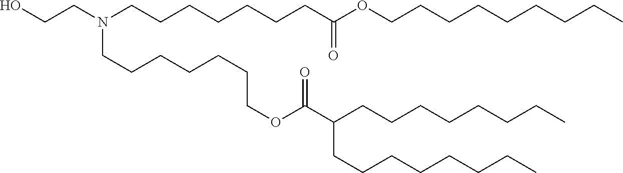 [SMILES: CCCCCCCCCOC(=O)CCCCCCCN(CCO)CCCCCCCOC(=O)C(CCCCCCCC)CCCCCCCC] |
| C | 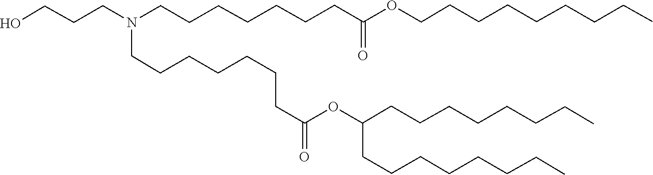 [SMILES: CCCCCCCCCOC(=O)CCCCCCCN(CCCO)CCCCCCCC(=O)OC(CCCCCCCC)CCCCCCCC] |
| D | 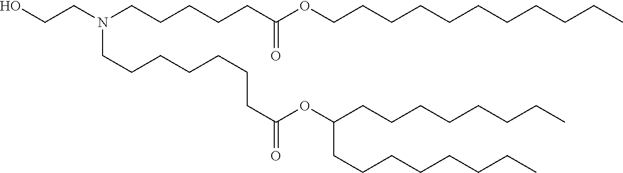 [SMILES: CCCCCCCCCCCOC(=O)CCCCCN(CCO)CCCCCCCC(=O)OC(CCCCCCCC)CCCCCCCC] |
| E | 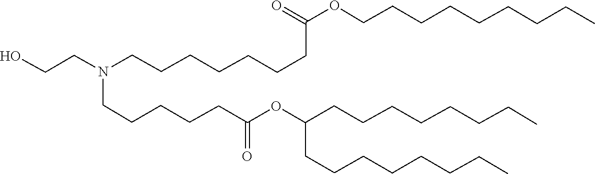 [SMILES: CCCCCCCCCOC(=O)CCCCCCCN(CCO)CCCCCC(=O)OC(CCCCCCCC)CCCCCCCC] |
| F | 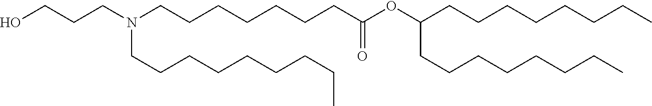 [SMILES: CCCCCCCCCN(CCCO)CCCCCCCC(=O)OC(CCCCCCCC)CCCCCCCC] |

In some embodiments, an LNP comprises a cationic lipid that is an ionizable lipid-like material (lipidoid). In some embodiments, a cationic lipid has the following structure:

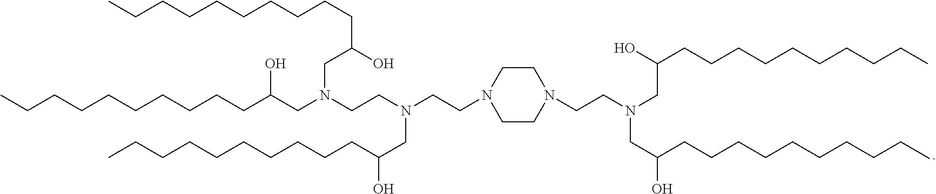 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

In some embodiments, lipid nanoparticles described herein may exhibit a polydispersity index less than about 0.5, less than about 0.4, less than about 0.3, or about 0.2 or less. By way of example, lipid nanoparticles can exhibit a polydispersity index in a range of about 0.1 to about 0.3 or about 0.2 to about 0.3. The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Lipid nanoparticles described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a lipid nanoparticle described herein has an N/P ratio greater than or equal to 5. In some embodiments, a lipid nanoparticle described herein has an N/P ratio that is about 5, 6, 7, 8, 9, or 10. In some embodiments, an N/P ratio for a lipid nanoparticle described herein is from about 10 to about 50. In some embodiments, an N/P ratio for a lipid nanoparticle described herein is from about 10 to about 70. In some embodiments, an N/P ratio for a lipid nanoparticle described herein is from about 10 to about 120.

### B. Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/ml in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or without RNAs) may be monitored by appropriate techniques such as, e.g., small-angle X-ray scattering (SAXS) and/or transmission electron cryomicroscopy (CryoTEM).

### V. Pharmaceutical Compositions

The present disclosure provides compositions, e.g., pharmaceutical compositions comprising one or more polyribonucleotides described herein. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art. In some embodiments, pharmaceutical compositions can be prepared as described herein and/or methods known in the art. In some embodiments, a pharmaceutical composition includes ALC-0315; ALC-0159; DSPC; Cholesterol; Sucrose; NaCl; KCl; Na₂HPO₄; KH₂PO₄; Water for injection. In some embodiments, normal saline (isotonic 0.9% NaCl) is used as diluent.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition is formulated (e.g., but not limited to, for intravenous, intramuscular, or subcutaneous administration) to deliver a dose of about 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### VI. Patient Populations

In some aspects, technologies of the present disclosure are used for therapeutic and/or prophylactic purposes. In some embodiments, technologies of the present disclosure are used in the treatment and/or prophylactic of an infection with a malaria parasite. Prophylactic purposes of the present disclosure comprise pre-exposure prophylaxis and/or post-exposure prophylaxis. In some such embodiments, a malaria parasite is, for example, Plasmodium falciparum, Plasmodium knowlesi, Plasmodium ovale, Plasmodium simiovale, Plasmodium vivax, Plasmodium malariae, Plasmodium ovale curtisi, Plasmodium ovale wallikeri, and/or Plasmodium berghei.

In some embodiments, technologies of the present disclosure are used in the treatment and/or prophylaxis of a disorder related to such an infection. A disordered related to such an infection comprises, for example, a typical symptom and/or a complication of a malaria infection.

In some embodiments, provided compositions (e.g., that are or comprise Plasmodium antigens) may be useful to detect and/or characterize one or more features of an anti-malarial immune response (e.g., by detecting binding to a provided antigen by serum from an infected subject).

In some embodiments, provided compositions (e.g., that are or comprise Plasmodium antigens) are useful to raise antibodies to one or more epitopes included therein; such antibodies may themselves be useful, for example for detection or treatment of malarial parasite(s) or infection thereby.

The present disclosure provides use of encoding nucleic acids (e.g., DNA or RNA) to produce encoded antigens and/or use of DNA constructs to produce RNA.

In some embodiments, technologies of the present disclosure are utilized in a non-limited subject population; in some embodiments, technologies of the present disclosure are utilized in particular subject populations.

In some embodiments, a subject population comprises an adult population. In some embodiments, an adult population comprises subjects between the ages of about 19 years and about 60 years of age (e.g., about 20, 25, 30, 35, 40, 45, 50, 55, or 60 years of age).). In some embodiments, an adult population comprises subjects between the ages of about 19 years and about 60 years of age (e.g., about 20, 25, 30, 35, 40, 45, 50, 55, or 60 years of age). In some embodiments, an adult population comprises subjects between the ages of about 18 years and about 55 years of age (e.g., about 20, 25, 30, 35, 40, 45, 50, 55, or 60 years of age).

In some embodiments, a subject population comprises an elderly population. In some embodiments, an elderly population comprises subjects of about 60 years of age, about 70 years of age, or older (e.g., about 65, 70, 75, 80, 85, 90, 95, or 100 years of age).

In some embodiments, a subject population comprises a pediatric population. In some embodiments, a pediatric population comprises subjects approximately 18 years old or younger. In some such embodiments, a pediatric population comprises subjects between the ages of about 1 year and about 18 years (e.g., 1, 2, 3, 4, 5, 6, 7, 8, 9, 10, 11, 12, 13, 14, 15, 16, 17, or 18 years of age).

In some embodiments, a subject population comprises a newborn population. In some embodiments, a newborn population comprises subjects about 12 months or younger (e.g., 12, 11, 10, 9, 8, 7, 6, 5, 4, 3, 2, 1 months or younger). In some embodiments, subject populations to be treated with technologies described herein include infants (e.g., about 12 months or younger) whose mothers did not receive such technologies described herein during pregnancy. In some embodiments, subject populations to be treated with technologies described herein may include pregnant women; in some embodiments, infants whose mothers were treated with disclosed technologies during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were treated with disclosed technologies during pregnancy (e.g., who received at least one dose, or alternatively only who received both doses), receive reduced treated with disclosed technologies (e.g., lower doses and/or smaller numbers of administrations—e.g., boosters—and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters—over a given period of time), In some embodiments, compositions as provided herein are administered to subject populations that do not include pregnant women.

In some embodiments, a subject population is or comprises children aged 6 weeks to up to 17 months of age.

In some embodiments, a subject has a body mass index over 15 kg/m² and under 40 kg/m². In some embodiments, a subject has a body mass index over 18.5 kg/m² and under 35 kg/m. In some embodiments, a subject's body mass index is determined at an initial visit with a health professional. In some embodiments, a subject's body mass index is determined when a first dose of a pharmaceutical composition (e.g., immunogenic composition, e.g., vaccine) as described herein is administered.

In some embodiments, a subject weighs at least 40 kg. In some embodiments, a subject weighs at least 45 kg. In some embodiments, a subject's weight is determined at an initial visit with a health professional. In some embodiments, a subject's weight is determined when a first dose of a pharmaceutical composition (e.g., immunogenic composition, e.g., vaccine) as described herein is administered.

In some embodiments, a subject has a body mass index over 15 kg/m² and under 40 kg/m² and weighs at least 40 kg. In some embodiments, a subject has a body mass index over 18.5 kg/m² and under 35 kg/m² and weighs at least 45 kg. In some embodiments, a subject's body mass index and weight are determined at an initial visit with a health professional. In some embodiments, a subject's body mass index and weight are determined when a first dose of a pharmaceutical composition (e.g., immunogenic composition, e.g., vaccine) as described herein is administered.

In some embodiments, a subject population comprises a population with a high risk of infection (e.g., Malaria). In some such embodiments, a population may be deemed to have a high risk of infection due to a local epidemic or a global pandemic. In some such embodiments, a population may be deemed to have a high risk of infection due to a subject population's geographic area. In some embodiments, a subject population comprises subjects that have been exposed to infection (e.g., Malaria).

In some embodiments, where a subject population is or includes pregnant women, provided technologies offer a particular advantage of interrupting malaria's transmission cycle, including, for example, in some embodiments, by reducing or eliminating transmission from pregnant mothers to their fetuses.

In some embodiments, a subject population is or comprises immunocompromised individuals. In some embodiments, a subject population does not include immunocompromised individuals.

In some embodiments, a provided pharmaceutical composition (e.g., immunogenic composition, e.g., vaccine) may be administered in combination with (i.e., so that subject(s) are simultaneously exposed to both) another pharmaceutical composition (e.g., immunogenic composition, e.g., vaccine) or therapeutic intervention, e.g., to treat or prevent malaria or another disease, disorder, or condition.

In some embodiments, a provided pharmaceutical composition (e.g., immunogenic composition, e.g., vaccine) may be administered with a protein vaccine, a DNA vaccine, an RNA vaccine, a cellular vaccine, a conjugate vaccine, etc. In some embodiments, one or more doses of a provided pharmaceutical composition (e.g., immunogenic composition, e.g., vaccine) may be administered together with (e.g., in a single visit) another vaccine or other therapy.

In some embodiments, a provided pharmaceutical composition (e.g., immunogenic composition, e.g., vaccine) may be administered to subjects who have been exposed, or expect they have been exposed, to malaria. In some embodiments, a provided pharmaceutical composition (e.g., immunogenic composition, e.g., vaccine) may be administered to subjects who do not have symptoms of malarial infection.

### VII. Treatment Methods

In some embodiments, technologies of the present disclosure may be administered to subjects according to a particular dosing regimen. In some embodiments, a dosing regimen may involve a single administration; in some embodiments, a dosing regimen may comprise one or more “booster” administrations after the initial administration. In some embodiments, initial and boost doses are the same amount; in some embodiments they differ. In some embodiments, two or more booster doses are administered. In some embodiments, a plurality of doses are administered at regular intervals. In some embodiments, periods of time between doses become longer. In some embodiments, one or more subsequent doses is administered if a particular clinical (e.g., reduction in neutralizing antibody levels) or situational (e.g., local development of a new strain) even arises or is detected.

In some embodiments, administered pharmaceutical compositions (e.g., immunogenic compositions, e.g., vaccines) comprising RNA constructs that encode Plasmodium T cell string polypeptide constructs are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, or about 100 μg. In some embodiments, an saRNA construct is administered at a lower dose (e.g., 2, 4, 5, 10 fold or more lower) than a modRNA or uRNA construct.

In some embodiments, a first booster dose is administered within a about six months of the initial dose, and preferably within about 5, 4, 3, 2, or 1 months. In some embodiments, a first booster dose is administered in a time period that begins about 1, 2, 3, or 4 weeks after the first dose, and ends about 2, 3, 4, 5, 6, 7, 8, 9, 10, 11, 12 weeks of the first dose (e.g., between about 1 and about 12 weeks after the first dose, or between about 2 or 3 weeks and about 5 and 6 weeks after the first dose, or about 3 weeks or about 4 weeks after the first dose).

In some embodiments, a plurality of booster doses (e.g., 2, 3, or 4) doses are administered within 6 months of the first dose, or within 12 months of the first dose.

In some embodiments, a first dose and a second dose are administered about 6, about 7, about 8, about 9, or about 10 weeks apart. In other words, in some embodiments, a second dose is administered about 6, about 7, about 8, about 9, or about 10 weeks after a first dose.

In some embodiments, a third dose is administered about 15, about 16, about 17, about 18, about 19, or about 20 weeks after a second dose.

In some embodiments, a second dose is administered about 8 weeks after a first dose, and a third dose is administered about 18 weeks after the second dose.

In some embodiments, 3 doses or fewer are required to achieve effective vaccination (e.g., greater than 60%, and in some embodiments greater than about 70%, about 75%, about 80%, about 85%, about 90% or more) reduction in risk of infection, or of serious disease. In some embodiments, not more than two doses are required. In some embodiments, a single dose is sufficient. In some embodiments, an RNA dose is about 60 μg or lower, 50 μg or lower, 40 μg or lower, 30 μg or lower, 20 μg or lower, 10 μg or lower, 5 μg or lower, 2.5 μg or lower, or 1 μg or lower. In some embodiments, an RNA dose is about 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg. In some embodiments, an RNA dose is about 0.25 μg to 60 μg, 0.5 μg to 55 μg, 1 μg to 50 μg, 5 μg to 40 μg, or 10 μg to 30 μg may be administered per dose. In some embodiments, an RNA dose is about 30 μg. In some embodiments, at least two such doses are administered. For example, a second dose may be administered about 21 days following administration of the first dose. In some embodiments, a first booster dose is administered about one month after an initial dose. In some such embodiments, at least one further booster is administered at one-month interval(s). In some embodiments, after 2 or 3 boosters, a longer interval is introduced and no further booster is administered for at least 6, 9, 12, 18, 24, or more months. In some embodiments, a single further booster is administered after about 18 months. In some embodiments, no further booster is required unless, for example, a material change in clinical or environmental situation is observed.

In some embodiments, one or more outcomes may be assessed following administration of one or more doses of a pharmaceutical composition provided herein. Exemplary outcomes include, but are not limited to: local reaction at the injection site (e.g., pain, erythema/redness, induration/swelling, etc.), frequency of solicited local reactions at the injection site (e.g., pain, erythema/redness, induration/swelling, etc.), systemic reactions (e.g., vomiting, diarrhea, headache, fatigue, muscle/joint pain, fever, etc.), frequency of solicited systemic reactions (e.g., vomiting, diarrhea, headache, fatigue, muscle/joint pain, fever, etc.), adverse events, medically attended adverse events, severe adverse events, frequency of subjects with at least one adverse event, frequency of subjects with at least one medically attended adverse events, frequency of subjects with at least one severe adverse events, number of subjects protected from blood stage parasitemia, proportion of subjects protected from blood stage parasitemia, and combinations thereof.

In some embodiments, blood stage parasitemia can be assessed by PCR, e.g., qPCR.

In some embodiments, statistics on antibody levels at assessed time points can be obtained following administration of one or more doses of a pharmaceutical composition provided herein. Time points can include at the time of a first dose, the time of a second dose, the time of a third dose, following a known or suspected malaria exposure, or following a known or suspected malaria infection.

### VIII. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (ψ), N1-methyl-pseudouridine (m1ψ), or 5-methyl-uridine (m5U) can be used to replace uridine triphosphate (UTP). In some embodiments, pseudouridine (ψ) can be used to replace uridine triphosphate (UTP). In some embodiments, N1-methyl-pseudouridine (m1ψ) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. For example, in some embodiments, a poly(A) tail comprises a nucleotide sequence of SEQ ID NO: 466. In some embodiments, a poly(A) tail comprises a plurality of A residues interrupted by a linker. In some embodiments, a linker comprises the nucleotide sequence of SEQ ID NO: 468.

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7 (3′OMeG)(5′)ppp(5′)(2′OMeA)pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments, polyribonucleotides may be processed through 0.2 μm filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising polyribonucleotides can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

### IX. DNA Constructs

Among other things, the present disclosure provides DNA constructs, for example that may encode one or more antibody agents as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.).

Expression vectors, which may be plasmid or viral or other vectors, typically include an expressible sequence of interest (e.g., a coding sequence) that is functionally linked with one or more control elements (e.g., promoters, enhancers, transcription terminators, etc.). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc.).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce a RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### Exemplary Numbered Embodiments

Embodiment 1. A polyribonucleotide encoding a polypeptide, wherein the polypeptide comprises one or more one or more Plasmodium T-cell antigens.

Embodiment 2. The polyribonucleotide of embodiment 1, wherein the one or more Plasmodium T-cell antigens comprises at least 2 and at most 10 Plasmodium T-cell antigens.

Embodiment 3. The polyribonucleotide of embodiment 1 or 2, wherein the polypeptide comprises at least 25 amino acids and at most 1100 amino acids.

Embodiment 4. The polyribonucleotide of any one of embodiments 1-3, wherein the polypeptide comprises at least 25 amino acids and at most 500 amino acids.

Embodiment 5. The polyribonucleotide of any one of embodiments 1-4, wherein the one or more Plasmodium T cell antigens comprises two or more of:
(i) an antigenic Plasmodium CSP polypeptide fragment;
(ii) an antigenic Plasmodium LSA-1 (a) polypeptide fragment;
(iii) an antigenic Plasmodium LSA-1 (b) polypeptide fragment;
(iv) an antigenic Plasmodium TRAP polypeptide fragment;
(v) an antigenic Plasmodium LSAP2 polypeptide fragment;
(vi) an antigenic Plasmodium UIS3 polypeptide fragment;
(vii) an antigenic Plasmodium ETRAMP10.3 polypeptide fragment;
(viii) an antigenic Plasmodium LISP-1 polypeptide fragment;
(ix) an antigenic Plasmodium LISP-2 polypeptide fragment; and
(x) an antigenic Plasmodium LSA-3 polypeptide fragment.

Embodiment 6. The polyribonucleotide of any one of embodiments 1-5, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment; and
(v) an antigenic Plasmodium LSAP2 polypeptide fragment.

Embodiment 7. The polyribonucleotide of embodiment 6, wherein the polypeptide comprises or consists of an amino acid sequence with at least 85% sequence identity to an amino acid sequence according to SEQ ID NO: 15.

Embodiment 8. The polyribonucleotide of any one of embodiments 1-6, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-3 polypeptide fragment;
(vii) an antigenic Plasmodium LSA-1 (a) polypeptide fragment; and
(viii) an antigenic Plasmodium LSA-1 (b) polypeptide fragment.

Embodiment 9. The polyribonucleotide of embodiment 8, wherein the polypeptide comprises or consists of an amino acid sequence with at least 85% sequence identity to an amino acid sequence according to SEQ ID NO: 18.

Embodiment 10. The polyribonucleotide of any one of embodiments 1-6, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment;
(viii) an antigenic Plasmodium LISP-2 polypeptide fragment; and
(ix) an antigenic Plasmodium LISP-1 polypeptide fragment.

Embodiment 11. The polyribonucleotide of embodiment 10, wherein the polypeptide comprises or consists of an amino acid sequence with at least 85% sequence identity to an amino acid sequence according to SEQ ID NO: 24.

Embodiment 12. The polyribonucleotide of any one of embodiments 1-6, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment; and
(viii) an antigenic Plasmodium LISP-1 polypeptide fragment.

Embodiment 13. The polyribonucleotide of embodiment 12, wherein the polypeptide comprises or consists of an amino acid sequence with at least 85% sequence identity to an amino acid sequence according to SEQ ID NO: 27.

Embodiment 14. The polyribonucleotide of any one of embodiments 1-6, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LISP-2 polypeptide fragment; and
(vii) an antigenic Plasmodium LISP-1 polypeptide fragment.

Embodiment 15. The polyribonucleotide of embodiment 14, wherein the polypeptide comprises or consists of an amino acid sequence with at least 85% sequence identity to an amino acid sequence according to SEQ ID NO: 30.

Embodiment 16. The polyribonucleotide of any one of embodiments 1-6, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (b) polypeptide fragment; and
(vii) an antigenic Plasmodium LISP-1 polypeptide fragment.

Embodiment 17. The polyribonucleotide of embodiment 16, wherein the polypeptide comprises or consists of an amino acid sequence with at least 85% sequence identity to an amino acid sequence according to SEQ ID NO: 33.

Embodiment 18. The polyribonucleotide of any one of embodiments 1-6, wherein the one or more Plasmodium T cell antigens comprises:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment;
(viii) an antigenic Plasmodium LISP-2 polypeptide fragment;
(ix) an antigenic Plasmodium LISP-1 polypeptide fragment; and
(x) an antigenic Plasmodium LSA-3 polypeptide fragment.

Embodiment 19. The polyribonucleotide of embodiment 18, wherein the polypeptide comprises or consists of an amino acid sequence with at least 85% sequence identity to an amino acid sequence according to SEQ ID NO: 36.

Embodiment 20. The polyribonucleotide of any one of embodiments 1-5, wherein the one or more Plasmodium T cell antigens comprises:
(i) an antigenic Plasmodium LSA-1 (a) polypeptide fragment;
(ii) an antigenic Plasmodium LSA-1 (b) polypeptide fragment;
(iii) an antigenic Plasmodium LISP-2 polypeptide fragment;
(iv) an antigenic Plasmodium LISP-1 polypeptide fragment; and
(v) an antigenic Plasmodium LSA-3 polypeptide fragment.

Embodiment 21. The polyribonucleotide of embodiment 20, wherein the polypeptide comprises or consists of an amino acid sequence with at least 85% sequence identity to an amino acid sequence according to SEQ ID NO: 48.

Embodiment 22. The polyribonucleotide of any one of embodiments 1-5, wherein the one or more Plasmodium T cell antigens comprises:
(i) an antigenic Plasmodium LSA-1 (a) polypeptide fragment;
(ii) an antigenic Plasmodium LSA-1 (b) polypeptide fragment;
(iii) an antigenic Plasmodium LISP-2 polypeptide fragment; and
(iv) an antigenic Plasmodium LISP-1 polypeptide fragment.

Embodiment 23. The polyribonucleotide of embodiment 22, wherein the polypeptide comprises or consists of an amino acid sequence with at least 85% sequence identity to an amino acid sequence according to SEQ ID NO: 45.

Embodiment 24. The polyribonucleotide of any one of embodiments 1-6, 8, 10, 12, 14, 16, and 18, wherein the one or more Plasmodium T cell antigens comprises the antigenic Plasmodium CSP polypeptide fragment, and wherein the antigenic Plasmodium CSP polypeptide fragment comprises a Plasmodium CSP N-terminal region.

Embodiment 25. The polyribonucleotide of embodiment 24, wherein the antigenic Plasmodium CSP polypeptide fragment further comprises a Plasmodium CSP N-terminal end region.

Embodiment 26. The polyribonucleotide of embodiment 24 or 25, wherein the antigenic Plasmodium CSP polypeptide fragment further comprises a Plasmodium CSP junction region.

Embodiment 27. The polyribonucleotide of any one of embodiments 24-26, wherein the antigenic Plasmodium CSP polypeptide fragment comprises or consists of an amino acid sequence with at least 85% sequence identity to an amino acid sequence according to SEQ ID NO: 133.

Embodiment 28. The polyribonucleotide of any one of embodiments 1-6, 8, 10, 12, 14, 16, 18, and 24-27, wherein the one or more Plasmodium T cell antigens do not comprise an antigenic Plasmodium berghei CSP polypeptide fragment.

Embodiment 29. The polyribonucleotide of any one of embodiments 1-5, 8, 10, 12, 18, 20, and 22, wherein the one or more Plasmodium T cell antigens comprises the antigenic Plasmodium LSA-1 (a) polypeptide fragment, and wherein the antigenic Plasmodium LSA-1 (a) polypeptide fragment comprises or consists of an amino acid sequence with at least 85% sequence identity to an amino acid sequence according to SEQ ID NO: 144.

Embodiment 30. The polyribonucleotide of any one of embodiments 1-5, 8, 10, 12, 16, 18, 20, and 22, wherein the one or more Plasmodium T cell antigens comprises the antigenic Plasmodium LSA-1 (b) polypeptide fragment, and wherein the antigenic Plasmodium LSA-1 (b) polypeptide fragment comprises or consists of an amino acid sequence with at least 85% sequence identity to an amino acid sequence according to SEQ ID NO: 155.

Embodiment 31. The polyribonucleotide of any one of embodiments 1-6, 8, 10, 12, 14, 16, and 18, wherein the one or more Plasmodium T cell antigens comprises the antigenic Plasmodium TRAP polypeptide fragment, and wherein the antigenic Plasmodium TRAP polypeptide fragment comprises or consists of an amino acid sequence with at least 85% sequence identity to an amino acid sequence according to SEQ ID NO: 171.

Embodiment 32. The polyribonucleotide of any one of embodiments 1-6, 8, 10, 12, 14, 16, and 18, wherein the one or more Plasmodium T cell antigens comprises the antigenic Plasmodium LSAP2 polypeptide fragment, and wherein the antigenic Plasmodium LSAP2 polypeptide fragment comprises or consists of an amino acid sequence with at least 85% sequence identity to an amino acid sequence according to SEQ ID NO: 198.

Embodiment 33. The polyribonucleotide of any one of embodiments 1-6, 8, 10, 12, 14, 16, and 18, wherein the one or more Plasmodium T cell antigens comprises the antigenic Plasmodium UIS3 polypeptide fragment, and wherein the antigenic Plasmodium UIS3 polypeptide fragment comprises or consists of an amino acid sequence with at least 85% sequence identity to an amino acid sequence according to SEQ ID NO: 212.

Embodiment 34. The polyribonucleotide of any one of embodiments 1-6, 8, 10, 12, 14, 16, and 18, wherein the one or more Plasmodium T cell antigens comprises the antigenic Plasmodium ETRAMP10.3 polypeptide fragment, and wherein the antigenic Plasmodium ETRAMP10.3 polypeptide fragment comprises or consists of an amino acid sequence with at least 85% sequence identity to an amino acid sequence according to SEQ ID NO: 219.

Embodiment 35. The polyribonucleotide of any one of embodiments 1-5, 10, 12, 14, 16, 18, 20, and 22, wherein the one or more Plasmodium T cell antigens comprises the antigenic Plasmodium LISP-1 polypeptide fragment, and wherein the antigenic Plasmodium LISP-1 polypeptide fragment comprises or consists of an amino acid sequence with at least 85% sequence identity to an amino acid sequence according to SEQ ID NO: 229.

Embodiment 36. The polyribonucleotide of any one of embodiments 1-5, 10, 14, 18, 20, and 22, wherein the one or more Plasmodium T cell antigens comprises the antigenic Plasmodium LISP-2 polypeptide fragment, and wherein the antigenic Plasmodium LISP-2 polypeptide fragment comprises or consists of an amino acid sequence with at least 85% sequence identity to an amino acid sequence according to SEQ ID NO: 238.

Embodiment 37. The polyribonucleotide of any one of embodiments 1-5, 8, 18, and 20, wherein the one or more Plasmodium T cell antigens comprises the antigenic Plasmodium LSA-3 polypeptide fragment, and wherein the antigenic Plasmodium LSA-3 polypeptide fragment comprises or consists of an amino acid sequence with at least 85% sequence identity to an amino acid sequence according to SEQ ID NO: 249.

Embodiment 38. The polyribonucleotide of any one of embodiments 1-37, wherein the one or more Plasmodium T cell antigens each comprise one or more T cell epitopes.

Embodiment 39. The polyribonucleotide of any one of embodiments 1-38, wherein the polypeptide does not comprise an antigenic fragment of a bacterial polypeptide.

Embodiment 40. The polyribonucleotide of any one of embodiments 1-39, wherein polypeptide does not comprise an antigenic bacillus Calmette-Guerin (BCG) polypeptide fragment, optionally wherein the antigenic BCG polypeptide fragment comprises an amino acid sequence according to SEQ ID NO: 461.

Embodiment 41. The polyribonucleotide of any one of embodiments 1-40, wherein polypeptide does not comprise an antigenic tetanus toxin (TT) polypeptide fragment, optionally wherein the antigenic TT polypeptide fragment comprises an amino acid sequence according to SEQ ID NO: 462.

Embodiment 42. The polyribonucleotide of any one of embodiments 1-41, wherein the one or more Plasmodium T cell antigens do not comprise an antigenic Plasmodium sporozoite threonine-asparagine-rich protein (STARP) polypeptide fragment, and optionally wherein the antigenic Plasmodium STARP polypeptide fragment comprises an amino acid sequence according to SEQ ID NO: 463.

Embodiment 43. The polyribonucleotide of any one of embodiments 1-42, wherein the polyribonucleotide further comprises an MHC class I trafficking signal (MITD).

Embodiment 44. The polyribonucleotide of embodiment 43, wherein the MITD comprises or consists of an amino acid sequence according to SEQ ID NO: 479.

Embodiment 45. The polyribonucleotide of any one of embodiments 1-44, wherein the polypeptide comprises a secretory signal.

Embodiment 46. The polyribonucleotide of embodiment 45, wherein the secretory signal comprises or consists a Plasmodium secretory signal.

Embodiment 47. The polyribonucleotide of embodiment 46, wherein the Plasmodium secretory signal comprises or consists of a Plasmodium CSP secretory signal.

Embodiment 48. The polyribonucleotide of embodiment 47, wherein the Plasmodium CSP secretory signal comprises or consists of an amino acid sequence according to SEQ ID NO: 397.

Embodiment 49. The polyribonucleotide of embodiment 45, wherein the secretory signal comprises or consists of a heterologous secretory signal.

Embodiment 50. The polyribonucleotide of embodiment 49, wherein the heterologous secretory signal comprises or consists of a non-human secretory signal.

Embodiment 51. The polyribonucleotide of embodiment 49 or 50, wherein the heterologous secretory signal comprises or consists of a viral secretory signal.

Embodiment 52. The polyribonucleotide of embodiment 51, wherein the viral secretory signal comprises or consists of an HSV secretory signal.

Embodiment 53. The polyribonucleotide of embodiment 52, wherein the HSV secretory signal comprises or consists of an HSV-1 or HSV-2 secretory signal.

Embodiment 54. The polyribonucleotide of embodiment 52 or 53, wherein the HSV secretory signal comprises or consists of an HSV glycoprotein D (gD) secretory signal.

Embodiment 55. The polyribonucleotide of embodiment 54, wherein the HSV gD secretory signal comprises or consists of an amino acid sequence according to SEQ ID NO: 382.

Embodiment 56. The polyribonucleotide of embodiment 54, wherein the HSV gD secretory signal comprises or consists of an amino acid sequence according to SEQ ID NO: 388.

Embodiment 57. The polyribonucleotide of embodiment 51, wherein the secretory signal comprises or consists of an Ebola virus secretory signal.

Embodiment 58. The polyribonucleotide of embodiment 57, wherein the Ebola virus secretory signal comprises or consists of an Ebola virus spike glycoprotein (SGP) secretory signal.

Embodiment 59. The polyribonucleotide of embodiment 58, wherein the Ebola virus SGP secretory signal comprises or consists of an amino acid sequence according to SEQ ID NO: 400.

Embodiment 60. The polyribonucleotide of any one of embodiments 45-59, wherein the secretory signal is located at the N-terminus of the polypeptide.

Embodiment 61. The polyribonucleotide of any one of embodiments 1-60, wherein the polypeptide comprises a transmembrane region.

Embodiment 62. The polyribonucleotide of embodiment 61, wherein the transmembrane region comprises or consists of a Plasmodium transmembrane region.

Embodiment 63. The polyribonucleotide of embodiment 62, wherein the Plasmodium transmembrane region comprises or consists of a Plasmodium CSP glycosylphosphatidylinositol (GPI) anchor region.

Embodiment 64. The polyribonucleotide of embodiment 63, wherein the Plasmodium CSP GPI anchor region comprises or consists of an amino acid sequence according to SEQ ID NO: 444.

Embodiment 65. The polyribonucleotide of embodiment 61, wherein the transmembrane region comprises or consists of a heterologous transmembrane region.

Embodiment 66. The polyribonucleotide of embodiment 65, wherein the heterologous transmembrane region does not comprise a hemagglutinin transmembrane region.

Embodiment 67. The polyribonucleotide of embodiment 65 or 66, wherein the heterologous transmembrane region comprises or consists of a non-human transmembrane region.

Embodiment 68. The polyribonucleotide of embodiment 65 or 67, wherein the heterologous transmembrane region comprises or consists of a viral transmembrane region.

Embodiment 69. The polyribonucleotide of any one of embodiments 65-68, wherein the heterologous transmembrane region comprises or consists of an HSV transmembrane region.

Embodiment 70. The polyribonucleotide of embodiment 69, wherein the HSV transmembrane region comprises or consists of an HSV-1 or HSV-2 transmembrane region.

Embodiment 71. The polyribonucleotide of embodiment 69 or 70, wherein the HSV transmembrane region comprises or consists of an HSV gD transmembrane region.

Embodiment 72. The polyribonucleotide of embodiment 71, wherein the HSV gD transmembrane region comprises or consists of an amino acid sequence according to SEQ ID NO: 447.

Embodiment 73. The polyribonucleotide of embodiment 65 or 66, wherein the transmembrane region comprises or consists of a human transmembrane region.

Embodiment 74. The polyribonucleotide of embodiment 73, wherein the human transmembrane region comprises or consists of a human decay accelerating factor glycosylphosphatidylinositol (hDAF-GPI) anchor region.

Embodiment 75. The polyribonucleotide of embodiment 74, wherein the hDAF-GPI anchor region comprises or consists of an amino acid sequence according to SEQ ID NO: 450.

Embodiment 76. The polyribonucleotide of any one of embodiments 1-44 and 61-75, wherein the polypeptide does not comprise a secretory signal.

Embodiment 77. The polyribonucleotide of any one of embodiments 1-60, wherein the polypeptide does not comprise a transmembrane region.

Embodiment 78. The polyribonucleotide of any one of embodiments 1-77, wherein the polypeptide comprises one or more linkers.

Embodiment 79. The polyribonucleotide of embodiment 78, wherein the one or more linkers comprise or consist of an amino acid sequence according to SEQ ID NO: 452.

Embodiment 80. The polyribonucleotide of embodiment 78, wherein the one or more linkers comprise or consist of an amino acid sequence according to SEQ ID NO: 459.

Embodiment 81. The polyribonucleotide of embodiment 78, wherein the one or more linkers comprise or consist of an amino acid sequence according to SEQ ID NO: 456.

Embodiment 82. The polyribonucleotide of embodiment 78, wherein the one or more linkers comprise or consist of an amino acid sequence according to SEQ ID NO: 460.

Embodiment 83. The polyribonucleotide of any one of embodiments 1-82, wherein the polypeptide comprises a linker between two Plasmodium T-cell antigens.

Embodiment 84. The polyribonucleotide of any one of embodiments 1-83, wherein the one or more Plasmodium T cell antigens are one or more P. falciparum T cell antigens.

Embodiment 85. The polyribonucleotide of embodiment 84, wherein the one or more P. falciparum T cell antigens are from P. falciparum isolate 3D7.

Embodiment 86. The polyribonucleotide of any one of embodiments 1-85, wherein the one or more Plasmodium T-cell antigens are from a Plasmodium species capable of infecting a human.

Embodiment 87. The polyribonucleotide of any one of embodiments 1-86, wherein each of the one or more Plasmodium T-cell antigens comprises at least 21 amino acids.

Embodiment 88. The polyribonucleotide of any one of embodiments 1-87, wherein the polyribonucleotide is an isolated polyribonucleotide.

Embodiment 89. The polyribonucleotide of any one of embodiments 1-88, wherein the polyribonucleotide is an engineered polyribonucleotide.

Embodiment 90. The polyribonucleotide of any one of embodiments 1-89, wherein the polyribonucleotide is a codon-optimized polyribonucleotide.

Embodiment 91. An RNA construct comprising in 5′ to 3′ order:
(i) a 5′ UTR that comprises or consists of a modified human alpha-globin 5′-UTR;
(ii) a polyribonucleotide of any one of embodiments 1-82;
(iii) a 3′ UTR that comprises or consists of a first sequence from the amino terminal enhancer of split (AES) messenger RNA and a second sequence from the mitochondrial encoded 12S ribosomal RNA; and
(iv) a polyA tail sequence.

Embodiment 92. The RNA construct of embodiment 91, wherein the 5′ UTR comprises or consists of a ribonucleic acid sequence according to SEQ ID NO: 465.

Embodiment 93. The RNA construct of embodiment 91 or 92, wherein the 3′ UTR comprises or consists of a ribonucleic acid sequence according to SEQ ID NO: 471.

Embodiment 94. The RNA construct of any one of embodiments 91-93, wherein the polyA tail sequence is a split polyA tail sequence.

Embodiment 95. The RNA construct of embodiment 94, wherein the split polyA tail sequence comprises or consists of a ribonucleic acid sequence according to SEQ ID NO: 467.

Embodiment 96. The RNA construct of any one of embodiments 91-95, further comprises a 5′ cap.

Embodiment 97. The RNA construct of any one of embodiments 91-96, further comprises a cap proximal sequence comprising positions +1, +2, +3, +4, and +5 of the polyribonucleotide.

Embodiment 98. The RNA construct of embodiment 96 or 97, wherein the 5′ cap comprises or consists of m7(3′OMeG)(5′)ppp(5′)(2′OMeA₁)pG₂, wherein A1 is position +1 of the polyribonucleotide, and G₂ is position +2 of the polyribonucleotide.

Embodiment 99. The RNA construct of any one of embodiments 96-98, wherein the cap proximal sequence comprises A₁ and G₂ of the Cap1 structure, and a sequence comprising: A3A4Us (SEQ ID NO: 480) at positions +3, +4 and +5 respectively of the polyribonucleotide.

Embodiment 100. A composition comprising one or more polyribonucleotides of any one of embodiments 1-90.

Embodiment 101. A composition comprising one or more RNA constructs of any one of 91-99.

Embodiment 102. The composition of embodiment 100 or 101, wherein the composition further comprises lipid nanoparticles, polyplexes (PLX), lipidated polyplexes (LPLX), or liposomes, wherein the one or more polyribonucleotides are fully or partially encapsulated within the lipid nanoparticles, polyplexes (PLX), lipidated polyplexes (LPLX), or liposomes.

Embodiment 103. The composition of any one of embodiments 100-102, wherein the composition further comprises lipid nanoparticles, wherein the one or more polyribonucleotides are encapsulated within the lipid nanoparticles.

Embodiment 104. The composition of embodiment 102 or 103, wherein the lipid nanoparticles target liver cells.

Embodiment 105. The composition of embodiment 102 or 103, wherein the lipid nanoparticles target secondary lymphoid organ cells.

Embodiment 106. The composition of embodiment any one of embodiments 102-105, wherein the lipid nanoparticles are cationic lipid nanoparticles.

Embodiment 107. The composition of any one of embodiments 102-106, wherein the lipid nanoparticles each comprise:
(a) a polymer-conjugated lipid;
(b) a cationically ionizable lipid; and
(c) one or more neutral lipids.

Embodiment 108. The composition of embodiment 107, wherein the polymer-conjugated lipid comprises a PEG-conjugated lipid.

Embodiment 109. The composition of embodiment 107 or 108, wherein the polymer-conjugated lipid comprises 2-[(polyethylene glycol)-2000]-N,N-ditetradecylacetamide.

Embodiment 110. The composition of any one of embodiments 107-109, wherein the one or more neutral lipids comprise 1,2-Distearoyl-sn-glycero-3-phosphocholine (DPSC).

Embodiment 111. The composition of any one of embodiments 107-110, wherein the one or more neutral lipids comprise cholesterol.

Embodiment 112. The composition of any one of embodiments 107-111, wherein the cationically ionizable lipid comprises [(4-Hydroxybutyl) azanediyl]di(hexane-6,1-diyl)bis(2-hexyldecanoate).

Embodiment 113. The composition of any one of embodiments 107-112, wherein the lipid nanoparticles have an average diameter of about 50-150 nm.

Embodiment 114.A pharmaceutical composition comprising the composition of any one of embodiments 100-113 and at least one pharmaceutically acceptable excipient.

Embodiment 115. The pharmaceutical composition of embodiment 114, wherein the pharmaceutical comprises a cryoprotectant, optionally wherein the cryoprotectant is sucrose.

Embodiment 116. The pharmaceutical composition of embodiment 114 or 115, wherein the pharmaceutical comprises an aqueous buffered solution, optionally wherein the aqueous buffered solution comprises one or more of Tris base, Tris HCl, NaCl, KCl, Na₂HPO₄, and KH₂PO₄.

Embodiment 117. A combination comprising:
(i) a first pharmaceutical composition comprising a first polyribonucleotide, wherein the first polyribonucleotide encodes a first polypeptide, and the first polypeptide comprises a one or more Plasmodium T-cell antigens; and
(ii) a second pharmaceutical composition comprising a second polyribonucleotide, wherein the second polyribonucleotide encodes a second polypeptide, and the second polypeptide comprises one or more Plasmodium antigenic polypeptide regions or portions thereof.

Embodiment 118. The combination of embodiment 117, wherein the first polyribonucleotide is a polyribonucleotide according to any one of embodiments 1-88.

Embodiment 119. The combination of embodiment 117 or 118, wherein the one or more Plasmodium antigenic polypeptide regions or portions thereof of the second polypeptide comprise one or more Plasmodium CSP regions or portions thereof.

Embodiment 120. A combination comprising:
(i) a first pharmaceutical composition comprising a polyribonucleotide encoding a first polypeptide, wherein the first polypeptide comprises a one or more Plasmodium T-cell antigens, and wherein the one or more Plasmodium T-cell antigens comprises a Plasmodium N-terminal region or portion thereof, but not a Plasmodium C-terminal region or portion thereof; and
(ii) a second pharmaceutical composition comprising a polyribonucleotide encoding a second polypeptide, wherein the second polypeptide comprises one or more Plasmodium CSP polypeptide regions or portions thereof, and wherein the one or more Plasmodium CSP polypeptide regions or portions thereof comprises a Plasmodium CSP C-terminal region or portion thereof, but not a Plasmodium CSP N-terminal region or portion thereof.

Embodiment 121. The combination of any one of embodiments 117-120, wherein the first pharmaceutical composition and the second pharmaceutical composition are not in the same composition.

Embodiment 122. A combination comprising:

(i) a first pharmaceutical composition comprising a first polyribonucleotide, wherein the first polyribonucleotide is a polyribonucleotide according to embodiment 6 or 7; and
(ii) a second pharmaceutical composition comprising a second polyribonucleotide, wherein the second polyribonucleotide is a polyribonucleotide according to embodiment 22 or 23.

Embodiment 123. A method comprising administering a polyribonucleotide according to any one of embodiments 1-90 to a subject.

Embodiment 124. A method comprising administering an RNA construct according to any one of embodiments 91-99 to a subject.

Embodiment 125. A method comprising administering a composition according to any one of embodiments 100-116 to a subject.

Embodiment 126. A method comprising administering one or more doses of the pharmaceutical composition of any one of embodiments 114-116 to a subject.

Embodiment 127. The pharmaceutical composition of any one of embodiments 114-116 for use in the treatment of a malaria infection comprising administering one or more doses of the pharmaceutical composition to a subject.

Embodiment 128. The pharmaceutical composition of any one of embodiments 114-116 for use in the prevention of a malaria infection comprising administering one or more doses of the pharmaceutical composition to a subject.

Embodiment 129. The method of embodiment 126 or the pharmaceutical composition for use of embodiment 127 or 128, comprising administering two or more doses of the pharmaceutical composition to a subject.

Embodiment 130. The method of embodiment 126 or 129, or the pharmaceutical composition for use of any one of embodiments 127-129, comprising administering three or more doses of the pharmaceutical composition to a subject.

Embodiment 131. The method or the pharmaceutical composition for use of embodiment 130, wherein the second of the three or more doses is administered to the subject at least 4 weeks after the first of the three or more doses is administered to the subject.

Embodiment 132. The method or the pharmaceutical composition for use of embodiment 130 or 131, wherein the third of the three or more doses is administered to the subject at least 4 weeks after the second of the three or more doses is administered to the subject.

Embodiment 133. The method of any one of embodiments 126 or 129-132, or the pharmaceutical composition for use of any one of embodiments 127-129, comprising administering a fourth dose of the pharmaceutical composition to a subject.

Embodiment 134. The method or the pharmaceutical composition for use of embodiment 133, wherein the fourth dose is administered to the subject at least one year after the third of the three or more doses is administered to the subject.

Embodiment 135. A method comprising administering a combination of any one of embodiments 117-122 to a subject.

Embodiment 136. The method of embodiment 135, wherein the first pharmaceutical composition and the second pharmaceutical composition are administered on the same day.

Embodiment 137. The method of embodiment 135, wherein the first pharmaceutical composition and the second pharmaceutical composition are administered on different days.

Embodiment 138. The method of any one of embodiments 135-137, wherein the first pharmaceutical composition and the second pharmaceutical composition are administered to the subject at different locations on the subject's body.

Embodiment 139. The method of any one of embodiments 135-138, wherein the method is a method of treating a malaria infection.

Embodiment 140. The method of any one of embodiments 135-139, wherein the method is a method of preventing a malaria infection.

Embodiment 141. The method of any one of embodiments 135-140, wherein the subject has or is at risk of developing a malaria infection.

Embodiment 142. The method of any one of embodiments 135-141, wherein the subject is a human.

Embodiment 143. The method of any one of embodiments 126 and 129-142, wherein administration induces an anti-malaria immune response in the subject.

Embodiment 144. The method of embodiment 143, wherein the anti-malaria immune response in the subject comprises an adaptive immune response.

Embodiment 145. The method of embodiment 143 or 144, wherein the anti-malaria immune response in the subject comprises a T-cell response.

Embodiment 146. The method of embodiment 145, wherein the T-cell response is or comprises a CD4+ T cell response.

Embodiment 147. The method of embodiment 145 or 146, wherein the T-cell response is or comprises a CD8+ T cell response.

Embodiment 148. The method of any one of embodiments 143-147, wherein the anti-malaria immune system response comprises a B-cell response.

Embodiment 149. The method of any one of embodiments 143-148, wherein the anti-malaria immune system response comprises the production of antibodies directed against the one or more Plasmodium antigens.

Embodiment 150. Use of the pharmaceutical composition of any one of embodiments 114-116 in the treatment of a malaria infection.

Embodiment 151. Use of the pharmaceutical composition of any one of embodiments 114-116 in the prevention of a malaria infection.

Embodiment 152. Use of the pharmaceutical composition of any one of embodiments 114-116 in inducing an anti-malaria immune response in a subject.

Embodiment 153. A polypeptide encoded by a polyribonucleotide of any one of embodiments 1-90.

Embodiment 154. A polypeptide encoded by an RNA construct of any one of embodiments 91-99.

Embodiment 155. A host cell comprising a polyribonucleotide of any one of embodiments 1-90.

Embodiment 156. A host cell comprising an RNA construct of any one of embodiments 91-99.

Embodiment 157. A host cell comprising a polypeptide of embodiment 153 or 154.

### Exemplification

### Example 1: Prediction and/or Characterization of MHC Presentation

The present Example describes identification, selection and/or characterization of certain Plasmodium protein sequences useful as or in (i.e., as part of) antigens as described herein.

FIG. 1 presents a flow diagram of a process used to identify, characterize, and/or select certain Plasmodium protein sequences (e.g., particular variants) and/or fragments or epitopes thereof, that may be particularly useful in the practice of the present invention. As depicted, proteins expressed prior to liver cell infiltration, that include one or more portions expected or known to interface with host cytoplasm are identified, for example by literature review, considering transcriptomic (i.e., RNA expression levels) and/or proteomic (i.e., expressed protein levels) data. Degree of conservation of candidate proteins across falciparum strains (e.g., in relevant geographic region) is considered.

Various lab and field isolate strains were considered for assess conserved proteins and T cell epitopes.

Whole genome single nucleotide polymorphism (SNP) comparison to global clinical strains revealed that three laboratory strains (NF54, 7G8, NF135/5.C₁₀) are representative of their geographic origin (Sub-saharan Africa, South America and South-east Asia) despite long term culture adaptation. See Moder et al Genome Med, 12:6, 2020, which is herein incorporated by reference in its entirety. Immunogenicity of conserved proteins was also considered, for example by review of literature and/or application of predictive algorithms as described herein.

In accordance with the present example, each of the following Plasmodium proteins were identified as of particular interest: CSP, TRAP, LSAP1, LSAP2 UIS3, ETRAMP10.3, LISP1, LISP2, LSA-3, LSA-1, CelTOS, EXP1, AMA1, RH5 and combinations thereof.

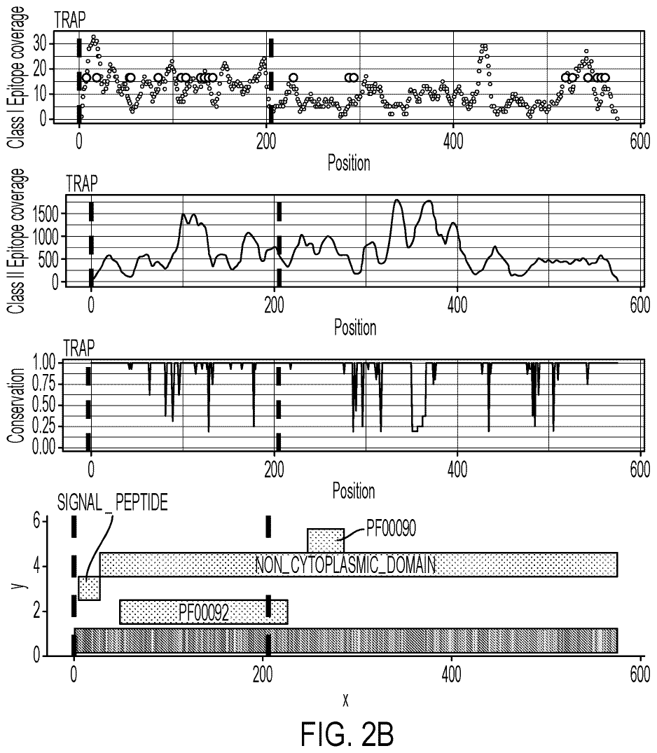

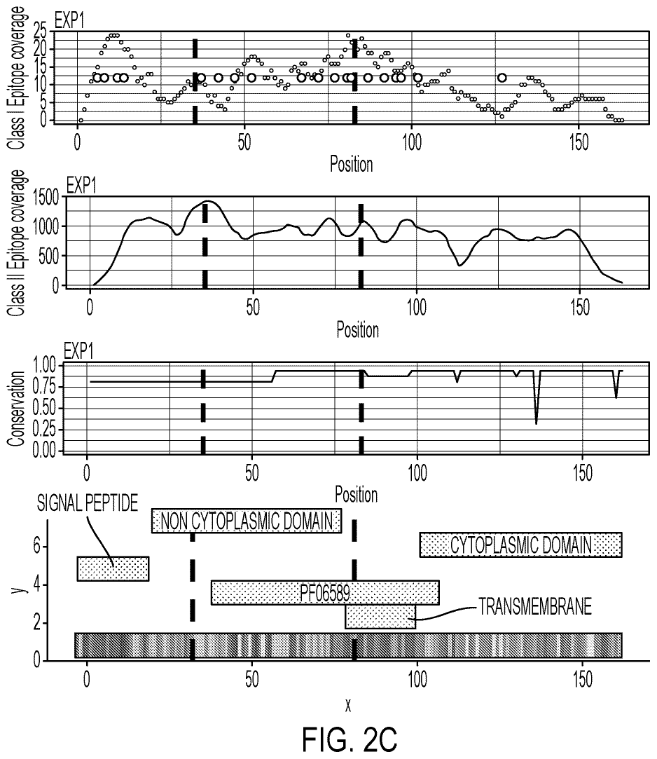

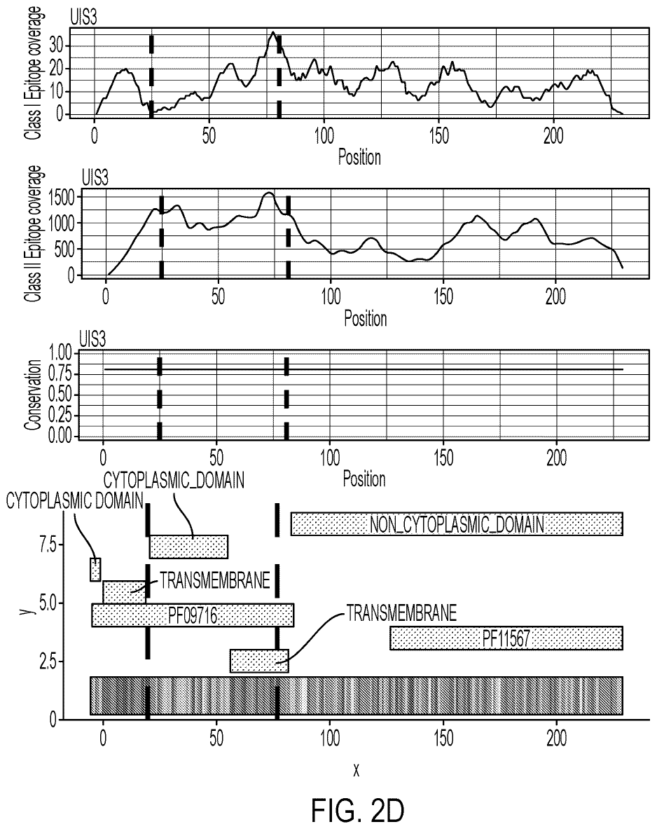

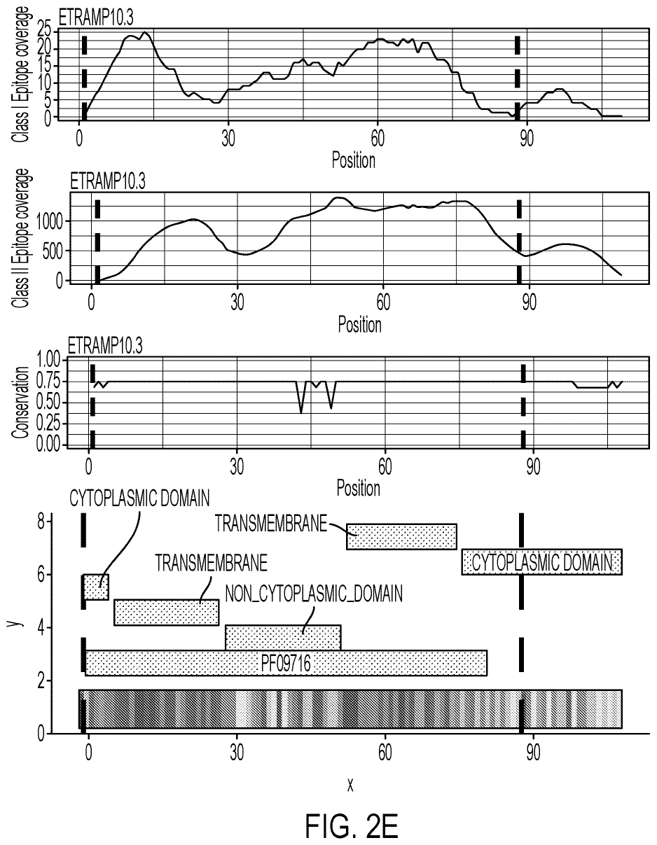

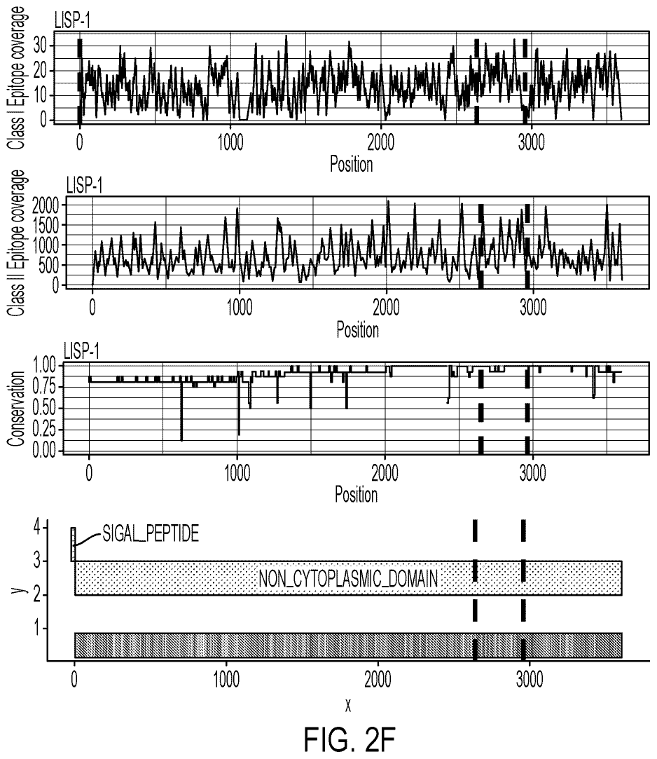

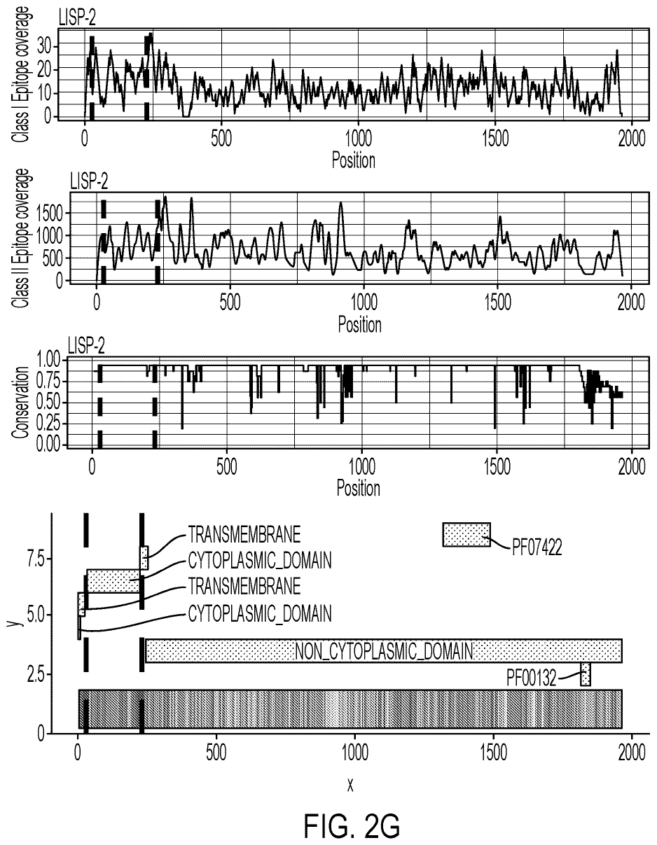

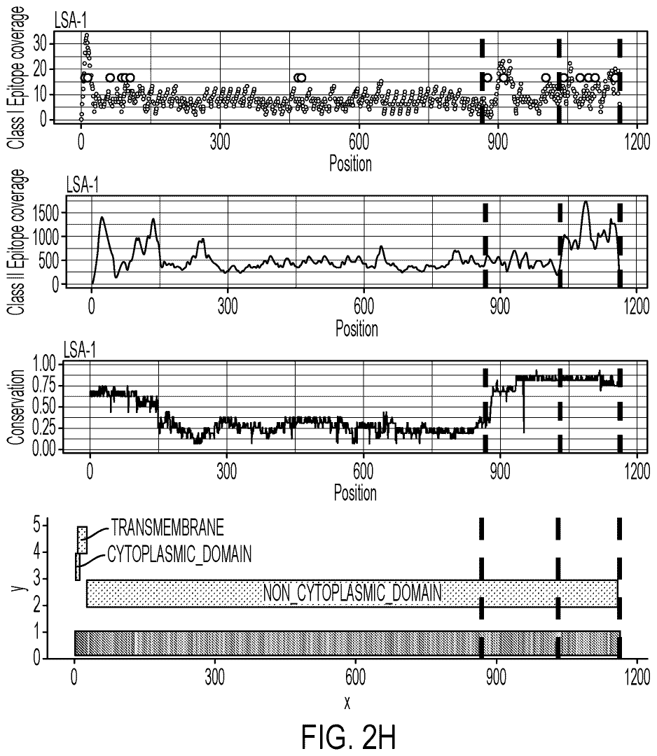

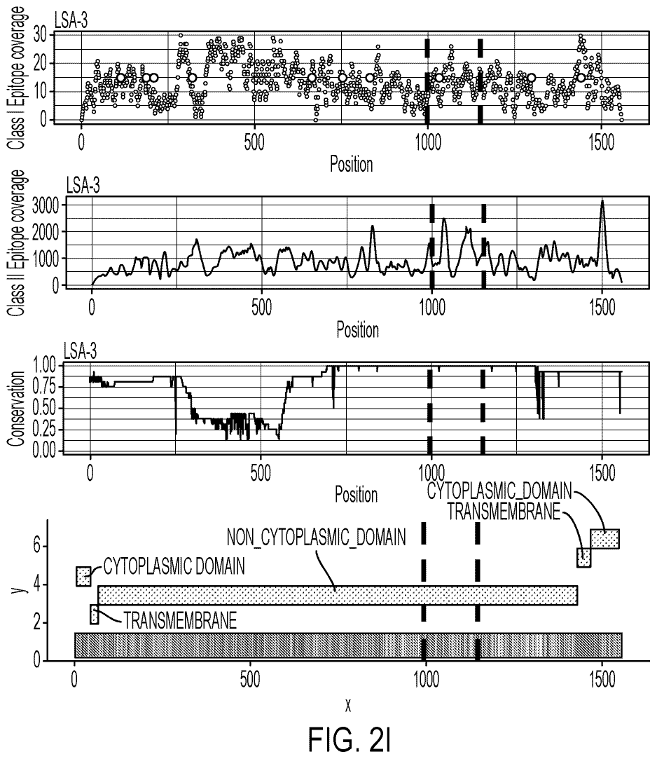

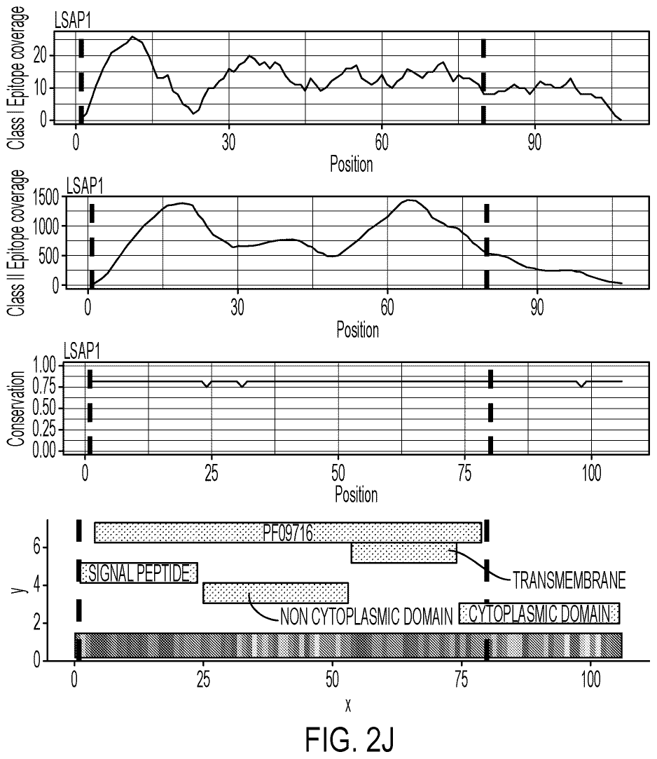

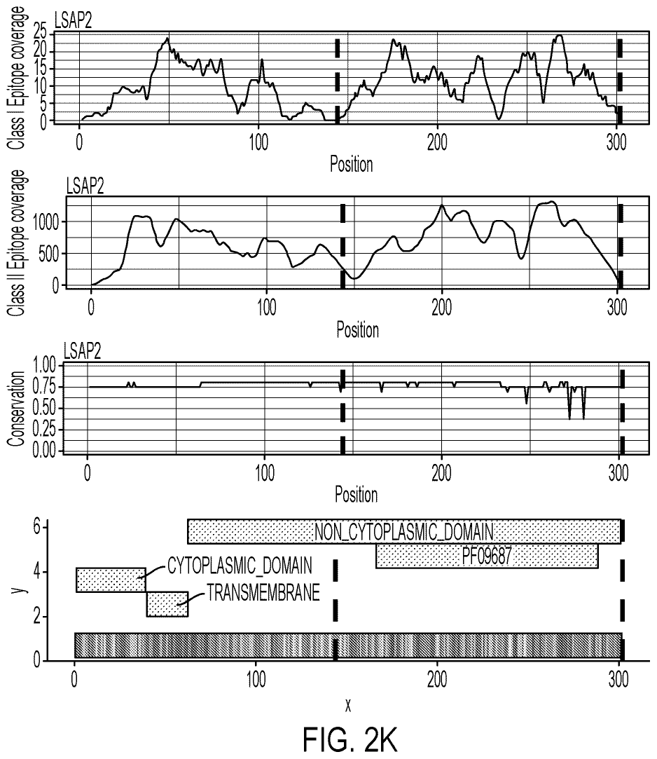

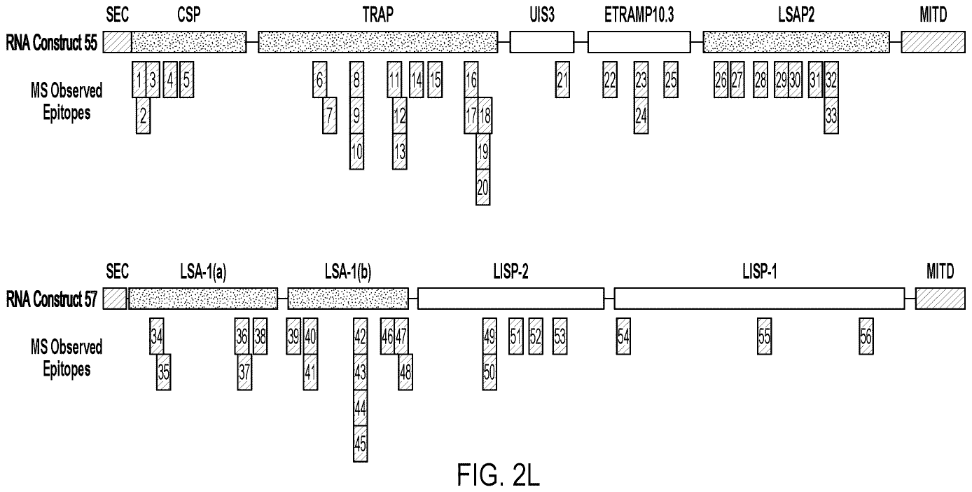

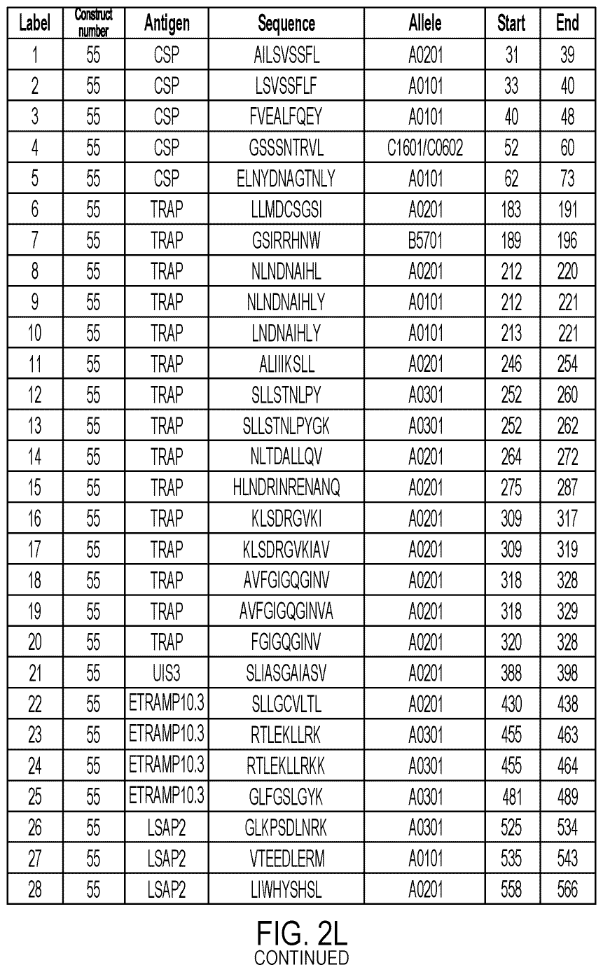

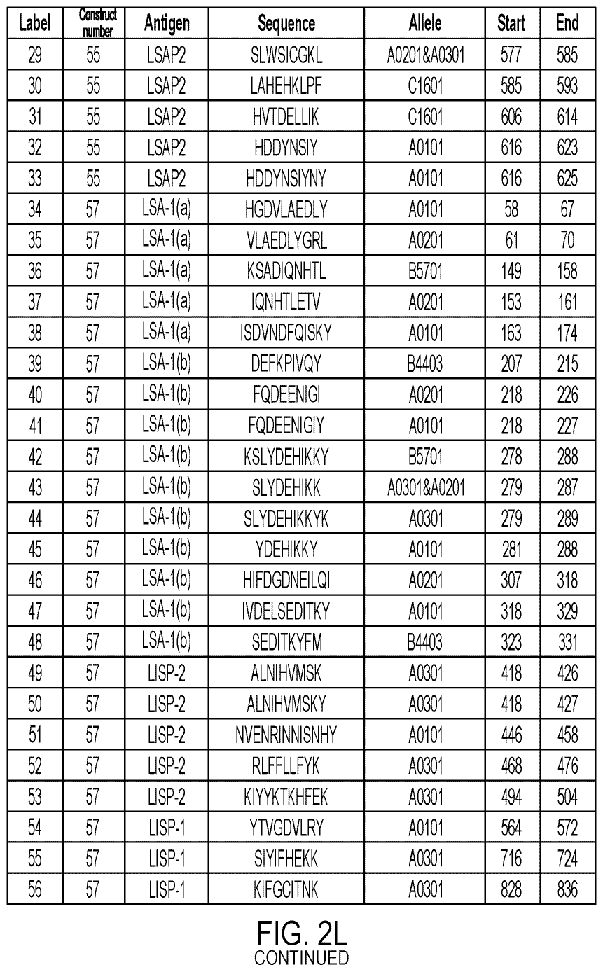

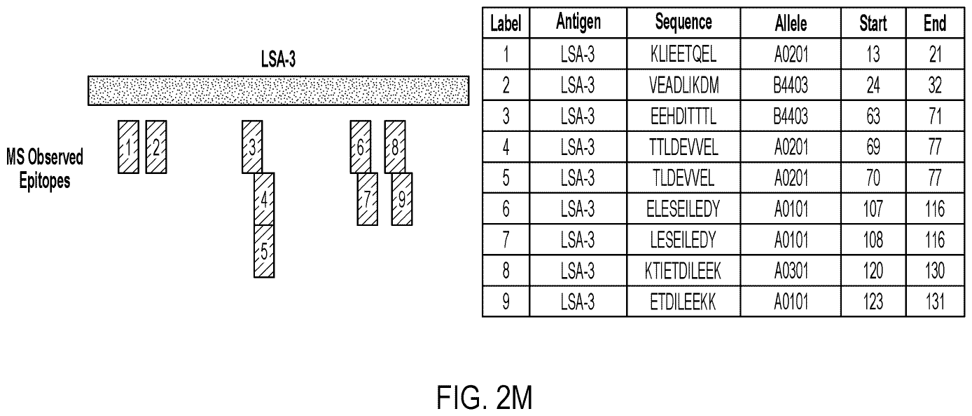

FIGS. 2A-2K show immunological characterization of eleven Plasmodium proteins (specifically, CSP, TRAP, EXP1, UIS3, ETRAMP10.3, LISP-1, LISP-2, LSA-1, LSA-3, LSAP1, and LSAP2), and also depicts fragments selected for inclusion in an antigen (e.g., a string construct antigen) for use in accordance with the present disclosure. For each Plasmodium protein, the top panel of the figure shows published HLA Class I epitopes as light green dots, and predicted HLA class I epitopes as dark green dots; the second panel shows predicted class II epitopes (i.e., binding), the third panel shows evolutionary conservation across the sixteen strains listed above, and the fourth panel shows structural features of the protein, including hydrophobicity (where darker stripes represent more hydrophobic residues). The blue bars on each panel indicate the portion of the protein identified herein as including sequences useful in vaccine antigen(s).

### Example 2: Immunogenicity Studies of Exemplary Peptide Strings RNA Constructs 48 and 57

The present Example documents the ability of certain T cell peptide string constructs, provided by the present disclosure, to induce a T cell response, as assessed in mice.

Provided T cell peptide string constructs can be assessed for their ability to induce a T-cell mediated immune response. In some embodiments, a T cell peptide string construct is determined to induce a useful immune response if splenocytes from a subject (e.g., a mouse) immunized with such construct, following incubation with peptide(s) as described herein, exhibit secretion of a pro-inflammatory cytokines (e.g., IFN-γ) in an Enzyme-linked immunospot (ELISpot), as described herein.

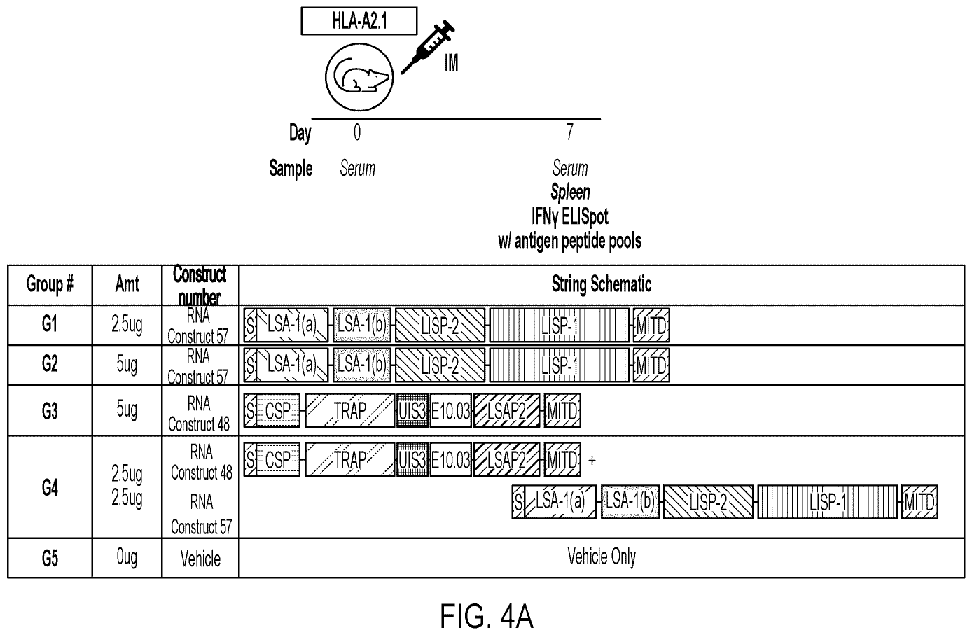

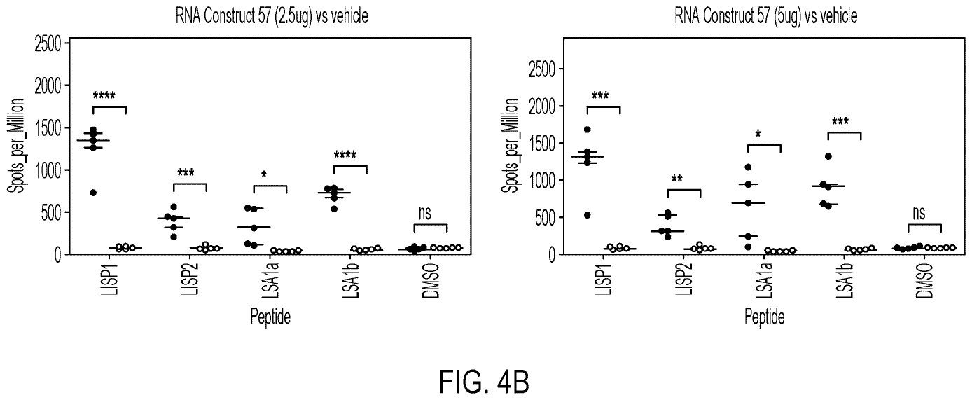

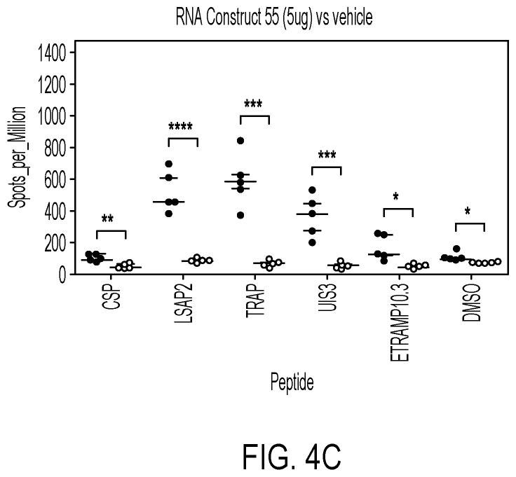

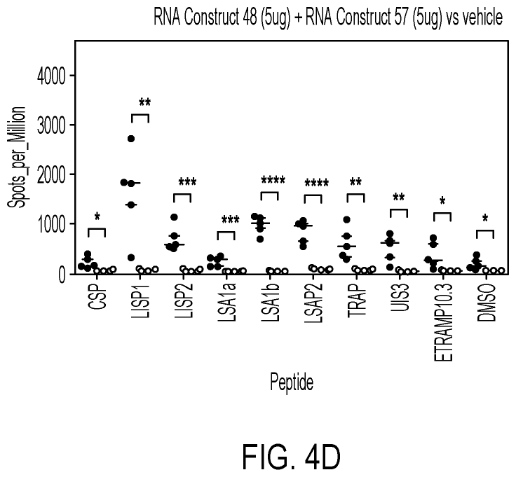

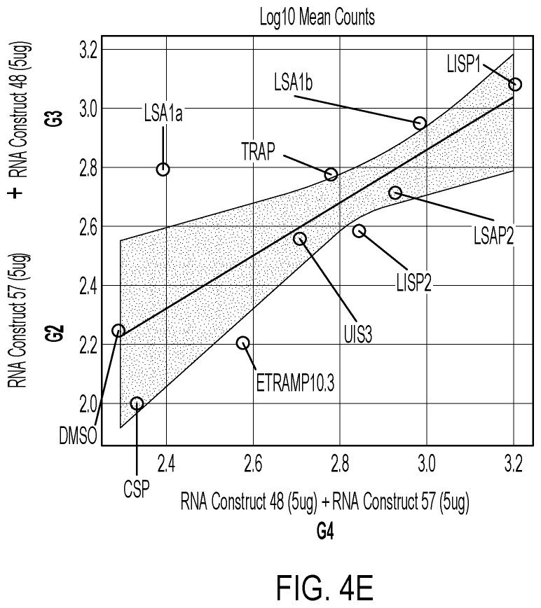

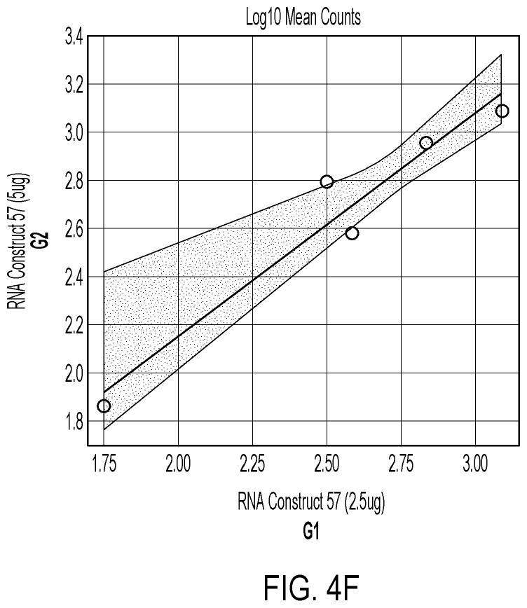

HLA-A2.1 mice were divided into multiple groups receiving treatment (as depicted in FIG. 4A) and were immunized intramuscularly (IM) with 2.5 μg or 5 μg of a T cell peptide string construct or injected with vehicle. At the end of the experiment (day 7), splenocytes were harvested and cryopreserved. Splenocytes were incubated overnight with construct specific antigen peptide pools (See Table 10 and Table 11), using the Mouse IFN-γ ELISpot Kit (R&D Systems, EL485) following the manufacturer's instructions, and assessed for IFN-γ responses. Specifically, splenocytes were incubated with individual antigen peptide pools (0.3 UM of each peptides within antigen pool) in 200 μl serum-free media (X-VIVO/1% Pen-Strep/1% Glutamax), for 20 hrs at 37 C in blocked pre-coated ELISpot plates in triplicate wells. Negative control included incubation with serum-free media and DMSO (matched volume to peptide pool) and positive control included incubation with 0.3 μM Concanavalin A (ConA). Post incubation, plates were washed with PBS/Tween and detection antibody (biotinylated anti-IFN-γ) was added to wells for overnight incubation at 4 C. Following incubation with the detection antibody, plates were washed with PBS/Tween and incubated with Streptavidin-AP for 2 hours at room temperature. Plates were then washed with PBS/Tween and 100 μl of BCIP/NBT Chromogen substrate was added to each well and incubated for 45 minutes at room temperature, protected from light. Chromagen solution was decanted from plates, wells were rinsed with distilled water and plates were air dried. Cell counts were recorded per well using a CTL Immunospot reader.

TABLE 10
Peptides used for splenocyte stimulation in the ELISpot Assay

| Target | Peptide Pool # | SEQ ID NO.: |
|---|---|---|
| CSP | 1 | 481 |
| CSP | 1 | 482 |
| CSP | 1 | 483 |
| CSP | 1 | 484 |
| CSP | 1 | 485 |
| CSP | 1 | 486 |
| CSP | 1 | 487 |
| CSP | 1 | 488 |
| CSP | 1 | 489 |
| CSP | 1 | 490 |
| CSP | 1 | 491 |
| CSP | 1 | 492 |
| CSP | 1 | 493 |
| CSP | 1 | 494 |
| CSP | 1 | 495 |
| CSP | 1 | 496 |
| CSP | 1 | 497 |
| CSP | 1 | 498 |
| CSP | 1 | 499 |
| CSP | 1 | 500 |
| CSP | 1 | 501 |
| CSP | 1 | 502 |
| LISP1 | 2 | 513 |
| LISP1 | 2 | 514 |
| LISP1 | 2 | 515 |
| LISP1 | 2 | 516 |
| LISP1 | 2 | 517 |
| LISP1 | 2 | 518 |
| LISP1 | 2 | 519 |
| LISP1 | 2 | 520 |
| LISP1 | 2 | 521 |
| LISP1 | 2 | 522 |
| LISP1 | 2 | 523 |
| LISP1 | 2 | 524 |
| LISP1 | 2 | 525 |
| LISP1 | 2 | 526 |
| LISP1 | 2 | 527 |
| LISP1 | 2 | 528 |
| LISP1 | 2 | 529 |
| LISP1 | 2 | 530 |
| LISP1 | 2 | 531 |
| LISP1 | 2 | 532 |
| LISP1 | 2 | 533 |
| LISP1 | 2 | 534 |
| LISP1 | 2 | 535 |
| LISP1 | 2 | 536 |
| LISP1 | 2 | 537 |
| LISP1 | 2 | 538 |
| LISP1 | 2 | 539 |
| LISP1 | 2 | 540 |
| LISP1 | 2 | 541 |
| LISP1 | 2 | 542 |
| LISP1 | 2 | 543 |
| LISP1 | 2 | 544 |
| LISP1 | 2 | 545 |
| LISP1 | 2 | 546 |
| LISP1 | 2 | 547 |
| LISP1 | 2 | 548 |
| LISP1 | 2 | 549 |
| LISP1 | 2 | 550 |
| LISP1 | 2 | 551 |
| LISP1 | 2 | 552 |
| LISP1 | 2 | 553 |
| LISP1 | 2 | 554 |
| LISP1 | 2 | 555 |
| LISP1 | 2 | 556 |
| LISP1 | 2 | 557 |
| LISP1 | 2 | 558 |
| LISP1 | 2 | 559 |
| LISP1 | 2 | 560 |
| LISP1 | 2 | 561 |
| LISP1 | 2 | 562 |
| LISP1 | 2 | 563 |
| LISP1 | 2 | 564 |
| LISP1 | 2 | 565 |
| LISP1 | 2 | 566 |
| LISP1 | 2 | 567 |
| LISP1 | 2 | 568 |
| LISP1 | 2 | 569 |
| LISP1 | 2 | 570 |
| LISP1 | 2 | 571 |
| LISP1 | 2 | 572 |
| LISP1 | 2 | 573 |
| LISP1 | 2 | 574 |
| LISP1 | 2 | 575 |
| LISP1 | 2 | 576 |
| LISP1 | 2 | 577 |
| LISP1 | 2 | 578 |
| LISP1 | 2 | 579 |
| LISP1 | 2 | 580 |
| LISP1 | 2 | 581 |
| LISP1 | 2 | 582 |
| LISP1 | 2 | 583 |
| LISP1 | 2 | 584 |
| LISP1 | 2 | 585 |
| LISP1 | 2 | 586 |
| LISP1 | 2 | 587 |
| LISP1 | 2 | 588 |
| LISP2 | 3 | 589 |
| LISP2 | 3 | 590 |
| LISP2 | 3 | 591 |
| LISP2 | 3 | 592 |
| LISP2 | 3 | 593 |
| LISP2 | 3 | 594 |
| LISPZ | 3 | 595 |
| LISP2 | 3 | 596 |
| LISP2 | 3 | 597 |
| LISP2 | 3 | 598 |
| LISP2 | 3 | 599 |
| LISP2 | 3 | 600 |
| LISP2 | 3 | 601 |
| LISP2 | 3 | 602 |
| LISP2 | 3 | 603 |
| LISP2 | 3 | 604 |
| LISP2 | 3 | 605 |
| LISP2 | 3 | 606 |
| LISP2 | 3 | 607 |
| LISP2 | 3 | 608 |
| LISP2 | 3 | 609 |
| LISP2 | 3 | 610 |
| LISP2 | 3 | 611 |
| LISP2 | 3 | 612 |
| LISP2 | 3 | 613 |
| LISP2 | 3 | 614 |
| LISP2 | 3 | 615 |
| LISP2 | 3 | 616 |
| LISP2 | 3 | 617 |
| LISP2 | 3 | 618 |
| LISP2 | 3 | 619 |
| LISP2 | 3 | 620 |
| LISP2 | 3 | 621 |
| LISP2 | 3 | 622 |
| LISP2 | 3 | 623 |
| LISP2 | 3 | 624 |
| LISP2 | 3 | 625 |
| LISP2 | 3 | 626 |
| LISP2 | 3 | 627 |
| LISP2 | 3 | 628 |
| LISP2 | 3 | 629 |
| LISP2 | 3 | 630 |
| LISP2 | 3 | 631 |
| LISP2 | 3 | 632 |
| LISP2 | 3 | 633 |
| LISP2 | 3 | 634 |
| LISP2 | 3 | 635 |
| LISP2 | 3 | 636 |
| LISP2 | 3 | 637 |
| LSA-1a | 4 | 638 |
| LSA-1a | 4 | 639 |
| LSA-1a | 4 | 640 |
| LSA-1a | 4 | 641 |
| LSA-1a | 4 | 642 |
| LSA-1a | 4 | 643 |
| LSA-1a | 4 | 644 |
| LSA-1a | 4 | 645 |
| LSA-1a | 4 | 646 |
| LSA-1a | 4 | 647 |
| LSA-1a | 4 | 648 |
| LSA-1a | 4 | 649 |
| LSA-1a | 4 | 650 |
| LSA-1a | 4 | 651 |
| LSA-1a | 4 | 652 |
| LSA-1a | 4 | 653 |
| LSA-1a | 4 | 654 |
| LSA-1a | 4 | 655 |
| LSA-1a | 4 | 656 |
| LSA-1a | 4 | 657 |
| LSA-1a | 4 | 658 |
| LSA-1a | 4 | 659 |
| LSA-1a | 4 | 660 |
| LSA-1a | 4 | 661 |
| LSA-1a | 4 | 662 |
| LSA-1a | 4 | 663 |
| LSA-1a | 4 | 664 |
| LSA-1a | 4 | 665 |
| LSA-1a | 4 | 666 |
| LSA-1a | 4 | 667 |
| LSA-1a | 4 | 668 |
| LSA-1a | 4 | 669 |
| LSA-1a | 4 | 670 |
| LSA-1a | 4 | 671 |
| LSA-1a | 4 | 672 |
| LSA-1a | 4 | 673 |
| LSA-1a | 4 | 674 |
| LSA-1a | 4 | 675 |
| LSA-1a | 4 | 676 |
| LSA-1b | 5 | 677 |
| LSA-1b | 5 | 678 |
| LSA-1b | 5 | 679 |
| LSA-1b | 5 | 680 |
| LSA-1b | 5 | 681 |
| LSA-1b | 5 | 682 |
| LSA-1b | 5 | 683 |
| LSA-1b | 5 | 684 |
| LSA-1b | 5 | 685 |
| LSA-1b | 5 | 686 |
| LSA-1b | 5 | 687 |
| LSA-1b | 5 | 688 |
| LSA-1b | 5 | 689 |
| LSA-1b | 5 | 690 |
| LSA-1b | 5 | 691 |
| LSA-1b | 5 | 692 |
| LSA-1b | 5 | 693 |
| LSA-1b | 5 | 694 |
| LSA-1b | 5 | 695 |
| LSA-1b | 5 | 696 |
| LSA-1b | 5 | 697 |
| LSA-1b | 5 | 698 |
| LSA-1b | 5 | 699 |
| LSA-1b | 5 | 700 |
| LSA-1b | 5 | 701 |
| LSA-1b | 5 | 702 |
| LSA-1b | 5 | 703 |
| LSA-1b | 5 | 704 |
| LSA-1b | 5 | 705 |
| LSA-1b | 5 | 706 |
| LSA-1b | 5 | 707 |
| LSAP2 | 6 | 759 |
| LSAP2 | 6 | 760 |
| LSAP2 | 6 | 761 |
| LSAP2 | 6 | 762 |
| LSAP2 | 6 | 763 |
| LSAP2 | 6 | 764 |
| LSAP2 | 6 | 765 |
| LSAP2 | 6 | 766 |
| LSAP2 | 6 | 767 |
| LSAP2 | 6 | 768 |
| LSAP2 | 6 | 769 |
| LSAP2 | 6 | 770 |
| LSAP2 | 6 | 771 |
| LSAP2 | 6 | 772 |
| LSAP2 | 6 | 773 |
| LSAP2 | 6 | 774 |
| LSAP2 | 6 | 775 |
| LSAP2 | 6 | 776 |
| LSAP2 | 6 | 777 |
| LSAP2 | 6 | 778 |
| LSAP2 | 6 | 779 |
| LSAP2 | 6 | 780 |
| LSAP2 | 6 | 781 |
| LSAP2 | 6 | 782 |
| LSAP2 | 6 | 783 |
| LSAP2 | 6 | 784 |
| LSAP2 | 6 | 785 |
| LSAP2 | 6 | 786 |
| LSAP2 | 6 | 787 |
| LSAP2 | 6 | 788 |
| LSAP2 | 6 | 789 |
| LSAP2 | 6 | 790 |
| LSAP2 | 6 | 791 |
| LSAP2 | 6 | 792 |
| LSAP2 | 6 | 793 |
| LSAP2 | 6 | 794 |
| LSAP2 | 6 | 795 |
| LSAP2 | 6 | 796 |
| LSAP2 | 6 | 797 |
| TRAP | 7 | 798 |
| TRAP | 7 | 799 |
| TRAP | 7 | 800 |
| TRAP | 7 | 801 |
| TRAP | 7 | 802 |
| TRAP | 7 | 803 |
| TRAP | 7 | 804 |
| TRAP | 7 | 805 |
| TRAP | 7 | 806 |
| TRAP | 7 | 807 |
| TRAP | 7 | 808 |
| TRAP | 7 | 809 |
| TRAP | 7 | 810 |
| TRAP | 7 | 811 |
| TRAP | 7 | 812 |
| TRAP | 7 | 813 |
| TRAP | 7 | 814 |
| TRAP | 7 | 815 |
| TRAP | 7 | 816 |
| TRAP | 7 | 817 |
| TRAP | 7 | 818 |
| TRAP | 7 | 819 |
| TRAP | 7 | 820 |
| TRAP | 7 | 821 |
| TRAP | 7 | 822 |
| TRAP | 7 | 823 |
| TRAP | 7 | 824 |
| TRAP | 7 | 825 |
| TRAP | 7 | 826 |
| TRAP | 7 | 827 |
| TRAP | 7 | 828 |
| TRAP | 7 | 829 |
| TRAP | 7 | 830 |
| TRAP | 7 | 831 |
| TRAP | 7 | 832 |
| TRAP | 7 | 833 |
| TRAP | 7 | 834 |
| TRAP | 7 | 835 |
| TRAP | 7 | 836 |
| TRAP | 7 | 837 |
| TRAP | 7 | 838 |
| TRAP | 7 | 839 |
| TRAP | 7 | 840 |
| TRAP | 7 | 841 |
| TRAP | 7 | 842 |
| TRAP | 7 | 843 |
| TRAP | 7 | 844 |
| TRAP | 7 | 845 |
| UIS3 | 8 | 846 |
| UIS3 | 8 | 847 |
| UIS3 | 8 | 848 |
| UIS3 | 8 | 849 |
| UIS3 | 8 | 850 |
| UIS3 | 8 | 851 |
| UIS3 | 8 | 852 |
| UIS3 | 8 | 853 |
| UIS3 | 8 | 854 |
| UIS3 | 8 | 855 |
| ETRAMP10.3 | 9 | 856 |
| ETRAMP10.3 | 9 | 857 |
| ETRAMP10.3 | 9 | 858 |
| ETRAMP10.3 | 9 | 859 |
| ETRAMP10.3 | 9 | 860 |
| ETRAMP10.3 | 9 | 861 |
| ETRAMP10.3 | 9 | 862 |
| ETRAMP10.3 | 9 | 863 |
| ETRAMP10.3 | 9 | 864 |
| ETRAMP10.3 | 9 | 865 |
| ETRAMP10.3 | 9 | 866 |
| ETRAMP10.3 | 9 | 867 |
| ETRAMP10.3 | 9 | 868 |
| ETRAMP10.3 | 9 | 869 |
| ETRAMP10.3 | 9 | 870 |
| ETRAMP10.3 | 9 | 871 |
| ETRAMP10.3 | 9 | 872 |

TABLE 12
Peptide pools tested for each T cell peptide string

| | CSP | LISP1 | LISP2 | LSA1a | LSA1b | LSAP2 | TRAP | UIS3 | ETRAMP10.3 |
|---|---|---|---|---|---|---|---|---|---|
| RNA | X | X | X | X | X | X | X | X | X |
| Construct | | | | | | | | | |
| 50 | | | | | | | | | |
| RNA | X | X | | X | X | X | X | X | X |
| Construct | | | | | | | | | |
| 51 | | | | | | | | | |
| RNA | X | X | X | | | X | X | X | X |
| Construct | | | | | | | | | |
| 52 | | | | | | | | | |
| RNA | X | X | | | X | X | X | X | X |
| Construct | | | | | | | | | |
| 53 | | | | | | | | | |
| RNA | X | | | | | X | X | X | X |
| Construct | | | | | | | | | |
| 48 | | | | | | | | | |
| RNA | | X | X | X | X | | | | |
| Construct | | | | | | | | | |
| 57 | | | | | | | | | |

As shown in FIG. 4C, upon stimulation with construct specific antigen peptide pools, IFN-γ producing cells were detected in splenocytes from mice immunized with T cell peptide string RNA construct 57. Splenocytes from mice immunized with either 2.5 μg or 5 μg T cell peptide string RNA construct 57 had an average of at least about 1000 spots per million after stimulation with a LISP1 peptide pool. Splenocytes from mice immunized with either 2.5 μg or 5 μg T cell peptide string RNA construct 57 had an average of at least about 250 spots per million after stimulation with a LISP2 peptide pool. Splenocytes from mice immunized with 5 μg T cell peptide string RNA construct 57 had an average of at least about 500 spots per million after stimulation with a LISP2 peptide pool, while splenocytes from mice immunized with 2.5 μg T cell peptide string RNA construct 57 had an average of at least about 500 spots per million after stimulation with a LSA1a peptide pool. Splenocytes from mice immunized with either 2.5 μg or 5 μg T cell peptide string RNA construct 57 had an average of at least about 250 spots per million after stimulation with a LSA1b peptide pool. In contrast, upon stimulation with construct specific antigen peptide pools detection of IFN-γ producing cells, there was almost no detection of IFN-γ producing cells in splenocytes from mice treated with vehicle. As shown in FIG. 4F, there was a near linear relationship between IFN-γ producing cells detected in splenocytes from mice immunized with ether 2.5 μg or 5 μg T cell peptide string RNA construct 57 after stimulation with construct specific antigen peptide pools. These experiments indicate that IFN-γ response could be induced at a lower dose (i.e., 2.5 μg) of T cell peptide string RNA construct 57.

As shown in FIG. 4D, upon stimulation with construct specific antigen peptide pools, IFN-γ producing cells were detected in splenocytes from mice immunized with T cell peptide string RNA construct 48. Splenocytes from mice immunized with 5 μg T cell peptide string RNA construct 48 had an average of at least about 100 spots per million after stimulation with a LISP1 peptide pool. Splenocytes from mice immunized with 5 μg T cell peptide string RNA construct 48 had an average of at least about 400 spots per million after stimulation with a LSAP2 peptide pool. Splenocytes from mice immunized with 5 μg T cell peptide string RNA construct 48 had an average of at least about 600 spots per million after stimulation with a TRAP peptide pool. Splenocytes from mice immunized with 5 μg T cell peptide string RNA construct 48 had an average of at least about 400 spots per million after stimulation with a UIS3 peptide pool. Splenocytes from mice immunized with 5 μg T cell peptide string RNA construct 48 had an average of at least about 100 spots per million after stimulation with a ETRAMP10.3 peptide pool. In contrast, upon stimulation with construct specific antigen peptide pools, there was almost no detection of IFN-γ producing cells in splenocytes from mice treated with vehicle.

As shown in FIG. 4E, upon stimulation with construct specific antigen peptide pools, IFN-γ producing cells were detected in splenocytes from mice immunized with a combination of T cell peptide string RNA constructs 48 and 57. Splenocytes from mice immunized with 5 μg T cell peptide string RNA construct 48 and 5 μg T cell peptide string RNA construct 57 had an average of at least about 100 spots per million after stimulation with a CSP peptide pool. Splenocytes from mice immunized with 5 μg T cell peptide string RNA construct 48 and 5 μg T cell peptide string RNA construct 57 had an average of at least about 1500 spots per million after stimulation with a LISP1 peptide pool. Splenocytes from mice immunized with 5 μg T cell peptide string RNA construct 48 and 5 μg T cell peptide string RNA construct 57 had an average of at least about 500 spots per million after stimulation with a LISP2 peptide pool. Splenocytes from mice immunized with 5 μg T cell peptide string RNA construct 48 and 5 μg T cell peptide string RNA construct 57 had an average of at least about 100 spots per million after stimulation with a LSA1a peptide pool. Splenocytes from mice immunized with 5 μg T cell peptide string RNA construct 48 and 5 μg T cell peptide string RNA construct 57 had an average of at least about 1000 spots per million after stimulation with a LSA1b peptide pool. Splenocytes from mice immunized with 5 μg T cell peptide string RNA construct 48 and 5 μg T cell peptide string RNA construct 57 had an average of at least about 1000 spots per million after stimulation with a LSAP2 peptide pool. Splenocytes from mice immunized with 5 μg T cell peptide string RNA construct 48 and 5 μg T cell peptide string RNA construct 57 had an average of at least about 500 spots per million after stimulation with a TRAP peptide pool. Splenocytes from mice immunized with 5 μg T cell peptide string RNA construct 48 and 5 μg T cell peptide string RNA construct 57 had an average of at least about 500 spots per million after stimulation with a UIS3 peptide pool. Splenocytes from mice immunized with 5 μg T cell peptide string RNA construct 48 and 5 μg T cell peptide string RNA construct 57 had an average of at least about 100 spots per million after stimulation with a ETRAMP10.3 peptide pool. In contrast, upon stimulation with construct specific antigen peptide pools, there was almost no detection of IFN-γ producing cells in splenocytes from mice treated with vehicle. As shown in FIG. 4G, upon stimulation with construct specific antigen peptide pools, detection of IFN-γ producing cells was similar in splenocytes from mice immunized with either a combination of T cell peptide string RNA constructs (i.e., 48 and 57) or individual T cell peptide string RNA constructs (i.e., 48 or 57).

Thus, the present Example demonstrates that certain T cell peptide string constructs effectively induce an immune response characterized by activation of T-cells secreting pro-inflammatory cytokines (e.g., IFN-γ), e.g., assessed using a ELISpot assay.

### Example 3: Immunogenicity Studies of Exemplary Additional Peptide Strings

The present further demonstrates the ability of certain T cell peptide string constructs, provided by the present disclosure, to induce a T cell response, as assessed in mice.

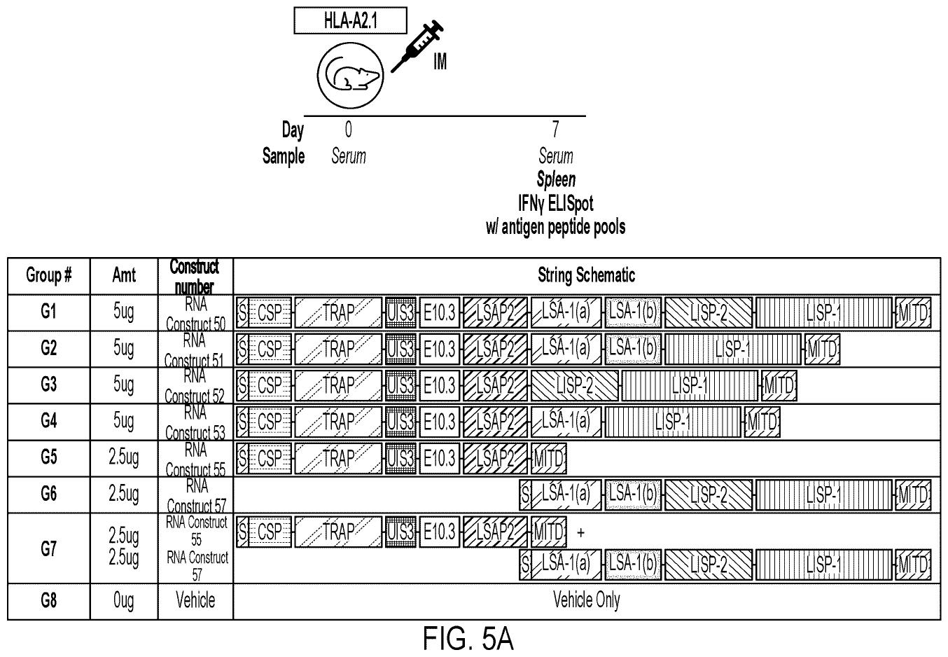

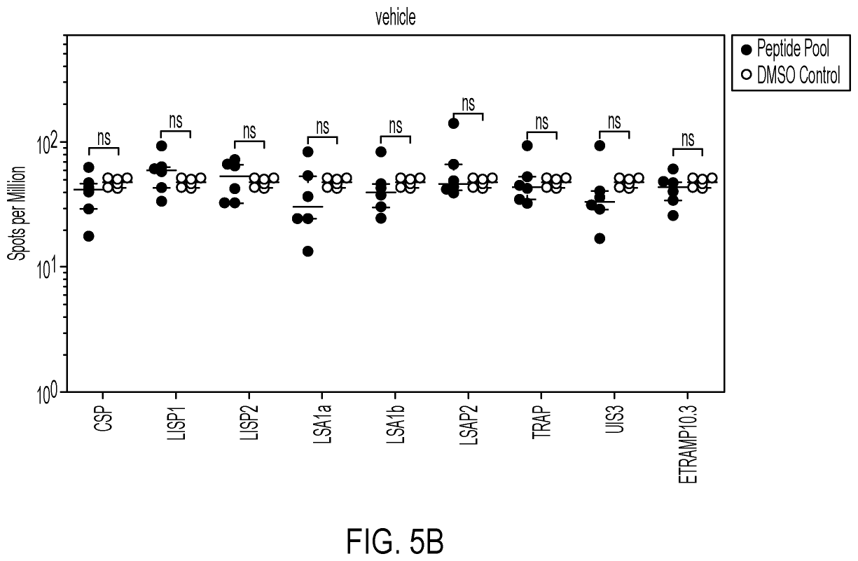

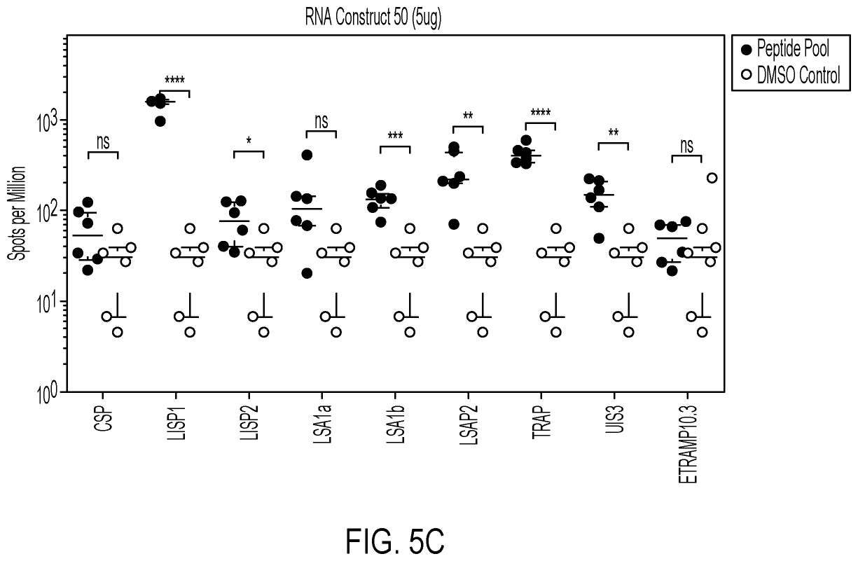

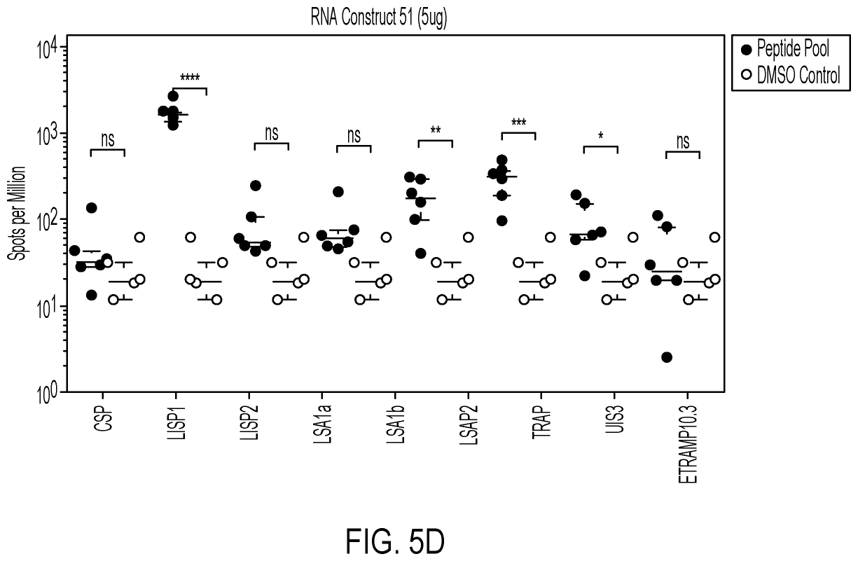

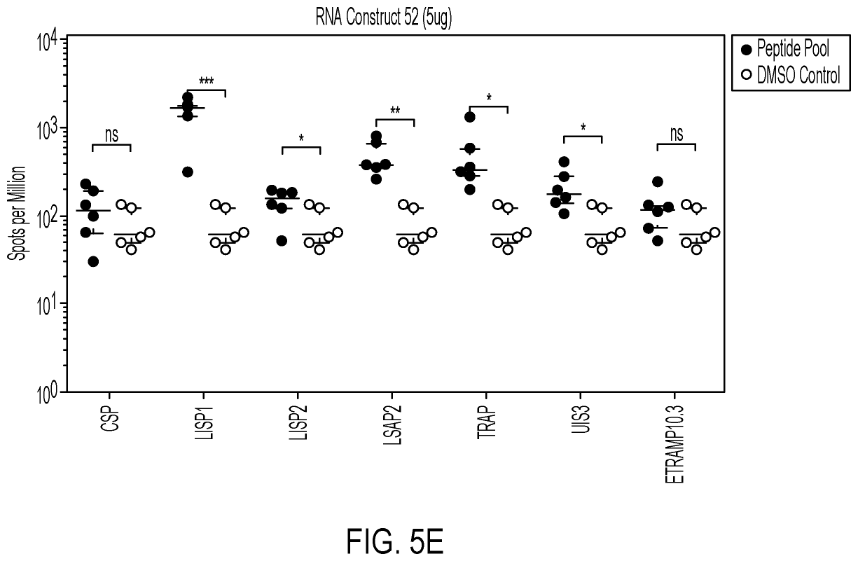

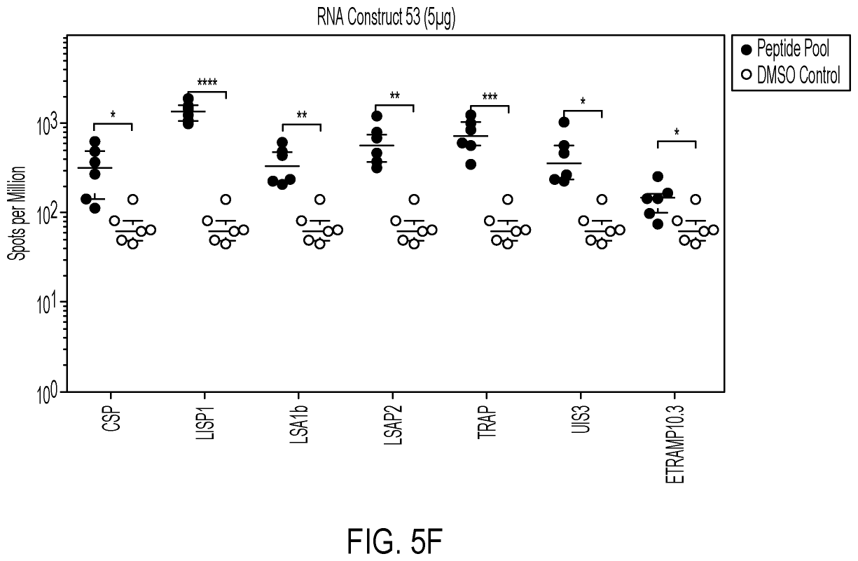

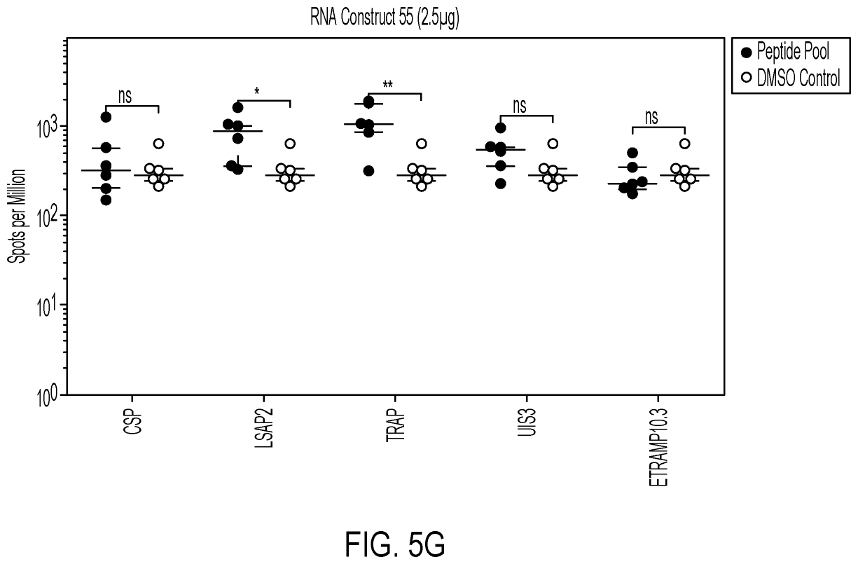

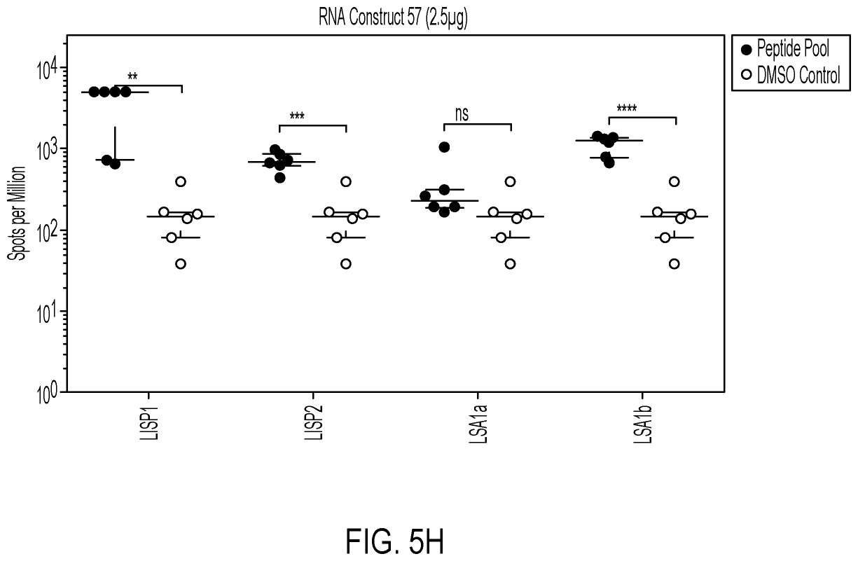

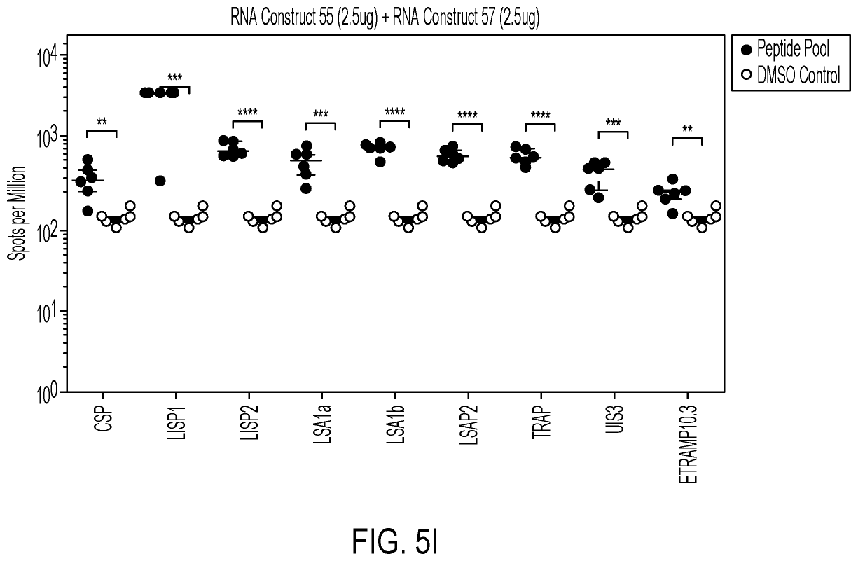

HLA-A2.1 mice were divided into multiple groups receiving treatment (as depicted in FIG. 5A) and were immunized intramuscularly (IM) with 2.5 μg or 5 μg of a T cell peptide string construct or injected with vehicle. At the end of the experiment (day 7), splenocytes were harvested and cryopreserved. Splenocytes were incubated overnight with construct specific antigen peptide pools (listed in Table 10 and Table 11), using the Mouse IFN-γ ELISpot Kit (R&D Systems, EL485) following the manufacturer's instructions, and assessed for IFN-γ responses (See Example 4).

As shown in FIG. 5C, IFN-γ producing cells were not detected in splenocytes from mice treated with vehicle, upon stimulation with CSP, LISP1, LISP2, LSA1a, LSA1b, LSAP2, TRAP, UIS3, or ETRAMP10.3 peptide pools. Splenocytes from mice immunized with tested T cell peptide string construct were considered to be responsive if after stimulation with construct specific peptide pools there was a significant differences (as determined by a p value of at least 0.05) in detection of IFN-γ producing cells, as compared to splenocytes from mice treated with vehicle.

As shown in FIG. 5D, statistically significant IFN-γ producing cells were detected in splenocytes from mice immunized with 5 μg T cell peptide string RNA construct 50, upon stimulation with LISP1, LISP2, LSA1b, LSAP2, TRAP or UIS3 peptide pools, but not with CSP, LSA1a, or ETRAMP10.3. Splenocytes from mice immunized with tested T cell peptide string RNA construct 50 had an average of at least about 102 spots per million after stimulation with LISP1, LISP2, LSA1b, LSAP2, TRAP, and UIS3 peptide pools. Splenocytes from mice immunized with tested T cell peptide string RNA construct 50 had the highest response after stimulation with LISP1 with an average of about 103 spots per million.

As shown in FIG. 5E, statistically significant IFN-γ producing cells were detected in splenocytes from mice immunized with 5 μg T cell peptide string RNA construct 51, upon stimulation with LISP1, LSAP2, TRAP or UIS3 peptide pools, but not with CSP, LSA1a, LSA1b or ETRAMP10.3. Splenocytes from mice immunized with tested T cell peptide string RNA construct 51 had an average of at least about 102 spots per million after stimulation with LISP1, LSAP2, TRAP, and UIS3 peptide pools. Splenocytes from mice immunized with tested T cell peptide string RNA construct 51 had the highest response after stimulation with LISP1 with an average of about 103 spots per million.

As shown in FIG. 5F, statistically significant IFN-γ producing cells were detected in splenocytes from mice immunized with 5 μg T cell peptide string RNA construct 52, upon stimulation with LISP1, LISP2, LSAP2, TRAP or UIS3 peptide pools, but not with CSP or ETRAMP10.3. Splenocytes from mice immunized with tested T cell peptide string RNA construct 52 had an average of at least about 102 spots per million after stimulation with LISP1, LISP2, LSAP2, TRAP, and UIS3 peptide pools. Splenocytes from mice immunized with tested T cell peptide string RNA construct 52 had the highest response after stimulation with LISP1 with an average of about 103 spots per million.

As shown in FIG. 5G, statistically significant IFN-γ producing cells were detected in splenocytes from mice immunized with 5 μg T cell peptide string RNA construct 53, upon stimulation with all included antigens peptide pools. Splenocytes from mice immunized with tested T cell peptide string RNA construct 53 had an average of at least about 102 spots per million after stimulation with CSP, LISP1, LSA1b, LSAP2, TRAP, UIS3 or ETRAMP10.3 peptide pools. Splenocytes from mice immunized with tested T cell peptide string RNA construct 53 had the highest response after stimulation with LISP1 with an average of about 103spots per million.

As shown in FIG. 5H, statistically significant IFN-γ producing cells were detected in splenocytes from mice immunized with 2.5 μg T cell peptide string RNA construct 55, upon stimulation with LSAP2 and TRAP peptide pools, but not with CSP, UIS3, or ETRAMP10.3. Splenocytes from mice immunized with tested T cell peptide string RNA construct 55 had an average of at least about 102 spots per million after stimulation with LSAP2 and TRAP peptide pools.

As shown in FIG. 5I, statistically significant IFN-γ producing cells were detected in splenocytes from mice immunized with 2.5 μg T cell peptide string RNA construct 57, upon stimulation with LISP1, LISP2, LSA1b peptide pools, but not with LSA1a peptide pool. Splenocytes from mice immunized with tested T cell peptide string RNA construct 57 had an average of at least about 103 spots per million after stimulation with LISP1 or LSA1b. Splenocytes from mice immunized with tested T cell peptide string RNA construct 57 had an average of about 102 spots per million after stimulation with LISP2.

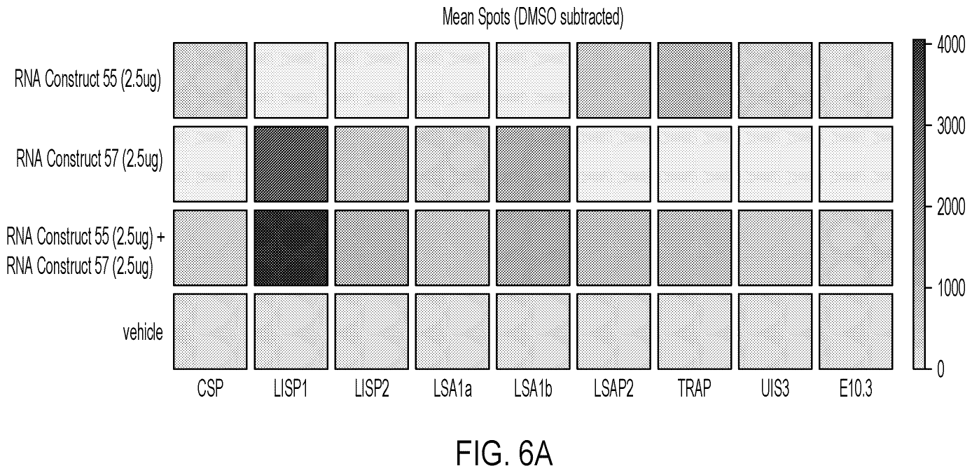

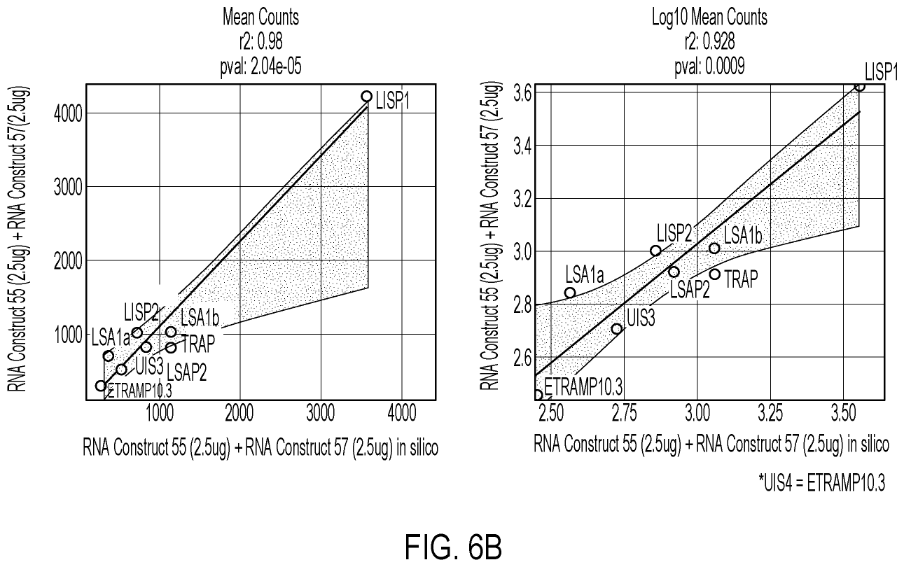

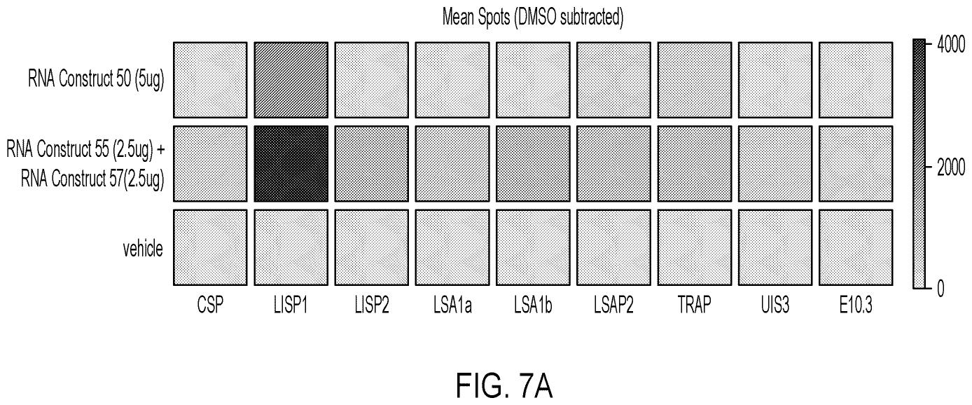

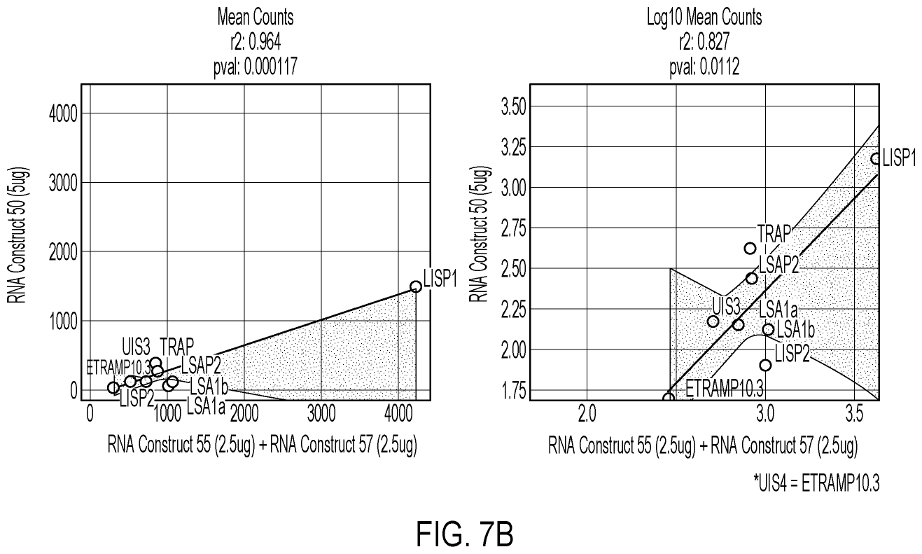

As shown in FIG. 5J, statistically significant IFN-γ producing cells were detected in splenocytes from mice immunized with a combination of 2.5 μg T cell peptide string RNA construct 55 and with 2.5 μg RNA construct 57, upon stimulation with all included antigens peptide pools. Splenocytes from mice immunized with a combination of tested T cell peptide string RNA constructs 55 and 57 had an average of at least about 103 spots per million after stimulation with LISP1, LISP2 or LSA1b. Splenocytes from mice immunized with a combination of tested T cell peptide string RNA constructs 55 and 57 had an average of at least about 102 spots per million after stimulation with CSP, LSA1a, LSAP2, TRAP, UIS3 or ETRAMP10.3. As shown in FIG. 6A, splenocytes from mice immunized with a combination of T cell peptide string RNA constructs 55 and 57, or individually with RNA construct 55 or RNA construct 57, exhibited similar responses after stimulation with CSP, LISP1, LISP2, LSA1a, LSA1b, LSAP2, TRAP, UIS3 or ETRAMP10.3. As shown in FIG. 7A, splenocytes from mice immunized with a combination of shorter T cell peptide string RNA constructs (55 and 57) exhibited an enhanced response after stimulation with CSP, LISP1, LISP2, LSA1a, LSA1b, LSAP2, TRAP, UIS3 or ETRAMP10.3, as compared to splenocytes from mice immunized with longer T cell peptide string RNA construct (50), which includes the same antigenic content as in RNA constructs 55 and 57 combined.

Thus, the present Example demonstrates that certain T cell peptide string constructs effectively induce an immune response characterized by activation of T-cells secreting pro-inflammatory cytokines (e.g., IFN-γ), e.g., assessed using a ELISpot assay.

In conclusion, in vivo immunogenicity studies of Example 2 and 3 confirmed that all antigen segments within a combination of RNA constructs 55 and 57 or within a combination of RNA constructs 48 and 57 elicit T-cell responses.

### Example 4: In Vitro Expression of Exemplary Polyribonucleotides Encoding Peptide Strings

In vitro expression studies using a MS-readout indicate that RNA constructs 55 and 57 are expressed upon transfection into a human cell line.

Briefly, HEK293T cells were co-transfected with 2.5 μg of each RNA constructs 55 and 57 and harvested 24 h later. Cells were lysed in 8M urea, total protein content was normalized across samples, and disulfide bonds were reduced and alkylated. Proteins in the lysate were digested with LysC and Trypsin prior to C₁₈ cleanup and parallel reaction monitoring analysis by MS. Heavy-isotopically labeled tryptic peptides were added to the sample prior to analysis to aid in the identification of RNA constructs 55 and 57-derived tryptic peptides.

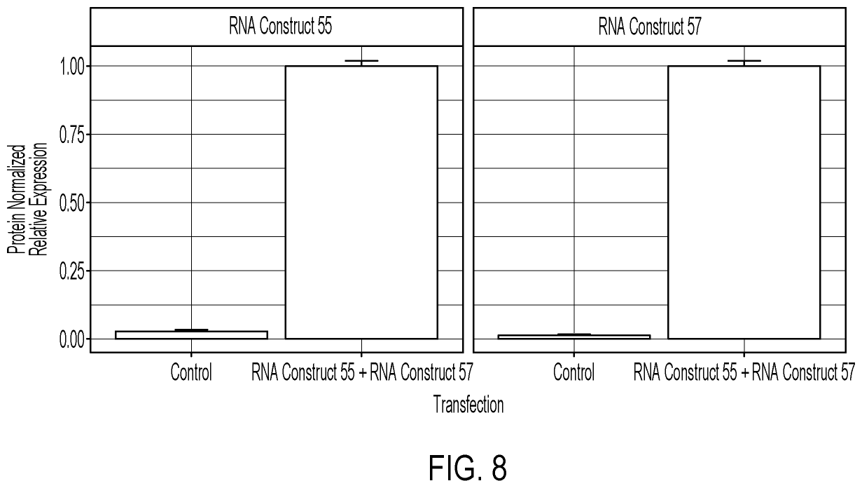

Both RNA construct 55 and 57 are detected upon transfection of the combination into HEK293T cells (FIG. 8).

In conclusion, in vitro expression (FIG. 8) and immunopeptidomics (FIGS. 2L and 2K) studies have confirmed that RNA constructs 55 and 57 are expressed and generate epitopes presented on HLA complexes when transfected into human cell lines.

### Example 5: In Vivo Immunogenicity in Humans Following Administration of Exemplary Polyribonucleotides Encoding Plasmodium T-Cell String Polypeptide Constructs

The present Example demonstrates that exemplary polyribonucleotides encoding different malarial polypeptides, as described herein, can be immunogenic in vivo in humans. Specifically, the present Example provides a randomized, dose-escalation trial for the evaluation of safety, tolerability, and immunogenicity of formulated RNA constructs and combination thereof as provided herein (“Part A”). The present Example also provides a randomized, dose-escalation trial for the evaluation of efficacy of formulated RNA constructs and combination thereof as provided herein (“Part B”).

The exemplary formulated RNA constructs to be assessed in this Example includes a combination of RNA Construct 55 and RNA Construct 57.

### Inclusion Criteria—Part A

Subjects to be included in Part A of a trial as described in this Example:
i. Are aged 18 to 55 years.
ii. Have a body mass index over 18.5 kg/m² and under 35 kg/m² and weigh at least 45 kg at an initial visit (“Visit 0”).
iii. Are healthy, in the clinical judgment of a health practitioner (e.g., a trial investigator) based on reported medical history data, physical examination, 12-lead electrocardiogram (ECG), vital signs, and clinical laboratory test outcomes. Healthy subjects with pre-existing stable disease (e.g., obesity, hypertension, etc.), defined as disease but not requiring significant change in therapy or hospitalization for worsening disease during the three months (e.g., 90 days) before Visit 0, can be included.
iv. Agree not to enroll in another trial with an investigational medicinal product (“IMP”) starting from Visit 0 and until 12 weeks after receiving a third dose of a formulated RNA construct or a combination of formulated RNA constructs.
V. Have not traveled and agree not to travel to a malaria-endemic region, as defined per Centers for Disease Control and Prevention (CDC) starting 6 months before Visit 0 and continuously until 28 days after receiving a third dose of a formulated RNA construct or a combination of formulated RNA constructs.
vi. Have tested negative human immunodeficiency virus (HIV)-1 and-2 blood test result at Visit 0.
vii. Have a negative Hepatitis B surface antigen (HBsAg) test result at Visit 0 and negative anti Hepatitis C virus (anti-HCV) antibodies, or negative HCV PCR test result if the anti-HCV is positive at Visit 0.
viii. If a subject has childbearing potential, the subject has a negative serum beta-human chorionic gonadotropin (β-HCG) pregnancy test result at Visit 0 and negative urine pregnancy test results before each administration of a formulated RNA construct or a combination of formulated RNA constructs. Subjects born female who are postmenopausal or permanently sterilized are not be considered to have childbearing potential.
ix. If a subject has childbearing potential, the subject agree to practice a highly effective form of contraception starting at Visit 0 and continuously until 90 days after receiving a third dose of a formulated RNA construct or a combination of formulated RNA constructs.
x. If a subject has childbearing potential, the subject agree not to donate or cryopreserve eggs (ova, oocytes) for the purposes of assisted reproduction during trial, starting at Visit 0 and continuously until 90 days after receiving a third dose of a formulated RNA construct or a combination of formulated RNA constructs.
xi. If a subject is male, does not have had a vasectomy and are sexually active with partners of childbearing potential agree to use condoms and to practice a highly effective form of contraception with their sexual partners of childbearing potential during the trial, starting at Visit 0 and continuously until 90 days after receiving a third dose of a formulated RNA construct or a combination of formulated RNA constructs.
xii. If a subject is male, will refrain from sperm donation, starting at Visit 0 and continuously until 90 days after receiving a third dose of a formulated RNA construct or a combination of formulated RNA constructs.

### Exclusion Criteria—Part A

Subjects with the following are excluded from Part A of a trial described in the present Example:
i. A history of Plasmodium parasitemia (any species) based on subject-reported medical history.
ii. A prior residence for greater than 6 months continuously in a malaria-endemic region as defined per CDC at any point during their lifetime.
iii. Participation in breastfeeding or an intention to become pregnant or to father children starting with Visit 0 and continuously until 90 after receiving a third dose of a formulated RNA construct or a combination of formulated RNA constructs.
iv. A history of any serious adverse reactions to vaccines or to vaccine components such as lipids, and including history of anaphylaxis and related symptoms such as hives, respiratory difficulty, angioedema, and/or abdominal pain (not excluded from participation: a subject who had an anaphylactic adverse reaction to pertussis vaccine as a child).
v. Existence or history of the following medical conditions:
   a. Uncontrolled or moderate or severe respiratory diseases (e.g., asthma, chronic obstructive pulmonary disease); symptoms of asthma severity as defined in the US National Asthma Education and Prevention Program Expert Panel report, 2020—e.g., exclude a volunteer who:
      i. Uses a short-acting rescue inhaler (typically a beta 2 agonist) daily, or
      ii. Uses high dose inhaled corticosteroids (per American Academy of Allergy Asthma & Immunology), or
      iii. In the past year has either of the following:
         1. Greater than one exacerbation of symptoms treated with oral/parenteral corticosteroids; or
         2. Needed hospitalization, or intubation for asthma;
   b. Diabetes mellitus type 1 or type 2, including cases controlled with diet alone or elevated hemoglobin A1C (HbA1c) ≥6.5% at screening (not excluded: history of isolated gestational diabetes);
   c. Hypertension:
      i. If a person has a history of hypertension, or elevated blood pressure detected during screening, exclude for blood pressure that is not well controlled. Well controlled blood pressure is defined as consistently≤140 mm Hg systolic and ≤90 mm Hg diastolic, with or without medication, with only isolated, brief instances of higher readings, which must be $150 mm Hg systolic and ≤100 mm Hg diastolic at screening and enrollment;
   d. Malignancy within 5 years of screening, excluding localized basal or squamous cell skin cancer;
   e. Any current or history of cardiovascular diseases, (e.g., myocarditis, pericarditis, myocardial infarction, congestive heart failure, cardiomyopathy or clinically significant arrhythmias), unless such disease is not considered relevant for participation in this trial in a health practitioner's (e.g., investigator's) judgment;
   f. An abnormal screening ECG (i.e., showing the corrected QT interval by Fridericia (QTcF)>150 ms; significant ST-T wave changes suggestive of myocardial ischemia or of an acute or indeterminate-age myocardial infarction; left ventricular hypertrophy; any non-sinus rhythm including isolated premature ventricular contractions; complete right or left bundle branch block [QRS>120 ms]; second- or third-degree atrioventricular [AV] block); or other clinically significant abnormalities on the ECG at the investigator's discretion;
   g. Bleeding disorder diagnosed by a doctor (e.g., factor deficiency, coagulopathy, or platelet disorder requiring special precautions); or
   h. Seizure disorder: History of seizure(s) within past 3 years. Also exclude if volunteer has used medications in order to prevent or treat seizure(s) at any time within the past 3 years.
vi. Documented major psychiatric illness, including bipolar disorder, major depressive disorder, schizophrenia, autism, and attention deficit-hyperactivity disorder that at the discretion of the investigator could interfere with participation and follow-up as outlined by the trial.
vii. The following diseases associated with immune dysregulation:
   a. Primary immunodeficiencies;
   b. History of solid organ or bone marrow transplantation;
   c. Asplenia: any condition resulting in the absence of a functional spleen; or
   d. Existence or history of autoimmune disease including and not limited to thyroid autoimmune disease, multiple sclerosis, psoriasis, etc.
viii. Previous vaccination with an approved or investigational malaria vaccine at any time or having taken part in a human malaria challenge study.
ix. Receipt of any investigational product within 28 days before Visit 0.
x. Any planned non-trial vaccinations starting at Visit 0 and continuously until 28 days after a third dose of a formulated RNA construct or a combination of formulated RNA constructs. Seasonal influenza and COVID-19 vaccines are allowed; however, they should be administered at least 14 days before or 28 days after any IMP administration. Emergency vaccinations, such as tetanus, are allowed to be administered when medically indicated.
xi. Received blood/plasma products, monoclonal antibodies or immunoglobulin within 120 days before Visit 1 or planned administration starting at Visit 0 and continuously until Visit 21 (e.g., 365 days after administration of a third dose).
xii. Received allergy treatment with antigen injections within 28 days before and after each IMP administration.
xiii. Current or planned treatment with immunosuppressive therapy, including systemic corticosteroids (if systemic corticosteroids are administered for ≥14 days at a dose of ≥20 mg/d of prednisone or equivalent) starting at Visit 0 and continuously until a third dose of a formulated RNA construct or a combination of formulated RNA constructs. Intraarticular, intrabursal, or topical (skin or eyes) corticosteroids are permitted.
xiv. Have a history of alcohol abuse or drug addiction within 1 year before Visit 0 or have a history (within the past 5 years) of substance abuse, which in the opinion of a health practitioner (e.g., an investigator), could compromise their wellbeing if they participate as a subject in a trial, or that could prevent, limit, or confound the protocol specified assessments.
xv. Any existing condition which may affect vaccine injection and/or assessment of local reactions at the injection site, e.g., tattoos, severe scars, etc.
xvi. Are vulnerable individuals as per International Council for Harmonisation (ICH) E6 definition, i.e., are individuals whose willingness to volunteer in a clinical trial may be unduly influenced by the expectation, whether justified or not, of benefits associated with participation, or of a retaliatory response from senior members of a hierarchy in case of refusal to participate.
xvii. Any screening hematology and/or blood chemistry laboratory value that meets the definition of a Grade ≥2 abnormality or a Grade 1 abnormality at a health practitioner's (e.g., an investigator's) discretion at Visit 0. Subjects with abnormal but not clinically significant parameters not included in the toxicity guidance may be considered eligible at discretion of a health practitioner (e.g., an investigator).

### Trial—Part A

In Part A, a combination of RNA Construct 55 and RNA Construct 57 is evaluated in different dose combinations in a 3 dose schedule to select a safe, tolerable, and immunogenic dose combination, and to assess the impact of third dose on immunogenicity.

To assess safety, tolerability, and immunogenicity of the combination, subjects are administered RNA Construct 55 and RNA Construct 57. Evaluation uses a staggered dose-escalation schema with sentinel subjects in the cohort. A placebo cohort that receives isotonic NaCl solution (0.9%) is also assessed.

A first dose of a composition comprising RNA Construct 55 and RNA Construct 57 is administered to subject on Day 1. Approximately eight weeks later, a second dose of the composition is administered to the subject. A third dose is administered approximately 18 weeks after the second dose.

Subjects are assessed for the following primary outcome measures after each dose:
i. Frequency of solicited local reactions at the injection site (e.g., pain, erythema/redness and/or induration/swelling) recorded up to 7 days after each dose;
ii. Frequency of solicited systemic reactions (vomiting, diarrhea, headache, fatigue, muscle/joint pain, and fever) recorded up to 7 days after each dose;
iii. Frequency of subjects with at least one AE occurring until 28 days after each dose; and
iv. Frequency of subjects with at least one medically attended adverse event (MAAE) occurring until 28 days after each dose.

Subjects are assessed to determine a frequency of subjects with at least one SAE occurring until 24 weeks after a third dose.

Descriptive statistics on antibody levels at assessed time points are obtained.

### EQUIVALENTS

Those skilled in the art will recognize, or be able to ascertain using no more than routine experimentation, many equivalents to the specific embodiments of technologies described herein. The scope of the present disclosure is not intended to be limited to the above Description, but rather is as set forth in the following claims.

1. A polyribonucleotide encoding a polypeptide, wherein the polypeptide comprises one or more one or more Plasmodium T-cell antigens, wherein the one or more Plasmodium T cell antigens comprise two or more of:
(i) an antigenic Plasmodium CSP polypeptide fragment;
(ii) an antigenic Plasmodium LSA-1 (a) polypeptide fragment;
(iii) an antigenic Plasmodium LSA-1 (b) polypeptide fragment;
(iv) an antigenic Plasmodium TRAP polypeptide fragment;
(v) an antigenic Plasmodium LSAP2 polypeptide fragment;
(vi) an antigenic Plasmodium UIS3 polypeptide fragment;
(vii) an antigenic Plasmodium ETRAMP10.3 polypeptide fragment;
(viii) an antigenic Plasmodium LISP-1 polypeptide fragment;
(ix) an antigenic Plasmodium LISP-2 polypeptide fragment; and
(x) an antigenic Plasmodium LSA-3 polypeptide fragment.

2. The polyribonucleotide of claim 1, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment; and
(v) an antigenic Plasmodium LSAP2 polypeptide fragment.

3. The polyribonucleotide of claim 1 or 2, wherein the polypeptide comprises or consists of an amino acid sequence with at least 85% sequence identity to an amino acid sequence according to SEQ ID NO: 15.

4. The polyribonucleotide of claim 1 or 2, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-3 polypeptide fragment;
(vii) an antigenic Plasmodium LSA-1 (a) polypeptide fragment; and
(viii) an antigenic Plasmodium LSA-1 (b) polypeptide fragment.

5. The polyribonucleotide of claim 1 or 2, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment;
(viii) an antigenic Plasmodium LISP-2 polypeptide fragment; and
(ix) an antigenic Plasmodium LISP-1 polypeptide fragment.

6. The polyribonucleotide of claim 1 or 2, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment; and
(viii) an antigenic Plasmodium LISP-1 polypeptide fragment.

7. The polyribonucleotide of claim 1 or 2, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LISP-2 polypeptide fragment; and
(vii) an antigenic Plasmodium LISP-1 polypeptide fragment.

8. The polyribonucleotide of claim 1 or 2, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (b) polypeptide fragment; and
(vii) an antigenic Plasmodium LISP-1 polypeptide fragment.

9. The polyribonucleotide of claim 1 or 2, wherein the one or more Plasmodium T cell antigens comprise or consist of:
(i) an antigenic Plasmodium CSP polypeptide fragment;
(ii) an antigenic Plasmodium TRAP polypeptide fragment;
(iii) an antigenic Plasmodium UIS3 polypeptide fragment;
(iv) an antigenic Plasmodium ETRAMP10.3 polypeptide fragment;
(v) an antigenic Plasmodium LSAP2 polypeptide fragment;
(vi) an antigenic Plasmodium LSA-1 (a) polypeptide fragment;
(vii) an antigenic Plasmodium LSA-1 (b) polypeptide fragment;
(viii) an antigenic Plasmodium LISP-2 polypeptide fragment;
(ix) an antigenic Plasmodium LISP-1 polypeptide fragment; and
(x) an antigenic Plasmodium LSA-3 polypeptide fragment.

10. The polyribonucleotide of claim 1 or 2, wherein the one or more Plasmodium T cell antigens comprise or consist of:
(i) an antigenic Plasmodium LSA-1 (a) polypeptide fragment;
(ii) an antigenic Plasmodium LSA-1 (b) polypeptide fragment;
(iii) an antigenic Plasmodium LISP-2 polypeptide fragment;
(iv) an antigenic Plasmodium LISP-1 polypeptide fragment; and
(v) an antigenic Plasmodium LSA-3 polypeptide fragment.

11. The polyribonucleotide claim 1 or 2, wherein the one or more Plasmodium T cell antigens comprise or consist of:
(i) an antigenic Plasmodium LSA-1 (a) polypeptide fragment;
(ii) an antigenic Plasmodium LSA-1 (b) polypeptide fragment;
(iii) an antigenic Plasmodium LISP-2 polypeptide fragment; and
(iv) an antigenic Plasmodium LISP-1 polypeptide fragment.

12. The polyribonucleotide of claim 11, wherein the polypeptide comprises or consists of an amino acid sequence with at least 85% sequence identity to an amino acid sequence according to SEQ ID NO: 45.

13. The polyribonucleotide of any one of claims 1-9, wherein the one or more Plasmodium T cell antigens comprise the antigenic Plasmodium CSP polypeptide fragment, and wherein the antigenic Plasmodium CSP polypeptide fragment comprises a Plasmodium CSP N-terminal region.

14. The polyribonucleotide of claim 13, wherein the antigenic Plasmodium CSP polypeptide fragment further comprises a Plasmodium CSP N-terminal end region.

15. The polyribonucleotide of claim 13 or 14, wherein the antigenic Plasmodium CSP polypeptide fragment further comprises a Plasmodium CSP junction region.

16. The polyribonucleotide of any one of claims 13-15, wherein the antigenic Plasmodium CSP polypeptide fragment comprises or consists of an amino acid sequence with at least 85% sequence identity to an amino acid sequence according to SEQ ID NO: 133.

17. The polyribonucleotide of any one of claims 1-9, wherein the one or more Plasmodium T cell antigens do not comprise an antigenic Plasmodium berghei CSP polypeptide fragment.

18. The polyribonucleotide of any one of claims 1, 4-6, and 9-17, wherein the one or more Plasmodium T cell antigens comprise the antigenic Plasmodium LSA-1 (a) polypeptide fragment, and wherein the antigenic Plasmodium LSA-1 (a) polypeptide fragment comprises or consists of an amino acid sequence with at least 85% sequence identity to an amino acid sequence according to SEQ ID NO: 144.

19. The polyribonucleotide of any one of claims 1, 4-6, and 8-18, wherein the one or more Plasmodium T cell antigens comprise the antigenic Plasmodium LSA-1 (b) polypeptide fragment, and wherein the antigenic Plasmodium LSA-1 (b) polypeptide fragment comprises or consists of an amino acid sequence with at least 85% sequence identity to an amino acid sequence according to SEQ ID NO: 155.

20. The polyribonucleotide of any one of claims 1-9 and 13-19, wherein the one or more Plasmodium T cell antigens comprise the antigenic Plasmodium TRAP polypeptide fragment, and wherein the antigenic Plasmodium TRAP polypeptide fragment comprises or consists of an amino acid sequence with at least 85% sequence identity to an amino acid sequence according to SEQ ID NO: 171.

21. The polyribonucleotide of any one of claims 1-9 and 13-20, wherein the one or more Plasmodium T cell antigens comprise the antigenic Plasmodium LSAP2 polypeptide fragment, and wherein the antigenic Plasmodium LSAP2 polypeptide fragment comprises or consists of an amino acid sequence with at least 85% sequence identity to an amino acid sequence according to SEQ ID NO: 198.

22. The polyribonucleotide of any one of claims 1-9 and 13-21, wherein the one or more Plasmodium T cell antigens comprise the antigenic Plasmodium UIS3 polypeptide fragment, and wherein the antigenic Plasmodium UIS3 polypeptide fragment comprises or consists of an amino acid sequence with at least 85% sequence identity to an amino acid sequence according to SEQ ID NO: 212.

23. The polyribonucleotide of any one of claims 1-9 and 13-22, wherein the one or more Plasmodium T cell antigens comprise the antigenic Plasmodium ETRAMP10.3 polypeptide fragment, and wherein the antigenic Plasmodium ETRAMP10.3 polypeptide fragment comprises or consists of an amino acid sequence with at least 85% sequence identity to an amino acid sequence according to SEQ ID NO: 219.

24. The polyribonucleotide of any one of claims 1 and 5-23, wherein the one or more Plasmodium T cell antigens comprise the antigenic Plasmodium LISP-1 polypeptide fragment, and wherein the antigenic Plasmodium LISP-1 polypeptide fragment comprises or consists of an amino acid sequence with at least 85% sequence identity to an amino acid sequence according to SEQ ID NO: 229.

25. The polyribonucleotide of any one of claims 1, 5, 7, and 9-24, wherein the one or more Plasmodium T cell antigens comprises the antigenic Plasmodium LISP-2 polypeptide fragment, and wherein the antigenic Plasmodium LISP-2 polypeptide fragment comprises or consists of an amino acid sequence with at least 85% sequence identity to an amino acid sequence according to SEQ ID NO: 238.

26. The polyribonucleotide of any one of claims 1, 4, 9, 10, and 13-25, wherein the one or more Plasmodium T cell antigens comprise the antigenic Plasmodium LSA-3 polypeptide fragment, and wherein the antigenic Plasmodium LSA-3 polypeptide fragment comprises or consists of an amino acid sequence with at least 85% sequence identity to an amino acid sequence according to SEQ ID NO: 249.

27. The polyribonucleotide of any one of claims 1-26, wherein the polypeptide does not comprise an antigenic fragment of a bacterial polypeptide.

28. The polyribonucleotide of any one of claims 1-27, wherein the one or more Plasmodium T cell antigens do not comprise an antigenic Plasmodium sporozoite threonine-asparagine-rich protein (STARP) polypeptide fragment, optionally wherein the antigenic Plasmodium STARP polypeptide fragment comprises an amino acid sequence according to SEQ ID NO: 463.

29. The polyribonucleotide of any one of claims 1-28, wherein the polyribonucleotide further comprises sequence that encodes an MHC class I trafficking signal (MITD).

30. The polyribonucleotide of any one of claims 1-29, wherein the polypeptide comprises a secretory signal.

31. The polyribonucleotide of claim 30, wherein the secretory signal comprises or consists of a Plasmodium secretory signal, preferably a Plasmodium CSP secretory signal.

32. The polyribonucleotide of claim 30, wherein the secretory signal comprises or consists of a heterologous secretory signal.

33. The polyribonucleotide of claim 32, wherein the heterologous secretory signal comprises or consists of a non-human secretory signal.

34. The polyribonucleotide of claim 32, wherein the heterologous secretory signal comprises or consists of a viral secretory signal, preferably wherein the viral secretory signal comprises or consists of:
(a) an HSV-1 or HSV-2 secretory signal, even more preferably wherein the viral secretory signal comprises or consists of an HSV glycoprotein D (gD) secretory signal, or
(b) an Ebola virus secretory signal, even more preferably wherein the viral secretory signal comprises or consists of an Ebola virus spike glycoprotein (SGP) secretory signal.

35. The polyribonucleotide of any one of claims 1-34, wherein the polypeptide comprises a transmembrane region.

36. The polyribonucleotide of claim 35, wherein the transmembrane region comprises or consists of a Plasmodium transmembrane region, preferably wherein the Plasmodium transmembrane region comprises or consists of a Plasmodium CSP glycosylphosphatidylinositol (GPI) anchor region.

37. The polyribonucleotide of claim 35, wherein the transmembrane region comprises or consists of a heterologous transmembrane region, preferably wherein the heterologous transmembrane region:
(a) does not comprise a hemagglutin transmembrane region,
(b) comprises or consists of a viral transmembrane region, preferably wherein the viral transmembrane region comprises or consists of an HSV-1 or HSV-2 transmembrane region, even more preferably wherein the HSV transmembrane region comprises or consists of an HSV gD transmembrane region, or
(c) comprises or consists of a human transmembrane region, preferably wherein the human transmembrane region comprises or consists of a human decay accelerating factor glycosylphosphatidylinositol (hDAF-GPI) anchor region.

38. The polyribonucleotide of any one of claims 1-29 and 35-37, wherein the polypeptide does not comprise a secretory signal.

39. The polyribonucleotide of any one of claims 1-34, wherein the polypeptide does not comprise a transmembrane region.

40. The polyribonucleotide of any one of claims 1-39, wherein the one or more Plasmodium T cell antigens are one or more P. falciparum T cell antigens, preferably wherein the one or more P. falciparum T cell antigens are from P. falciparum isolate 3D7.

41. The polyribonucleotide of any one of claims 1-40, wherein the polyribonucleotide is an isolated polyribonucleotide.

42. The polyribonucleotide of any one of claims 1-41, wherein the polyribonucleotide is an engineered polyribonucleotide.

43. The polyribonucleotide of any one of claims 1-42, wherein the polyribonucleotide is a codon-optimized polyribonucleotide.

44. An RNA construct comprising in 5′ to 3′ order:
(i) a 5′ UTR that comprises or consists of a modified human alpha-globin 5′-UTR;
(ii) a polyribonucleotide of any one of claims 1-43;
(iii) a 3′ UTR that comprises or consists of a first sequence from the amino terminal enhancer of split (AES) messenger RNA and a second sequence from the mitochondrial encoded 12S ribosomal RNA; and
(iv) a polyA tail sequence.

45. The RNA construct of claim 44, further comprises a 5′ cap.

46. A composition comprising one or more polyribonucleotides of any one of claims 1-43 or an RNA construct of claim 44 or 45.

47. The composition of claim 46, further comprising lipid nanoparticles, polyplexes (PLX), lipidated polyplexes (LPLX), or liposomes,
wherein the one or more polyribonucleotides are fully or partially encapsulated within the lipid nanoparticles, polyplexes (PLX), lipidated polyplexes (LPLX), or liposomes.

48. A pharmaceutical composition comprising the composition of claim 46 or 47 and at least one pharmaceutically acceptable excipient.

49. A combination comprising:
(i) a first pharmaceutical composition comprising a first polyribonucleotide, wherein the first polyribonucleotide encodes a first polypeptide, and the first polypeptide comprises one or more Plasmodium T-cell antigens; and
(ii) a second pharmaceutical composition comprising a second polyribonucleotide, wherein the second polyribonucleotide encodes a second polypeptide, and the second polypeptide comprises one or more Plasmodium antigenic polypeptide regions or portions thereof.

50. The combination of claim 49, wherein the first polyribonucleotide is a polyribonucleotide according to any one of claims 1-43.

51. The combination of claim 49 or 50, wherein the one or more Plasmodium antigenic polypeptide regions or portions thereof of the second polypeptide comprise one or more Plasmodium CSP regions or portions thereof.

52. A combination comprising:
(i) a first pharmaceutical composition comprising a polyribonucleotide encoding a first polypeptide, wherein the first polypeptide comprises one or more Plasmodium T-cell antigens, and wherein the one or more Plasmodium T-cell antigens comprise a Plasmodium N-terminal region or portion thereof, but not a Plasmodium C-terminal region or portion thereof; and
(ii) a second pharmaceutical composition comprising a polyribonucleotide encoding a second polypeptide, wherein the second polypeptide comprises one or more Plasmodium CSP polypeptide regions or portions thereof, and wherein the one or more Plasmodium CSP polypeptide regions or portions thereof comprise a Plasmodium CSP C-terminal region or portion thereof, but not a Plasmodium CSP N-terminal region or portion thereof.

53. A combination comprising:
(i) a first pharmaceutical composition comprising a first polyribonucleotide, wherein the first polyribonucleotide is a polyribonucleotide according to claim 2 or 3; and
(ii) a second pharmaceutical composition comprising a second polyribonucleotide, wherein the second polyribonucleotide is a polyribonucleotide according to claim 11 or 12.

54. A method of treating or preventing a malaria infection comprising administering to a subject a polyribonucleotide according to any one of claims 1-43, an RNA construct according to claim 44 or 45, a composition according to claim 46 or 47, a pharmaceutical composition of claim 48, or a combination according to any one of claims 49-53.

55. The pharmaceutical composition of claim 48 for use in the treatment or prevention of a malaria infection comprising administering one or more doses of the pharmaceutical composition to a subject.

56. The combination of any one of claims 49-53 for use in the treatment or prevention of a malaria infection comprising administering one or more doses of the combination to a subject.
